(19) United States
(12) Patent Application Publication
(10) Pub. No.: US 2026/0000659 A1
(43) Pub. Date: Jan. 1, 2026

(54) LIPIDATED PEPTIDE INHIBITORS OF INTERLEUKIN-23 RECEPTOR

(71) Applicants: JANSSEN BIOTECH, INC., Horsham, PA (US); PROTAGONIST THERAPEUTICS, INC., Newark, CA (US)

(72) Inventors: Santhosh NEELAMKAVIL, Spring House, PA (US); Chengzao SUN, Spring House, PA (US); Sandeep SOMANI, Spring House, PA (US); Stephanie A. BARROS, Spring House, PA (US); Raymond J. PATCH, Spring House, PA (US); Jing ZHANG, Spring House, PA (US); Douglas RIEXINGER, Spring House, PA (US); Charles HENDRICK, Spring House, PA (US); Elisabetta BIANCHI, Pomezia (Rome) (IT); Roberto COSTANTE, Pomezia (Rome) (IT); Federica ROSOLIA, Pomezia (Rome) (IT); Martina LOLLOBRIGIDA, Pomezia (Rome) (IT); Sonia DEL RIZZO, Pomezia (Rome) (IT); Danila BRANCA, Pomezia (Rome) (IT); Ashok BHANDARI, Newark, CA (US); James DANIEL, Newark, CA (US); Tran Trung TRAN, Newark, CA (US); Brian FREDERICK, Newark, CA (US)

(21) Appl. No.: 19/248,166

(22) Filed: Jun. 24, 2025

Related U.S. Application Data
(63) Continuation of application No. 18/495,457, filed on Oct. 26, 2023.
Application No. 18/495,457 is a continuation of application No. PCT/US2022/037205, filed on Jul. 14, 2022.
(60) Provisional application No. 63/221,697, filed on Jul. 14, 2021.

(51) Int. Cl. A61K 31/4439; A61P 37/02

(52) U.S. Cl. CPC A61K 31/4439; A61P 37/02

(57) ABSTRACT

The present invention relates to novel lipidated peptide inhibitors of the interleukin-23 receptor (IL-23R) or pharmaceutically acceptable salts, solvates and/or other forms thereof, corresponding pharmaceutical compositions, methods and/or uses of the IL-23R inhibitors for treatment of autoimmune inflammation diseases and/or related disorders.

### CROSS-REFERENCE TO RELATED APPLICATION

This application is a continuation of U.S. application Ser. No. 18/495,457, filed Oct. 26, 2023, which is a continuation of International Patent Application No. PCT/US2022/037205, filed Jul. 14, 2022, which claims the benefit under 35 U.S.C. § 119 of U.S. Provisional Patent Application Ser. No. 63/221,697, filed Jul. 14, 2021, which are herein incorporated by reference in their entirety, including their respective sequence listings.

### PARTIES TO A JOINT RESEARCH AGREEMENT

The present disclosure was made by, or on behalf of, the below listed parties to a joint research agreement. The joint research agreement was in effect on or before the date the claimed invention was made, and the claimed invention was part of the joint research agreement and made as a result of activities undertaken within the scope of the joint research agreement. The parties to the joint research agreement are JANSSEN BIOTECH, INC. and PROTAGONIST THERAPEUTICS, INC.

### INCORPORATION OF SEQUENCE LISTING

The sequence listing in ST.26 XML format entitled 759727_NTT-6598PCCON2_ST26.xml, created on Dec. 3, 2024, comprising 2,981,893 bytes, prepared according to 37 CFR 1.822 to 1.824, is incorporated herein by reference in its entirety.

### FIELD OF THE INVENTION

The present invention relates to novel lipidated peptide inhibitors of the interleukin-23 receptor (IL-23R) or pharmaceutically acceptable salts, solvates and/or other forms thereof, invention relates to corresponding pharmaceutical compositions, methods and/or uses of the IL-23R inhibitors for treatment of autoimmune inflammation diseases and/or related disorders.

### BACKGROUND

The interleukin-23 (IL-23) cytokine has been implicated as playing a crucial role in the pathogenesis of autoimmune inflammation and related diseases and disorders, such as multiple sclerosis, asthma, rheumatoid arthritis, psoriasis, and inflammatory bowel diseases (IBDs), for example, ulcerative colitis and Crohn's disease. Studies in acute and chronic mouse models of IBD revealed a primary role of interleukin-23 receptor (IL-23R) and downstream effector cytokines in disease pathogenesis. IL-23R is expressed on various adaptive and innate immune cells including Th17 cells, γδ T cells, natural killer (NK) cells, dendritic cells, macrophages, and innate lymphoid cells, which are found abundantly in the intestine. At the intestine mucosal surface, the gene expression and protein levels of IL-23R are found to be elevated in IBD patients. It is believed that IL-23 mediates this effect by promoting the development of a pathogenic CD4⁺ T cell population that produces IL-6, IL-17, and tumor necrosis factor (TNF).

Production of IL-23 is enriched in the intestine, where it is believed to play a key role in regulating the balance between tolerance and immunity through T-cell-dependent and T-cell-independent pathways of intestinal inflammation through effects on T-helper 1 (Th1) and Th17-associated cytokines, as well as restraining regulatory T-cell responses in the gut, favoring inflammation. In addition, polymorphisms in the IL-23 receptor (IL-23R) have been associated with susceptibility to inflammatory bowel diseases (IBDs), further establishing the critical role of the IL-23 pathway in intestinal homeostasis.

Psoriasis, a chronic skin disease affecting about 2%-3% of the general population has been shown to be mediated by the body's T cell inflammatory response mechanisms. IL-23 has one of several interleukins implicated as a key player in the pathogenesis of psoriasis, purportedly by maintaining chronic autoimmune inflammation via the induction of interleukin-17, regulation of T memory cells, and activation of macrophages. Expression of IL-23 and IL-23R has been shown to be increased in tissues of patients with psoriasis, and antibodies that neutralize IL-23 showed IL-23-dependent inhibition of psoriasis development in animal models of psoriasis.

IL-23 is a heterodimer composed of a unique p19 subunit and the p40 subunit shared with IL-12, which is a cytokine involved in the development of interferon-γ (IFN-γ)-producing T helper 1 (T_{H}1) cells. Although IL-23 and IL-12 both contain the p40 subunit, they have different phenotypic properties. For example, animals deficient in IL-12 are susceptible to inflammatory autoimmune diseases, whereas IL-23 deficient animals are resistant, presumably due to a reduced number of CD4⁺ T cells producing IL-6, IL-17, and TNF in the CNS of IL-23-deficient animals. IL-23 binds to IL-23R, which is a heterodimeric receptor composed of IL-12Rβ1 and IL-23R subunits. Binding of IL-23 to IL-23R activates the Jak-Stat signaling molecules, Jak2, Tyk2, and Stat1, Stat 3, Stat 4, and Stat 5, although Stat4 activation is substantially weaker and different DNA-binding Stat complexes form in response to IL-23 as compared with IL-12. IL-23R associates constitutively with Jak2 and in a ligand-dependent manner with Stat3. In contrast to IL-12, which acts mainly on naive CD4(+) T cells, IL-23 preferentially acts on memory CD4(+) T cells.

Therapeutic moieties that inhibit the IL-23 pathway have been developed for use in treating IL-23-related diseases and disorders. A number of antibodies that bind to IL-23 or IL-23R have been identified, including ustekinumab, which has been approved for the treatment of moderate to severe plaque psoriasis (PSO), active psoriatic arthritis (PSA), moderately to severely active Crohn's disease (CD) and moderately to severely active ulcerative colitis (UC). Examples of such identified antibodies, include: Tildrakizumab, an anti-IL23 antibody approved for treatment of plaque psoriasis, Guselkumab, an anti-IL23 antibody approved for treatment of psoriatic arthritis and Risankizumab, an anti-IL23 antibody approved for the treatment of plaque psoriasis in the US, and generalized pustular psoriasis, erythrodermic psoriasis and psoriatic arthritis in Japan.

Although targeted IL-23 antibody therapeutics are used clinically, there are no small-molecule therapeutics that selectively inhibit IL-23 signaling. There are some identified polypeptide inhibitors that bind to IL-23R and inhibit binding of IL-23 to IL-23R (see, e.g., US Patent Application Publication No. US2013/0029907).

Lipidation of therapeutically useful polypeptides can offer advantageous physicochemical properties as compared to the corresponding unmodified polypeptides. Lipidated polypeptides can exhibit improved half-life, reduced immunogenicity, enhanced intracellular uptake and/or enhanced delivery across epithelia.

Thus, there remains a significant need in the art for effective small-molecule and/or polypeptide therapeutic agents to treat and/or prevent IL-23-associated and/or IL23R-associated diseases and disorders, which include, but are not limited to, psoriasis, psoriatic arthritis, inflammatory bowel diseases, ulcerative colitis, and Crohn's disease. In particular:
compounds and methods for specific targeting of IL-23R from the luminal side of the gut may provide therapeutic benefit to IBD patients suffering from local inflammation of the intestinal tissue; and/or
orally bioavailable small molecule and/or polypeptide inhibitors of IL-23 may provide both a non-steroidal treatment option for patients with mild to moderate psoriasis psoriasis and treatment for moderate to severe psoriasis that does not require delivery by infusion.

Compounds and methods for specific targeting of the IL-23R from the luminal side of the gut may provide therapeutic benefit to IBD patients suffering from local inflammation of the intestinal tissue. In addition, orally bioavailable small molecule and/or polypeptide inhibitors of IL-23 may provide both a non-steroidal treatment option for patients with mild to moderate psoriasis and treatment for moderate to severe psoriasis that does not require delivery by infusion.

The present invention is directed to addressing these needs by providing lipidated cyclic peptide inhibitors or pharmaceutically acceptable salts, solvates and/or other forms thereof, that bind IL-23R to inhibit IL-23 binding and signaling, via different suitable routes of administration, which may include but is not limited to oral administration.

### BRIEF SUMMARY

In general, the present invention relates to novel lipidated peptide inhibitors of the interleukin-23 receptor (IL-23R) or pharmaceutically acceptable salts, solvates and/or other forms thereof, corresponding pharmaceutical compositions, methods and/or uses of the IL-23R inhibitors for treatment of autoimmune inflammation diseases and/or related disorders.

invention In particular, the present invention relates to a compound of Formulas (I′), (I) to (X)), or pharmaceutically acceptable salts, solvates and/or other forms thereof, corresponding pharmaceutical compositions, methods and/or uses for treatment of autoimmune inflammation diseases and related disorders.

The cyclic peptide inhibitor(s) of the IL-23R of the present invention is represented by linear form structure of Formula (I′): R1-X3-X4-X5-X6-X7-X8-X9-X10-X11-X12-X13-X14-X15-X16-X17-R2 (I′). The linear form structure of Formula (I′) is intended for exemplary and non-limiting purposes, which will be apparent from examples set forth and exemplified throughout the instant specification, e.g., each such structure may be longer or shorter than the length of fifteen amino acids and/or other corresponding chemical moieties or functional group substituents as defined herein. Specifically in Formula (I′):
X3-X17, respectively and individually, represent individual amino acid (aa) residues or other corresponding chemical moieties or functional group substituents as described below and in the instant invention;
R1 represents the N-terminal end, which may be, for example a hydrogen or a chemical moiety or functional group substituted on the amino group;
Similarly, R2 represents the carboxyl end, which may be, for example the OH of the carboxyl or a chemical moiety or functional group attached thereto or substituted for the OH group (e.g., an amino group to give a terminal carboxylic acid or amide e.g., —C(O)HN₂);
certain residues as shown in the linear form structures set forth herein may be present or absent, e.g., X3 and/or X17—may be absent;
The peptide inhibitors have a bond between positions X4 and X9 (e.g., a pair of Pen residues forming a disulfide or an Abu and Cys residue pair forming a thioether) resulting in the formation of a ring structure; and/or
The bond forming the ring of the structure may, however, be located between other amino acids or chemical moieties besides X4 and X9.
   The cyclic IL-23R inhibitors of the present disclosure bear one or more lipid-like substituents (e.g., a lipid or lipid-like group that comprises a hydrophobic moiety), optionally attached by a linker (e.g., a PEG containing linker)).

Lipid-like substituents, referred to herein as “Z” groups, may be attached at various positions of the IL-23 R inhibitors including, but not limited to, R1, X3, X4, X6, X8, X10, X12, X13, X16, X17 and R2, provided the amino acid at the position to be modified has a suitable functional group (e.g., an amine) for lipid attachment. Some suitable amino acids having an amine that can be utilized for lipid attachment include, but are not limited to, K, dK, hK, dhK, Orn, dOrn, Dab, dDab, Dap, and dDap. In addition, lipid-like substituents may be an R1 group and/or an R2 group in any of the IL-23 inhibitors described herein.

Lipids can also be attached to the inhibitor to form branched structures, and a linker e.g., molecule comprised of PEG, may be included between the branch point and the inhibitor. The branch point is generally a diamino carboxylic acid denoted “Xaa”. Linker groups with branch points may have the form shown in Z5 provided below.

Such Z groups may have a variety of forms including those set forth as Z1 through Z5 below. Accordingly, each Z present in a molecule may be a Z1, Z2, Z3, Z4 or Z5 that is selected independently. Z1 to Z4 are unbranched and include:
Z1 is

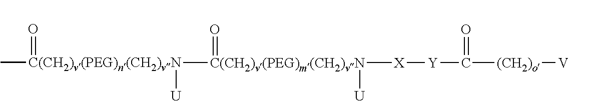

wherein:
   PEG is —OCH₂CH₂—;
   n′=0 or 2-24, when n′ is 0 the group is absent and replaced by a bond;
   m′=0 or 2-24, when m′ is 0 the group is absent and replaced by a bond;
   v′ is independently selected from the range of 1-4 for each occurrence;
   v″ is independently selected from the range of 0-4 for each occurrence, when v″ is 0 the group is replaced by a bond;
   x=gE, dgE, 4SB, p, P, ppp, PPP, gE-(c), gE-(C), sp6, gDab, eK, Trx, or absent;
   o′=6-18;
   Y=gE, sp6, GolA, Pro, D-Pro, meG, Dab, Trx, or absent;
   U is hydrogen or methyl;
   V=—COOH, tetrazole, GolB, mXOH, pXOH, OPhenyl, carnitine, d-carnitine, or hydrogen.
Z2 is

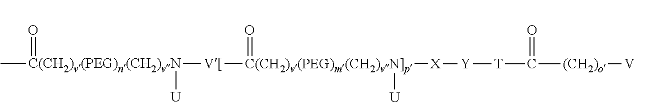

wherein:
   PEG is —OCH₂CH₂—;
   n′=0 or 2-24, when n′ is 0 the group is absent and replaced by a bond;
   m′ is independently selected from 0 or the range of 2-24 for each occurrence, when m′ is
   0 the group is replaced by a bond;
   v′ is independently selected from the range of 1-4 for each occurrence;
   v″ is independently selected from the range of 0-4 for each occurrence, when v″ is 0 the group is replaced by a bond;
   p′ is 1-3;
   V′ is sp6, gEgE
   X=gE, dgE, 4SB, p, P, ppp, PPP, gE-(c), gE-(C), sp6, gDab, eK, Trx, or absent;
   Y=gE, sp6, GolA, Pro, D-Pro, meG, Dab, Trx, or absent;
   X=Trx;
   U is hydrogen or methyl;
   o′=6-18;
   V=—COOH, tetrazole, GolB, mXOH, pXOH, OPhenyl, carnitine, d-carnitine, or hydrogen;
Z3 is
   -gE-C(O)(CH₂)₆₋₁₀CH₃, or -gE-C(O)(CH₂)₁₁₋₁₈CH₃;
Z4 is
   —C(O)(CH₂)₆₋₁₈COOH or —C(O)(CH₂)₆₋₁₈COO(C1-4 alkyl);
Z5 is branched and is:

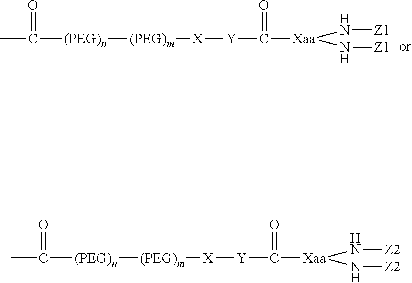

wherein:
   n and m are independently selected from the range of 0 to 24;
   X is absent or is selected from the group consisting of E, dgE, 4SB, gE-(c), gE-(C), sp6, gDab, eK, or Trx;
   Y is absent or is selected from the group consisting of E, dgE, 4SB, gE-(c), gE-(C), sp6, gDab, eK, or Trx;
   Xaa is a diamino-carboxylic acid; and
   Z1 an Z2 are defined above.

In any of Groups I to X the Z group(s) present in the IL-23 inhibitor compounds may comprise one or more Z1 substituents. In any of Groups I to X the Z group(s) present in the IL-23 inhibitor compounds may comprise one or more Z2 substituents. In any of Groups I to X the Z group(s) present in the IL-23 inhibitor compounds may comprise one or more Z3 substituents. In any of Groups I to X the Z group(s) present in the IL-23 inhibitor compounds may comprise one or more Z4 substituents. In any of Groups I to X the Z group(s) present in the IL-23 inhibitor compounds may comprise one or more Z5 substituents. In any of Groups I to X the Z group(s) present in the IL-23 inhibitor compounds may comprise one or more substituent selected independently from those set forth in Z1, Z2, X3, or Z4. In any of Groups I to X the Z group(s) present in the IL-23 inhibitor compounds may comprise one or more substituent selected independently from those set forth in Z1, Z2, X3, or Z5. Where more than one Z group is present in a molecule the Z groups may be selected independently.

The present invention relates to compounds of Formulas (I′), (I) to (X) pharmaceutically acceptable salts, solvates and/or other forms thereof, corresponding pharmaceutical compositions, methods and/or uses for treatment of autoimmune inflammation diseases and related disorders.

In particular, the present invention relates to peptide inhibitor of the IL-23R or a pharmaceutically acceptable salt(s), solvate(s) and/or other form(s) thereof, corresponding pharmaceutical compositions, methods and/or uses for treatment of disease including autoimmune inflammation diseases and related disorders; where:
the inhibitor of the IL-23R of the present invention is identified by Formulas (I′). (I) to (III); or
in Table 1A, Table 1B, Table 1C, Table 1D, Table 1E, Table 1F, Table 1G, Table 1H, Table 1I, Table 1J, Table 1K, Table 1L, or Table 1M respectively,
   in the present specification.

In one aspects, lipidated peptide inhibitors of the IL-23 receptor are linear.

In another aspects, the lipidated peptide inhibitors of the IL-23 receptor are monocyclic.

In other aspects, the lipidated peptide inhibitors of the IL-23 receptor are bicyclic. The present invention relates to novel lipidated peptide inhibitors of the interleukin-23 receptor (IL-23R) or pharmaceutically acceptable salts, solvates and/or other forms thereof, corresponding pharmaceutical compositions, methods and/or uses of the IL-23R inhibitors for treatment of autoimmune inflammation diseases and/or related disorders.

The present invention relates to compounds which are cyclic inhibitors of an IL-23 receptor comprising an amino acid sequence of Formula (I).

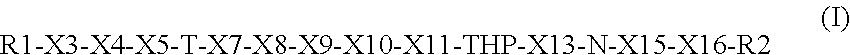

wherein:
R1 is hydrogen, C₁ to C₄ alkyl C(O)—, or C₁ to C₄ alkyl C(O)— substituted with Cl, F, or cyano, or cPEG3aCO;
   X3 is dR, R, K, dK, or absent;
   X4 is Pen, Abu, aMeC, or C;
   X5 is K-Z or dK-Z;
   X7 is 7MeW, W, 3Pya, 7(2ClPh)W, 7(3(1NMepip)pyraz)W, 7(3(6AzaInd1Me))W, 7(3CF3TAZP)W, 7(3NAcPh)W, 7(3NPyrazPh)W, 7(3NpyrlonePh)W, 7(3UrPh)W, 7(4(CpCNPh))W, 7(4CF3Ph)W, 7(4NAcPh)W, 7(40CF3Ph)W, 7(4OMePh)W, 7(4Paz)W, 7(5(2(4OMePh)Pyr))W, 7(5(Ina7Pyr))W, 7(6(1)7dMeNDAZ))W, 7(6(2MeNDAZ))W, 7(7(124TAZP))W, 7(7Imzpy)W, 7BrW, 7EtW, 7PhW, 7PyrW, A, DT, or D7MeW;
   X8 is KAc, dK(Ac), K, or dK;
   X9 is Pen, Abu, aMeC, or C;
   X10 is AEF or dAEF;
   X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy; X15 is 3Pya, 3MeH, H, F, hF, Y, dY, Y(CHF2), PAF, oAMPhe, F(CF3), dPaf, D3Pya, ACIPA(SR), 6OH3Pya, 5PyrimidAla, 5MePyridinAla, 5MeH, 5AmPyridinAla, 4TriazolAla, 4PyridinAla, 4Pya, 3QuinolAla, 3OHPhe, 3AmPyrazolAla, 2AmTyr, or 1MeH;
   X13 is K(Ac), d(KAc), E, or dE;
   X15 is absent, 3pya, 3MeH, H, F, hF, Y, dY, Y(CHF2), PAF, oAMPhe, F(CF3), dPaf, D3Pya, ACIPA(SR), 6OH3Pya, 5PyrimidAla, 5MePyridinAla, 5MeH, 5AmPyridinAla, 4TriazolAla, 4PyridinAla, 4Pya, 3QuinolAla, 3OHPhe, 3AmPyrazolAla, 2AmTyr, or 1MeH;
   X16 is meG, 4(R)HydroxyPro, 4(S)AminoPro, 4diFPro, 5(R)diMePro, aMeP, N(3AmBenzyl)Gly, N(Cyclohexyl)Gly, N(Isobutyl)Gly, P, or dP;
   R2 is —OH, —NH2, —NH(C1 to C4 alkyl), —NH(C1-C4 alkyl), or —N(C1 to C4 alkyl)₂, each alkyl optionally substituted with Cl, F, or cyano; and
   Z is group comprising a lipid moiety; and
wherein the IL-23R inhibitor is cyclized by a disulfide or thioether first bond between X4 and X9.

The present invention also relates to compounds of Formula I, their salts, solvates, or forms thereof, corresponding pharmaceutical compositions, and methods and/or uses for treatment of autoimmune inflammation diseases and related disorders.

The present invention relates to compounds which are bicyclic inhibitors of an IL-23 receptor comprising an amino acid sequence of Formula (X).

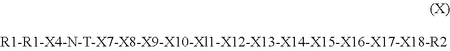

wherein:
R1 is hydrogen, C1 to C4 alkyl C(O)—, or C1 to C4 alkyl C(O)— substituted with Cl, F, or cyano, 7Ahp, 6Ahx, 5Ava, 6Ava, AEEP, GABA, succinyl carnitine. cPEG3aCO, ClAcPEG4CO, C18gEPEG2PEG2, PEG2PEG2gEC18OH, PentCO, PEG12_OMe, PEG4_OMe, HOC18gEPEG2PEG2, PEG2PEG2gE16OH, C14gEPEG2PEG2CO, C12gEPEG2PEG2CO, PEG4_Decyl, PEG4_Lauryl, PEG4_Capryl, PEG4_Hexyl, PEG2_Palm, PEG2_Myristyl, PEG2_Lauryl, Hexyl, Decyl, PEG2_Decyl, PEG2_Capryl, Oct, HOC16gEPEG2PEG2orn, or C12gEPEG2PEG2CO;
X3 is dR, dK, dK-Z, or absent;
X4 is Pen, aMeC, Abu, or C;
X5 is N, L, Q, K, E, aMeN, dN, dL, dQ, dK, dE, K-Z, or dK-Z;
X7 is 7MeW, W, 3Pya, 7(2ClPh)W, 7(3(1NMepip)pyraz)W, 7(3(6AzaInd1Me))W, 7(3CF3TAZP)W, 7(3NAcPh)W, 7(3NPyrazPh)W, 7(3NpyrlonePh)W, 7(3UrPh)W, 7(4(CpCNPh))W, 7(4CF3Ph)W, 7(4NAcPh)W, 7(40CF3Ph)W, 7(4OMePh)W, 7(4Paz)W, 7(5(2(4OMePh)Pyr))W, 7(5(Ina7Pyr))W, 7(6(1)7dMeNDAZ))W, 7(6(2MeNDAZ))W, 7(7(124TAZP))W, 7(7Imzpy)W, 7BrW, 7EtW, 7PhW, 7PyrW, A, DT, or D7MeW; X8 is KAc, or Q;
X9 is Pen, C, aMeC, or Abu;
X10 is AEF, F4OMe, F(4-CONH2), TMAPF, AEF(G), or F;
X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
X12 is THP, aMeL, Acvc, or Acpx, or MeK;
X13 is KAc, E, L, dK(Ac), dE, or dL;
X14 is N, K, or K-Z;
X15 is 3Pya, 3MeH, H, F, hF, Y, dY, Y(CHF2), PAF, oAMPhe, F(CF3), dPaf, D3Pya, ACIPA(SR), 6OH3Pya, 5PyrimidAla, 5MePyridinAla, 5MeH, 5AmPyridinAla, 4TriazolAla, 4PyridinAla, 4Pya, 3QuinolAla, 3OHPhe, 3AmPyrazolAla, 2AmTyr, THP, NH(2-(pyridin-3-yl)ethyl), bAla, or aMeF, or 1MeH;
X16 is Sarc, K-Z, NMeK-Z, or absent;
X17 is K-Z, dK-Z, or absent;
R2 is —OH, —NH2, —NH(C1 to C4 alkyl), —NH(C1-C4 alkyl), or —N(C1 to C4 alkyl)₂, each alkyl optionally substituted with Cl, F, cyano or Z;
Z is group comprising a lipid moiety; and
wherein the IL-23R inhibitor is cyclized by a disulfide or thioether first bond between X4 and X9, and an amide second bond (i) between X5 and X10 when X5 is E and X10 is AEF, or (ii) between X13 and R1 when X13 is E and R1 is 7Ahp, 6Ahx, 5Ava, 6Ava, AEEP, or GABA.

The present invention also relates to compounds of Formula X, their salts, solvates, or forms thereof, corresponding pharmaceutical compositions, and methods and/or uses for treatment of autoimmune inflammation diseases and related disorders.

The present invention relates to compounds which are cyclic inhibitors of an IL-23 receptor comprising an amino acid sequence of Formulas II-IX.

The present invention also relates to compounds of Formula II-IX, their salts, solvates, or forms thereof, corresponding pharmaceutical compositions, and methods and/or uses for treatment of autoimmune inflammation diseases and related disorders

The present invention relates to methods or processes of making compound of Formulas (I) to (X) or Tables 1A to 1M.

The present invention also relates to pharmaceutical composition(s), which comprises a herein-described peptide inhibitor compound of the Il-23R or a pharmaceutically acceptable salt, solvate, or form thereof as described herein, and a pharmaceutically acceptable carrier, excipient, or diluent. The pharmaceutical compositions may comprise or may exclude an absorption enhancer depending on the intended route of delivery or use thereof for treatment of specific indications. The absorption enhancer may be permeation enhancer or intestinal permeation enhancer. In an aspect the absorption enhancer improves oral bioavailability.

The present invention relates to method(s) for treating and/or uses(s) for inflammatory disease(s) in a subject, which comprises administering a therapeutically effective amount of one or more herein-described peptide inhibitor compounds of the IL-23R or pharmaceutically acceptable salts, or solvates thereof, or a corresponding pharmaceutical composition as described herein, respectively to a subject in need thereof. Such inflammatory diseases and related disorders may include, but are not limited to, inflammatory bowel disease (IBD), Crohn's disease (CD), ulcerative colitis (UC), psoriasis (PsO), or psoriatic arthritis (PsA) and the like.

The present invention provides for the use of one or more herein-described compounds (e.g., compounds of formulas (I) to (X) or Tables 1A to 1M) for the preparation of pharmaceutical compositions for use in the treatment of inflammatory diseases and related disorders including, but not limited to, inflammatory bowel disease (IBD), Crohn's disease (CD), ulcerative colitis (UC), psoriasis (PsO), and psoriatic arthritis (PsA).

The present invention provides for the use of one or more herein-described compounds of formulas (I) to (X) or Tables 1A to 1M in the treatment of inflammatory diseases and related disorders including, but not limited to, inflammatory bowel disease (IBD), Crohn's disease (CD), ulcerative colitis (UC), psoriasis (PsO), and psoriatic arthritis (PsA).

The present provides for kits comprising one or more herein-described compounds of formulas (I) to (X) or Tables 1A to 1L and instructions for use in treating a disease in a patient. The disease may be an inflammatory diseases or related disorder including, but not limited to, inflammatory bowel disease (IBD), Crohn's disease (CD), ulcerative colitis (UC), psoriasis (PsO), and psoriatic arthritis (PsA)

### DETAILED DESCRIPTION

### I. General

The present invention relates to novel lipidated peptide inhibitors of the interleukin-23 receptor (IL-23R) or pharmaceutically acceptable salts, solvates and/or other forms thereof, corresponding pharmaceutical compositions, methods and/or uses of the IL-23R inhibitors for treatment of autoimmune inflammation diseases and/or related disorders.

invention The present invention to relates to lipidated cyclic peptide inhibitors of an IL-23R. The lipidated cyclic peptide inhibitors of the present invention may exhibit enhanced properties, such as longer in vivo half-life, compared to the corresponding cyclic peptide inhibitor of an IL-23R without a covalently bound lipid (e.g., fatty acid).

### II. Definitions

Unless otherwise defined herein, scientific and technical terms used in this application shall have the meanings that are commonly understood by those of ordinary skill in the art.

“About” when referring to a value includes the stated value+/−10% of the stated value. For example, about 50% includes a range of from 45% to 55%, while about 20 molar equivalents includes a range of from 18 to 22 molar equivalents. Accordingly, when referring to a range, “about” refers to each of the stated values+/−10% of the stated value of each end of the range. For instance, a ratio of from about 1 to about 3 (weight/weight) includes a range of from 0.9 to 3.3.

“Patient” or “subject”, which are used interchangeably, refer to a living organism, which includes, but is not limited to a human subject suffering from or prone to a disease or condition that can be treated by administration of a pharmaceutical composition as provided herein. Further non-limiting examples may include, but is not limited to humans, other mammals, bovines, rats, mice, dogs, monkeys, goat, sheep, cows, deer, horse, and other mammalian animals and the like. In some aspects, the patient is human.

Unless indicated otherwise the names of naturally occurring and non-naturally occurring aminoacyl residues used herein follow the naming conventions suggested by the IUPAC Commission on the Nomenclature of Organic Chemistry and the IUPAC-IUB Commission on Biochemical Nomenclature as set out in “Nomenclature of α-Amino Acids (Recommendations, 1974)” Biochemistry, 14(2), (1975). To the extent that the names and abbreviations of amino acids and aminoacyl residues employed in this specification and appended claims differ from those suggestions, they will be made clear to the reader. In sequences of amino acids that represent IL-23 inhibitors the individual amino acids are separated by a hyphen “-”.

Throughout the present specification, unless naturally occurring amino acids are referred to by their full name (e.g., alanine, arginine, etc.), they are designated by their conventional three-letter or single-letter abbreviations (e.g., Ala or A for alanine, Arg or R for arginine, etc.). Unless otherwise indicated, three-letter and single-letter abbreviations of amino acids refer to the L-isomeric form of the amino acid in question. The term “L-amino acid,” as used herein, refers to the “L” isomeric form of a peptide, and conversely the term “D-amino acid” refers to the “D” isomeric form of a peptide (e.g., (D)Asp or D-Asp; (D)Phe or D-Phe). Amino acid residues in the D isomeric form can be substituted for any L-amino acid residue, as long as the desired function is retained by the peptide. D-amino acids may be indicated as customary in lower case when referred to using single-letter abbreviations. For example, L-arginine can be represented as “Arg” or “R,” while D-arginine can be represented as “arg” or “r.” Similarly, L-lysine can be represented as “Lys” or “K,” while D-lysine can be represented as “lys” or “k.” Alternatively, a lower case “d” in front of an amino acid can be used to indicate that it is of the D isomeric form, for example D-lysine can be represented by dK. Where “gE” appears in modified aa residues, particularly modified lysine residues (e.g., KPEG2PEG2gEC200H or KPEG6PEG6gEC18OH) it denotes isoglutamic acid and any potential conflict can be resolved by reference to the computer readable form of the structure (e.g., Smiles string) associated with most of he structures provided herein.

In the case of less common or non-naturally occurring amino acids, unless they are referred to by their full name (e.g. sarcosine, ornithine, etc.), frequently employed three- or four-character codes are employed for residues thereof, including, Sar or Sarc (sarcosine, i.e. N-methylglycine), Aib (α-aminoisobutyric acid), Dab (2,4-diaminobutanoic acid), Dap (2,3-diaminopropanoic acid), γ-Glu (γ-glutamic acid), Gaba (γ-aminobutanoic acid), β-Pro (pyrrolidine-3-carboxylic acid), and Abu (2-amino butyric acid).

Amino acids of the D-isomeric form may be located at any of the positions in the IL-23R inhibitors set forth herein (any of X1-X18 appearing in the molecule). In an aspects, amino acids of the D-isomeric form may be located only at any one or more of X3, X5, X6, X8, X13, and optionally one additional position. In other aspects, amino acids of the D-isomeric form may be located only at any one or more of X3, X8, X13, and optionally one additional position. In other aspects, amino acids of the D-isomeric form may be located only at X3, and optionally one additional position. In other aspects, amino acids of the D-isomeric form may be located only at X3, and optionally two or three additional positions. In other aspects, amino acids of the D-isomeric form may be located at only one or two of positions X1 to X18 appearing in the IL-23R inhibitors set forth herein. In other aspects, amino acids of the D-isomeric form may be located at only three or four of positions X1 to X18 appearing in the IL-23R inhibitors set forth herein. For example, an IL-23R inhibitors set forth herein having only positions X3 to X15 present may have amino acids of the D-form present in 3 or four of those positions. In other aspects, amino acids of the D-isomeric form may be located at only five or six of positions X1 to X18 appearing in the IL-23R inhibitors set forth herein.

As conventionally understood in the art or to the skilled artisan, the peptide sequences disclosed herein are shown proceeding from left to right, with the left end of the sequence being the N-terminus of the peptide and the right end of the sequence being the C-terminus of the peptide.

Among sequences disclosed herein are sequences incorporating either an “—OH” moiety or an “—NH₂” moiety at the carboxy terminus (C-terminus) of the sequence. In such cases, and unless otherwise indicated, an “—OH” or an “—NH₂” moiety at the C-terminus of the sequence indicates a hydroxy group or an amino group, corresponding to the presence of a carboxylic acid (COOH) or an amido (CONH₂) group at the C-terminus, respectively. In each sequence of the invention, a C-terminal “—OH” moiety may be substituted for a C-terminal “—NH₂” moiety, and vice-versa.

One of skill in the art will appreciate that certain amino acids and other chemical moieties are modified when bound to another molecule. For example, an amino acid side chain may be modified when it forms an intramolecular bridge with another amino acid side chain, e.g., one or more hydrogen may be removed or replaced by the bond.

A “compound of the invention”, an “inhibitor of the present invention”, an “IL-23R inhibitor of the present invention”, a “compound described herein”, and a “herein-described compound” include the novel compounds disclosed herein, for example the compounds of any of the Examples, including compounds of Formula (I) to (X) such as those found in Table 1A, Table 1B, Table 1C, Table 1D, Table 1E, Table 1F, Table 1G Table 1H, Table 1I, Table 1J, Table 1K, Table 1L or Table 1M.

“Pharmaceutically effective amount” refers to an amount of a compound of the invention in a composition or combination thereof that provides the desired therapeutic or pharmaceutical result.

By “pharmaceutically acceptable” it is meant the carrier(s), diluent(s), salts, or excipient(s) must be compatible with the other components or ingredients of the compositions of the present invention, i.e., that which is useful, safe, non-toxic acceptable for pharmaceutical use. In accordance with the present invention pharmaceutically acceptable means approved or approvable as is listed in the U.S. Pharmacopoeia or other generally recognized pharmacopoeia for use in animals, and more particularly, in humans.

“Pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Absorption enhancer” refers to a component that improves or facilitates the mucosal absorption of a drug in the gastrointestinal tract, such as a permeation enhancer or intestinal permeation enhancer. As conventionally understood in the art, permeation enhancers (PEs) are agents aimed to improve oral delivery of therapeutic drugs with poor bioavailability. PEs are capable of increasing the paracellular and/or transcellular passage of drugs.

Pharmaceutical excipients that can increase permeation have been termed “absorption modifying excipients” (AMEs). AMEs may be used in oral compositions, for example, as wetting agents (sodium dodecyl sulfate), antioxidants (e.g., EDTA), and emulsifiers (e.g., macrogol glycerides), and may be specifically included in compositions as PEs to improve bioavailability. PEs can be categorized as to how they alter barrier integrity via paracellular or transcellular routes.

“Intestinal permeation enhancer (IPE)” refers to a component that improves the bioavailability of a component. Suitable representative IPEs for use in the present invention, include, but are not limited to, various surfactants, fatty acids, medium chain glycerides, steroidal detergents, acyl carnitine and alkanoylcholines, N-acetylated alpha-amino acids and N-acetylated non-alpha-amino acids, and chitosans, other mucoadhesive polymers and the like. For example, a suitable IPE for use in the present invention may be sodium caprate.

“Composition” or “Pharmaceutical Composition” as used herein is intended to encompass an invention or product comprising the specified active product ingredient (API), which may include pharmaceutically acceptable excipients, carriers or diluents as described herein, such as in specified amounts defined throughout the invention. Compositions or Pharmaceutical Compositions result from combination of specific components, such as specified ingredients in the specified amounts as described herein.

Compositions or pharmaceutical compositions of the present invention may be in different pharmaceutically acceptable forms, which may include, but are not limited to a liquid composition, a tablet or matrix composition, a capsule composition, etc. and the like. When the composition is a tablet composition, the tablet may include, but is not limited to different layers two or more different phases, including an internal phase and an external phase that can comprise a core. The tablet composition can also include but is not limited to one or more coatings.

“Solvate” as used herein, means a physical association of the compound of the present invention with one or more solvent molecules. This physical association involves varying degrees bonding, including hydrogen bonding. In certain instances, the solvate will be capable of isolation. The term “solvate” is intended to encompass both solution-phase and isolatable solvates. Non-limiting examples of suitable solvates include hydrates.

Provided are also pharmaceutically acceptable salts and tautomeric forms of the compounds described herein. “Pharmaceutically acceptable” or “physiologically acceptable” refer to compounds, salts, compositions, dosage forms and other materials which are useful in preparing a pharmaceutical composition that is suitable for veterinary or human pharmaceutical use.

The IL-23R inhibitors of the present invention, or their pharmaceutically acceptable salts or solvates may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present invention is meant to include all such possible isomers, as well as their racemic and optically pure forms of the IL-23R inhibitors of the present invention. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included. Where compounds are represented in their chiral form, it is understood that the aspect encompasses, but is not limited to, the specific diastereomerically or enantiomerically enriched form. Where chirality is not specified but is present, it is understood that the aspect is directed to either the specific diastereomerically or enantiomerically enriched form; or a racemic or scalemic mixture of such compound(s). As used herein, “scalemic mixture” is a mixture of stereoisomers enantiomers at a ratio other than 1:1.

“Racemates” refers to a mixture of enantiomers. The mixture can include equal or unequal amounts of each enantiomer.

“Stereoisomer” and “stereoisomers” refer to compounds that differ in the chirality of one or more stereocenters. Stereoisomers include enantiomers and diastereomers. The compounds may exist in stereoisomeric form if they possess one or more asymmetric centers or a double bond with asymmetric substitution and, therefore, can be produced as individual stereoisomers or as mixtures. Unless otherwise indicated, the description is intended to include individual stereoisomers as well as mixtures. The methods for the determination of stereochemistry and the separation of stereoisomers are well-known in the art (see, e.g., Chapter 4 of Advanced Organic Chemistry, 4th ed., J. March, John Wiley and Sons, New York, 1992).

“Tautomer” refers to alternate forms of a compound that differ in the position of a proton, such as enol-keto and imine-enamine tautomers, or the tautomeric forms of heteroaryl groups containing a ring atom attached to both a ring —NH— and a ring ═N— such as pyrazoles, imidazoles, benzimidazoles, triazoles, and tetrazoles.

Unless defined otherwise, all technical and scientific terms used herein have the same meaning as commonly or conventionally understood by one of ordinary skill in the art. In the Chemical Arts. a dash at the front or end of a chemical group is a matter of convenience; chemical groups may be depicted with or without one or more dashes without losing their ordinary meaning. A wavy line drawn through a line in a structure indicates a point of attachment of a group. A dashed line indicates an optional bond. Unless chemically or structurally required, no directionality is indicated or implied by the order in which a chemical group is written or the point at which it is attached to the remainder of the molecule. For instance, the group “—SO₂CH₂—” is equivalent to “—CH₂SO₂—” and both may be connected in either direction. Similarly, an “arylalkyl” group, for example, may be attached to the remainder of the molecule at either an aryl or an alkyl portion of the group. A prefix such as “Cᵤ₋ᵥ” or (Cᵤ-Cᵥ) indicates that the following group has from u to v carbon atoms. For example, “C₁₋₆alkyl” and “C₁-C₆ alkyl” both indicate that the alkyl group has from 1 to 6 carbon atoms.

“Fatty acid” as used herein is an unbranched alkanoic acid of at least six carbons, for example, 7, 8, 9, 10, 11, 12, 13, 14, 15, 16, 17, 18, 19, 20, 21, 22, or more carbons, in length. The fatty acid can contain 1, 2, 3, or more carboxylic acid groups. The fatty acid can include other functional groups, such as but not limited to, amides and phenyl rings. Exemplary fatty acids include hexanoic acid, octanoic acid, decanoic acid, dodecanoic acid, tetradecanoic acid, hexadecanoic acid, octadecanoic acid, 1,6-hexanedioic acid, 1,8-octanedioic acid, 1,10-decanedioic acid, 1,12-dodecanedioic acid, 1,14-tetradecanedioic acid, 1,16-hexadecanedioic acid, and 1,18-octadecanedioic acid.

“Lipidation” refers to a process of covalently attaching one or more fatty acids directly or indirectly to a cyclic peptide inhibitor of an interleukin-23 receptor described herein. A cyclic peptide inhibitor of an interleukin-23 receptor that has undergone lipidation is said to be lipidated. The process of covalent attachment can convert the carboxylic acid into another functional group, such as a secondary amide, or can occur at another functional group present on the fatty acid in order to retain the carboxylic acid present in the original fatty acid. The covalent attachment of the one or more fatty acids can be directly attached to a compound, or indirectly attached through a divalent linker moiety between the one or more fatty acids and the cyclic peptide inhibitor of an interleukin-23 receptor. A divalent linker moiety can include one or more amino acids, a polyethylene glycol (PEG), or a combination thereof. A linker moiety containing a PEG can further exhibit other functional groups, such as an amide, as needed for covalent attachment. Linker moieties comprising one or more amino acids can be attached via the C-terminus, the N-terminus, the side chain, or any combination thereof.

“Polyethylene glycol” or “PEG” is a polyether monovalent radical of general formula —(O—CH₂—CH₂)ₙ—OH, or divalent radical of formula —(O—CH₂—CH₂)ₙ—O—, wherein n is an integer greater than 1. When followed by a number, the PEG indicates the number of repeated units in the moiety. For instance, PEG3 can correspond with a divalent radical of formula —(O—CH₂—CH₂)₃—O—, while PEG8 can correspond with a monovalent radical of formula —(O—CH₂—CH₂)₈—OH.

PEGs are prepared by polymerization of ethylene oxide and are commercially available over a range of molecular weights from 300 Da to 10,000,000 Da. Lower molecular weight PEGs are generally available as pure oligomers, referred to as monodisperse, uniform, or discrete. These are used in certain aspects of the present invention. In certain aspects, the PEG is PEG2, PEG3, PEG4, PEG5, PEG6, PEG7, PEG8, PEG9, PEG10, PEG11, PEG12, PEG18, or PEG24. In certain aspects, the PEG is PEG2, PEG6, or PEG24.

“Treatment” or “treat” or “treating” as used herein refers to an approach for obtaining beneficial or desired results. For purposes of the present invention, beneficial or desired results include, but are not limited to, alleviation of a symptom and/or diminishment of the extent of a symptom and/or preventing a worsening of a symptom associated with a disease or condition. In one aspect, “treatment” or “treating” includes one or more of the following: (a) inhibiting the disease or condition (e.g., decreasing one or more symptoms resulting from the disease or condition, and/or diminishing the extent of the disease or condition); (b) slowing or arresting the development of one or more symptoms associated with the disease or condition (e.g., stabilizing the disease or condition, delaying the worsening or progression of the disease or condition); and (c) relieving the disease or condition, e.g., causing the regression of clinical symptoms, ameliorating the disease state, delaying the progression of the disease, increasing the quality of life, and/or prolonging survival.

“Therapeutically effective amount” or “effective amount” as used herein refers to an amount that is effective to elicit the desired biological or medical response, including the amount of a compound that, when administered to a subject for treating a disease, is sufficient to affect such treatment for the disease. The effective amount will vary depending on the compound, the disease, and its severity and the age, weight, etc., of the subject to be treated. The effective amount can include a range of amounts. As is understood in the art, an effective amount may be in one or more doses, i.e., a single dose or multiple doses may be required to achieve the desired treatment endpoint. An effective amount may be considered in the context of administering one or more therapeutic agents, and a single agent may be considered to be given in an effective amount if, in conjunction with one or more other agents, a desirable or beneficial result may be or is achieved. Suitable doses of any co-administered compounds may optionally be lowered due to the combined action (e.g., additive or synergistic effects) of the compounds.

“Co-administration” as used herein refers to administration of unit dosages of the compounds disclosed herein before or after administration of unit dosages of one or more additional therapeutic agents, for example, administration of the compound disclosed herein within seconds, minutes, or hours of the administration of one or more additional therapeutic agents. For example, in some respects, a unit dose of a compound of the invention is administered first, followed within seconds or minutes by administration of a unit dose of one or more additional therapeutic agents. Alternatively, in other aspects, a unit dose of one or more additional therapeutic agents is administered first, followed by administration of a unit dose of a compound of the invention within seconds or minutes. In some respects, a unit dose of a compound of the invention is administered first, followed, after a period of hours (e.g., 1-12 hours), by administration of a unit dose of one or more additional therapeutic agents. In other aspects, a unit dose of one or more additional therapeutic agents is administered first, followed, after a period of hours (e.g., 1-12 hours), by administration of a unit dose of a compound of the invention. Co-administration of a compound disclosed herein with one or more additional therapeutic agents generally refers to simultaneous or sequential administration of a compound disclosed herein and one or more additional therapeutic agents, such that therapeutically effective amounts of each agent are present in the body of the patient.

Abbreviation, “(V/V)” refers to the phrase “volume for volume”, i.e., the proportion of a particular substance within a mixture, as measured by volume or a volume amount of a component of the composition disclosed herein relative to the total volume amount of the composition. Accordingly, the quantity is unit less and represents a volume percentage amount of a component relative to the total volume of the composition. For example, a 2% (V/V) solvent mixture can indicate 2 mL of one solvent is present in 100 mL of the solvent mixture.

Abbreviation, “(w/w)” refers to the phrase “weight for weight”, i.e., the proportion of a particular substance within a mixture, as measured by weight or mass or a weight amount of a component of the composition disclosed herein relative to the total weight amount of the composition. Accordingly, the quantity is unit less and represents a weight percentage amount of a component relative to the total weight of the composition. For example, a 2% (w/w) solution can indicate 2 grams of solute is dissolved in 100 grams of solution.

Systemic routes of administration as conventionally understood in the medicinal or pharmaceutical arts, refer to or are defined as a route of administration of drug, a pharmaceutical composition or formulation, or other substance into the circulatory system so that various body tissues and organs are exposed to the drug, formulation or other substance. As conventionally understood in the art, administration can take place orally (where drug or oral preparations are taken by mouth, and absorbed via the gastrointestinal tract), via enteral administration (absorption of the drug also occurs through the gastrointestinal tract) or parenteral administration (generally injection, infusion, or implantation, etc.

“Systemically active” peptide drug therapy as it relates to the present invention generally refers to treatment by means of a pharmaceutical composition comprising a peptide active ingredient, wherein said peptide resists immediate metabolism and/or excretion resulting in its exposure in various body tissues and organs, such as the cardiovascular, respiratory, gastrointestinal, nervous or immune systems.

Systemic drug activity in the present invention also refers to treatment using substances that travel through the bloodstream, reaching and affecting cells in various body tissues and organs. Systemic active drugs are transported to their site of action and work throughout the body to attack the physiological processes that cause inflammatory diseases.

“Bioavailability” refers to the extent and rate at which the active moiety (drug or metabolite) enters systemic circulation, thereby accessing the site of action. Bioavailability of a drug is impacted by the properties of the dosage form, which depend partly on its design and manufacture.

“Digestive tract tissue” as used herein refers to all the tissues that comprise the organs of the alimentary canal. For example, only, and without limitation, “digestive tract tissue” includes tissues of the mouth, esophagus, stomach, small intestine, large intestine, duodenum, and anus.

### III. Compounds

The present invention relates to novel lipidated cyclic peptide inhibitors of the interleukin-23 receptor (IL-23R) or pharmaceutically acceptable salt thereof.

In particular, the present invention relates to a lipidated cyclic peptide inhibitor compound of the interleukin-23 receptor (IL-23R) or a pharmaceutically acceptable salt thereof, where each compound structure is as identified in Table 1A, Table 1B, Table 1C, Table 1D, Table 1E, Table 1F, Table 1G, Table 1H, Table 1I, Table 1J, Table 1K, or Table 1L of the present specification.

In one aspect, a lipidated cyclic peptide inhibitor compound of the interleukin-23 receptor (IL-23R) compound, or a pharmaceutically acceptable salt thereof, has a structure of a compound in Table 1A.

In another aspect, a lipidated cyclic peptide inhibitor compound of the interleukin-23 receptor (IL-23R) compound or a pharmaceutically acceptable salt thereof, has a structure of a compound in Table 1B.

In another aspect, a lipidated cyclic peptide inhibitor compound of the interleukin-23 receptor (IL-23R) compound or a pharmaceutically acceptable salt thereof, has a structure of a compound in Table 1C.

In another aspect, a lipidated cyclic peptide inhibitor compound of the interleukin-23 receptor (IL-23R) compound or a pharmaceutically acceptable salt thereof, has a structure of a compound in Table 1D.

In another aspect, a lipidated cyclic peptide inhibitor compound of the interleukin-23 receptor (IL-23R) compound or a pharmaceutically acceptable salt thereof, has a structure of a compound in Table 1E.

In another aspect, a lipidated cyclic peptide inhibitor compound of the interleukin-23 receptor (IL-23R) compound or a pharmaceutically acceptable salt thereof, has a structure of a compound in Table 1F.

In another aspect, a lipidated cyclic peptide inhibitor compound of the interleukin-23 receptor (IL-23R) compound or a pharmaceutically acceptable salt thereof, has a structure of a compound in Table 1G.

In another aspect, a lipidated cyclic peptide inhibitor compound of the interleukin-23 receptor (IL-23R) compound or a pharmaceutically acceptable salt thereof, has a structure of a compound in Table 1H.

In another aspect, a lipidated cyclic peptide inhibitor compound of the interleukin-23 receptor (IL-23R) compound or a pharmaceutically acceptable salt thereof, has a structure of a compound in Table 1I.

In another aspect, a lipidated cyclic peptide inhibitor compound of the interleukin-23 receptor (IL-23R) compound or a pharmaceutically acceptable salt thereof, has a structure of a compound in Table 1J.

In another aspect, a lipidated cyclic peptide inhibitor compound of the interleukin-23 receptor (IL-23R) compound or a pharmaceutically acceptable salt thereof, has a structure of a compound in Table 1K.

In another aspect, a lipidated cyclic peptide inhibitor compound of the interleukin-23 receptor (IL-23R) compound or a pharmaceutically acceptable salt thereof, has a structure of a compound in Table 1L.

In another aspect, a lipidated cyclic peptide inhibitor compound of the interleukin-23 receptor (IL-23R) compound or a pharmaceutically acceptable salt thereof, has a structure of a compound in Table 1M.

Lengthy table referenced here
US20260000659A1-20260101-T00001
Please refer to the end of the specification for access instructions.

wherein Pen-Pen forms a disulfide bond, or Abu-C form a thioether bond.

Lengthy table referenced here
US20260000659A1-20260101-T00002
Please refer to the end of the specification for access instructions.

Lengthy table referenced here
US20260000659A1-20260101-T00003
Please refer to the end of the specification for access instructions.

Lengthy table referenced here
US20260000659A1-20260101-T00004
Please refer to the end of the specification for access instructions.

Lengthy table referenced here
US20260000659A1-20260101-T00005
Please refer to the end of the specification for access instructions.

Lengthy table referenced here
US20260000659A1-20260101-T00006
Please refer to the end of the specification for access instructions.

Lengthy table referenced here
US20260000659A1-20260101-T00007
Please refer to the end of the specification for access instructions.

Lengthy table referenced here
US20260000659A1-20260101-T00008
Please refer to the end of the specification for access instructions.

Lengthy table referenced here
US20260000659A1-20260101-T00009
Please refer to the end of the specification for access instructions.

Lengthy table referenced here
US20260000659A1-20260101-T00010
Please refer to the end of the specification for access instructions.

Lengthy table referenced here
US20260000659A1-20260101-T00011
Please refer to the end of the specification for access instructions.

Lengthy table referenced here
US20260000659A1-20260101-T00012
Please refer to the end of the specification for access instructions.

Lengthy table referenced here
US20260000659A1-20260101-T00013
Please refer to the end of the specification for access instructions.

### Synthesis

The compounds described herein may be synthesized by many techniques that are known to those skilled in the art. In certain aspects, monomer subunits are synthesized and purified using the techniques described in the accompanying Examples.

In some aspects, the present invention provides a method of producing a compound (or monomer subunit thereof) of the invention, comprising chemically synthesizing a peptide having an amino acid sequence described herein, including but not limited to any of the amino acid sequences set forth in the compounds of Formula (I) to Formula (X), Table 1A, Table 1B, Table 1C, Table 1D, Table 1E, Table 1F, Table 1G, Table 1H, Table 1I, Table 1J, Table 1K, Table 1L, and Table 1M herein. In some aspects, a portion of the peptide is recombinantly synthesized, instead of being chemically synthesized. In some aspects, methods of producing a compound further include cyclizing the compound precursor after the constituent subunits have been attached. In particular aspects, cyclization is accomplished via any of the various methods described herein.

The present invention may include, but is not limited to, polynucleotides and vectors (e.g., expression vectors) that encode a portion of the amino acid sequence of a compound described herein, for instance, in the accompanying Examples, Table 1A, Table 1B, Table 1C, Table 1D, Table 1E, Table 1F, Table 1G, Table 1H, Table 1I, Table 1J, Table 1K, or Table 1L.

The present invention further describes synthesis of lipidated compounds described herein, such as the compounds of Formula (I) to Formula (X), and the compounds of Table 1A, Table 1B, Table 1C, Table 1D, Table 1E, Table 1F, Table 1G, Table 1H, Table 1I, Table 1J, Table 1K, Table 1L, and Table 1M.

In some aspects, one or more of the amino acid residues or amino acid monomers are lipidated and then covalently attached to one another to form a compound of the invention.

In some aspects, one or more of the amino acid residues or amino acid monomers are covalently attached to one another and lipidated at an intermediate oligomer stage before attaching additional amino acids and cyclization to form a compound of the invention.

In some aspects, a cyclic peptide is synthesized and then lipidated to form a compound of the invention. Illustrative synthetic methods are described in the Examples.

The present invention further describes synthesis of compounds described herein, such as the compounds of Formulas (I) to (X) and the compounds of Table 1A, Table 1B, Table 1C, Table 1D, Table 1E, Table 1F, Table 1G, Table 1H, Table 1I, Table 1J, Table 1K, Table 1L, and Table 1M. Illustrative synthetic methods are described in the Examples.

### IV. Pharmaceutical Compositions

The present invention relates to pharmaceutical composition which comprises an IL-23R inhibitor of the present invention.

The present invention includes pharmaceutical compositions comprising one or more inhibitors of the present invention and a pharmaceutically acceptable carrier, diluent or excipient.

The pharmaceutically acceptable carrier, diluent or excipient may be a solid, semi-solid or liquid filler, diluent, encapsulating material or formulation auxiliary of any type. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like.

The pharmaceutical compositions may be administered orally, parenterally, intracisternally, intravaginally, intraperitoneally, intrarectally, topically (as by powders, ointments, drops, suppository, or transdermal patch), by inhalation (such as intranasal spray), ocularly (such as intraocularly) or buccally. The term “parenteral” as used herein refers to modes of administration which include intravenous, intramuscular, intraperitoneal, intrasternal, subcutaneous, intradermal and intraarticular injection and infusion. Accordingly, in certain embodiments, the compositions are formulated for delivery by any of these routes of administration. A pharmaceutical composition may be formulated for and administered orally. A pharmaceutical composition may be formulated for and administered parenterally.

In particular aspects, an IL-23R inhibitor of the present invention, is suspended in a sustained-release matrix. A sustained-release matrix, as used herein, is a matrix made of materials, usually polymers, which are degradable by enzymatic or acid-base hydrolysis or by dissolution. Once inserted into the body, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid) polyanhydrides, poly(ortho)esters, polypeptides, hyaluronic acid, collagen, chondroitin sulfate, carboxylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. One embodiment of a biodegradable matrix is a matrix of one of either polylactide, polyglycolide, or polylactide co-glycolide (co-polymers of lactic acid and glycolic acid).

The IL-23R inhibitors of the present invention may be prepared and/or formulated as pharmaceutically acceptable salts or when appropriate in neutral form. Pharmaceutically acceptable salts are non-toxic salts of a neutral form of a compound that possess the desired pharmacological activity of the neutral form. These salts may be derived from inorganic or organic acids or bases. For example, a compound that contains a basic nitrogen may be prepared as a pharmaceutically acceptable salt by contacting the compound with an inorganic or organic acid. Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in Remington: The Science and Practice of Pharmacy, 21^{st} Edition, Lippincott Williams and Wilkins, Philadelphia, Pa., 2006.

Examples of “pharmaceutically acceptable salts” of the compounds disclosed herein also include salts derived from an appropriate base, such as an alkali metal (for example, sodium, potassium), an alkaline earth metal (for example, magnesium), ammonium and NX₄⁺ (wherein X is C₁-C₄ alkyl). Also included are base addition salts, such as sodium or potassium salts.

The present invention relates to pharmaceutical compositions comprising an IL-23R inhibitor of the present invention or pharmaceutically acceptable salts, isomers, or a mixture thereof, in which from 1 to n hydrogen atoms attached to a carbon atom may be replaced by a deuterium atom or D, in which n is the number of hydrogen atoms in the molecule. As known in the art, the deuterium atom is a non-radioactive isotope of the hydrogen atom. Such compounds may increase resistance to metabolism, and thus may be useful for increasing the half-life of the compounds described herein or pharmaceutically acceptable salts, isomer, or a mixture thereof when administered to a mammal. See, e.g., Foster, “Deuterium Isotope Effects in Studies of Drug Metabolism,” Trends Pharmacol. Sci., 5(12):524-527 (1984). Such compounds are synthesized by means well known in the art, for example by employing starting materials in which one or more hydrogen atoms have been replaced by deuterium.

Examples of isotopes that can be incorporated into the disclosed compounds also include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Substitution with positron emitting isotopes, such as ¹¹C, ¹⁸F, ¹⁵O and ¹³N, can be useful in Positron Emission Topography (PET) studies for examining substrate receptor occupancy. Isotopically labeled compounds of Formula (I), can generally be prepared by conventional techniques known to those skilled in the art or by processes analogous to those described in the Examples as set out below using an appropriate isotopically labeled reagent in place of the non-labeled reagent previously employed.

In some aspects, pharmaceutical compositions for parenteral injection comprise pharmaceutically acceptable sterile aqueous or nonaqueous solutions, dispersions, suspensions or emulsions, or sterile powders, for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, β-cyclodextrin, vegetable oils (such as olive oil), and injectable organic esters such as ethyl oleate. Proper fluidity may be maintained, for example, by the use of coating materials such as lecithin, by the maintenance of the required particle size in the case of dispersions, and by the use of surfactants. These compositions may also contain adjuvants such as preservative, wetting agents, emulsifying agents, and dispersing agents. Prolonged absorption of an injectable pharmaceutical form may be brought about by the inclusion of agents which delay absorption, such as aluminum monostearate and gelatin.

Injectable depot forms include those made by forming microencapsulated matrices of the peptide inhibitor in one or more biodegradable polymers such as polylactide-polyglycolide, poly(orthoesters), poly(anhydrides), and (poly)glycols, such as PEG. Depending upon the ratio of peptide to polymer and the nature of the particular polymer employed, the rate of release of the peptide inhibitor can be controlled. Depot injectable Formulations are also prepared by entrapping the peptide inhibitor in liposomes or microemulsions compatible with body tissues.

The injectable formulations may be sterilized, for example, by filtration through a bacterial-retaining filter, or by incorporating sterilizing agents in the form of sterile solid compositions which can be dissolved or dispersed in sterile water or other sterile injectable medium just prior to use.

Topical administration includes administration to the skin or mucosa, including surfaces of the lung and eye. Compositions for topical lung administration, including those for inhalation and intranasal, may involve solutions and suspensions in aqueous and non-aqueous Formulations and can be prepared as a dry powder which may be pressurized or non-pressurized. In non-pressurized powder compositions, the active ingredient may be finely divided form may be used in admixture with a larger sized pharmaceutically acceptable inert carrier comprising particles having a size, for example, of up to 100 micrometers in diameter. Suitable inert carriers include sugars such as lactose.

Alternatively, a pharmaceutical composition of the present invention may be pressurized and contain a compressed gas, such as nitrogen or a liquefied gas propellant. The liquefied propellant medium and indeed the total composition may be such that the active ingredient does not dissolve therein to any substantial extent. The pressurized composition may also contain a surface-active agent, such as a liquid or solid non-ionic surface-active agent or may be a solid anionic surface-active agent. It is preferred to use the solid anionic surface-active agent in the form of a sodium salt.

A further form of topical administration is to the eye. A peptide inhibitor of the present disclosure may be delivered in a pharmaceutically acceptable ophthalmic vehicle, such that the peptide inhibitor is maintained in contact with the ocular surface for a sufficient time period to allow the peptide inhibitor to penetrate the corneal and internal regions of the eye, as for example the anterior chamber, posterior chamber, vitreous body, aqueous humor, vitreous humor, cornea, iris/ciliary, lens, choroid/retina and sclera. The pharmaceutically acceptable ophthalmic vehicle may, for example, be an ointment, vegetable oil or an encapsulating material. Alternatively, the peptide inhibitors of the invention may be injected directly into the vitreous and aqueous humor.

Compositions for rectal or vaginal administration include suppositories which may be prepared by mixing the peptide inhibitors of this invention with suitable non-irritating excipients or carriers such as cocoa butter, polyethylene glycol or a suppository wax, which are solid at room temperature but liquid at body temperature and, therefore, melt in the rectum or vaginal cavity and release the active compound.

Peptide inhibitors of the present invention may also be administered in liposomes or other lipid-based carriers. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to a peptide inhibitor of the present invention, stabilizers, preservatives, excipients, and the like. In certain embodiments, the lipids comprise phospholipids, including the phosphatidyl cholines (lecithins) and serines, both natural and synthetic. Methods to form liposomes are known in the art.

Pharmaceutical compositions suitable for parenteral administration in a method or use described herein may comprise sterile aqueous solutions and/or suspensions of the IL:-23R inhibitors made isotonic with the blood of the recipient, generally using sodium chloride, glycerin, glucose, mannitol, sorbitol, and the like.

The present invention provides a pharmaceutical composition for oral delivery. Compositions and peptide inhibitors of the present invention may be prepared for oral administration according to any of the methods, techniques, and/or delivery vehicles described herein. Further, one having skill in the art will appreciate that the peptide inhibitors of the instant invention may be modified or integrated into a system or delivery vehicle that is not disclosed herein yet is well known in the art and compatible for use in oral delivery of peptides.

Formulations for oral administration may comprise adjuvants (e.g., resorcinols and/or nonionic surfactants such as polyoxyethylene oleyl ether and n-hexadecylpolyethylene ether) to artificially increase the permeability of the intestinal walls, and/or enzymatic inhibitors (e.g., pancreatic trypsin inhibitors, diisopropylfluorophosphate (DFF) or trasylol) to inhibit enzymatic degradation. In certain embodiments, the peptide inhibitor of a solid-type dosage form for oral administration can be mixed with at least one additive, such as sucrose, lactose, cellulose, mannitol, trehalose, raffinose, maltitol, dextran, starches, agar, alginates, chitins, chitosans, pectins, gum tragacanth, gum arabic, gelatin, collagen, casein, albumin, synthetic or semisynthetic polymer, or glyceride. These formulations for oral administration can also contain other type(s) of additives, e.g., inactive diluting agent, lubricant such as magnesium stearate, paraben, preserving agent such as sorbic acid, ascorbic acid, alpha-tocopherol, antioxidants such as cysteine, disintegrators, binders, thickeners, buffering agents, pH adjusting agents, sweetening agents, flavoring agents or perfuming agents.

In particular aspects, oral dosage forms or unit doses compatible for use with the peptide inhibitors of the present invention may include a mixture of peptide inhibitor and nondrug components or excipients, as well as other non-reusable materials that may be considered either as an ingredient or packaging. Oral compositions may include at least one of a liquid, a solid, and a semi-solid dosage forms. In some embodiments, an oral dosage form is provided comprising an effective amount of peptide inhibitor, wherein the dosage form comprises at least one of a pill, a tablet, a capsule, a gel, a paste, a drink, a syrup, ointment, and suppository. In some instances, an oral dosage form is provided that is designed and configured to achieve delayed release of the peptide inhibitor in the subject's small intestine and/or colon.

Tablets may contain excipients, glidants, fillers, binders and the like. Aqueous compositions are prepared in sterile form, and when intended for delivery by other than oral administration generally will be isotonic. Compositions may optionally contain excipients such as those set forth in the “Handbook of Pharmaceutical Excipients” (1986). Excipients include ascorbic acid and other antioxidants, chelating agents such as EDTA, carbohydrates such as dextran, hydroxyalkylcellulose, hydroxyalkylmethylcellulose, stearic acid and the like. The pH of the compositions ranges from, for example, about 3 to about 11. The pH of the compositions may, for example, range from about 5 to about 7 or from about 7 to about 10.

An oral pharmaceutical composition of the present invention may comprise an IL-23R inhibitor of the present invention may comprise an enteric coating that is designed to delay release of the IL-23R inhibitor in the small intestine. The present invention relates to a pharmaceutical composition that comprises an IL-23R inhibitor of the present invention and a protease inhibitor, such as aprotinin, in a delayed release pharmaceutical formulation. Pharmaceutical compositions (e.g., oral pharmaceutical compositions) may comprise an enteric coat that is soluble in gastric juice at a pH of about 5.0 or higher. Such enteric coatings may comprise a polymer having dissociable carboxylic groups, such as derivatives of cellulose, including hydroxypropylmethyl cellulose phthalate, cellulose acetate phthalate and cellulose acetate trimellitate and similar derivatives of cellulose and other carbohydrate polymers.

An oral pharmaceutical composition comprising an IL-23R inhibitor of the present invention that comprises an IL-23R inhibitor which may comprise an enteric coating that is designed to protect and release the pharmaceutical composition in a controlled manner within the subject's lower gastrointestinal system, and to avoid systemic side effects. In addition to enteric coatings, the peptide inhibitors of the instant invention may be encapsulated, coated, engaged or otherwise associated within any compatible oral drug delivery system or component. For example, in some embodiments an IL-23R inhibitor of the present invention is provided in a lipid carrier system comprising at least one of polymeric hydrogels, nanoparticles, microspheres, micelles, and other lipid systems.

To overcome peptide degradation of an IL-23R inhibitor of the present invention in the small intestine, the pharmaceutical compositions may comprise a hydrogel polymer carrier system in which a peptide inhibitor of the present invention is contained, whereby the hydrogel polymer protects the IL-23R inhibitor from proteolysis in the small intestine and/or colon. An IL-23R inhibitor may further be formulated for compatible use with a carrier system that is designed to increase the dissolution kinetics and enhance intestinal absorption of the peptide. These methods include the use of liposomes, micelles and nanoparticles to increase GI tract permeation of peptides.

Various bioresponsive systems may also be combined with one or more an IL-23R inhibitors of the present invention to provide a pharmaceutical agent for oral delivery. For example, an IL-23R inhibitor of the present invention may be used in combination with a bioresponsive system, such as hydrogels and mucoadhesive polymers with hydrogen bonding groups (e.g., PEG, poly(methacrylic) acid [PMAA], cellulose, Eudragit®, chitosan and alginate) to provide a therapeutic agent for oral administration.

In certain aspects, pharmaceutical composition and formulations may include an IL-23R inhibitor of the present invention and one or more absorption enhancers, enzyme inhibitors, or mucoso adhesive polymers. In an embodiment, the absorption enhancer may be an intestinal permeation enhancer.

IL-23R inhibitors of the present invention may be formulated in a formulation vehicle, such as, e.g., emulsions, liposomes, microsphere or nanoparticles.

The present invention provides for a method for treating a subject with an IL-23R inhibitor of the present invention having an increased half-life. In one aspect, the present invention provides a peptide inhibitor having a half-life of at least several hours to one day in vitro or in vivo (e.g., when administered to a human subject) sufficient for daily (q.d.) or twice daily (b.i.d.) dosing of a therapeutically effective amount. In certain embodiments, the IL-23R inhibitor has a half-life of three days or longer sufficient for weekly (q.w.) dosing of a therapeutically effective amount. In certain embodiments, the IL-23R inhibitor has a half-life of eight days or longer sufficient for bi-weekly (b.i.w.) or monthly dosing of a therapeutically effective amount. In certain embodiments, the IL-23R inhibitor is derivatized or modified such that is has a longer half-life as compared to the underivatized or unmodified peptide inhibitor. In certain embodiments, the IL-23R inhibitor contains one or more chemical modifications to increase serum half-life.

When used in at least one of the treatments or delivery systems described herein, a peptide inhibitor of the present invention may be employed in pure form or, where such forms exist, in pharmaceutically acceptable salt form.

The total daily usage of the IL-23R inhibitor and compositions of the present invention can be decided by the attending physician within the scope of sound medical judgment. The specific therapeutically effective dose level for any particular subject will depend upon a variety of factors including: a) the disorder being treated and the severity of the disorder; b) activity of the specific compound employed; c) the specific composition employed, the age, body weight, general health, sex and diet of the patient; d) the time of administration, route of administration, and rate of excretion of the specific peptide inhibitor employed; e) the duration of the treatment; f) drugs used in combination or coincidental with the specific peptide inhibitor employed, and like factors well known in the medical arts.

In particular embodiments, the total daily dose of an IL-23R inhibitor of the present invention to be administered to a human or other mammal host in single or divided doses may be in amounts, for example, from 0.0001 to 300 mg/kg body weight daily or 1 to 300 mg/kg body weight daily.

The compositions may conveniently be presented in unit dosage form and can be prepared by any of the methods well known in the art of pharmacy. Techniques and compositions generally are found in Remington's Pharmaceutical Sciences (Mack Publishing Co., Easton, PA). Such methods include the step of bringing into association the active ingredient with the carrier which constitutes one or more accessory ingredients. In general, the compositions are prepared by uniformly and intimately bringing into association the active ingredient with liquid carriers or finely divided solid carriers or both, and then, if necessary, shaping the product.

Compositions suitable for oral administration can be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste. The active ingredient may also be administered as a buccal or sublingual formulation. Buccal or sublingal formulations may comprise an active ingredient in a matrix that releases the active ingredient for transport across the buccal and/or sublingual membranes. The buccal or sublingual formulation may further include a rate controlling matrix that releases the active compounds at a predetermined rate for transport across the buccal and/or sublingual membranes. The buccal or sublingual formulation may further include one or more compounds selected from the group consisting of (i) taste masking agents, (ii) enhancers, (iii) complexing agents, and mixtures thereof, and (iv) other pharmaceutically acceptable carriers and/or excipients. The enhancer may be a permeation enhancer.

A tablet is made by compression or molding, optionally with one or more accessory ingredients. Compressed tablets can be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets can optionally be coated or scored and optionally are formulated so as to provide slow or controlled release of the active ingredient therefrom.

### V. Non-Invasive Detection of Intestinal Inflammation

The IL-23R inhibitors of the present invention may be used for detection, assessment and diagnosis of intestinal inflammation by microPET imaging, wherein the peptide inhibitor is labeled with a chelating group or a detectable label, as part of a non-invasive diagnostic procedure. In certain embodiments, an IL-23R inhibitor of the present invention is conjugated with a bifunctional chelator. In certain embodiments, an IL-23R inhibitor of the present invention is radiolabeled. The labeled an IL-23R inhibitor is then administered to a subject orally or rectally. In certain embodiments, an IL-23R inhibitor is included in drinking water. Following uptake of an IL-23R inhibitor, microPET imaging may be used to visualize inflammation throughout the subject's bowels and digestive track.

### VI. Methods of Treatments and/or Uses

The present invention relates to relates to methods for treating a subject afflicted with a condition or indication associated with IL-23 or IL-23R (e.g., activation of the IL-23/IL-23R signaling pathway), where the method comprises administering to the subject an IL-23R inhibitor disclosed herein. In one aspect, the present invention relates to a method for treating a subject afflicted with a condition or indication characterized by inappropriate, deregulated, or increased IL-23 or IL-23R activity or signaling, comprising administering to the individual a peptide inhibitor of the present invention in an amount sufficient to inhibit (partially or fully) binding of IL-23 to an IL-23R in the subject. The inhibition of IL-23 binding to IL-23R may occur in particular organs or tissues of the subject, e.g., the stomach, small intestine, large intestine/colon, intestinal mucosa, lamina propria, Peyer's Patches, mesenteric lymph nodes, or lymphatic ducts.

The present invention relates to methods comprising providing a peptide inhibitor described herein to a subject in need thereof. The subject in need thereof may be a subject that has been diagnosed with or has been determined to be at risk of developing a disease or disorder associated with IL-23/IL-23R. The subject may be a mammal. The subject may be, in particular, a human.

The disease or disorder to be treated by treatment with an IL-23R inhibitor of the present invention may be autoimmune inflammation and related diseases and disorders, such as multiple sclerosis, asthma, rheumatoid arthritis, inflammation of the gut, inflammatory bowel diseases (IBDs), juvenile IBD, adolescent IBD, Crohn's disease, ulcerative colitis, sarcoidosis, Systemic Lupus Erythematosus, ankylosing spondylitis (axial spondyloarthritis), psoriatic arthritis, or psoriasis. In particular, the disease or disorder may be psoriasis (e.g., plaque psoriasis, guttate psoriasis, inverse psoriasis, pustular psoriasis, Palmo-Plantar Pustulosis, psoriasis vulgaris, or erythrodermic psoriasis), atopic dermatitis, acne ectopica, ulcerative colitis, Crohn's disease, Celiac disease (nontropical Sprue), enteropathy associated with seronegative arthropathies, microscopic colitis, collagenous colitis, eosinophilic gastroenteritis/esophagitis, colitis associated with radio- or chemo-therapy, colitis associated with disorders of innate immunity as in leukocyte adhesion deficiency-1, chronic granulomatous disease, glycogen storage disease type 1b, Hermansky-Pudlak syndrome, Chediak-Higashi syndrome, Wiskott-Aldrich Syndrome, pouchitis, pouchitis resulting after proctocolectomy and ileoanal anastomosis, gastrointestinal cancer, pancreatitis, insulin-dependent diabetes mellitus, mastitis, cholecystitis, cholangitis, primary biliary cirrhosis, viral-associated enteropathy, pericholangitis, chronic bronchitis, chronic sinusitis, asthma, uveitis, or graft versus host disease.

The present invention relates to a method or use of an IL-23R inhibitor for treating an inflammatory disease in a subject that includes administering to the subject a therapeutically effective amount of an IL-23R inhibitor of the present invention or pharmaceutically acceptable solvate or salt thereof, or a composition disclosed herein comprising an IL-23 inhibitor of the present invention. In some aspects, the present invention provides a method of treating an inflammatory disease in a subject that includes administering to the subject a therapeutically effective amount of an IL-23R inhibitor of the present invention or pharmaceutically acceptable solvate or salt thereof, or a composition of the present invention. Suitable inflammatory diseases for treatment with a compound or pharmaceutically acceptable salt thereof, or a composition of the present invention, may include, but are not limited to inflammatory bowel disease (IBD), Crohn's disease (CD), ulcerative colitis (UC), psoriasis (PsO), or psoriatic arthritis (PsA) and the like. The inflammatory disease to be treated may be inflammatory bowel disease (IBD), Crohn's disease, or ulcerative colitis. The inflammatory disease to be treated may be selected from psoriasis, or psoriatic arthritis. The inflammatory disease to be treated may be psoriasis The inflammatory disease to be treated may be psoriatic arthritis. The inflammatory disease to be treated may be IBD.

The present invention relates to methods for treating an inflammatory disease in a subject in need thereof, comprising administering to the subject an IL-23R inhibitor disclosed herein (e.g., a peptide inhibitor or the IL-23R of Formula (I) to Formula (X) or any of Tables 1A to 1M. The inflammatory disease may be IBD, Crohn's disease, or ulcerative colitis. In aspect, the IBD may be ulcerative colitis. In an aspect, the IBD may be Crohn's disease. In an aspect, the inflammatory disease may be psoriasis (PsO), or psoriatic arthritis (PsA).

The present invention relates to methods for treating an inflammatory disease in a subject in need thereof, comprising administering to the subject an IL-23R inhibitor of Formula (I). The inflammatory disease may be IBD, Crohn's disease, or ulcerative colitis. In aspect, the IBD may be ulcerative colitis. In an aspect, the IBD may be Crohn's disease. In an aspect, the inflammatory disease may be psoriasis (PsO), or psoriatic arthritis (PsA).

The present invention relates to methods for treating an inflammatory disease in a subject in need thereof, comprising administering to the subject an IL-23R inhibitor of Formula (X). The inflammatory disease may be IBD, Crohn's disease, or ulcerative colitis. In aspect, the IBD may be ulcerative colitis. In an aspect, the IBD may be Crohn's disease. In an aspect, the inflammatory disease may be psoriasis (PsO), or psoriatic arthritis (PsA).

The present invention relates to methods for treating an inflammatory bowel disease (IBD) in a subject in need thereof, comprising administering to the subject an IL-23R inhibitor of: Example 2 (Compound 2, SEQ ID NO:2); Example (SEQ ID NO:4); Example 11 (SEQ ID NO: 11); Example 17 (SEQ ID NO:17); Example 18 (SEQ ID NO: 18); Example 19 (SEQ ID NO: 19); Example 20 SEQ ID NO:20); Example 21 SEQ ID NO:21); Example 23 (SEQ ID NO:23); or Example 24 (SEQ ID NO:24). The inflammatory disease may be IBD, Crohn's disease, or ulcerative colitis. The IBD may be ulcerative colitis. The IBD may be Crohn's disease. The inflammatory disease may be psoriasis (PsO), or psoriatic arthritis (PsA).

The present invention relates to methods of inhibiting IL-23 binding to an IL-23R on a cell, comprising contacting the IL-23R with a peptide inhibitor of the receptor disclosed herein. The cell may be a mammalian cell. The method may be performed in vitro or in vivo. Inhibition of binding may be determined by a variety of routine experimental methods and assays known in the art.

The present invention relates to a method of selectively inhibiting IL-23 or IL-23R signaling (or the binding of IL-23 to IL-23R) in a subject (e.g., in a subject in need thereof), comprising providing to the subject a peptide inhibitor of the IL-23R described herein. The present invention includes and provides a method of selectively inhibiting IL-23 or IL-23R signaling (or the binding of IL-23 to IL-23R) in the GI tract of a subject (e.g., a subject in need thereof), comprising providing to the subject a peptide inhibitor of the IL-23R of the present invention by oral administration. The exposure of GI tissues (e.g., small intestine or colon) to the administered peptide inhibitor may be at least 10-fold, at least 20-fold, at least 50-fold, or at least 100-fold greater than the exposure (level) in the blood. In particular embodiments, the present invention includes a method of selectively inhibiting IL23 or IL23R signaling (or the binding of IL23 to IL23R) in the GI tract of a subject (e.g., a subject in need thereof), comprising providing to the subject a peptide inhibitor, wherein the peptide inhibitor does not block the interaction between IL-6 and IL-6R or antagonize the IL-12 signaling pathway. In a further related embodiment, the present invention includes a method of inhibiting GI inflammation and/or neutrophil infiltration to the GI, comprising providing to a subject in need thereof a peptide inhibitor of the present invention. In some embodiments, methods of the present invention comprise providing a peptide inhibitor of the present invention (i.e., a first therapeutic agent) to a subject (e.g., a subject in need thereof) in combination with a second therapeutic agent. In certain embodiments, the second therapeutic agent is provided to the subject before and/or simultaneously with and/or after the peptide inhibitor is administered to the subject. In particular embodiments, the second therapeutic agent is an anti-inflammatory agent. In certain embodiments, the second therapeutic agent is a non-steroidal anti-inflammatory drug, steroid, or immune modulating agent. In certain embodiments, the method comprises administering to the subject a third therapeutic agent. In certain embodiments, the second therapeutic agent is an antibody that binds IL-23 or IL-23R.

The present invention relates to methods of inhibiting IL-23 signaling by a cell, comprising contacting the IL-23R with a peptide inhibitor described herein. In certain embodiments, the cell is a mammalian cell. In particular embodiments, the method is performed in vitro or in vivo. In particular embodiments, the inhibition of IL-23 signaling may be determined by measuring changes in phospho-STAT3 levels in the cell.

In any of the foregoing methods, IL-23R inhibitor administration to a subject may be conducted orally, but other routes of administration are not excluded. Other routes of administration include, but are not limited to, parenteral, subcutaneous, intravenous, intramuscular, intraperitoneal, transdermal, topical, buccal or ocular routes. Dosages of a peptide inhibitor or the IL-23R described herein (e.g., a compound of Formula (I) to Formula (X) or any of Tables 1A to 1M), or salt or solvate thereof to be administered to a subject may be determined by a person of skill in the art taking into account the disease or condition being treated including its severity, and factors including the age weight, sex, and the like. Exemplary dose ranges include, but are not limited to, from about 1 mg to about 1000 mg, or from about 1 mg to about 500 mg, from about 1 mg to about 100 mg, from about 10 mg to about 50 mg, from about 20 mg to about 40 mg, or from about 20 mg to about 30 mg. A dose range of a peptide inhibitor or the IL-23R described herein may be from about 600 mg to about 1000 mg. A dose range of a peptide inhibitor or the IL-23R described herein may be from about 300 mg to about 600 mg. A dose range of a peptide inhibitor or the IL-23R described herein may be from about 5 mg to about 300 mg. A dose range of a peptide inhibitor or the IL-23R described herein may be from about 25 mg to about 150 mg. A dose range of a peptide inhibitor or the IL-23R described herein may be from about 25 mg to about 100 mg. A dose range of a peptide inhibitor or the IL-23R described herein may be present in a dose range of from about 1 mg to about 100 mg. A dose range of a peptide inhibitor or the IL-23R described herein may be present in a dose range of from about 20 mg to about 40 mg. A dose range of a peptide inhibitor or the IL-23R described herein may be present in a dose range of from about 20 mg to about 30 mg.

### VII. Certain Aspects

The following aspects illustrate the invention. These aspects are not intended to limit the scope of the present invention, but rather to provide guidance to the skilled artisan to prepare and use the compounds, compositions, and methods of the present invention. While particular aspects of the present invention are described, the skilled artisan will appreciate that various changes and modifications can be made without departing from the spirit and scope of the invention.

### Formula I

1. An interleukin-23 receptor inhibitor which comprises an amino acid sequence of Formula I

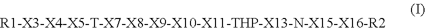

wherein:
   R1 is hydrogen, C₁ to C₄ alkyl C(O)—, or C₁ to C₄ alkyl C(O)— substituted with Cl, F, or cyano, or cPEG3aCO;
      X3 is dR, R, K, dK, or absent;
      X4 is Pen, Abu, aMeC, or C;
      X5 is K-Z or dK-Z;
      X7 is 7MeW, W, 3Pya, 7(2ClPh)W, 7(3(1NMepip)pyraz)W, 7(3(6AzaInd1Me))W, 7(3CF3TAZP)W, 7(3NAcPh)W, 7(3NPyrazPh)W, 7(3NpyrlonePh)W, 7(3UrPh)W, 7(4(CpCNPh))W, 7(4CF3Ph)W, 7(4NAcPh)W, 7(40CF3Ph)W, 7(4OMePh)W, 7(4Paz)W, 7(5(2(4OMePh)Pyr))W, 7(5(Ina7Pyr))W, 7(6(1)7dMeNDAZ))W, 7(6(2MeNDAZ))W, 7(7(124TAZP))W, 7(7Imzpy)W, 7BrW, 7EtW, 7PhW, 7PyrW, A, DT, or D7MeW;
      X8 is KAc, dK(Ac), K or dK;
      X9 is Pen, Abu, aMeC, or C;
      X10 is AEF or dAEF;
      X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy; X15 is 3Pya, 3MeH, H, F, hF, Y, dY, Y(CHF2), PAF, oAMPhe, F(CF3), dPaf, D3Pya, ACIPA(SR), 6OH3Pya, 5PyrimidAla, 5MePyridinAla, 5MeH, 5AmPyridinAla, 4TriazolAla, 4PyridinAla, 4Pya, 3QuinolAla, 3OHPhe, 3AmPyrazolAla, 2AmTyr, or 1MeH;
      X13 is K(Ac), d(KAc), E, or dE;
      X15 is absent, 3pya, 3MeH, H, F, hF, Y, dY, Y(CHF2), PAF, oAMPhe, F(CF3), dPaf, D3Pya, ACIPA(SR), 6OH3Pya, 5PyrimidAla, 5MePyridinAla, 5MeH, 5AmPyridinAla, 4TriazolAla, 4PyridinAla, 4Pya, 3QuinolAla, 3OHPhe, 3AmPyrazolAla, 2AmTyr, or 1MeH;
      X16 is meG, 4(R)HydroxyPro, 4(S)AminoPro, 4diFPro, 5(R)diMePro, aMeP, N(3AmBenzyl)Gly, N(Cyclohexyl)Gly, N(Isobutyl)Gly, P, or dP;
      R2 is —OH, —NH2, —NH(C1 to C4 alkyl), —NH(C1-C4 alkyl), or —N(C1 to C4 alkyl)₂, each alkyl optionally substituted with Cl, F, or cyano; and
      Z is group comprising a lipid moiety; and
   wherein the IL-23R inhibitor is cyclized by a disulfide or thioether first bond between X4 and X9.
2. The IL-23R inhibitor of aspect 1, wherein
   X7 is 7MeW or W;
   X11 is 2Nal.
   X15 is 3Pya; and
   X16 is meGly or dmeGly.
3. The IL-23R inhibitor of aspect 1 or aspect 2, wherein.
   X4 is Pen; and X5 is Pen.
4. The IL-23R inhibitor of aspect of any of aspects 1-3, wherein X5 is dK(gEC16), k(gEC18), dK(PEG2PEG2gEC10OH), dK(PEG2PEG2-gEC16OH), dK(PEG2PEG2-gEC18OH), dK(PEG2PEG2-gEC20OH), dK(1PEG2_1PEG2_IsoGlu_C16_Diacid), K(1PEG2_1PEG2_IsoGlu_C18_Diacid), K(gEC16), K(gEC18), K(gEC18OH), K(PEG2gE C18OH), K(PEG2PEG2-C18OH), K(PEG2PEG2gEC18OH), K(PEG2-PEG2gE-C18OH), K(PEG2PEG2gEC20OH), K(PEG2PEG2pgEC18OH), K(PEG2PEG2PgEC18OH), K(PEG2PEG2-pppgE-C18OH), K(PEG2PEG2-PPPgE-C18OH), K(PEG2PEG6 gE C18OH), or K(PEG6gEC18OH.

### Formula II

5. An interleukin-23 receptor inhibitor which comprises an amino acid sequence of Formula II

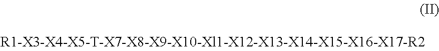

wherein:
   R1 is hydrogen, C1 to C4 alkyl C(O)—, or C1 to C4 alkyl C(O)— substituted with Cl, F, or cyano, 5Ava, AEEP, cPEG3aCO, C12gEPEG2PEG2CO, C14gEPEG2PEG2CO or Z;
   X3 is dR, dK, dK(d), or absent;
   X4 is Pen, Abu, aMeC, or C;
   X5 is L, N, aMeN, dK, dK(d), E, or K;
   X7 is 7MeW, W, 3Pya, 7(2ClPh)W, 7(3(1NMepip)pyraz)W, 7(3(6AzaInd1Me))W, 7(3CF3TAZP)W, 7(3NAcPh)W, 7(3NPyrazPh)W, 7(3NpyrlonePh)W, 7(3UrPh)W, 7(4(CpCNPh))W, 7(4CF3Ph)W, 7(4NAcPh)W, 7(40CF3Ph)W, 7(4OMePh)W, 7(4Paz)W, 7(5(2(4OMePh)Pyr))W, 7(5(Ina7Pyr))W, 7(6(1)7dMeNDAZ))W, 7(6(2MeNDAZ))W, 7(7(124TAZP))W, 7(7Imzpy)W, 7BrW, 7EtW, 7PhW, 7PyrW, A, DT, or D7MeW;
   X8 is K dK, K-Z, or dK-Z;
   X9 is Pen, C, aMeC, Abu;
   X10 is AEF, F, or F4OMe;
   X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
   X12 is THP or aMeL;
   X13 is E, L, KAc, dK, K, dL, dKAc, or dE;
   X14 is N, L, dN, or dL;
   X15 is 3Pya, 3MeH, H, F, hF, Y, dY, Y(CHF2), PAF, oAMPhe, F(CF3), dPaf, D3Pya, ACIPA(SR), 6OH3Pya, 5PyrimidAla, 5MePyridinAla, 5MeH, 5AmPyridinAla, 4TriazolAla, 4PyridinAla, 4Pya, 3QuinolAla, 3OHPhe, 3AmPyrazolAla, 2AmTyr, 1MeH or NH(2-(pyridine-3-yl)ethyl);
   X16 is meG, 4(R)HydroxyPro, 4(S)AminoPro, 4diFPro, 5(R)diMePro, aMeP, N(3AmBenzyl)Gly, N(Cyclohexyl)Gly, N(Isobutyl)Gly, P, or dP, or absent;
   X17 is absent or (PEG2PEG2PEG2PEG2gEC12), K(PEG2PEG2gEC12); and
   R2 is —OH —NH2, —NH(C1 to C4 alkyl), —H(C1-C4 alkyl), —N(C1 to C4 alkyl)₂, each alkyl optionally substituted with Cl, F, or cyano or K(PEG2PEG2gEC12); and
   Z is group comprising a lipid moiety; and wherein the IL-23R inhibitor is cyclized by a disulfide or thioether first bond between X4 and X9, and an amide second bond when X5 is E and X10 is AEF.
6. The IL-23R inhibitor of aspect 5, wherein:
   X3 is absent;
   X4 is Pen, Abu, aMeC, or C;
   X5 is L, N, aMeN, dK, dK(d), E, or K;
   X7 is W or 7MeW;
   X8 is K dK, K-Z, or dK-Z;
   X9 is Pen, C, aMeC, Abu;
   X10 is AEF, F, or F4OMe;
   X11 is 2Nal;
   X12 is THP or aMeL;
   X13 is E, L, KAc, dK, or K;
   X14 is N, L, dN, or dL;
   X15 is 3Pya or NH(2-(pyridin-3-yl)ethyl);
   X16 is Sarc or absent;
   X17 is absent or K(PEG2PEG2gEC12).
7. The IL-23R inhibitor of aspect 5 or 6, wherein:
   X4 is Pen, aMeC, or C;
   X9 is Pen, C, or aMeC; and
   the IL-23R inhibitor is cyclized by a disulfide or thioether first bond between X4 and X9.
8. The IL-23R inhibitor of aspect 5, wherein X8 is K(PEG12_C18_Diacid, K(PEG4_C18_Diacid, K(IsoGlu_C18_Diacid, K(IsoGlu_Palm), K(PEG4_IsoGlu_Palm), K(PEG4_IsoGlu_C18_Diacid, K(PEG12_IsoGlu_Palm), K(PEG12_IsoGlu_C18_Diacid, K(PEG12_OMe), K(PEG2PEG2gEC18OH), K(PEG2PEG2gEC20OH), K(PEG2PEG2gEC12), K(PEG2PEG2gEC14), or K(C14), K(gEC14).
9. The IL-23R inhibitor of any of aspects 5-8, further comprising a second bond between 5Ava or AEEP at R1 and E at position X13.

### Formula III

10. An interleukin-23 receptor inhibitor which comprises an amino acid sequence of Formula III

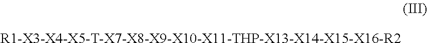

wherein:
   R1 is hydrogen, C1 to C4 alkyl C(O)—, or C1 to C4 alkyl C(O)— substituted with Cl, F, or cyano, or
   X3 is dR or absent;
   X4 is Pen, Abu, aMeC, C;
   X5 is N or dN;
   X7 is 7MeW, W, 3Pya, 7(2ClPh)W, 7(3(1NMepip)pyraz)W, 7(3(6AzaInd1Me))W, 7(3CF3TAZP)W, 7(3NAcPh)W, 7(3NPyrazPh)W, 7(3NpyrlonePh)W, 7(3UrPh)W, 7(4(CpCNPh))W, 7(4CF3Ph)W, 7(4NAcPh)W, 7(40CF3Ph)W, 7(4OMePh)W, 7(4Paz)W, 7(5(2(4OMePh)Pyr))W, 7(5(Ina7Pyr))W, 7(6(1)7dMeNDAZ))W, 7(6(2MeNDAZ))W, 7(7(124TAZP))W, 7(7Imzpy)W, 7BrW, 7EtW, 7PhW, 7PyrW, A, DT, or D7MeW;
   X8 is KAc;
   X9 is Pen, Abu, aMeC, C;
   X10 is F—Z or AEF-Z;
   X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
   X13 is K(Ac) dK(Ac). dE, or E;
   X14 is L orN;
   X15 is 3Pya, 3MeH, H, F, hF, Y, dY, Y(CHF2), PAF, oAMPhe, F(CF3), dPaf, D3Pya, ACIPA(SR), 6OH3Pya, 5PyrimidAla, 5MePyridinAla, 5MeH, 5AmPyridinAla, 4TriazolAla, 4PyridinAla, 4Pya, 3QuinolAla, 3OHPhe, 3AmPyrazolAla, 2AmTyr, or 1MeH;
   X16 is meG, 4(R)HydroxyPro, 4(S)AminoPro, 4diFPro, 5(R)diMePro, aMeP, N(3AmBenzyl)Gly, N(Cyclohexyl)Gly, N(Isobutyl)Gly, P, or dP; and
   Z is group comprising a lipid moiety; and
   R2 is —OH, —NH2, —NH(C1 to C4 alkyl), —NH(C1-C4 alkyl), or —N(C1 to C4 alkyl)₂, each alkyl optionally substituted with Cl, F, or cyano; and
   wherein the IL-23R inhibitor is cyclized by a disulfide or thioether first bond between X4 and X9.
11. The IL-23R inhibitor of aspect 10, wherein:
   X7 is 7MeW or W;
   X11 is 2Nal;
   X15 is 3Pya; and
   X16 is Sarc or NmeKdCar (N-methyl D-carnitine).
12. The IL-23R inhibitor of aspect 10 or 11, wherein:
   X4 is Pen, aMeC, or C; and
   X9 is Pen, C, or aMeC; and
   the IL-23R inhibitor is cyclized by a disulfide or thioether first bond between X4 and X9.
13. The IL-23R inhibitor of any of aspects 10-12, wherein X10 (PEG2PEG2gEC18OH), AEF(PEG2PEG2-gEC16OH), AEF(PEG2PEG2gEC18OH), F(4-(2-(1PEG2_1PEG2_IsoGlu_Palm)aminoethoxy)), F(4-(2-(1PEG2_1PEG2_IsoGlu_C18_Diacid)aminoethoxy)), F(4-(2-(PEG4_PEG4_IsoGlu_Palm)aminoethoxy)), F(4-(2-(PEG12_IsoGlu_Palm)aminoethoxy)), F(4-(2-(PEG4_PEG4_IsoGlu_C18_Diacid)aminoethoxy)), or F(4-(2-(PEG12_IsoGlu_C18_Diacid)aminoethoxy)).

### Formula IV

14. An interleukin-23 receptor inhibitor which comprises an amino acid sequence of Formula IV

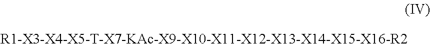

wherein:
   R1 is hydrogen, C1 to C4 alkyl C(O)—, or C1 to C4 alkyl C(O)— substituted with Cl, F, or cyano, or;
   X3 is dR or absent;
   X4 is Pen, aMeC, Abu, C;
   X5 is N, A, dN, dA;
   X7 is 7MeW, W, 3Pya, 7(2ClPh)W, 7(3(1NMepip)pyraz)W, 7(3(6AzaInd1Me))W, 7(3CF3TAZP)W, 7(3NAcPh)W, 7(3NPyrazPh)W, 7(3NpyrlonePh)W, 7(3UrPh)W, 7(4(CpCNPh))W, 7(4CF3Ph)W, 7(4NAcPh)W, 7(40CF3Ph)W, 7(4OMePh)W, 7(4Paz)W, 7(5(2(4OMePh)Pyr))W, 7(5(Ina7Pyr))W, 7(6(1)7dMeNDAZ))W, 7(6(2MeNDAZ))W, 7(7(124TAZP))W, 7(7Imzpy)W, 7BrW, 7EtW, 7PhW, 7PyrW, A, DT, or D7MeW;
   X9 is Pen, Abu, aMeC, or C;
   X10 is F4OMe, F4CONH2, F, 2Nal, AEF, 4AmF, or 4OMeF;
   X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
   X12 is aMeK-Z, Spiral Pip, or K-Z;
   X13 is KAc, E, A, L, dK, dKAc, dE, or dA;
   X14 is N, L, A, dN, dL, or dA;
   X15 is 3Pya, 3MeH, H, F, hF, Y, dY, Y(CHF2), PAF, oAMPhe, F(CF3), dPaf, D3Pya, ACIPA(SR), 6OH3Pya, 5PyrimidAla, 5MePyridinAla, 5MeH, 5AmPyridinAla, 4TriazolAla, 4PyridinAla, 4Pya, 3QuinolAla, 3OHPhe, 3AmPyrazolAla, 2AmTyr, or 1MeH;
   X16 is meG, 4(R)HydroxyPro, 4(S)AminoPro, 4diFPro, 5(R)diMePro, aMeP, N(3AmBenzyl)Gly, N(Cyclohexyl)Gly, N(Isobutyl)Gly, P, or dP; and
   R2 is —OH, —NH2, NH(C1 to C4 alkyl), —NH(C1-C4 alkyl), —N(C1 to C4 alkyl)₂, each alkyl optionally substituted with Cl, F, or cyano; and
   Z is group comprising a lipid moiety; and
   wherein the IL-23R inhibitor is cyclized by a disulfide or thioether first bond between X4 and X9.
15. The IL-23R inhibitor of aspect 14, wherein:
   R1 is C1 to C4 alkyl C(O)—;
   X3 is absent;
   X5 is N or A;
   X7 is 7MeW or W;
   X11 is 2Nal;
   X15 is 3Pya; and
   X16 is Sarc.
16. The IL-23R inhibitor of aspect 14 or 15, wherein:
   X4 is Pen, aMeC, or C;
   X9 is Pen, C, or aMeC; and
   the IL-23R inhibitor is cyclized by a disulfide first bond between X4 and X9.
17. The IL-23R inhibitor of any of aspects 14-16, wherein X12 is dKaMeK(PEG12IsoGluPalm), aMeK(PEG12IsoGluC18Diacid), K(PEG12IsoGluPalm), SpiralPipPEG12IsoGluPalm, K(PEG12IsoGluC18Diacid, aMeK(Peg4IsoGluC18Diacid), aMeK(PEG12C18Diacid), aMeK(Peg4IsoGluPalm), aMeK(IsoGluPalm), aMeK(IsoGluC18Diacid), aMeK(Peg4C18Diacid), aMeK(PEG2PEG2gEC18OH), aMeK(PEG2PEG2gEC16OH), or aMeK(PEG12gEC16).

### Formula V

18. An interleukin-23 receptor inhibitor which comprises an amino acid sequence of Formula V

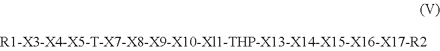

wherein:
   R1 is hydrogen, C₁ to C₄ alkyl C(O)—, C₁ to C₄ alkyl C(O)— substituted with Cl, F, or cyano;
   X3 is dR, dK, or absent;
   X4 is Pen, Abu, or C;
   X5 is N, K, Q, L, dN, dK, dL, or dQ;
   X7 is 7MeW, W, 3Pya, 7(2ClPh)W, 7(3(1NMepip)pyraz)W, 7(3(6AzaIndIMe))W, 7(3CF3TAZP)W, 7(3NAcPh)W, 7(3NPyrazPh)W, 7(3NpyrlonePh)W, 7(3UrPh)W, 7(4(CpCNPh))W, 7(4CF3Ph)W, 7(4NAcPh)W, 7(40CF3Ph)W, 7(4OMePh)W, 7(4Paz)W, 7(5(2(4OMePh)Pyr))W, 7(5(Ina7Pyr))W, 7(6(1)7dMeNDAZ))W, 7(6(2MeNDAZ))W, 7(7(124TAZP))W, 7(7Imzpy)W, 7BrW, 7EtW, 7PhW, 7PyrW, A, DT, or D7MeW;
   X8 is KAc, Q, K, dKAc, or dQ;
   X9 is Pen, aMeC, Abu, or C;
   X10 is AEF, AEF(G) or F4OMe;
   X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
   X13 is K-Z, or dK-Z;
   X14 is N, L, dN, or dL;
   X15 is 3Pya, 3MeH, H, F, bAla, hF, Y, dY, Y(CHF2), PAF, oAMPhe, F(CF3), dPaf, D3Pya, ACIPA(SR), 6OH3Pya, 5PyrimidAla, 5MePyridinAla, 5MeH, 5AmPyridinAla, 4TriazolAla, 4PyridinAla, 4Pya, 3QuinolAla, 3OHPhe, 3AmPyrazolAla, 2AmTyr, or 1MeH;
   X16 is meG, 4(R)HydroxyPro, 4(S)AminoPro, 4diFPro, 5(R)diMePro, aMeP, N(3AmBenzyl)Gly, N(Cyclohexyl)Gly, N(Isobutyl)Gly, P, dP or absent;
   X17 is absent, or K-Z;
   R2 is —OH, —NH2, —NH(C1 to C4 alkyl), —NH(C1-C4 alkyl), or —N(C1 to C4 alkyl)₂, each alkyl optionally substituted with Cl, F, or cyano; and
   Z is group comprising a lipid moiety; and
   wherein the IL-23R inhibitor is cyclized by a disulfide or thioether first bond between X4 and X9.
19. The IL-23R inhibitor of aspect 18 wherein:
   X3 is absent;
   X5 is N or A;
   X7 is 7MeW or W;
   X11 is 2Nal;
   X13 is K-Z;
   X15 is 3Pya, bAla, or F; and
   X16 is Sarc or absent.
20. The IL-23R inhibitor of aspect 18 or 19: wherein:
   (i) R1 further comprises a Z group;
   (ii) either the K or dK group of X5 is substituted by a Z group to give K-Z or dK-Z; and/or
   (iii) X17 is K(PEG2PEG2gEC16OH) or K(PEG2PEG2gEC18OH).
21. The IL-23R inhibitor of any of aspects 18 to 20, wherein:
   X4 is Pen, aMeC, or C;
   X9 is Pen, C, or aMeC; and
   the IL-23R inhibitor is cyclized by a disulfide first bond between X4 and X9.
22. The IL-23R inhibitor of any of aspects 18-21, wherein X13 is K(1PEG2_1PEG2_IsoGlu_C16_Diacid), K(1PEG2_1PEG2_IsoGlu_C18_Diacid), K(COPent), K(COPent), K(PEG2PEG2gEC10OH), K(PEG2PEG2gEC10OH), K(gEC10OH), K(FITCPEG4), K(PEG2PEG2gEC12), K(PEG2PEG2gEC14), K(PEG2PEG2gEC12), K(PEG2PEG2gEC12), K(PEG2PEG2gEC12), K(PEG2PEG2gEC14), K(PEG2PEG2gEC12), K(PEG2PEG2gEC12), K(PEG2PEG2gEC12), K(PEG2PEG2gEC14), K(C14), or K(gEC14).

### Formula VI

23. An interleukin-23 receptor inhibitor which comprises an amino acid sequence of Formula VI

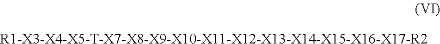

wherein
   R1 is hydrogen, C1 to C4 alkyl C(O)—, or C1 to C4 alkyl C(O)— substituted with Cl, F, or cyano, cPEG3aCO, or 6Ahx;
   X3 is dR, R, K, dK, dK-Z, K-Z, or absent;
   X4 is Pen, Abu, aMeC or C;
   X5 is N, or L;
   X7 is 7MeW, W, 3Pya, 7(2ClPh)W, 7(3(1NMepip)pyraz)W, 7(3(6AzaInd1Me))W, 7(3CF3TAZP)W, 7(3NAcPh)W, 7(3NPyrazPh)W, 7(3NpyrlonePh)W, 7(3UrPh)W, 7(4(CpCNPh))W, 7(4CF3Ph)W, 7(4NAcPh)W, 7(40CF3Ph)W, 7(4OMePh)W, 7(4Paz)W, 7(5(2(4OMePh)Pyr))W, 7(5(Ina7Pyr))W, 7(6(1)7dMeNDAZ))W, 7(6(2MeNDAZ))W, 7(7(124TAZP))W, 7(7Imzpy)W, 7BrW, 7EtW, 7PhW, 7PyrW, A, DT, or D7MeW;
   X8 is KAc, Q, dKAc, or dQ;
   X9 is Pen, C, aMeC, or Abu;
   X10 is AEF, F4OMe, or TMAPF;
   X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
   X12 is THP or Acvc, or Acpx;
   X13 is KAc, dKAc, dE or E;
   X14 is N or L;
   X15 is 3Pya, 3MeH, H, F, hF, Y, dY, Y(CHF2), PAF, oAMPhe, F(CF3), dPaf, D3Pya, ACIPA(SR), 6OH3Pya, 5PyrimidAla, 5MePyridinAla, 5MeH, 5AmPyridinAla, 4TriazolAla, 4PyridinAla, 4Pya, 3QuinolAla, 3OHPhe, 3AmPyrazolAla, 2AmTyr, THP, or 1MeH;
   X16 is K-Z, nMeK-Z, N—Z, Sarc-Z, dK-Z;
   X17 is absent or K-Z; and
   R2 is —OH, —NH2, —NH(C1 to C4 alkyl), —NH(C1-C4 alkyl), or —N(C1 to C4 alkyl)₂, each alkyl optionally substituted with Cl, F, or cyano; and
   Z is group comprising a lipid moiety; and
   wherein the IL-23R inhibitor is cyclized by a disulfide or thioether first bond between X4 and X9, and an amide second bond between R1 and X13 when R1 is 6Ahx and X13 is E.
24. The IL-23R inhibitor of aspect 23, wherein:
   X3 is dR, dK-Z, or absent;
   X5 is N or A;
   X7 is 7MeW or W;
   X8 is KAc, or Q;
   X11 is 2Nal;
   X13 is KAc or E; and
   X15 is 3Pya or THP.
25. The IL-23R inhibitor of any of aspects 23 to 24, wherein:
   X4 is Pen, aMeC, or C;
   X9 is Pen, C, or aMeC; and
   the IL-23R inhibitor is cyclized by a disulfide first bond between X4 and X9.
26. The IL-23R inhibitor of any of aspects 23-25, wherein X16 is N(4Am-Benzyl)-Gly, N(4AmBenzyl)Gly, 4diFPro, NMeK(PEG2PEG2PEG2PEG2gEC12), NMeK(PEG2PEG2gEC18OH), K(PEG2PEG2gEC18OH)Gly, K(PEG2PEG2-gEC18OH), NMeK(PEG2PEG2-gEC16OH), K(PEG2PEG2-gEC16OH), NMeK(PEG2PEG2-gEC18OH), dK(PEG12C18Diacid), dK(PEG12IsoGluPalm), dK(PEG12IsoGluC18Diacid), K(1PEG21PEG2IsoGluC18Diacid), K(1PEG21PEG2IsoGluC18), K(PEG2PEG2gEC18), K(PEG2PEG2gEC18OH).
27. The IL-23R inhibitor of any of aspects 23 to 26, wherein X3 is dK(gEC18OH), dK(PEG2gEC18OH), dK(PEG2PEG2gEC18OH), dK(PEG2PEG2gEC18OH), or dK(PEG2PEG2PEG2PEG2gEC12)
28. The IL-23R inhibitor of any of aspects 22 to 26, wherein X3 is absent or dR.

### Formula VII

29. An interleukin-23 receptor inhibitor which comprises an amino acid sequence of Formula VII

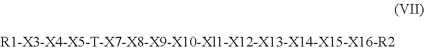

wherein:
   R1 is hydrogen, C1 to C4 alkyl C(O)—, or C1 to C4 alkyl C(O)— substituted with Cl, F, or cyano, GABA, CF3CO, succinic carnitine, or cPEG3aCO,
   X3 is dK, K, dK-Z, or K-Z;
   X4 is Pen, aMeC, or C;
   X5 is N, L, or E;
   X7 is 7MeW, W, 3Pya, 7(2ClPh)W, 7(3(1NMepip)pyraz)W, 7(3(6AzaInd1Me))W, 7(3CF3TAZP)W, 7(3NAcPh)W, 7(3NPyrazPh)W, 7(3NpyrlonePh)W, 7(3UrPh)W, 7(4(CpCNPh))W, 7(4CF3Ph)W, 7(4NAcPh)W, 7(40CF3Ph)W, 7(4OMePh)W, 7(4Paz)W, 7(5(2(4OMePh)Pyr))W, 7(5(Ina7Pyr))W, 7(6(1)7dMeNDAZ))W, 7(6(2MeNDAZ))W, 7(7(124TAZP))W, 7(7Imzpy)W, 7BrW, 7EtW, 7PhW, 7PyrW, A, DT, or D7MeW;
   X8 is KAc, K, K(Me)3, dKAc, or dK;
   X9 is Pen, aMeC, or C;
   X10 is AEF, F, F(4-OMe), or TMAPF;
   X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
   X12 is THP, aMeL, Acvc, or Acpx;
   X13 is KAc, dKAc, L, E, dE, K(NMeAc), dK(Me)3, or K(Me)3;
   X14 is N or L;
   X15 is 3Pya, THP, 3MeH, H, F, hF, Y, dY, Y(CHF2), PAF, oAMPhe, F(CF3), dPaf, D3Pya, ACIPA(SR), 6OH3Pya, 5PyrimidAla, 5MePyridinAla, 5MeH, 5AmPyridinAla, 4TriazolAla, 4PyridinAla, 4Pya, 3QuinolAla, 3OHPhe, 3AmPyrazolAla, 2AmTyr, or 1MeH;
   X16 is meG, 4(R)HydroxyPro, 4(S)AminoPro, 4diFPro, 5(R)diMePro, aMeP, N(3AmBenzyl)Gly, N(Cyclohexyl)Gly, N(Isobutyl)Gly, P, dP, Sarc, or absent;
   R2 is —OH, —NH2, —NH(C1 to C4 alkyl), —NH(C1-C4 alkyl), or —N(C1 to C4 alkyl)₂, each alkyl optionally substituted with Cl, F, or cyano; and
   Z is group comprising a lipid moiety; and
   wherein the IL-23R inhibitor is cyclized by a disulfide first bond between X4 and X9.
30. The IL-23R inhibitor of aspect 29, wherein:
   X7 is 7MeW or W;
   X8 is KAc, K, or K(Me)₃;
   X11 is 2Nal;
   X15 is 3Pya or THP; and
   X16 is Sarc, or absent.
31. The IL-23R inhibitor of any of aspects aspect 29 to 30, wherein:
   R1 further comprises a Z group.
32. The IL-23R inhibitor of aspect 31, wherein the Z group is C12gEPEG2PEG2CO, or C14gEPEG2PEG2CO.
33. The IL-23R inhibitor of any of aspects aspect 29 to 32, wherein: when X5 is E and X10 is AEF, the IL-23R inhibitor further comprises an amide second bond cyclizing the inhibitor.
34. The IL-23R inhibitor of any of aspects aspect 29 to 32, wherein: when R1 comprises GABA and X13 is E, the IL-23R inhibitor further comprises an amide second bond cyclizing the inhibitor.
35. The IL-23R inhibitor of any of aspects 29-34, wherein X3 is dK(1PEG21PEG2IsoGluC16Diacid), dK(1PEG21PEG2IsoGluC18Diacid), dK(DAP(C16OH)2), dK(gEC16), dK(gEC16), dK(gEC18), dK(gEC18), dK(gEC18OH), dK(GolAC16), dK(GolAC16OH), dK(GolAC18OH), dK(IsoGluC18Diacid), dK(PEG12C18Diacid), dK(PEG12IsoGluC18Diacid), dK(PEG12IsoGluPalm), K(PEG120Me), dK(PEG2 Sp6 PEG2 gE C18OH), dK(PEG2gEC18OH), dK(PEG2PEG2 C18OH), dK(PEG2PEG2 gE C18OH (c), dK(PEG2PEG2 gE C18OH (C), dK(PEG2PEG2 gE Sp6 C18OH), dK(PEG2PEG2 gE(C) C18OH, dK(PEG2PEG2GolAC18OH), dK(PEG2PEG2-C18GolB), dK(PEG2PEG2C18OH), dK(PEG2PEG2gE(C)C12), dK(PEG2PEG2gE(c)C18OH), dK(PEG2PEG2gEC10OH), dK(PEG2PEG2-gEC10OH), dK(PEG2PEG2gEC12), dK(PEG2PEG2gEC120H(C)), dK(PEG2PEG2gEC120H(c)), dK(PEG2PEG2gEC14), dK(PEG2PEG2-gEC16), dK(PEG2PEG2gEC16OH), dK(PEG2PEG2-gEC16OH), dK(PEG2PEG2gEC18), dK(PEG2PEG2-gEC18), dK(PEG2PEG2gEC18OH), dK(PEG2PEG2-gEC18OH), K(PEG2PEG2gEC20OH), dK(PEG2PEG2gEDab(mXOH)2), K(PEG2PEG2-gEDAP(pXOH)2), dK(PEG2PEG2gEmXOH), dK(PEG2PEG2-gEmXOH), dK(PEG2PEG2-gEpXOH), dK(PEG2PEG2-gETrxC18OH), dK(PEG2PEG2-gETrxC20OH), dK(PEG2PEG2-PEG2PEG2gEC12), dK(PEG2PEG2-PgEC18OH), dK(PEG2PEG2-pgEC18OH), dK(PEG2PEG2-PPPgEC18OH), dK(PEG2PEG2-pppgEC18OH), dK(PEG2PEG2SP6gEC18OH), dK(PEG2PEG2-TrxgEC18OH), dK(PEG2PEG6-gEC18OH), K(PEG4), dK(Peg4C18Diacid, dK(Peg4IsoGluC18Diacid), dK(PEG6 gE C18OH), dK(Sp6 PEG2PEG2gE C18OH), or dKPEG2PEG2-gEDAP(C16OH)₂.

### Formula VIII

36. An interleukin-23 receptor inhibitor which comprises an amino acid sequence of VIII

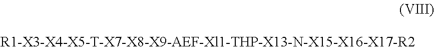

wherein:
   R1 is hydrogen, C1 to C4 alkyl C(O)—, or C1 to C4 alkyl C(O)— substituted with Cl, F, or cyano, C12gEPEG2PEG2CO, ClAcPEG4CO;
   X3 is dR, R, dK(SP6), K(SP6), K, or dK;
   X4 is Pen, Abu, aMeC or C;
   X5 is N or E;
   X7 is 7MeW, W, 3Pya, 7(2C1Ph)W, 7(3(1NMepip)pyraz)W, 7(3(6AzaInd1Me))W, 7(3CF3TAZP)W, 7(3NAcPh)W, 7(3NPyrazPh)W, 7(3NpyrlonePh)W, 7(3UrPh)W, 7(4(CpCNPh))W, 7(4CF3Ph)W, 7(4NAcPh)W, 7(40CF3Ph)W, 7(4OMePh)W, 7(4Paz)W, 7(5(2(4OMePh)Pyr))W, 7(5(Ina7Pyr))W, 7(6(1)7dMeNDAZ))W, 7(6(2MeNDAZ))W, 7(7(124TAZP))W, 7(7Imzpy)W, 7BrW, 7EtW, 7PhW, 7PyrW, A, DT, or D7MeW;
   X8 is Kac;
   X9 is Pen, C, aMeC, or Abu;
   X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
   X13 is E, dE, K, or dK;
   X15 is 3Pya, 3MeH, H, F, hF, Y, dY, Y(CHF2), PAF, oAMPhe, F(CF3), dPaf, D3Pya, ACIPA(SR), 6OH3Pya, 5PyrimidAla, 5MePyridinAla, 5MeH, 5AmPyridinAla, 4TriazolAla, 4PyridinAla, 4Pya, 3QuinolAla, 3OHPhe, 3AmPyrazolAla, 2AmTyr, or 1MeH;
   X16 is meG, 4(R)HydroxyPro, 4(S)AminoPro, 4diFPro, 5(R)diMePro, aMeP, N(3AmBenzyl)Gly, N(Cyclohexyl)Gly, N(Isobutyl)Gly, P, dP, or absent;
   X17 is K-Z or dK-Z; or
   R2 is —OH, —NH2, —NH(C1 to C4 alkyl), —NH(C1-C4 alkyl), or —N(C1 to C4 alkyl)₂, each alkyl optionally substituted with Cl, F, or cyano; and
   Z is group comprising a lipid moiety; and
   wherein the IL-23R inhibitor is cyclized by a disulfide or thioether first bond between X4 and X9, and an amide second bond when X5 is E and X10 is AEF.
37. The IL-23R inhibitor of aspect 36, wherein:
   X7 is 7MeW or W;
   X11 is 2Nal;
   X15 is 3Pya; and
   X16 is sarc or absent.
38. The IL-23R inhibitor of any of aspects 36 to 37, wherein:
   X4 is Pen, aMeC, or C;
   X9 is Pen, C, or aMeC; and
   the IL-23R inhibitor is cyclized by a disulfide first bond between X4 and X9
39. The IL-23R inhibitor of any of aspects 36 to 38, wherein X17 is K(PEG2PEG2gEC18OH), K(PEG2PEG2-gEC16OH), K(1PEG21PEG2IsoGluC16Diacid), K(1PEG21PEG2IsoGluC18Diacid), K(PEG2PEG2gEC20OH), K(PEG2PEG2gEC12), or K(PEG2NMePEG2NMegENMeC18Tetrazole).

### Formula IX

40. An interleukin-23 receptor inhibitor which comprises an amino acid sequence of Formula IX

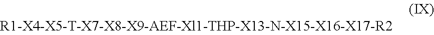

wherein:
   R1 is hydrogen, C1 to C4 alkyl C(O)—, or C1 to C4 alkyl C(O)— substituted with Cl, F, or cyano, 5Ava, AEEP or C14gEPEG2PEG2CO;
   X4 is Pen, Abu, C, aMeC, or absent;
   X5 is N or absent;
   X7 is 7MeW, W, 3Pya, 7(2ClPh)W, 7(3(1NMepip)pyraz)W, 7(3(6AzaInd1Me))W, 7(3CF3TAZP)W, 7(3NAcPh)W, 7(3NPyrazPh)W, 7(3NpyrlonePh)W, 7(3UrPh)W, 7(4(CpCNPh))W, 7(4CF3Ph)W, 7(4NAcPh)W, 7(40CF3Ph)W, 7(4OMePh)W, 7(4Paz)W, 7(5(2(4OMePh)Pyr))W, 7(5(Ina7Pyr))W, 7(6(1)7dMeNDAZ))W, 7(6(2MeNDAZ))W, 7(7(124TAZP))W, 7(7Imzpy)W, 7BrW, 7EtW, 7PhW, 7PyrW, A, DT, or D7MeW;
   X8 is KAc, dK, dQ, or Q;
   X9 is Pen, S5H, C, or aMeC;
   X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
   X13 is E, KAc, dK(d), S5H, dE, dK(Ac), dK, or R5H;
   X15 is 3Pya, 3MeH, H, F, hF, Y, dY, Y(CHF2), PAF, oAMPhe, F(CF3), dPaf, D3Pya, ACIPA(SR), 6OH3Pya, 5PyrimidAla, 5MePyridinAla, 5MeH, 5AmPyridinAla, 4TriazolAla, 4PyridinAla, 4Pya, 3QuinolAla, 3OHPhe, 3AmPyrazolAla, 2AmTyr, or 1MeH;
   X16 is Sarc, 4(R)HydroxyPro, 4(S)AminoPro, 4diFPro, 5(R)diMePro, aMeP, N(3AmBenzyl)Gly, N(Cyclohexyl)Gly, N(Isobutyl)Gly, P, or dP;
   X17 is K-Z;
   R2 is —OH, —NH2, —NH(C1 to C4 alkyl), —NH(C1-C4 alkyl), or —N(C1 to C4 alkyl)₂, each alkyl optionally substituted with Cl, F, or cyano; and
   Z is group comprising a lipid moiety; and
   wherein the IL-23R inhibitor is cyclized by a disulfide or thioether first bond between X4 and X9 or an aliphatic bond (generated from a Ring Closing Metathesis “RCM” reaction) between X9 and X13 when both residues are S5H.
41. The IL-23R inhibitor of aspect 40, wherein:
   X7 is 7MeW or W;
   X11 is 2Nal;
   X15 is 3Pya; and
   X16 is Sarc.
42. The IL-23R inhibitor of any of aspects 40 to 41, wherein:
   the IL-23R inhibitor comprises a second amide bond between R1 and X13 when R1 is 5Ava or AEEP and X13 is E.
43. The IL-23R inhibitor of any of aspects 40 to 42, wherein:
   R1 further comprises a Z group.
44. The IL-23R inhibitor of any of aspects 40 to 43, wherein X17 is K(PEG2PEG2gEC18OH), K(PEG2PEG2gEC16OH), K(PEG2PEG2gEC20OH), K(PEG2PEG2gEC14), K(PEG2PEG2gEC12), K(gEC14), K(C14), K(gEC12), K(PEG2PEG2gEDProC14), K(PEG2PEG2C14), K(GSGSGSGC14), K(PEG2PEG2SP6C14), K(PEG2C14), K(PEG2PEG2gESarC14), or K(PEG2PEG2gEProC14.
45. The IL-23R inhibitor of any of aspects 40 to 43, wherein X17 is K(PEG2PEG2gEC18OH), K(PEG2PEG2gEC16OH), K(PEG2PEG2gEC20OH), K(PEG2PEG2gEC14), K(PEG2PEG2gEC12), K(C14), K(gEC12), K(PEG2PEG2gEDProC14), K(PEG2PEG2C14), K(GSGSGSGC14), K(PEG2PEG2SP6C14), K(PEG2C14), K(PEG2PEG2gESarC14), or K(PEG2PEG2gEProC14).

### Formula X

46. An interleukin-23 receptor inhibitor which comprises an amino acid sequence of Formula X

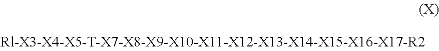

wherein:
   R1 is hydrogen, C1 to C4 alkyl C(O)—, or C1 to C4 alkyl C(O)— substituted with Cl, F, or cyano, 7Ahp, 6Ahx, 5Ava, 6Ava, AEEP, GABA, succinyl carnitine. cPEG3aCO, ClAcPEG4CO, 1PEG2_1PEG2_IsoGlu_C18, 1PEG2_1PEG2_IsoGlu_C18_Diacid, PentCO, PEG12_OMe, HOC18gEPEG2PEG2, PEG2PEG2gEC16OH, PEG4_Decyl, PEG4_Lauryl, PEG4_Capryl, PEG4_Hexyl, PEG2_Palm, PEG2_Myristyl, PEG2_Lauryl, Hexyl, Decyl, PEG2_Decyl, PEG2_Capryl, Oct, PEG4_Palm, Palm, Lauryl, 1PEG2_1PEG2_IsoGlu_C16_Diacid, HOC16gEPEG2PEG2orn, or Z;
   X3 is dR, dK, dK-Z, or absent;
   X4 is Pen, aMeC, Abu, or C;
   X5 is N, L, Q, K, E, aMeN, dN, dL, dQ, dK, dE, K-Z, or dK-Z;
   X7 is 7MeW, W, 3Pya, 7(2ClPh)W, 7(3(1NMepip)pyraz)W, 7(3(6AzaInd1Me))W, 7(3CF3TAZP)W, 7(3NAcPh)W, 7(3NPyrazPh)W, 7(3NpyrlonePh)W, 7(3UrPh)W, 7(4(CpCNPh))W, 7(4CF3Ph)W, 7(4NAcPh)W, 7(40CF3Ph)W, 7(4OMePh)W, 7(4Paz)W, 7(5(2(4OMePh)Pyr))W, 7(5(Ina7Pyr))W, 7(6(1)7dMeNDAZ))W, 7(6(2MeNDAZ))W, 7(7(124TAZP))W, 7(7Imzpy)W, 7BrW, 7EtW, 7PhW, 7PyrW, A, DT, or D7MeW;
   X8 is KAc, dK(Ac), dQ, or Q;
   X9 is Pen, C, aMeC, or Abu;
   X10 is AEF, F4OMe, F(4-CONH2), TMAPF, AEF(G), or F;
   X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
   X12 is THP, aMeL, Acvc, Acpx, aMeK, or aMeK-Z;
   X13 is K(Ac), dK(Ac), E, dE, L, dL, dK-Z, or K-Z;
   X14 is N, K, or K-Z;
   X15 is 3Pya, 3MeH, H, F, hF, Y, dY, Y(CHF2), PAF, oAMPhe, F(CF3), dPaf, D3Pya, ACIPA(SR), 6OH3Pya, 5PyrimidAla, 5MePyridinAla, 5MeH, 5AmPyridinAla, 4TriazolAla, 4PyridinAla, 4Pya, 3QuinolAla, 3OHPhe, 3AmPyrazolAla, 2AmTyr, THP, NH(2-(pyridin-3-yl)ethyl), bAla, THP, aMeF, or 1MeH;
   X16 is Sarc, K-Z, NMeK-Z, or absent;
   X17 is K-Z, dK-Z, or absent;
   R2 is —OH, —NH2, —NH(C1 to C4 alkyl), —NH(C1-C4 alkyl), or —N(C1 to C4 alkyl)₂, each alkyl optionally substituted with Cl, F, cyano or Z;
   Z is group comprising a lipid moiety; and
   wherein the IL-23R inhibitor is cyclized by a disulfide or thioether first bond between X4 and X9, and an amide second bond (i) between X5 and X10 when X5 is E and X10 is AEF, or (ii) between X13 and R1 when X13 is E and R1 is 7Ahp, 6Ahx, 5Ava, 6Ava, AEEP, or GABA.
47. The IL-23R inhibitor of aspect 46, wherein:
   R1 is hydrogen, C1 to C4 alkyl C(O)—, or C1 to C4 alkyl C(O)— substituted with Cl, F, or cyano;
   X3 is dR, or dK-Z;
   X4 is Pen, aMeC, or C;
   X5 is N, L, Q, or K;
   X7 is 7MeW, W, 3Pya, 7(2ClPh)W, 7(3(1NMepip)pyraz)W, 7(3(6AzaInd1Me))W, 7(3CF3TAZP)W, 7(3NAcPh)W, 7(3NPyrazPh)W, 7(3NpyrlonePh)W, 7(3UrPh)W, 7(4(CpCNPh))W, 7(4CF3Ph)W, 7(4NAcPh)W, 7(40CF3Ph)W, 7(4OMePh)W, 7(4Paz)W, 7(5(2(4OMePh)Pyr))W, 7(5(Ina7Pyr))W, 7(6(1)7dMeNDAZ))W, 7(6(2MeNDAZ))W, 7(7(124TAZP))W, 7(7Imzpy)W, 7BrW, 7EtW, 7PhW, 7PyrW, A, DT, or D7MeW; X8 is KAc, or Q;
   X9 is Pen, C, or aMeC;
   X10 is AEF, F4OMe, F(4-CONH2), or F;
   X11 is 2-Nal, Phe(2-Me), Phe(3-Me), Phe(4-Me), Phe(3,4-dimethoxy), 2Quin, 3Quin, or 1-Nal;
   X12 is THP;
   X13 is KAc, E, or L;
   X14 is N, or K;
   X15 is 3Pya, 3MeH, H, F, hF, Y, dY, Y(CHF2), PAF, oAMPhe, F(CF3), dPaf, D3Pya, ACIPA(SR), 6OH3Pya, 5PyrimidAla, 5MePyridinAla, 5MeH, 5AmPyridinAla, 4TriazolAla, 4PyridinAla, 4Pya, 3QuinolAla, 3OHPhe, 3AmPyrazolAla, 2AmTyr, THP, NH(2-(pyridin-3-yl)ethyl), bAla, or aMeF, or 1MeH;
   X16 is Sarc or absent;
   X17 is K-Z, or dK-Z;
   R2 is —OH, —NH2, —NH(C1 to C4 alkyl), —NH(C1-C4 alkyl), or —N(C1 to C4 alkyl)₂, each alkyl optionally substituted with Cl, F, or cyano
   Z is group comprising a lipid moiety; and
   wherein the IL-23R inhibitor is cyclized by a disulfide or thioether first bond between X4 and X9.
48. The IL-23R inhibitor of any of aspects 46 to 47, wherein:
   X7 is 7MeW or W;
   X11 is 2Nal or 3Quin;
   X15 is 3Pya, THP, H, NH(2-(pyridin-3-yl)ethyl), bAla, F, or aMeF; and
   X16 is Sarc; and
   R2 is —OH —NH2, —N(H)C1-C4 alkyl.
49. The IL-23R inhibitor of any of aspects 46 to 47, wherein X7 is 7MeW or W.
50. The IL-23R inhibitor of any of aspects 46 to 47, wherein X11 is 2Nal or 3Quin.
51. The IL-23R inhibitor of any of aspects 46 to 47, wherein X11 is 2Nal or 3Quin.
52. The IL-23R inhibitor of any of aspects 46 to 51, wherein the Z group of X17 is selected from the group consisting of PEG2, PEG2PEG2gEC18OH, PEG2PEG2eKC18OH, PEG2PEG2gDabC18OH, dK(PEG12IsoGluC18Diacid), dK(Peg4IsoGluPalm), dK(IsoGluPalm), dK(PEG12C18Diacid), dK(Peg4IsoGluC18Diacid), and dK(PEG12IsoGluPalm), dK(Peg4C18Diacid, dK(IsoGluC18Diacid).
53. The IL-23R inhibitor of any of aspects 46 to 52, wherein the Z group of X17 is selected from the group consisting of PEG2PEG2gEC18OH, PEG2PEG2eKC18OH, PEG2PEG2gDabC18OH, dK(PEG12IsoGluC18Diacid), and dK(Peg4IsoGluPalm).
54. An IL-23R inhibitor of any of aspects 1-50, wherein each Z is selected independently from a Z1 to Z5 group:
   Z1 is

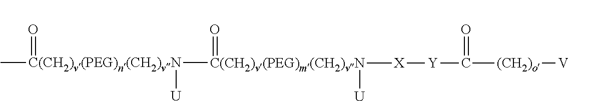

wherein:
   PEG is —OCH₂CH₂—;
      n′=0 or 2-24, when n′ is 0 the group is absent and replaced by a bond;
      m′=0 or 2-24, when m′ is 0 the group is absent and replaced by a bond;
      v′ is independently selected from the range of 1-4 for each occurrence;
      v″ is independently selected from the range of 0-4 for each occurrence, when v″ is 0 the group is replaced by a bond;
      X=gE, dgE, 4SB, p, P, ppp, PPP, gE-(c), gE-(C), sp6, gDab, eK, Trx, or absent;
      o′=6-18;
      Y=gE, sp6, GolA, Pro, D-Pro, meG, Dab, Trx, or absent;
      U is hydrogen or methyl;
      V=—COOH, tetrazole, GolB, mXOH, pXOH, OPhenyl, carnitine, d-carnitine, or hydrogen.
   Z2 is

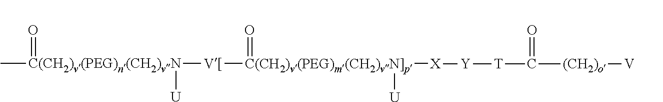

wherein:
   PEG is —OCH₂CH₂—;
      n′=0 or 2-24, when n′ is 0 the group is absent and replaced by a bond;
      m′ is independently selected from 0 or the range of 2-24 for each occurrence, when m′ is 0 the group is replaced by a bond;
      v′ is independently selected from the range of 1-4 for each occurrence;
      v″ is independently selected from the range of 0-4 for each occurrence, when v″ is 0 the group is replaced by a bond;
      p′ is 1-3;
      V′ is sp6, gEgE
      X=gE, dgE, 4SB, p, P, ppp, PPP, gE-(c), gE-(C), sp6, gDab, eK, Trx, or absent;
      Y=gE, sp6, GolA, Pro, D-Pro, meG, Dab, Trx, or absent;
      X=Trx;
      U is hydrogen or methyl;
      o′=6-18;
      V=—COOH, tetrazole, GolB, mXOH, pXOH, OPhenyl, carnitine, d-carnitine, or hydrogen;
   Z3 is
      -gE-C(O)(CH₂)₆₋₁₀CH₃, or -gE-C(O)(CH₂)₁₁₋₁₈CH₃;
   Z4 is
      —C(O)(CH₂)₆₋₁₈COOH or —C(O)(CH₂)₆₋₁₈COO(C1-4 alkyl);
   Z5 is:

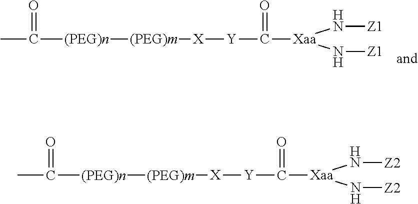

wherein:
   n and m are independently selected from the range of 0 to 24;
   X is absent or is selected from the group consisting of E, dgE, 4SB, gE-(c), gE-(C), sp6, gDab, eK, or Trx;
   Y is absent or is selected from the group consisting of E, dgE, 4SB, gE-(c), gE-(C), sp6, gDab, eK, or Trx;
   Xaa is a diamino-carboxylic acid; and
   Z1 an Z2 are defined above.
55. An IL-23R inhibitor of any of aspects 1-50, wherein at least one Z is selected from Z1, Z2, Z3, and Z4.
56. A IL-23R inhibitor of any of aspects 1-50, wherein at least one Z is a Z5.
57. An IL-23R inhibitor selected from Table 1A, Table 1B, Table 1C, Table 1D, Table 1E, Table 1F, Table 1G, Table 1H, Table 1I, Table 1J, Table 1K, Table 1L, or Table 1M respectively.
58. An IL-23R inhibitor selected from the group consisting of: Example 2 (compound 2 SEQ ID NO:2); Example (SEQ ID NO:4); Example 11 (SEQ ID NO: 11); Example 17 (SEQ ID NO: 17); Example 18 (SEQ ID NO: 18); Example 19 (SEQ ID NO: 19); Example 20 SEQ ID NO:20); Example 21 SEQ ID NO:21); Example 23 (SEQ ID NO:23); and Example 24 (SEQ ID NO:24).
59. The IL-23R inhibitor of any preceding aspect wherein the interleukin-23 receptor is a human interleukin receptor.
60. A pharmaceutically acceptable salt, solvate, or form thereof of an IL-23R inhibitor of any of aspects 1-59.
61. A pharmaceutical composition which comprises:
   (i) peptide inhibitor of an interleukin-23 receptor or pharmaceutically acceptable salt, solvate, or form thereof according to any one of aspects 1-56, and
   (ii) a pharmaceutically acceptable carrier, excipient, or diluent.
62. A pharmaceutical composition which comprises:
   (i) peptide inhibitor of an interleukin-23 receptor or pharmaceutically acceptable salt, solvate, or form thereof according to any one of aspect 57, and
   (ii) a pharmaceutically acceptable carrier, excipient, or diluent.
63. A pharmaceutical composition which comprises:
   (i) peptide inhibitor of an interleukin-23 receptor or pharmaceutically acceptable salt, solvate, or form thereof according to aspect 58; and
   (ii) a pharmaceutically acceptable carrier, excipient, or diluent.
64. The use of a peptide inhibitor of an interleukin-23 receptor according to any of aspects 1-59 for the preparation of a medicament.
65. The use of a peptide inhibitor of an interleukin-23 receptor according to any of aspects 1-59, or a pharmaceutical composition according to any of aspects 60-63, for the preparation of a medicament for the treatment of an inflammatory disorder or autoimmune inflammatory disorder.
66. The use of a peptide inhibitor of an interleukin-23 receptor according to any of aspects 1-59, or a pharmaceutical composition according to any of aspects 60-63, for the preparation of a medicament for the treatment of autoimmune inflammation and related diseases and disorders including, but not limited to: multiple sclerosis, asthma, rheumatoid arthritis, inflammation of the gut, inflammatory bowel diseases (IBDs), juvenile IBD, adolescent IBD, Crohn's disease, ulcerative colitis, Celiac disease (nontropical Sprue), microscopic colitis, collagenous colitis, eosinophilic gastroenteritis/esophagitis, colitis associated with radio- or chemo-therapy, colitis associated with disorders of innate immunity as in leukocyte adhesion deficiency-1, sarcoidosis, Systemic Lupus Erythematosus, ankylosing spondylitis (axial spondyloarthritis), psoriatic arthritis, psoriasis (e.g., plaque psoriasis, guttate psoriasis, inverse psoriasis, pustular psoriasis, Palmo-Plantar Pustulosis, psoriasis vulgaris, or erythrodermic psoriasis), atopic dermatitis, acne ectopica, enteropathy associated with seronegative arthropathies, chronic granulomatous disease, glycogen storage disease type 1b, Hermansky-Pudlak syndrome, Chediak-Higashi syndrome, Wiskott-Aldrich Syndrome, pouchitis, pouchitis resulting after proctocolectomy and ileoanal anastomosis, gastrointestinal cancer, pancreatitis, insulin-dependent diabetes mellitus, mastitis, cholecystitis, cholangitis, primary biliary cirrhosis, viral-associated enteropathy, pericholangitis, chronic bronchitis, chronic sinusitis, asthma, uveitis, or graft versus host disease.
67. The use of aspect 66, wherein the diseases or disorders are selected from Inflammatory Bowel Disease (IBD), Ulcerative colitis (UC), Crohn's Disease (CD), psoriasis (PsO) or psoriatic arthritis (PsA).
68. A method for treating a disease or disorder associated with Interleukin 23 (IL-23) or the Interleukin 23 Receptor (IL-23R), which comprises administering:
   (i) an effective amount of a peptide inhibitor of an interleukin-23 receptor, or a pharmaceutically acceptable salt, solvate, or form thereof according to any one of aspects 1-59; or
   (ii) a pharmaceutical composition according to any one of aspects 60-63, respectively to a patient in need thereof.
69. The method of aspect 68, wherein the disease or disorder is associated with autoimmune inflammation.
70. The method of aspect 68, wherein the disease or disorder is associated with multiple sclerosis, asthma, rheumatoid arthritis, inflammation of the gut, inflammatory bowel diseases (IBDs), juvenile IBD, adolescent IBD, Crohn's disease, ulcerative colitis, Celiac disease (nontropical Sprue), microscopic colitis, collagenous colitis, eosinophilic gastroenteritis/esophagitis, colitis associated with radio- or chemo-therapy, colitis associated with disorders of innate immunity as in leukocyte adhesion deficiency-1, sarcoidosis, Systemic Lupus Erythematosus, ankylosing spondylitis (axial spondyloarthritis), psoriatic arthritis, psoriasis (e.g., plaque psoriasis, guttate psoriasis, inverse psoriasis, pustular psoriasis, Palmo-Plantar Pustulosis, psoriasis vulgaris, or erythrodermic psoriasis), atopic dermatitis, acne ectopica, enteropathy associated with seronegative arthropathies, chronic granulomatous disease, glycogen storage disease type 1b, Hermansky-Pudlak syndrome, Chediak-Higashi syndrome, Wiskott-Aldrich Syndrome, pouchitis, pouchitis resulting after proctocolectomy and ileoanal anastomosis, gastrointestinal cancer, pancreatitis, insulin-dependent diabetes mellitus, mastitis, cholecystitis, cholangitis, primary biliary cirrhosis, viral-associated enteropathy, pericholangitis, chronic bronchitis, chronic sinusitis, asthma, uveitis, or graft versus host disease.
71. The method of aspect 68, wherein the disease or disorder is associated with Ulcerative colitis (UC), Crohn's Disease (CD), psoriasis (PsO), or psoriatic arthritis (PsA).
72. The method of aspect 68, wherein the disease or disorder is Ulcerative colitis (UC).
73. The method of aspect 68, wherein the disease or disorder is Crohn's Disease (CD).
74. The method of aspect 68, wherein the disease or disorder is psoriasis (PsO).
75. The method of aspect 68, wherein the disease or disorder is psoriatic arthritis (PsA).
76. A kit which comprises a peptide inhibitor of an interleukin-23 receptor of any of aspects 1-59, or a pharmaceutical composition according to any of aspects 60 to 63, and instructions for the use of the inhibitor of an interleukin-23 receptor or pharmaceutical composition.
77. The kit of aspect 76, wherein the instructions are directed to the treatment of an inflammatory disease or disorder.
78. The kit of aspect 77, wherein the disease is inflammatory bowel disease (IBD), Crohn's disease (CD), ulcerative colitis (UC), psoriasis (PsO), and psoriatic arthritis (PsA).

The IL-23R inhibitors of aspects 1-60 may comprise amino aids of the D-isomer configuration at one or more positions. The IL-23R inhibitors of aspects 1-60, may comprise D-isomer only at: (i) one or more of positions X3, X5, X6, X8 and X13, and optionally one of positions X1-X2, X4, X7, X9 to X12, X14-X18 present in the inhibitor; or (ii) one or more of positions X3, X8 and X13, and optionally at one of positions X1-X2, X4-X7, X9 to X12, X14-X18 present in the inhibitor. The IL-23R inhibitors of aspects 1-60, may comprise D-isomer only at (i) X3, and optionally at one of positions X1-X2, X4-X18 present in the inhibitor; or (ii) one of positions X3, and X8, and optionally one of positions X1-X2, X4-X7, X9-X18 present in the inhibitor. The IL-23R inhibitors of aspects 1-60, may comprise D-isomer only at one or two of positions X1 to X18 appearing in the IL-23R inhibitors set forth herein. The IL-23R inhibitors of aspects 1-60, may comprise D-isomer only at only three or four of positions X1 to X18 appearing in the IL-23R inhibitors set forth herein. The IL-23R inhibitors of aspects 1-60, may comprise D-isomer at only five or six of positions X1 to X18 appearing in the IL-23R inhibitors set forth herein. IL-23R inhibitors with amino aids of the D-isomer configuration may be used in any of the pharmaceutical formulations, methods or uses of aspects 61-78.

### VIII. Examples

The following examples illustrate the invention. These examples are not intended to limit the scope of the present invention, but rather to provide guidance to the skilled artisan to prepare and use the compounds, compositions, and methods of the present invention. While particular aspects of the present invention are described, the skilled artisan will appreciate that various changes and modifications can be made without departing from the spirit and scope of the invention.

Some abbreviations useful in describing the invention are defined below in the following Table 2A and Table 2B.

TABLE 2A
Amino Acid Abbreviations

| Abbreviation | Definition | Smiles |
|---|---|---|
| dR, arg, or r | D-Arginine | |
| dK, (D)Lys, (D)- | D-lysine | |
| Lys, lys, or k | | |
| 5 Apa | 5AminoPentanoicAcid | |
| 2-Nal or 2Nal | 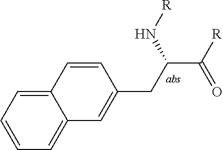 | O═C([C@H](Cc1cc2ccccc2cc1)N [R])[R] |
| | C₁₃H₁₁NOR₂ | |
| 3MeH | 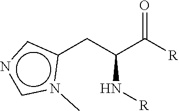 | Cn1cncc1C[C@H](N[R])C([R])═O |
| | 3-methyl-L-histidine | |
| 3Pya, 3Pal, 3-(2- pyridyl)-alanine | 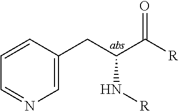 | O═C([C@H](Cclcnccc1)N[R])[R] |
| THP, 4- aminotetrahydro- 2H-pyran-4- carboxylic acid | 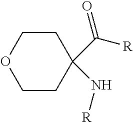 | O═C(C1(CCOCC1)N[R])[R] |
| | 4-amino-4-carboxy- | |
| | tetrahydropyran | |
| 7PhW, 7PhTrp or W(7-Ph) | 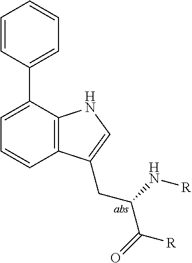 | O═C([C@H](Cclc[nH]c2c1cccc2- c1ccccc1)N[R])[R] |
| 7MeW, 7(MeW), 7MeTrp, 7- methyl-L- tryptophan | 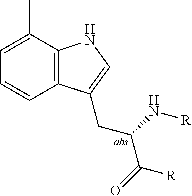 | Cc1cccc2c1[nH]cc2C[C@@H] (C([R])═O)N[R] |
| | 7-phenyl-L-tryptophan | |
| Abu | 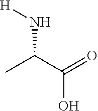 | C[C@@H](C═O)N |
| | 2-aminobutyric acid | |
| AEF, Phe(4-(2- aminoethoxy)), or F(4-2ae) | 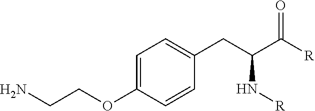 | NCCOc1ccc(C[C@@H](C([R])═ O)N[R])cc1 |
| | 4-(2-aminoethoxy)-L- | |
| | phenylalanine | |
| Ahp, 7Ahp, 7AHP, or 7AHP(2) | 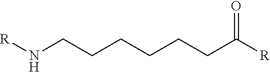 | O═C([R])CCCCCCN[R] |
| | 7-aminoheptanoic acid | |
| Ahx or 6Ahx, 6Ahx, 6Ahx(2), 6-aminohexanoic acid | 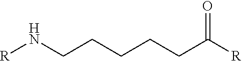 | O═C(CCCCCN[R])[R] |
| | 6-aminohexanoic acid | |
| aMeF, aMePhe, or aMe-Phe | 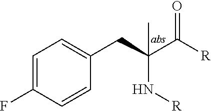 | C[C@](Cc(cc1)ccc1F)(C([R])═O) N[R] |
| | alpha-methyl L-phenylalanine | |
| aMeK, aMeLys, or | alpha-methyl L-lysine | |
| aMe-Lys | | |
| Arg or R | L-arginine | |
| dR, arg, r or | D-arginine | |
| (D)Arg | | |
| Asn or N | L-asparagine | |
| Ava, 5Ava(2), or 5Ava | 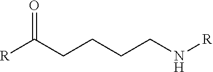 | O═C(CCCCN[R])[R] |
| | 5-Aminovaleric Acid | |
| bAla, b-ALA, beta-Alanine, bA | 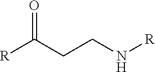 | O═C(CCN[R])[R] |
| | beta-alanine | |
| Bis-amino-PEG2 | 1,2-bis(2-aminoethoxy)ethane | |
| Cys or C | L-cysteine | |
| Dbu, Dab, (S)-2,4- diaminobutanoic acid, or DAB | 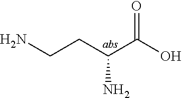 | NCC[C@@H](C(O)═O)N |
| | L-2,4-diaminobutyric acid | |
| Dap, Dap, DAP, Dpr or (S)-2,3- diaminopropanoic acid | 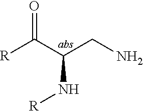 | NC[C@@H](C([R])═O)N[R] |
| | L-2,3-diaminopropionic acid | |
| dDab, D(Dab), dDpr, (R)-2,3- diaminopropanoic acid | 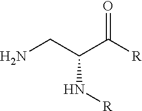 | NC[C@H](C([R])═O)N[R] |
| | D-2,4-diaminobutyric acid | |
| dDap, D(Dap), dDap, dap, dDbu, (R)-2,3- diaminopropanoic acid | 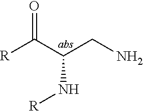 | NC[C@H](C([R])═O)N[R] |
| | D-2,3-diaminopropionic acid | |
| Fmoc-2Nal | 2-((((9H-fluoren-9- | |
| | yl)methoxy)carbonyl)amino)- | |
| | 3-(naphthalen-2-yl)propanoic | |
| | acid | |
| Fmoc-3Pya | (S)-2-((((9H-fluoren-9- | |
| | yl)methoxy)carbonyl)amino)- | |
| | 4-(pyridin-3-yl)butanoic acid | |
| Fmoc-7MeW | (S)-2-((((9H-fluoren-9- | |
| | yl)methoxy)carbonyl)amino)- | |
| | 3-(7-methyl-1H-indol-3- | |
| | yl)propanoic acid | |
| Fmoc-AEF | (S)-2-((((9H-fluoren-9- | |
| | yl)methoxy)carbonyl)amino)- | |
| | 3-(4-(2-((tert- | |
| | butoxycarbonyl)amino)ethoxy) | |
| | phenyl)propanoic acid | |
| Fmoc-aMePhe | (((9H-fluoren-9- | |
| | yl)methoxy)carbonyl)- | |
| | alphamethyl-L-phenylalanine | |
| Fmoc-arg or | N-alpha-(9- | |
| Fmoc-r | fluorenylmethyloxycarbonyl)- | |
| | N′-2,2,4,6,7- | |
| | pentamethyldihydrobenzofuran- | |
| | 5-sulfonyl-D-arginine | |
| Fmoc-Asn or | N²-(((9H-fluoren-9- | |
| Fmoc-N | yl)methoxy)carbonyl)-N4- | |
| | trityl-L-asparagine | |
| Fmoc-Dap(DDe) | N2-(Fmoc)-N6-(1-(4,4- | |
| | dimethyl-3,5- | |
| | dioxocyclohexylidene)ethyl)- | |
| | L-Dap | |
| Fmoc-DDe- | N6-(((9H-fluoren-9- | |
| Lys(Fmoc)-OH | yl)methoxy)carbonyl)-N2-(1- | |
| | (4,4-dimethyl-3,5- | |
| | dioxocyclohexylidene)ethyl)- | |
| | L-lysine | |
| Fmoc-Glu or | (S)-2-((((9H-fluoren-9- | |
| Fmoc-E | yl)methoxy)carbonyl)amino)- | |
| | 5-(tert-butoxy)-2-methyl-5- | |
| | oxopentanoic acid | |
| Fmoc-Lys(Ac) or | N2-(((9H-fluoren-9- | |
| Fmoc-K(Ac) | yl)methoxy)carbonyl)-N6- | |
| | acetyl-L-lysine | |
| Fmoc-Lys(DDe) | N2-(Fmoc)-N6-(1-(4,4- | |
| or Fmoc-K(DDe) | dimethyl-3,5- | |
| | dioxocyclohexylidene)ethyl)- | |
| | L-lysine | |
| Fmoc- | N2-(((9H-fluoren-9- | |
| Lys(NMeAc) or | yl)methoxy)carbonyl)-N6- | |
| Fmoc-K(NMeAc) | acetyl-N6-methyl-L-lysine | |
| Fmoc- | (9H-fluoren-9-yl)methyl (1- | |
| NMeLys(DDe) or | amino-6-((1-(4,4-dimethyl-3,5- | |
| Fmoc- | dioxocyclohexylidene)ethyl)amino)- | |
| NMeK(DDe) | 1-oxohexan-2- | |
| | yl)(methyl)carbamate | |
| Fmoc-Pen-Trt | (R)-2-((((9H-fluoren-9- | |
| | yl)methoxy)carbonyl)amino)- | |
| | 3-methyl-3-(tritylthio)butanoic | |
| | acid | |
| Fmoc-Pro or | Fmoc-proline-OH | |
| Fmoc-P | | |
| Fmoc-pro or | Fmoc-D-proline-OH | |
| Fmoc-p | | |
| Fmoc-R5H | (R)-2-((((9H-fluoren-9- | |
| | yl)methoxy)carbonyl)amino)hept- | |
| | 6-enoic acid | |
| Fmoc-Sar or | N-(((9H-fluoren-9- | |
| Fmoc-Sarc | yl)methoxy)carbonyl)-N- | |
| | methylglycine | |
| Fmoc-THP | 4-((((9H-fluoren-9- | |
| | yl)methoxy)carbonyl)amino) | |
| | tetrahydro-2H-pyran-4- | |
| | carboxylic acid | |
| Fmoc-Thr or | N-(((9H-fluoren-9- | |
| Fmoc-T | yl)methoxy)carbonyl)-O-(tert- | |
| | butyl)-L-threonine | |
| GABA, Gaba, Gaba(2), Gaba2, or 4Abu | 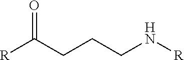 | O═C(CCCN[R])[R] |
| | 4-aminobutyric acid | |
| Glu or E | L-glutamic acid | |
| glu or e or D(Glu) | D-glutamic acid | |
| His or H | L-histidine | |
| Lys or K | L-lysine | |
| lys or k or (D)Lys | D-lysine | |
| hCys, hC | 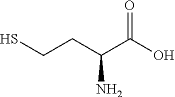 | C(CS)[C@@H](C(═O)O)N |
| | L-Homocysteine | |
| KAc, Lys(Ac), K(Ac), K(COMe), or K-Ac | 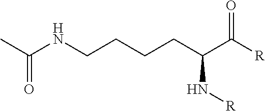 | CC(NCCCC[C@@H](C([R])═O) N[R])═O |
| | N-ε-acetyl-L-Lysine | |
| | N6-Acetyl-L-lysine | |
| MeK, N-MeLys, | N-methyl-Lysine | |
| NMeLys, NMeK, | (2S)-2-amino-6- | |
| or MeLys | (methylamino)hexanoic acid | |
| Pen | 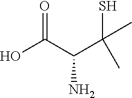 | CC(C)([C@@H](C(O)═O)N)S |
| | L-penicillamine, 3-Mercapto-L- | |
| | valine | |
| | (R)-2-Amino-3-mercapto-3- | |
| | methylbutanoic acid | |
| F4CONH2, Phe(4- CONH₂) or Phe(4- CONH2) or Phe(Cmd) or Phe_4Ad | 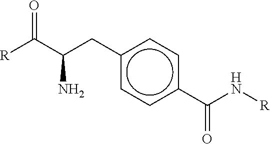 | N[C@H](C([R])═O)Ccccc(C(N[R])═O)cc1 |
| | 4-carbamoyl-L-phenylalanine | |
| | (S)-2-amino-3-(4- | |
| | carbamoylphenyl)propanoic acid | |
| F4OMe, Phe(4- OMe), or Phe_4OMe | 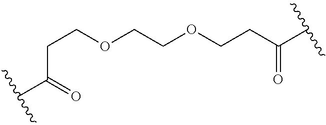 [SMILES: *C(=O)CCOCCOCCC(*)=O] | N[C@@H](CC1═CC═C(OC)C═C1)C(O)═O |
| | 4-methoxy-L-phenylalanine | |
| Quin, 3Quin, 3- Quin, 3QuinolAla, or 3QuinA | 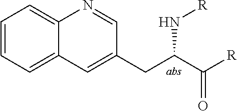 | O═C([C@H](Cc1cc2ccccc2nc1)N [R])[R] |
| | (S)-2-amino-3-(quinolin-3- | |
| | yl)propanoic acid | |
| R5H, | 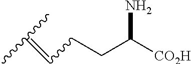 [SMILES: *=CCC[C@@H](N)C(=O)O] | |
| | (R)-2-aminopentanoic acid 5-diyl | |
| R6H, (R,E)-2- amino-8- hydroxyoct-7- enoic acid | 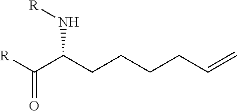 | C═CCCCC[C@H](C([R])═O)N[R] |
| | R (R)-2-aminohexanoic acid 6-diyl | |
| R7H, (R,E)-2- amino-9- hydroxynon-8- enoic acid | 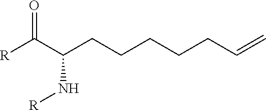 | C═CCCCCC[C@H](C([R])═O)N[R] |
| | (R)-2-aminoheptanoic acid 7-diyl | |
| S5H | 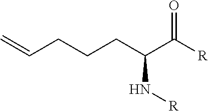 | C═CCCC[C@H](N[R])C([R])═O |
| | (S)-2-aminopentanoic acid 5-diyl | |
| meG, Sarc, MeGly, Sar, Sarc, MeGly, Sarcosine, Methylamino- Acetic Acid, N- methylglycine | 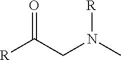 sarcosine or N-methylglycine | CN(CC([R])═O)[R] |
| Thr or T | L-threonine | |
| nFEtOH, Phe(4- | Fc1c(F)c([H])c(F)c(F)c1NC[C | N[C@@H](C═O)c(cc1)ccc1O |
| OCH2COOH, or | @@H](C([R])═O)N[R] | CC═O |
| 2-amino-2-[4- | (R)-2-amino-2-(4- | |
| (carboxymethoxy) | (carboxymethoxy)phenyl)acetic | |
| phenyl]acetic acid, | acid | |
| DappF6 Dap(pF(6)) | 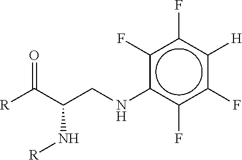 | Fc1c(F)c([H])c(F)c(F)c1NC [C@@H](C([R])═O)N[R] |
| | tetra-fluoro-phenylalanine | |

TABLE 2B
Abbreviations for Substituents, Reagents, and Solvents

| Abbreviation | Definition | Smiles |
|---|---|---|
| Ac or | acetyl | |
| MeCO | | |
| ACN | acetonitrile | |
| Boc | tert-butoxy-carbonyl | |
| CONH₂ | carboxamide | |
| COOH | carboxylic Acid | |
| DCM | dichloromethane | |
| Dde | N-(1-(4,4-dimethyl-2,6-dioxocyclohexylidene)ethyl | |
| DIC | N,N′-diisopropylcarbodiimide | |
| DMF | N,N-dimethylformamide | |
| Et₂O | di-ethylether | |
| FMOC or | ((9H-fluoren-9-yl)methoxy)carbonyl | |
| Fmoc | | |
| HOAT or | 1-hydroxy-7-azabenzotriazole | |
| HOAt | | |
| MeOH | methanol | |
| MTBE | methyl tert-butyl ether | |
| MW | microwave | |
| Oxyma | ethyl cyanohydroxyiminoacetate | |
| PEG2_DiA cid or PEG2DA | 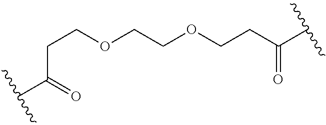 [SMILES: *C(=O)CCOCCOCCC(*)=O] | |
| | 3,3′-(ethane-1,2-diylbis(oxy))dipropanecarbonyl | |
| pF | 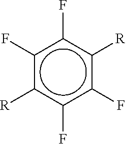 | Fc1c(F)c([R])c(F)c(F)c1[R] |
| | 2,3,5,6-tetrafluorophen-1,4-diyl linker | |
| pFS | 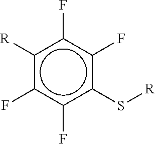 | Fc(c(S[R])c(c(F)c1[R1)F)c1F |
| | 2,3,5,6-tetrafluoro-4-mercaptophenol | |
| RT | room temperature | |
| TFA | trifluoroacetic acid | |
| TIPS | triisopropylsilane | |

TABLE 2C
Monomers

| # | Symbol/Name | Structure | Smiles |
|---|---|---|---|
| 1 | bMeW(2S3R) bMeW(2S,3R) | 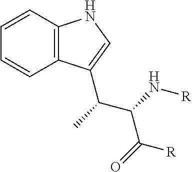 | C[C@H](C1═CN C2═C1C═CC═C2) [C@H](N[R])C ([R])═O |
| | | C₁₂H₁₂N₂OR₂ | |
| 2 | bMeW(2S3S), bMeW(2S,3S) | 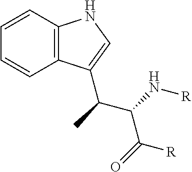 | C[C@@H](C1═C NC2═C1C═CC═C 2)[C@H](N[R])C ([R])═O |
| | | C₁₂H₁₂N₂OR₂ | |
| 3 | 6OH2Nal | 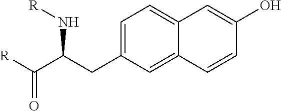 | [R]C([C@H](CC1 ═CC═C(C═C(O)C ═C2)C2═C1)N[R]) ═O |
| | | (S)-2-amino-3-(6-hydroxynaphthalen-2-yl)propanoic acid | |
| 4 | NMe7MeW | 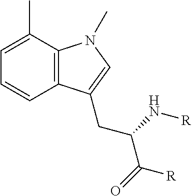 | [R]C([C@@H](N [R])CC1═CN(C)C2 ═C1C═CC═C2C)═ O |
| | | C₁₃H₁₄N₂OR₂ | |
| 5 | 7(4Paz)W | 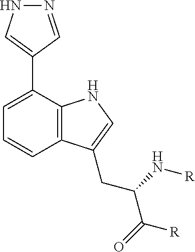 | [R]C([C@@H](N [R])CC1═CNC2═C 1C═CC═C2C3═C NN═C3)═O |
| | | C₁₄H₁₂N₄OR₂ | |
| 6 | 7(7(124TAZP))W | 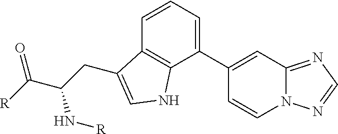 | [R]C([C@@H](N [R])CC1═CNC2═ C1C═CC═C2C3═ CC4═NC═NN4C═ C3)═O |
| | | C₁₇H₁₃N₅OR₂ | |
| 7 | 7(3UrPh)W | 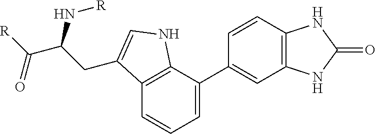 | [R]C([C@@H](N [R])CC1═CNC2═ C1C═CC═C2C3═C C(NC(N4)═O)═C4 C═C3)═O |
| | | C₁₈H₁₄N₄O₂R₂ | |
| 8 | 7(7Imzpy)W | C₁₈H₁₄N₄OR₂ | [R]C([C@@H](N [R])CC1═CNC2═ C1C═CC═C2C3═ CC4═NC═CN4C═ C3)═O] |
| 9 | 7(4OMePh)W | 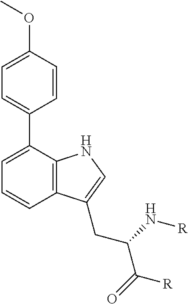 | [R]C([C@@H](N [R])CC1═CNC2═ C1C═CC═C2C3═ CC═C(OC)C═C3)═ O |
| | | C₁₈H₁₆N₂O₂R₂ | |
| 10 | 7(3(6AzaInd1Me))W | 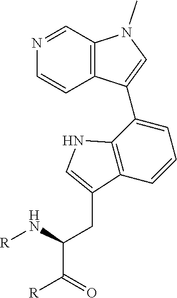 | [R]C([C@@H](N [R])CC1═CNC2═ C1C═CC═C2C3═ CN(C)C4═C3C═CN ═C4)═O |
| | | C₁₉H₁₆N₄OR₂ | |
| 11 | 7(6(2MeNDAZ))W | 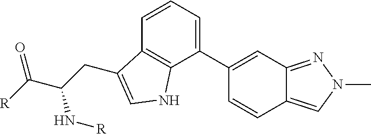 | [R]C([C@@H](N [R])CC1═CNC2═C 1C═CC═C2C3═C C4═NN(C)C═C4C ═C3)═O |
| | | C₁₉H₁₆N₄OR₂ | |
| 12 | NMebAla | 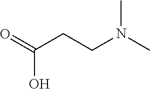 | CN(C)CCC═O |
| | | C₅H₁₁NO | |
| 13 | AcMorp, Ethyl- morpholino | 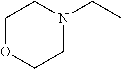 | CN1CCOCC1 |
| | | C₅H₁₁NO | |
| 14 | dOrn, D-Orn D-Ornithine | 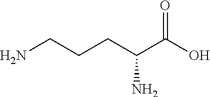 | NCCC[C@H](C(O) ═O)N |
| | | C₅H₁₂N₂O | |
| 15 | 3Hyp, 3-Hydroxy-L- proline | 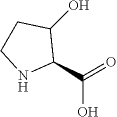 | OC1[c@@H](C═O) NCC1 |
| | | C₅H₉NO₂ | |
| 16 | aMeE aMeGlu, alpha- methyl glutamic acid | 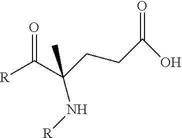 | C[C@](CCC(O)═O) (C([R])═O)N[R] C[c@](CCC(O)═O) (C═O)N |
| | | C₆H₁₁NO₂ | |
| 17 | hGlu, (S)-2- aminohexanedioic acid | 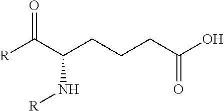 | N[C@@H](CCCC ═O)C═O OC(CCC[C@@H] (C([R])═O)N[R])═O |
| | | C₆H₁₁NO₂ | |
| 18 | CON(NMePip) | 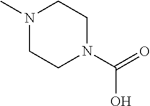 | CN(CC1)CCN1C═O |
| | | C₆H₁₂N₂O | |
| 19 | -CODiFPip, CO(DiFPip) | 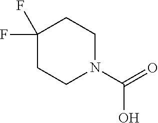 [SMILES: O=C(O)N1CCC(F)(F)CC1] | O═CN(CC1)CCC1 (F)F |
| | | C₆H₉F₂NO | |
| 20 | CO(OAZBO) | 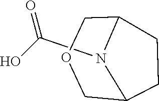 [SMILES: O=C(O)N1C2CCC1COC2] | CC(N1C2COCC1C C2)═O |
| | | C₈H₁₃NO₂ | |
| 21 | Me1Pya, (S)-3-(2- amino-2- carboxyethyl)-1- methylpyridin-1-ium | 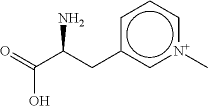 | C[n+]1cccc(C[C@ @H](C═O)N)c1 |
| | | C₉H₁₃N₂O⁺ | |
| 22 | DappF6, tetra-fluoro- phenylalanine | 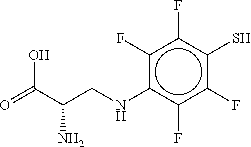 | N[C@@H](CNc(c (F)c(c(S)c1F)F)c1F) C═O |
| | | C₉H₈F₄N₂O | |
| 23 | bMePhe(2S,3R) bMePhe(SR), bMePhe(2S,3R) | 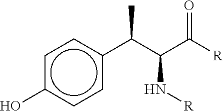 | C[C@@H]([[C@ @H](C([R])═O)N [R])c1ccccc1 |
| | | C₁₀H₁₁NOR₂ | |
| 24 | N4AmBenzylGly, N(4AmBenzyl)Gly | 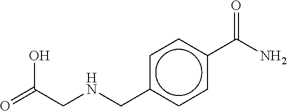 | NC(c1ccc(CNCC═ O)cc1)═O |
| | | C₁₀H₁₂N₂O₂ | |
| 25 | -Dec, 1,10- Decanedioic Acid | 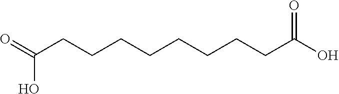 [SMILES: O=C(O)CCCCCCCCC(=O)O] | OC(CCCCCCCC C═O)═O |
| | | C₁₀H₁₈O₃ | |
| 26 | 2OH3Pyrimid5Ala | 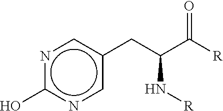 | CC(C)(C)Oc1ncc (C[c@@H](C([R]) ═O)N[R])cn1 |
| | | C₁₁H₁₅N₃O₂R₂ | |
| 27 | KacMorph, K(AcMorph), KAcMorph, L- Lysine(ac- Morpholino | 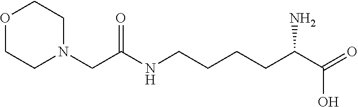 [SMILES: N[C@@H](CCCCNC(=O)CN1CCOCC1)C(=O)O] | N[C@@H](CCCC NC(CN1CCOCC1) ═O)C═O |
| | | C₁₂H₂₃N₃O₃ | |
| 28 | 6OH2Nal | 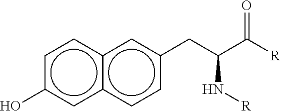 | N[C@@H](Cc1cc 2ccccc2cc1)C═O N[C@@H](Cc1cc 2ccccc2cc1)C═O Oc1ccc(cc(C[C@ @H](C([R])═O)N [R])cc2)c2c1 |
| | | C₁₃H₁₃NO | |
| 29 | DabNMecarn, Dab(NMecarn) | 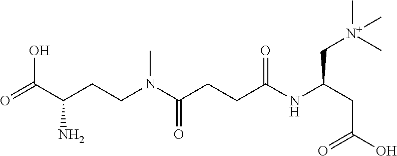 [SMILES: CN(CC[C@H](N)C(=O)O)C(=O)CCC(=O)N[C@H](CC(=O)O)C[N+](C)(C)C] | CN(CC[C@@H] (C═O)N)C(CCC(N [C@H](CC═O)C [N+](C)(C)C)═O)═O CN(CC[C@@H] (C═O)N)C(CCC(N [C@H](CC═O)C [N+](C)(C)C)═O)═O CN(CC[C@@H] (C═O)N)C(CCC(N |
| | | C₁₆H₃₁N₄0₄⁺ | [C@H](CC(O)═O) |
| | | | C[N+](C)(C)C)═O) |
| | | | ═O |
| 30 | DabNMeCarn, Dab(NMeCarn) | 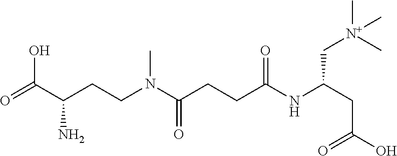 [SMILES: CN(CC[C@H](N)C(=O)O)C(=O)CCC(=O)N[C@@H](CC(=O)O)C[N+](C)(C)C] | CN(CC[C@@H] (C═O)N)C(CCC(N [C@@H](CC(O)═ O)C[N+](C)(C)C) ═O)═O |
| | | C₁₆H₃₁N₄O₅⁺ | |
| 31 | F(4TzlTMA4) | 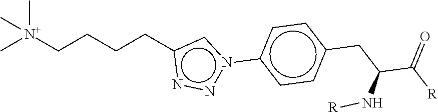 | C[N+](C)(C)CCC Cc1cn(- c2ccc(C[C@@H] (C([R])═O)N[R])cc 2)nn1 |
| | | C₁₈H₂₆N₅OR₂⁺ | |
| 32 | NMeK(d), NMeKdCar | 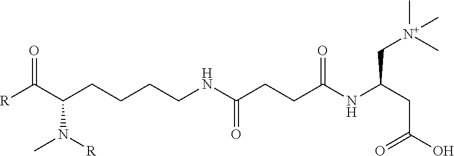 | CN([C@@H](CC CCNC(CCC(N[C @H](CC(O)═O)C [N+](C)(C)C)═O)═ O)C([R])═O)[R] |
| | | C₁₈H₃₃N₄O₅R₂⁺ | |
| 33 | 7(5(Ina7Pyr))W | 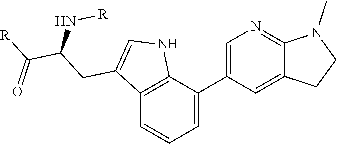 | [R]C([C@@H](N[ R])CC1═CNC2═C 1C═CC═C2C3═C N═C(N(C)CC4) C4═C3)═O |
| | | C₁₉H₁₈N₄OR₂ | |
| 34 | F(4TzlTMA5) | 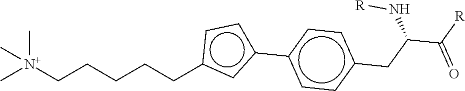 | C[N+](C)(C)CCC CCc1cn(- c2ccc(C[C@@H] (C([R])═O)N[R])cc 2)nn1 |
| | | C₁₉H₂₈N₅OR₂⁺ | |
| 35 | CF3CO F3CO | 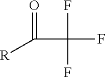 | O═C(C(F)(F)F)[R] |
| | | C₂F₃OR | |
| 36 | CF3Propylamide | 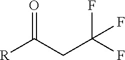 | O═C(CC(F)(F)F)[R] |
| | | C₃H₂F₃OR | |
| 37 | C(1*) (*pure but configuration unknown) | 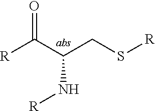 | O═C([C@H](CS[R]) N[R])[R] |
| | | C₃H₄NOSR₃ | |
| 38 | bAla, b-ALA, beta- Alanine, bA | 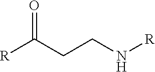 | O═C(CCN[R])[R] |
| | | C₃H₅NOR₂ | |
| 39 | CON(Me)2 | 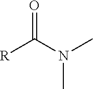 | CN(C)C([R])═O |
| | | C₃H₆NOR | |
| 40 | D(2) | 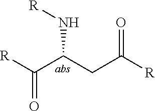 | O═C(C[c@@H](C ([R])═O)N[R])[R] |
| | | C₄H₄NO₂R₃ | |
| 41 | cPrCO | 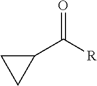 | O═C(C1CC1)[R] |
| | | C₄H₅OR | |
| 42 | hS, hS, , Hse, L- homoserine, homoS, or homoSer | 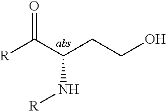 | OCC[C@@H](C ([R])═O)N[R] |
| | | C₄H₇NO₂R₂ | |
| 43 | T, dThr, dT | 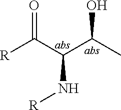 | C[C@H]([C@H](C ([R])═O)N[R])O |
| | | C₄H₇NO₂R₂ | |
| 44 | 4sb, 4SB | 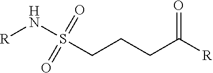 | O═C([R])CCCS(═ O)(N[R])═O |
| | | C₄H₇NO₃SR₂ | |
| 45 | Aib, AIB, 2- Aminoisobutyric acid, Alpha- aminoisobutyric acid, (2-aminoalanine) | 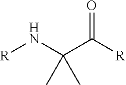 | CC(C)(C([R])═O)N [R] |
| | | C₄H₇NOR₂ | |
| 46 | | | |
| 47 | NMebAla | 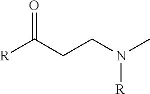 | CN(CCC([R])═O)[R] |
| | | C₄H₇NOR₂ | |
| 48 | aMeC | 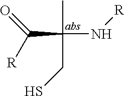 | C[C@](CS)(C([R]) ═O)N[R] C[C@](CS)(C═O) N |
| | | C₄H₇NOSR₂ | |
| 49 | hC, hCys, homoC, or homoCys | 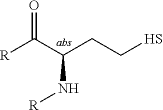 | O═C([C@H](CCS) N[R])[R] |
| | | C₄H₇NOSR₂ | |
| 50 | iPrCO | 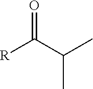 | CC(C)C([R])═O |
| | | C₄H₇OR | |
| 51 | dDab, dab, (R)-2,4- diaminobutanoic acid | 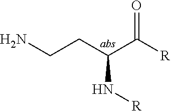 | NCC[C@H](C([R]) ═O)N[R] |
| | | C₄H₈N₂OR₂ | |
| 52 | homobAla | 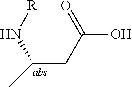 | C[c@@H](CC(O) ═O)N[R] |
| | | C₄H₈NO₂R | |
| 53 | Bua, Butanoic acid | 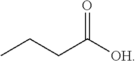 | CCCC(O)═O |
| | | C₄H₈O₂ | |
| 54 | Orn, ORN, Ornithine | 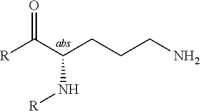 | NCCC[C@@H](C ([R])═O)N[R] |
| | | C₅H₁₀N₂OR₂ | |
| 55 | | | |
| 56 | Orn, L-ornithine | 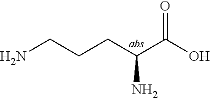 | NCCC[C@@H](C (O)═O)N |
| | | C₅H₁₂N₂O₂ | |
| 57 | 4diFPro | 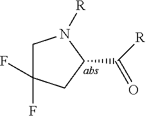 | O═C([C@H](CC(C 1)(F)F)N1[R])[R] O═C[C@H](C1)N CC1(F)F |
| | | C₅H₅F₂NOR₂ | |
| 58 | prG, prG, Fmoc-L- propargyl-Gly-OH, Pra | 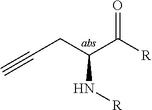 | C#CC[C@@H](C ([R])═O)N[R] |
| | | C₅H₅NOR₂ | |
| 59 | 4TriazolAla | 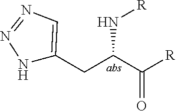 | O═C([C@H](Cc1c nn[nH]1)N[R])[R] |
| | | C₅H₆N₄OR₂ | |
| 60 | Tzl | 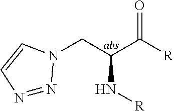 | O═C([C@H](CnIn ncc1)N[R])[R] |
| | | C₅H₆N₄OR₂ | |
| 61 | PyE, PyE (S)-5-oxopyrrolidine- 2-carboxylic acid | 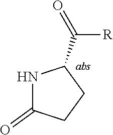 | O═C([C@H](CC1) NC1═O)[R] O═C[C@H](CC1) NC1═O |
| | | C₅H₆NO₂R | |
| 62 | E(2) | 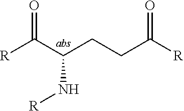 | O═C(CC[c@@H] (C([R])═O)N[R])[R] |
| | | C₅H₆NO₂R₃ | |
| 63 | Tetrazole | 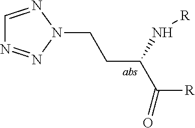 | O═C([C@H](CCn1 nncn1)N[R])[R] N[C@@H](CCn1n ncn1)C═O |
| | | C₅H₇N₅OR₂ | |
| 64 | 3OHPro | 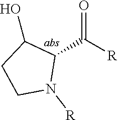 | OC(CC1)[C@@H] (C([R])═O)N1[R] |
| | | C₅H₇NO₂R₂ | |
| 65 | 4(R)HydroxyPro | 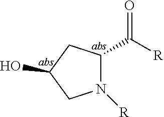 | O[C@H](C[C@H] 1C([R])═O)CN1[R] |
| | | C₅H₇NO₂R₂ | |
| 66 | Hyp | 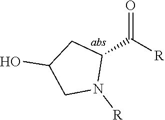 | OC(C[C@H]1C([R 1)═O)CN1[R] |
| | | C₅H₇NO₂R₂ | |
| 67 | AllylGly | 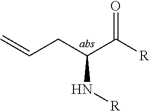 | C═CC[c@@H](C ([R])═O)N[R] |
| | | C₅H₇NOR₂ | |
| 68 | Dap(Ac) | 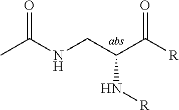 | CC(NC[C@@H](C ([R])═O)N[R])═O |
| | | C₅H₈N₂O₂R₂ | |
| 69 | N(NMe), NNMe, NMeAsn | 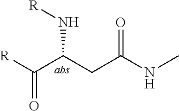 | CNC(C[C@@H](C ([R])═O)N[R])═O |
| | | C₅H₈N₂O₂R₂ | |
| 70 | aMeN•aMeAsn | 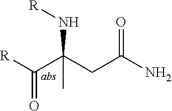 | C[C@](CC(N)═O) (C([R])═O)N[R] |
| | | C₅H₈N₂O₂R₂ | |
| 71 | 4(S)AminoPro | 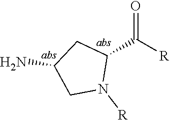 | N[C@@H](C[C@ H]1C([R])═O)CN1 [R] |
| | | C₅H₈N₂OR₂ | |
| 72 | CO(Morph) | 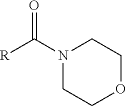 | O═C(N1CCOCC1) [R] |
| | | C₅H₈NO₂R | |
| 73 | -COMorph, CO(Morph) | 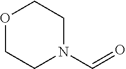 | O═CN1CCOCC1 |
| | | C₅H₉NO₂ | |
| 74 | | | |
| 75 | Nva | 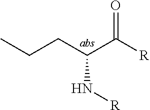 | CCC[C@@H](C ([R])═O)N[R] |
| | | C₅H₉NOR₂ | |
| 76 | dM, dMet, D- Methionine | 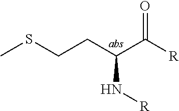 | CSCC[C@H](C([R]) ═O)N[R] |
| | | C₅H₉NOSR₂ | |
| 77 | dPen, pen | 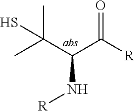 | CC(C)([C@H](C ([R])═O)N[R])S |
| | | C₅H₉NOSR₂ | |
| 78 | BuCO | 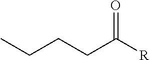 | CCCCC([R])═O |
| | | C₅H₉OR | |
| 79 | iBuCO | 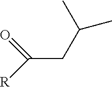 | CC(C)CC([R])═O CC[C@H](C)C([R]) ═O |
| | | C₅H₉OR | |
| 80 | tBuCO | 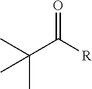 | CC(C)(C)C([R])═O |
| | | C₅H₉OR | |
| 81 | N(N(Me)2), NNMe2 | 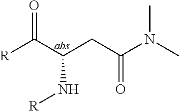 | CN(C)C(C[C@@H] (C([R])═O)N[R])═O |
| | | C₆H₁₀N₂O₂R₂ | |
| 82 | MorphCO, 2- morpholinoacetic acid | 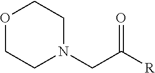 | O═C(CNICCOCC1) [R] |
| | | C₆H₁₀NO₂R | |
| 83 | CON(NMePip) | 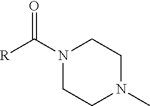 | CN(CC1)CCN1C ([R])═O |
| | | C₆H₁₁N₂OR | |
| 84 | eK | 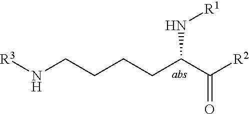 | O═C(O)[C@@H] (N[R])CCCCN[R] |
| | | C₆H₁₁N₂OR₃ | |
| 85 | Cit, Citrulline | 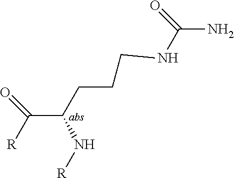 | NC(NCCC[C@@H] (C([R])═O)N[R])═O N[C@@H](CCCN C(N)═O)C(O)═O |
| | | C₆H₁₁N₃O₂R₂ | |
| 86 | D(NEtNH2) | 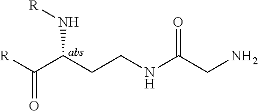 | NCCNC(C[C@@H] (C([R])═O)N[R])═O |
| | | C₆H₁₁N₃O₂R₂ | |
| 87 | Aad, 2-Aminoadipic acid | 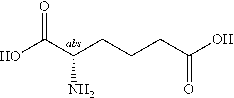 [SMILES: N[C@@H](CCCC(=O)O)C(=O)O] | N[C@@H](CCCC (O)═O)C(O)═O |
| | | C₆H₁₁NO₄ | |
| 88 | N(Isobutyl)Gly | 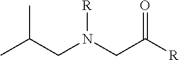 | CC(C)CN(CC([R]) ═O)[R] |
| | | C₆H₁₁NOR₂ | |
| 89 | PentCO | 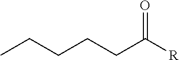 | CCCCCC([R])═O |
| | | C₆H₁₁OR | |
| 90 | NMeQ, NMeGln, N- Methyl-Glutamine | 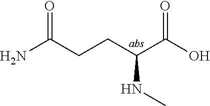 | CN[C@@H](CCC (N)═O)C(O)═O |
| | | C₆H₁₂N₂O₃ | |
| 91 | SP6 | 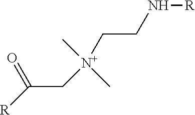 | C[N+](C)(CCN[R]) CC([R])═O C[N+](C)(CCN)CC═O |
| | | C₆H₁₃N₂OR₂⁺ | |
| 92 | 3IOxa4Ala | 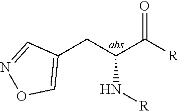 | O═C([C@H](Cc1c onc1)N[R])[R] |
| | | C₆H₆N₂O₂R₂ | |
| 93 | 3Oxa4Ala | 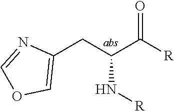 | O═C([C@H](Cc1c ocn1)N[R])[R] |
| | | C₆H₆N₂O₂R₂ | |
| 94 | diFCpx | 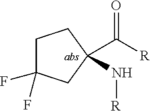 | O═C([C@](CC1)(C C1(F)F)N[R])[R] |
| | | C₆H₇F₂NOR₂ | |
| 95 | aMePra | 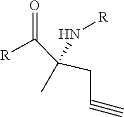 | C[C@](CC#C)(C ([R])═O)N[R] |
| | | C₆H₇NOR₂ | |
| 96 | CO(DiFPip) | 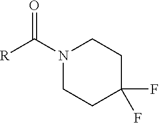 | O═C(N(CC1)CCC1 (F)F)[R] |
| | | C₆H₈F₂NOR | |
| 97 | dab(COCH2(1*)) dab(COCH2)(1*) | 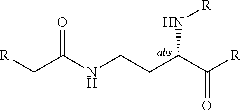 | O═C(C[R])NCC[C @@H](C([R])═O) N[R] |
| | | C₆H₉N₂O₂R₃ | |
| 98 | Tetrazole(NMe) | 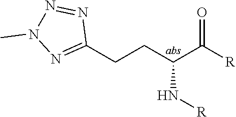 | Cnlnnc(CC[C@@ H](C([R])═O)N[R]) n1 |
| | | C₆H₉N₅OR₂ | |
| 99 | | | |
| 100 | dhE | 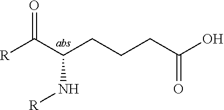 | OC(CCC[C@H](C ([R])═O)N[R])═O |
| | | C₆H₉NO₃R₂ | |
| 101 | Acpx | 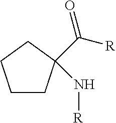 | O═C(C1(CCCC1)N [R])[R] NC1(CCCC1)C═O |
| | | C₆H₉NOR₂ | |
| 102 | aMeP, aMePro | 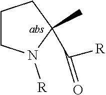 | C[c@](CCC1)(C ([R])═O)N1[R] |
| | | C₆H₉NOR₂ | |
| 103 | D(N2AmIm) | 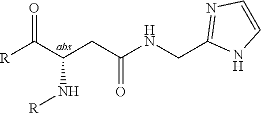 | O═C(C[c@@H](C ([R])═O)N[R])NCc 1ncc[nH]1 |
| | | C₈H₁₀N₄O₂R₂ | |
| 104 | KTfa, K(Tfa), L- Lys(Tfa) | 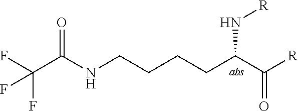 | O═C([C@H](CCC CNC(C(F)(F)F)═O) N[R])[R] |
| | | C₈H₁₁F₃N₂O₂R₂ | |
| 105 | E(OAll) | 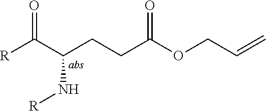 | C═CCOC(CC[C@ @H](C([R])═O)N [R])═O |
| | | C₈H₁₁NO₃R₂ | |
| 106 | D(NPyr) | 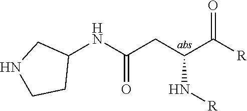 | O═C(C[C@@H](C ([R])═O)N[R])NC1 CNCC1 |
| | | C₈H₁₃N₃O₂R₂ | |
| 107 | Chg | 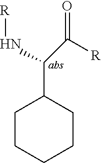 | O═C([C@H](C1C CCCC1)N[R])[R] |
| | | C₈H₁₃NOR₂ | |
| 108 | R5Me, aMeR5H | 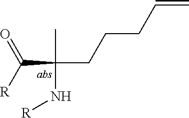 | C[C@@](CCCC═C C([R])═O)N[R] |
| | | C₈H₁₃NOR₂ | |
| 109 | R6H, (R,E)-2-amino- 8-hydroxyoct-7-enoic acid | 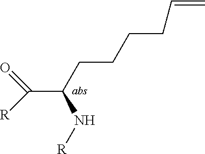 | C═CCCCC[C@H] (C([R])═O)N[R] C═CCCCC[C@H] (C═O)N |
| | | C₈H₁₃NOR₂ | |
| 110 | S5Me aMeS5H | 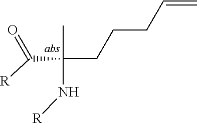 | C[C@](CCCC═C) (C([R])═O)N[R] |
| | | C₈H₁₃NOR₂ | |
| 111 | S6H | 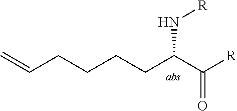 | C═CCCCC[C@@ H](C([R])═O)N[R] |
| | | C₈H₁₃NOR₂ | |
| 112 | KAc, K(Ac), K(COMe), K-Ac, N6-acetyl-L-Lysine | 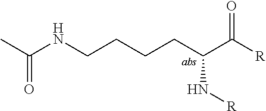 | CC(NCCCC[C@@ H](C([R])═O)N[R]) ═O |
| | | C₈H₁₄N₂O₂R₂ | |
| 113 | Pip(NMe2) | 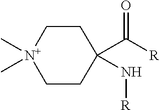 | C[N+](C)(CC1)CC C1(C([R])═O)N[R] |
| | | C₈H₁₅N₂OR₂⁺ | |
| 114 | K(Gly) | 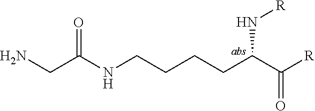 | NCC(NCCCC[C@ @H](C([R])═O)N [R])═O |
| | | C₈H₁₅N₃O₂R₂ | |
| 115 | 8Aoc, 8Aoc(2) | 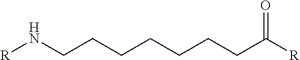 | O═C(CCCCCCCN [R])[R] |
| | | C₈H₁₅NOR₂ | |
| 116 | 2Benzyl | 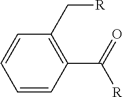 | O═C(c1c(C[R])ccc c1)[R] |
| | | C₈H₆OR₂ | |
| 117 | 6OH3Pya | 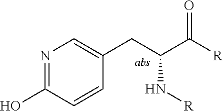 | Oc1ncc(C[C@@H] (C([R])═O)N[R])cc1 |
| | | C₈H₃N₂O₂R₂ | |
| 118 | 3Pya, 3Pal, 3-(2- pyridyl)-alanine | 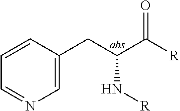 | O═C([C@H](Cc1c nccc1)N[R])[R] |
| | | C₈H₈N₂OR₂ | |
| 119 | 4Pya, 4Pya, 4Pal, (S)- 2-amino-3-(pyridin- 4-yl)propanoic acid 4PyridinAla | 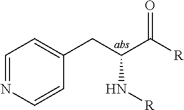 | O═C([C@H](Cc1c cncc1)N[R])[R] |
| | | C₈H₈N₂OR₂ | |
| 120 | dPal, dpal, d3Pya, 3pya, 3- pyridylalanine, (R)-2- amino-3-(pyridin-3- yl)propanoic acid | 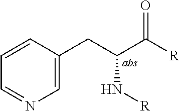 | O═C([C@@H](Cc 1cnccc1)N[R])[R] |
| | | C₈H₈N₂OR₂ | |
| 121 | 6MePyridazAla | 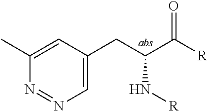 | Cc1cc(C[C@@H] (C([R])═O)N[R])cn n1 |
| | | C₈H₉N₃OR₂ | |
| 122 | 5MePyridinAla | 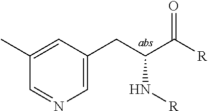 | Cc1cc(C[C@@H] (C([R])═O)N[R])cnc1 |
| | | C₉H₁₀N₂OR₂ | |
| 123 | J, Aph, 4- aminophenylalanine | 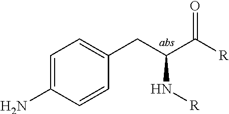 | Nc1ccc(C[C@@H] (C([R])═O)N[R])cc1 |
| | | C₉H₁₀N₂OR₂ | |
| 124 | NMe3Pya | 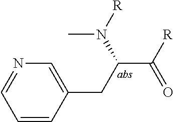 | CN([C@@H](Cc1c nccc1)C([R])═O)[R] CN[C@@H](Cc1c nccc1)C═O |
| | | C₉H₁₀N₂OR₂ | |
| 125 | SMSBCO | 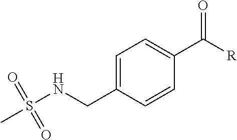 | CS(NCc(cc1)ccc1C ([R])═O)(═O)═O |
| | | C₉H₁₀NO₃SR | |
| 126 | Me3Pya | 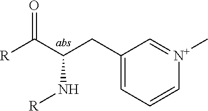 | C[n+]1cccc(C[C@ @H](C([R])═O)N [RT)c1 |
| | | C₉H₁₁N₂OR₂⁺ | |
| 127 | D(Pip), (S)-2-amino- 4-oxo-4-(piperidin-1- yl)butanoic acid | 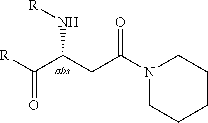 | O═C(C[C@@H](C ([R])═O)N[R])N1C CCCC1 |
| | | C₉H₁₄N₂O₂R₂ | |
| 128 | D(NPip) | 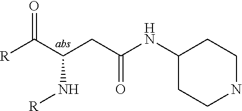 | O═C(C[C@@H](C ([R])═O)N[R])NC1 CCNCC1 |
| | | C₉H₁₅N₃O₂R₂ | |
| 129 | N(Cyclohexyl)Gly | 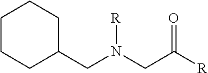 | O═C(CN(CC1CCC CC1)[R])[R] |
| | | C₉H₁₅NOR₂ | |
| 130 | R7H, (R,E)-2-amino- 9-hydroxynon-8- enoic acid | 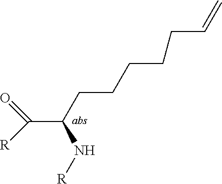 | C═CCCCCC[C@H]) ([R])═O)N[R] C═CCCCCC[C@H] (C═O)N |
| | | C₉H₁₅NOR₂ | |
| 131 | K(COEt) | 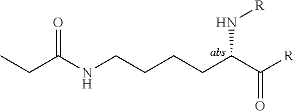 | CCC(NCCCC[C@ @H](C([R])═O)N [R])═O |
| | | C₉H₁₆N₂O₂R₂ | |
| 132 | K(NMeAc), KNMeAc | 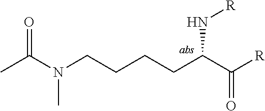 | CC(N(C)CCCC[C @@H](C([R])═O) N[R])═O |
| | | C₉H₁₆N₂O₂R₂ | |
| 133 | Q(NHtBu) | 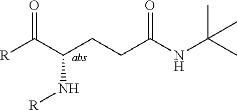 | CC(C)(C)NC(CC[C @@H](C([R])═O) N[R])═O |
| | | C₉H₁₆N₂O₂R₂ | |
| 134 | K(Me)3 | 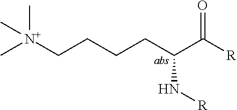 | C[N+](C)(C)CCCC [C@@H](C([R])═ O)N[R] |
| | | C₉H₁₉N₂OR₂⁺ | |
| 135 | dK(Me)3, k(Me)3 | 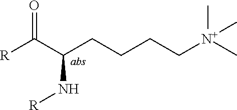 | C[N+](C)(C)CCCC [C@H](C([R])═O) N[R] |
| | | C₉H₁₉N₂OR₂⁺ | |
| 136 | 5cpaCO | 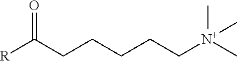 | C[N+](C)(C)CCCC CC([R])═O |
| | | C₉H₁₉NOR⁺ | |
| 137 | tetraFPhe | 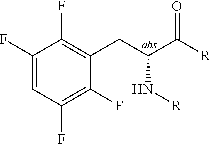 | O═C([C@H](Cc(c (F)c(cc1F)F)c1F)N [R])[R] |
| | | CH₅F₄NOR₂ | |
| 138 | 5CF33Pya | 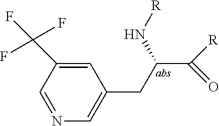 | O═C([C@H](Cc1c ncc(C(F)(F)F)c1)N [R])[R] N[C@@H](Cc1cc (C(F)(F)F)cnc1)C═O |
| | | C₉H₇F₃N₂OR₂ | |
| 139 | 3,4diFPhe, 4diFPhe | 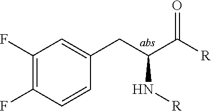 | O═C([C@H](Cc(cc 1)cc(F)c1F)N[R]) [R] |
| | | C₉H₇F₂NOR₂ | |
| 140 | F(4N3) | 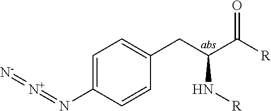 | [N-]] ═[N+]═Nc1ccc(C [C@@H](C([R])═O) N[R])cc1 |
| | | C₉H₈N₄OR₂ | |
| 141 | 3FTyr | 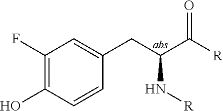 | Oc(ccc(C[C@@H] (C([R])═O)N[R])c1) c1F |
| | | C₉H₈FNO₂R₂ | |
| 142 | 2BrPhe, 2BrF | 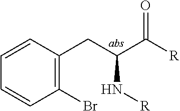 | O═C([C@H](Cc(cc cc1)c1Br)N[R])[R] |
| | | C₉H₈BrNOR₂ | |
| 143 | 2FPHE, 2FPhe | 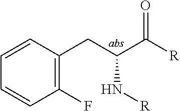 | O═C([C@H](Cc(cc cc1)c1F)N[R])[R] |
| | | C₉H₈FNOR₂ | |
| 144 | 3FPHE, 3FPhe | 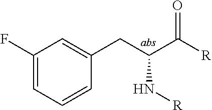 | O═C([C@H](Cc1c c(F)ccc1)N[R])[R] |
| | | C₉H₈FNOR₂ | |
| 145 | BHCO | 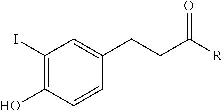 | Oc(ccc(CCC([R])═ O)c1)c1I |
| | | C₉H₈IO2_{R} | |
| 146 | 5AmPyridinAla | 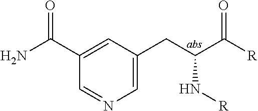 | NC(c1cc(C[C@@ H](C([R])═O)N[R]) cnc1)═O |
| | | CH₉N₃O₂R₂ | |
| 147 | mTYR, mY, mTyr | 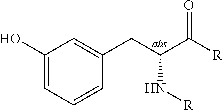 | Oc1cccc(C[C@@H] (C([R])═O)N[R])c1 |
| | | C₉H₉NO₂R₂ | |
| 148 | 6OHQuin | 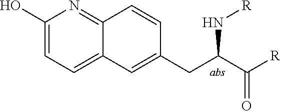 | Oc1ccc(cc(C[C@ @H](C([R])═O)N [R])cc2)c2n1 |
| | | C₁₂H₁₀N₂O₂R₂ | |
| 149 | 4AmF, 4AmPhe | 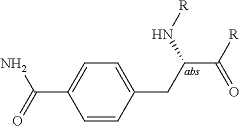 | NC(c1ccc(C[C@ @H](C([R])═O)N R])cc1)═O N[C@@H](Cc(cc 1)ccc1C(N)═O)C═O |
| | | C₁₀H₁₀N₂O₂R₂ | |
| 150 | AEF(NMe(2)) | 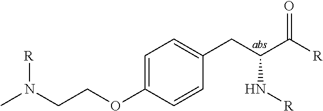 | CN(CCOc1ccc(C[ c@@H](C([R])═ O)N[R])cc1)[R] |
| | | C₁₂H₁₅N₂O₂R₃ | |
| 151 | aMeY01 | 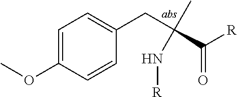 | C[c@](Cc(cc1)cc c1OC)(C([R])═O) N[R] |
| | | C₁₁H₁₃NO₂R₂ | |
| 152 | BiF | 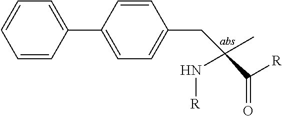 | C[C@](Cc(cc1)cc c1- c1ccccc1)(C([R])═ O)N[R] |
| | | C₁₆H₁₅NOR₂ | |
| 153 | hdKMe3, hk(Me)3 | 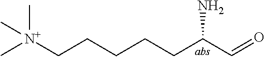 | C[N+](C)(C)CCC CC[C@H](C═O)N |
| | | C₁₀H₂₃N₂O⁺ | |
| 154 | Y(OTzl) | 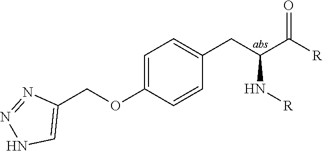 | O═C([C@H](Cc(c c1)ccc1OCc1c[nH] nn1)N[R])[R] |
| | | C₁₂H₁₂N₄O₂R₂ | |
| 155 | 3CONH2F | 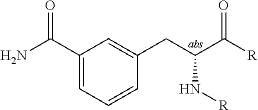 | NC(c1cccc(C[C@ @H](C([R])═O)N [R])c1)═O |
| | | C₁₀H₁₀N₂O₂R₂ | |
| 156 | 4AmDF, 4AmDPhe | 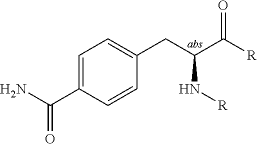 | NC(c1ccc(C[C@H] (C([R])═O)N[R]) cc1)═O |
| | | C₁₀H₁₀N₂O₂R₂ | |
| 157 | 4AmF, 4AmPhe | 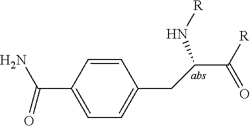 | NC(c1ccc(C[C@ @H](C([R])═O)N [R])cc1)═O N[C@@H](Cc(cc 1)ccc1C(N)═O)C═O |
| | | C₁₀H₁₀N₂O2R₂ | |
| 158 | D(NPh) | 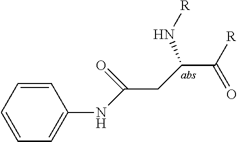 | O═C(C[C@@H] (C([R])═O)N[R])N c1ccccc1 |
| | | C₁₀H₁₀N₂O₂R₂ | |
| 159 | N(3AmBenzyl)Gly | 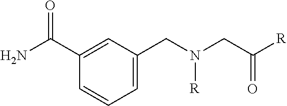 | NC(c1cccc(CN(C C([R])═O)[R])c1) ═O |
| | | C₁₀H₁₀N₂O₂R₂ | |
| 160 | N(4AmBenzyl)Gly | 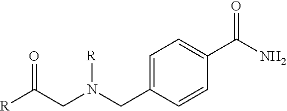 | NC(c1ccc(CN(CC ([R])═O)[R])cc1)═O |
| | | C₁₀H₁₀N₂O₂R₂ | |
| 161 | 2AmTyr | 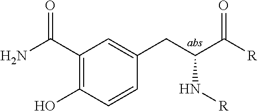 | NC(c(cc(C[C@@ H](C([R])═O)N[R 1)cc1)c1O)═O |
| | | C₁₀H₁₀N₂O₃R₂ | |
| 162 | aMeFPhe | 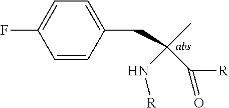 | C[C@](Cc(cc1)cc c1F)(C([R])═O)N [R] |
| | | C₁₀H₁₀FNOR₂ | |
| 163 | D(NmAn) | 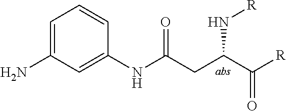 | Nc1cccc(NC(C[C @@H](C([R])═O) N[R])═O)c1 |
| | | C₁₀H₁₁N₃O₂R₂ | |
| 164 | D(NoAn) | 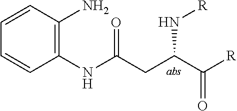 | Nc(cccc1)c1NC(C [C@@H](C([R])═ O)N[R])═O |
| | | C₁₀H₁₁N₃O₂R₂ | |
| 165 | D(NpAn) | 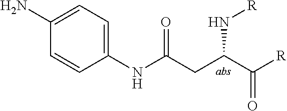 | Nc(cc1)cccINC(C [C@@H](C([R])═ O)N[R])═O |
| | | C₁₀H₁₁N₃O₂R₂ | |
| 166 | 4MeOF | 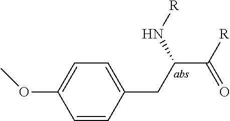 | COc1ccc(C[C@@ H](C([R])═O)N[R 1)cc1 COc1ccc(C[C@@ H](C═O)N)cc1 |
| | | C₁₀H₁₁NO₂R₂ | |
| 167 | NMeDTyr, NMeDY, NMedTyr, NMedY, N-Methyl-D-tyrosine, dNMeTyr dNMeY | 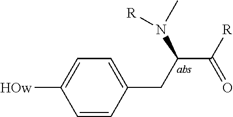 | CN([C@H](Cc(cc 1)ccc10)C([R])═ O)[R] |
| | | C₁₀H₁₁NO₂R₂ | |
| 168 | aMe3OHPhe | 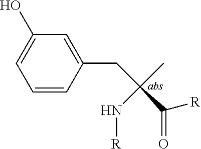 | C[c@](Cc1cc(O)c cc1)(C([R])═O)N [R] |
| | | C₁₀H₁₁NO₂R₂ | |
| 169 | aMeY, aMeTyr | 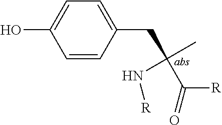 | C[C@](Cc(cc1)cc c10)(C([R])═O)N [R] |
| | | C₁₀H₁₁NO₂R₂ | |
| 170 | bMeDTyr(2R3S) bMeDTyr(2R,3S) | 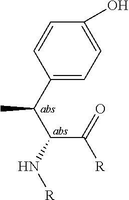 | C[C@H]([C@H] (C([R])═O)N[R])c (cc1)ccc1O |
| | | C₁₀H₁₁NO₂R₂ | |
| 171 | 4MeF | 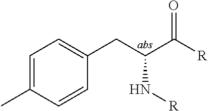 | Cc1ccc(C[C@@H] (C([R])═O)N[R]) cc1 |
| | | C₁₀H₁₁NOR₂ | |
| 172 | aMeF, aMeF alpha-methyl phenylalanine | 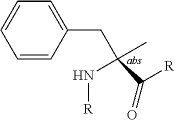 | C[c@](Cc1ccccc1) (C([R])═O)N[R] C[c@](Cc1ccccc1) (C═O)N |
| | | C₁₀H₁₁NOR₂ | |
| 173 | bMePhe | 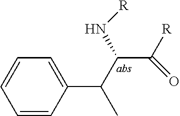 | CC([C@@H](C ([R])═O)N[R])c1ccc cc1 |
| | | C₁₀H₁₁NOR₂ | |
| 174 | bMePhe(2S3S) bMePhe(2S,3S) | 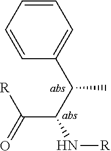 | C[C@H]([C@@H] (C([R])═O)N[R]) c1ccccc1 |
| | | C₁₀H₁₁NOR₂ | |
| 175 | hF, hPhe, homoF, homoPhe | 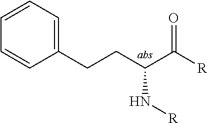 | O═C([C@H](CCc 1ccccc1)N[R])[R] |
| | | C₁₀H₁₁NOR₂ | |
| 176 | F4CONH2, 4- carbamoyl-L- phenylalanine | 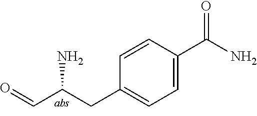 [SMILES: NC(=O)c1ccc(C[C@@H](N)C=O)cc1] | N[C@@H](Cc(cc 1)ccc1C(N)═O)C═O |
| | | C₁₀H₁₂N₂O₂ | |
| 177 | Maf | 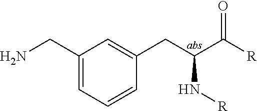 | NCc1cccc(C[C@ @H](C([R])═O)N [RD)c1 |
| | | C₁₀H₁₂N₂OR₂ | |
| 178 | Paf | 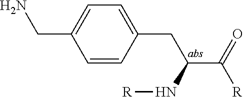 | NCc1ccc(C[C@@ H](C([R])═O)N[R 1)cc 1 NCc1ccc(C[C@@ H](C═O)N)cc1 |
| | | C₁₀H₁₂N₂OR₂ | |
| 179 | dMaf, maf | 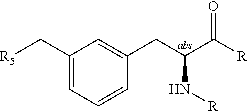 | NCc1cccc(C[C@ H](C([R])═O)N[R 1)c1 |
| | | C₁₀H₁₂N₂OR₂ | |
| 180 | dPaf | 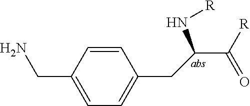 | NCc1ccc(C[C@H] (C([R])═O)N[R])c c1 |
| | | C₁₀H₁₂N₂OR₂ | |
| 181 | oAMPhe | 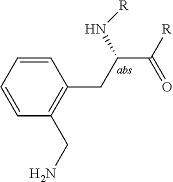 | NCc1c(C[C@@H] (C([R])═O)N[R]) cccc1 |
| | | C₁₀H₁₂N₂OR₂ | |
| | F(G) | 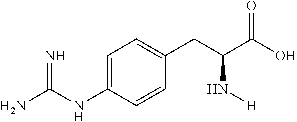 [SMILES: N=C(N)Nc1ccc(C[C@H](N)C(=O)O)cc1] | OC([C@@H](N[ H])CC1═CC═C(C═ C1)NC(N)═N)═O |
| | | (S)-2-amino-3-(4-guanidinophenyl)propanoic acid | |
| 182 | F(4G) | 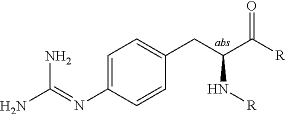 | NC(N)═Nc1ccc(C [C@@H](C([R])═ O)N[R])cc1 |
| | | C₁₀H₁₂N₄OR₂ | |
| 183 | NMeDTyr | 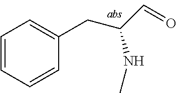 | CN[C@H](Cc1ccc cc1)C═O |
| | | C₁₀H₁₃NO | |
| 184 | dNMeTyr dNMeY, D-N-methyl tyrosine N-Methyl-D-tyrosine | 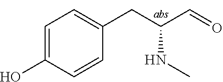 | CN[C@H](Cc(cc1) ccc10)C═O |
| | | C₁₀H₁₃NO₂ | |
| 185 | biotin | 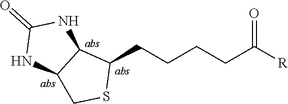 | O═C(CCCC[c@ @H]([C@H]IN2) SC[c@@H]1NC2═ O)[R] |
| | | C₁₀H₁₅N₂O₂SR | |
| 186 | K(CO2allyl) | 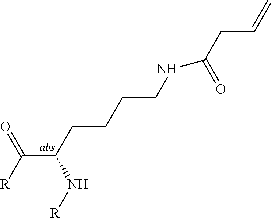 | C═CCC(NCCCC[ C@@H](C([R])═ O)N[R])═O C═CCOC(NCCC C[c@@H](C([R])═ O)N[R])═O |
| | | C₁₀H₁₆N₂O₂R₂ | |
| 187 | K(COcPr) | 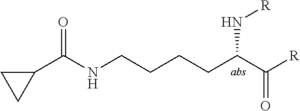 | O═C([C@H](CCC CNC(C1CC1)═O) N[R])[R] |
| | | C₁₀H₁₆N₂O₂R₂ | |
| 188 | DAGSuc | 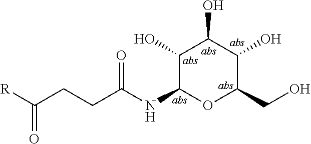 | OC[C@H]([C@H] ([C@@H]([C@H] 1O)O)O)O[C@H] 1NC(CCC([R])═ O)═O |
| | | C₁₀H₁₆NO₇R | |
| 189 | K(COPr) | 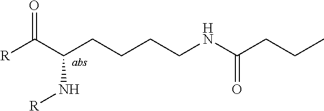 | CCCC(NCCCC[C @@H](C([R])═O) N[R])═O |
| | | C₁₀H₁₈N₂O₂R₂ | |
| 190 | K(COiPr) | 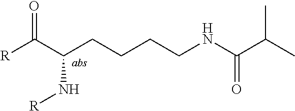 | CC(C)C(NCCCC [C@@H](C([R])═ O)N[R])═O |
| | | C₁₀H₁₈N₂O₂R₂ | |
| 191 | Tzl(Ch) | 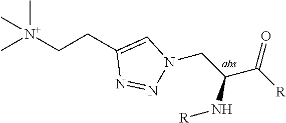 | C[N+](C)(C)CCc1 cn(C[C@@H](C ([R])═O)N[R])nn1 |
| | | C₁₀H₁₈N₅OR₂⁺ | |
| 192 | hK(Me)3, hKMe3 | 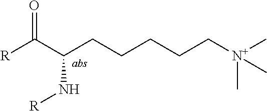 | C[N+](C)(C)CCC CC[c@@H](C([R])═ O)N[R] |
| | | C₁₀H₂₁N₂OR₂⁺ | |
| 193 | hdK(Me)3, hk(Me)3, hdKMe3 | 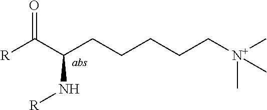 | C[N+](C)(C)CCC CC[C@H](C([R])═ O)N[R] |
| | | C₁₀H₂₁N₂OR₂⁺ | |
| 194 | Dap(pF(6)) | 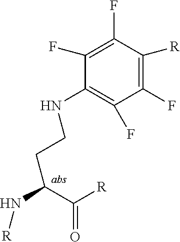 | O═C([C@H](CCN c(c(F)c(c([R])c1F) F)c1F)N[R])[R] |
| | | C₁₀H₇F₄N₂OR₃ | |
| 195 | 40CF3DPhe | 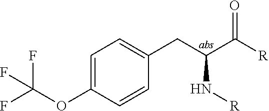 | O═C([C@@H](Cc (cc1)ccc1OC(F)(F) F)N[R])[R] |
| | | C₁₀H₈F₃NO₂R₂ | |
| 196 | CF3F | 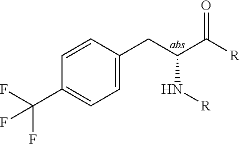 | O═C([C@H](Cc1c cc(C(F)(F)F)cc1) N[R])[R] |
| | | C₁₀H₈F₃NOR₂ | |
| 197 | 7AzaW | 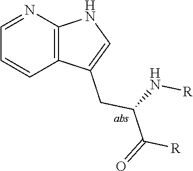 | O═C([C@H](Cc1c [nH]c2c1cccn2)N [R])[R] |
| | | C₁₀H₉N₃OR₂ | |
| 198 | Y(CHF2) | 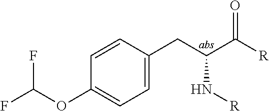 | O═C([C@H](Cc(c c1)ccc1OC(F)F)N [R])[R] |
| | | C₁₀H₉F₂NO₂R₂ | |
| 199 | CXF | 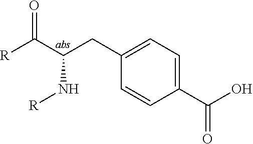 | OC(c1ccc(C[C@ @H](C([R])═O)N [R])cc1)═O |
| | | C₁₀H₉NO₃R₂ | |
| 200 | CHF2Phe | 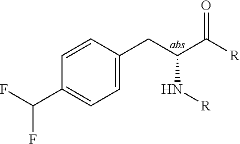 | O═C([C@H](Cc1c cc(C(F)F)cc1)N[R D[R] |
| | | C₁₀H₉F₂NOR₂ | |
| 201 | TetraFAEF | 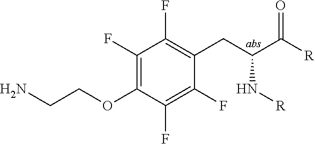 | NCCOc(c(F)c(c(C [C@@H](C([R])═ O)N[R])c1F)F)c1 F |
| | | C₁₁H₁₀F₄N₂O₂R₂ | |
| 202 | 50HW | 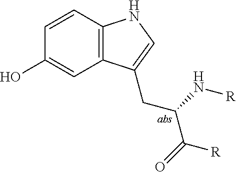 | Oc(cc1)cc2c1[nH] cc2C[C@@H](C ([R])═O)N[R] |
| | | C₁₁H₁₀N₂O₂R₂ | |
| 203 | 4AcDPhe | 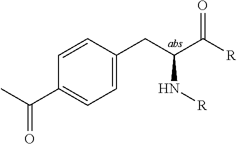 | CC(c1ccc(C[C@H] (C([R])═O)N[R]) cc1)═O |
| | | C₁₁H₁₁NO₂R₂ | |
| 204 | D(NBzl) | 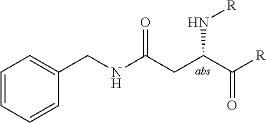 | O═C(C[c@@H] (C([R])═O)N[R])N Cc1ccccc1 |
| | | C₁₁H₁₂N₂O₂R₂ | |
| 205 | aMe2AmTyr | 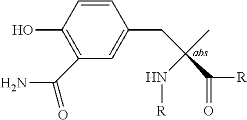 | C[C@](Cc(cc1)cc (C(N)═O)c1O)(C ([R])═O)N[R] |
| | | C₁₁H₁₂N₂O₃R₂ | |
| 206 | psiW | 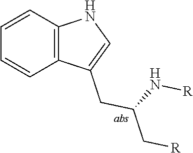 | [R]C[C@H](Cc1c [nH]c2c1cccc2)N [R] |
| | | C₁₁H₁₂N₂R₂ | |
| 207 | aMeY01 | 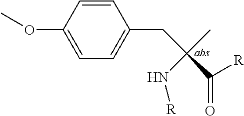 | C[C@](Cc(cc1)cc c1OC)(C([R])═O) N[R] |
| | | C₁₁H₁₃NO₂R₂ | |
| 208 | 3OMeY01 | 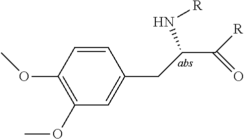 | COc(ccc(C[C@@ H](C([R])═O)N[R 1)c1)c1OC |
| | | C₁₁H₁₃NO₃R₂ | |
| 209 | | | |
| 210 | dAEF | 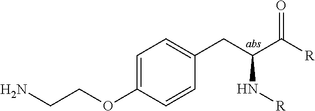 | NCCOc1ccc(C[C @H](C([R])═O)N [R])cc1 |
| | | C₁₁H₁₄N₂O₂R₂ | |
| 211 | K(COBu) | 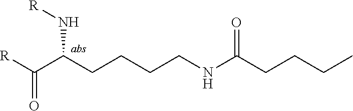 | CCCCC(NCCCC [c@@H](C([R])═ O)N[R])═O |
| | | C₁₁H₂₀N₂O₂R₂ | |
| 212 | K(COiBu) | 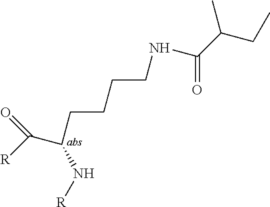 | CCC(C)C(NCCC C[c@@H](C([R])═ O)N[R])═O CC(C)CC(NCCC C[c@@H](C([R])═ O)N[R])═O |
| | | C₁₁H₂₀N₂O₂R₂ | |
| 213 | K(COtBu) | 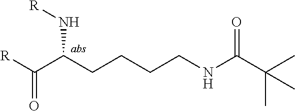 | CC(C)(C)C(NCC CC[c@@H](C([R])═ O)N[R])═O |
| | | C₁₁H₂₀N₂O₂R₂ | |
| 214 | succiniccarn | 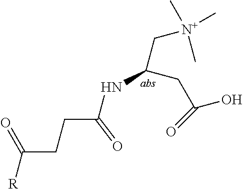 | C[N+](C)(C)C[C @@H](CC(O)═O) NC(CCC([R])═O)═ O |
| | | C₁₁H₂₀N₂O₄R⁺ | |
| 215 | Aun | 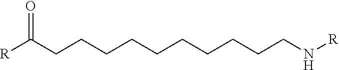 | O═C(CCCCCCCC CCN[R])[R] |
| | | C₁₁H₂₁NOR₂ | |
| 216 | 5BrW, 5BrTrp | 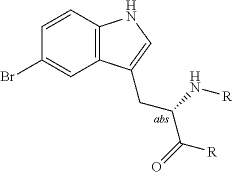 | O═C([C@H](Cc1c [nH]c(cc2)c1cc2B r)N[R])[R] |
| | | C₁₁H₉BrN₂OR₂ | |
| 217 | 7BrTrp, 7BrW | 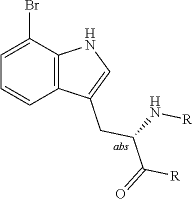 | O═C([C@H](Cc1c [nH]c2c1cccc2Br) N[R])[R] |
| | | C₁₁H₉BrN₂OR₂ | |
| 218 | 7ClW, 7ClTrp | 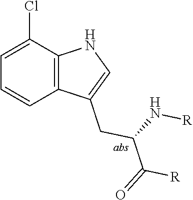 | O═C([C@H](Cc1c [nH]c2c1cccc2Cl) N[R])[R] |
| | | C₁₁H₉ClN₂OR₂ | |
| 219 | 5FW, 5FTrp | 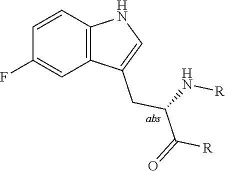 | O═C([C@H](Cc1c [nH]c(cc2)c1cc2F) N[R])[R] |
| | | C₁₁H₉FN₂OR₂ | |
| 220 | 7FW, 7FTrp | 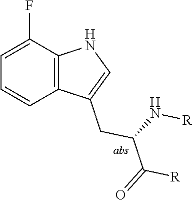 | O═C([C@H](Cc1c [nH]c2c1cccc2F) N[R][R] |
| | | C₁₁H₉FN₂OR₂ | |
| 221 | BT, L-3- Benzothienylalanine | 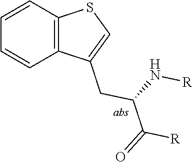 | O═C([C@H](Cc1c sc2c1cccc2)N[R]) [R] |
| | | C₁₁H₉NOSR₂ | |
| 222 | 2Quin 60HQui | 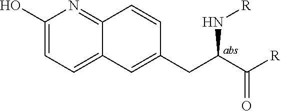 | Oc1ccc(cc(C[C@ @H](C([R])═O)N [R])cc2)c2n1 |
| | | C₁₂H₁₀N₂O₂R₂ | |
| 223 | 7CF2H | 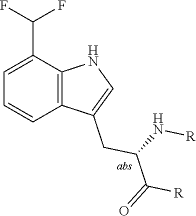 | O═C([C@H](Cc1c [nH]c2c1cccc2C(F) F)N[R])[R] |
| | | C₁₂H₁₀F₂N₂OR₂ | |
| 224 | 3QuinolAla | 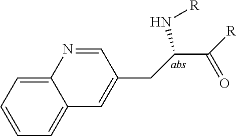 | O═C([C@H](Cc1c c2ccccc2nc1)N[R]) [R] |
| | | C₁₂H₁₀N₂OR₂ | |
| 225 | 2MeTrp, 2MeW | 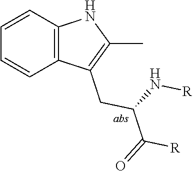 | Cc1c(C[C@@H] (C([R])═O)N[R])c (cccc2)c2[nH]1 |
| | | C₁₂H₁₂N₂OR₂ | |
| 226 | 5MeW, 5MeTrp | 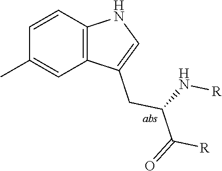 | Cc(cc1)cc2c1[nH] cc2C[C@@H](C ([R])═O)N[R] |
| | | C₁₂H₁₂N₂OR₂ | |
| 227 | 7MeW, 7(MeW), 7MeTrp | 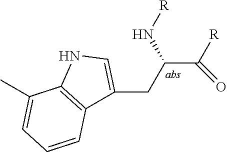 | Cc1cccc2c1[nH]cc 2C[c@@H](C([R 1)═O)N[R] Cc1cccc2c1[nH]cc 2C[c@@H](C═O) N |
| | | C₁₂H₁₂N₂OR₂ | |
| 228 | aMeW | 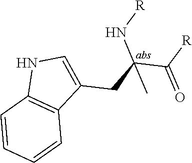 | C[C@](Cc1c[nH]c 2c1cccc2)(C([R])═ O)N[R] |
| | | C₁₂H₁₂N₂OR₂ | |
| 229 | dW7Me, 7Mew, 7MedW | 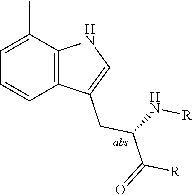 | Cc1cccc2c1[nH]cc 2C[C@H](C([R]) ═O)N[R] |
| | | C₁₂H₁₂N₂OR₂ | |
| 230 | Y(OTzl) | 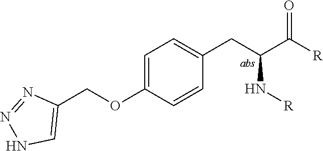 | O═C([C@H](Cc(c c1)ccc1OCc1c[nH] nn1)N[R])[R] |
| | | C₁₂H₁₂N₄O₂R₂ | |
| 231 | 4AllylY | 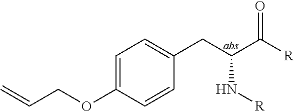 | C═CCOc1ccc(C[C @@H](C([R])═O) N[R])cc1 |
| | | C₁₂H₁₃NO₂R₂ | |
| 232 | 4AllyIF | 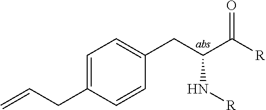 | CCCc1ccc(C[C @@H](C([R])═O) N[R])cc1 |
| | | C₁₂H₁₃NOR₂ | |
| 233 | meW, NMeW, NMeTrp, N-Methyl- Tryptophan | 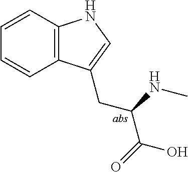 [SMILES: CN[C@H](Cc1c[nH]c2ccccc12)C(=O)O] | CN[C@@H](Cc1 c[nH]c2c1cccc2)C (O)═O |
| | | C₁₂H₁₄N₂O₂ | |
| | AEF(G) | 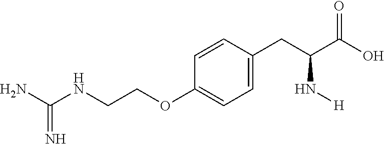 [SMILES: N=C(N)NCCOc1ccc(C[C@H](N)C(=O)O)cc1] | [R]C([C@H](CC1 ═CC═C(OCCNC (N)═N)C═C1)N[R]) ═O |
| 234 | AAMPhe | 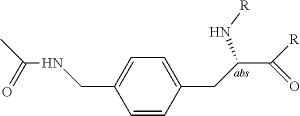 | CC(NCc1ccc(C[C @@H](C([R])═O) N[R])cc1)═O CC(NCc1ccc(C[C @@H](C═O)N)cc1) ═O |
| | | C₁₂H₁₄N₂O₂R₂ | |
| 235 | hC(pXyl) | 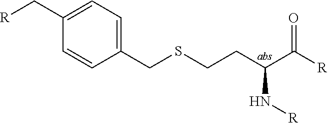 | O═C([C@H](CCS Cc1ccc(C[R])cc1) N[R])[R] |
| | | C₁₂H₁₄NOSR₃ | |
| 236 | AEF(NMe(2)) | 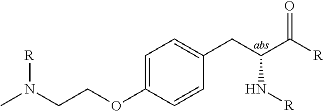 | CN(CCOc1ccc(C [c@@H](C([R])═ O)N[R])cc1)[R] |
| | | C₁₂H₁₅N₂O₂R₃ | |
| 237 | DY02 | 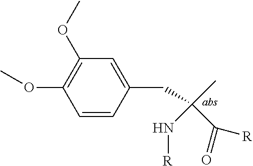 | C[c@@](Cc(cc1) cc(OC)c1OC)(C ([R])═O)N[R] |
| | | C₁₂H₁₅NO₃R₂ | |
| 238 | Y02 | 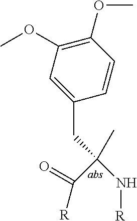 | C[C@](Cc(cc1)cc (OC)c1OC)(C([R]) ═O)N[R] |
| | | C₁₂H₁₅NO₃R₂ | |
| 239 | AEF(NMe) | 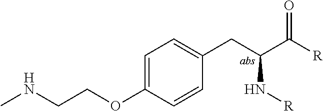 | CNCCOc1ccc(C [c@@H](C([R])═ O)N[R])cc1 |
| | | C₁₂H₁₆N₂O₂R₂ | |
| 240 | NMeAEF | 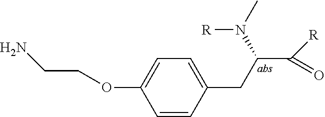 | CN([C@@H](Cc (cc1)ccc1OCCN)C ([R])═O)[R] CN[C@@H](Cc(c c1)ccc1OCCN)C═O |
| | | C₁₂H₁₆N₂O₂R₂ | |
| 241 | aMeAEF | 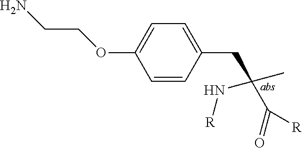 | C[C@](Cc(cc1)cc c1OCCN)(C([R])═ O)N[R] CC(C)(C)OC(NC COc1ccc(C[C@@] (C)(C([R])═O)N [R])cc1)═O |
| | | C₁₂H₁₆N₂O₂R₂ | |
| 242 | bMeAEF | 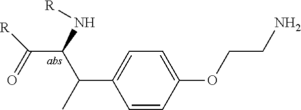 | CC([C@@H](C ([R])═O)N[R])c(cc1) ccc1OCCN |
| | | C₁₂H₁₆N₂O₂R₂ | |
| 243 | bMeAEF(2S,3R*), bMeAEF(2S3R*) (*pure but configuration unknown) | 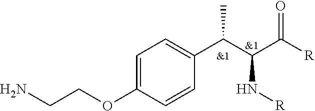 | C[c@@H]([C@ @H](C([R])═O)N [Rl)c(cc1)ccc1OC CN |
| | | C₁₂H₁₆N₂O₂R₂ | |
| 244 | bMeAEF(2S3S*), bMeAEF(2S,3S*) (*pure but configuration unknown) | 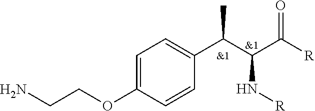 | C[C@H]([C@@H] (C([R]═O)N[R]) c(cc1)ccc1OCCN |
| | | C₁₂H₁₆N₂O₂R₂ | |
| 245 | K(Morph) | 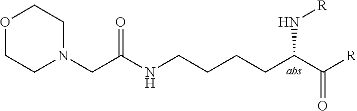 | O═C(CNICCOCC 1)NCCCC[C@@ H](C([R])═O)N[R] |
| | | C₁₂H₂₁N₃O₃R₂ | |
| 246 | K(COPent) | 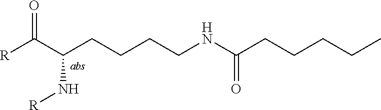 | CCCCCC(NCCC C[c@@H](C([R]) ═O)N[R])═O |
| | | C₁₂H₂₂N₂O₂R₂ | |
| 247 | aMeK(Boc) | 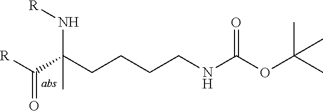 | CC(C)(C)OC(NC CCC[c@@](C)(C ([R])═O)N[R])═O |
| | | C₁₂H₂₂N₂O₃R₂ | |
| 248 | E(C) | 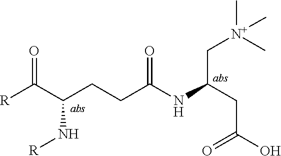 | C[N+](C)(C)C[C @H](CC(O)═O)N C(CC[c@@H](C ([R])═O)N[R])═O |
| | | C₁₂H₂₂N₃O₄R₂⁺ | |
| 249 | E(c) (R)-2-((R)-4-amino- 4- carboxybutanamido)- 3-carboxy-N,N,N- trimethylpropan-1- aminium, E(c) | 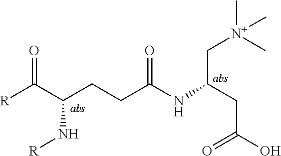 | C[N+](C)(C)C[C @@H](CC(O)═O) NC(CC[C@@H] (C([R])═O)N[R])═O |
| | | C₁₂H₂₂N₃O₄R₂⁺ | |
| 250 | e(C), dE(C) | 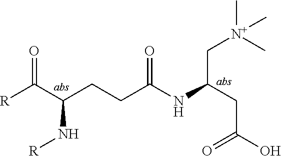 | C[N+](C)(C)C[C @H](CC(O)═O)N C(CC[C@H](C([R]) ═O)N[R])═O |
| | | C₁₂H₂₂N₃O₄R₂+ | |
| 251 | e(c), dE(c) | 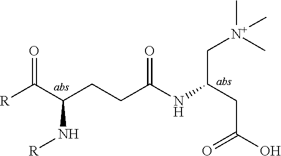 | C[N+](C)(C)C[C @@H](CC(O)═O) NC(CC[C@H](C ([R])═O)N[R])═O |
| | | C₁₂H₂₂N₃O₄R₂+ | |
| 252 | dK(SP6), k(SP6) | 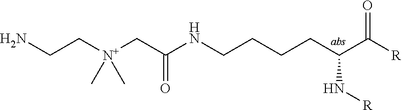 | C[N+](C)(CCN)C C(NCCCC[C@H] (C([R])═O)N[R])═O |
| | | C₁₂H₂₅N₄O₂R₂+ | |
| 253 | 7CF3W, (S)-2- amino-3-(7- (trifluoromethyl)-1H- indol-3-yl)propanoic acid | 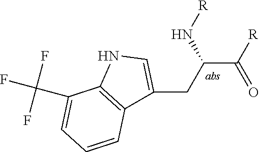 | O═C([C@H](Cc1c [nH]c2c1cccc2C(F) (F)F)N[R])[R] N[C@@H](Cc1c[ nH]c2c(C(F)(F)F) cccc12)C═O N[C@@H](Cc1c [nH]c2c(C(F)(F)F) cccc12)C═O |
| | | C₁₂H₉F₃N₂OR₂ | |
| 254 | 5Br2Nal | 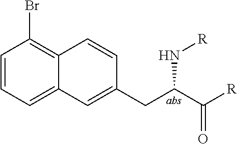 | O═C([C@H](Cc1c c2cccc(Br)c2cc1) N[R])[R] |
| | | C₁₃H₁₀BrNOR₂ | |
| 255 | 6Br2Nal | 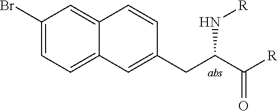 | O═C([C@H](Cc(c cc1c2)cc1ccc2Br) N[R])[R] |
| | | C₁₃H₁₀BrNOR₂ | |
| 256 | 7Br2Nal | 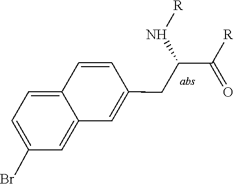 | O═C([C@H](Cc1c c2cc(Br)ccc2cc1) N[R])[R] |
| | | C₁₃H₁₀BrNOR₂ | |
| 257 | 6F2Nal | 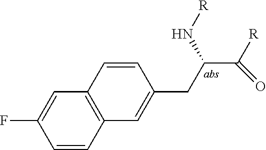 | O═C([C@H](Cc(c cc1c2)cc1ccc2F)N [R])[R] N[C@@H](Cc1cc c(cc(cc2)F)c2c1)C ═O |
| | | C₁₃H₁₀FNOR₂ | |
| 258 | 7OH2Nal | 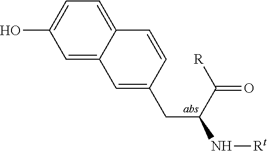 | Oc1ccc(ccc(C[C@ @H](C([R])═O)N [R1])c2)c2c1 |
| | | C₁₃H₁₁NO₂R₂ | |
| 259 | 1Nal, Nal, | 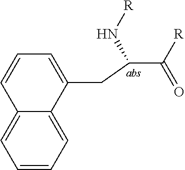 | O═C([C@H](Cc1c ccc2ccccc12)N[R]) [R] |
| | | C₁₃H₁₁NOR₂ | |
| 260 | 2Nal | 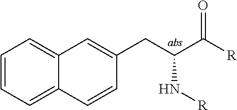 | O═C([C@H](Cc1c c2ccccc2cc1)N[R]) [R] |
| | | C₁₃H₁₁NOR₂ | |
| 261 | dNal, d2Nal | 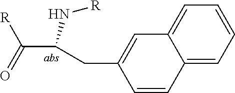 | O═C([C@@H](Cc 1cc2ccccc2cc1)N [R])[R] |
| | | C₁₃H₁₁NOR₂ | |
| 262 | 6MeQui | 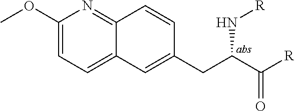 | COc1ccc(cc(C[C @@H](C([R])═O) N[R])cc2)c2nl |
| | | C₁₃H₁₂N₂O₂R₂ | |
| 263 | D(N5In) | 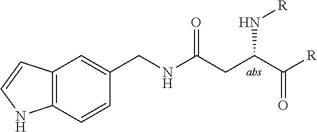 | O═C(C[C@@H] (C([R])═O)N[R])N Cc(cc1)cc2c1[nH] cc2 |
| | | C₁₃H₁₃N₃O₂R₂ | |
| 264 | psi2Nal | 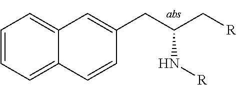 | [R]C[C@H](Cc1c c2ccccc2cc1)N[R] |
| | | C₁₃H₁₃NR₂ | |
| 265 | 7EtW | 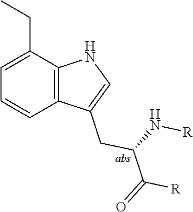 | CCc1cccc2c1[nH] cc2C[C@@H](C ([R])═O)N[R] |
| | | C₁₃H₁₄N₂OR₂ | |
| 266 | F(4TzlMME) | 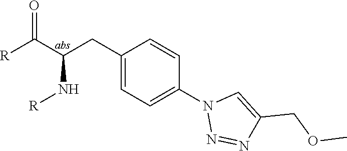 | COCc1cn(- c2ccc(C[C@@H] (C([R])-O)N[R])cc 2)nn1 |
| | | C₁₃H₁₄N₄O₂R₂ | |
| 267 | AcAEF | 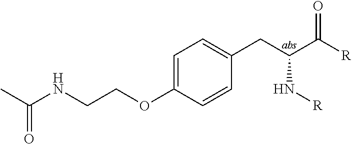 | CC(NCCOc1ccc (C[c@@H](C([R]) ═O)N[R])cc1)═O |
| | | C₁₃H₁₆N₂O₃R₂ | |
| 268 | tButY, Y(tBu) | 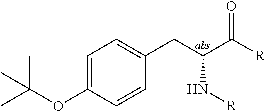 | CC(C)(C)Oc1ccc (C[C@H](C([R])═ O)N[R])cc1 |
| | | C₁₃H₁₇NO₂R₂ | |
| 269 | AEF(Me)2 | 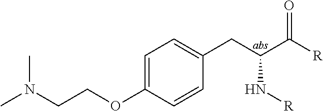 | CN(C)CCOc1ccc (C[c@@H](C([R]) ═O)N[R])cc1 |
| | | C₁₃H₁₈N₂O₂R₂ | |
| 270 | Z, Amp | 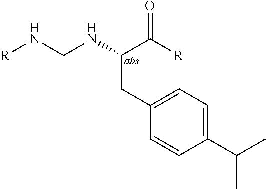 | CC(C)c1ccc(C[C @@H](C([R])═O) NCN[R])cc1 |
| | | C₁₃H₁₈N₂OR₂ | |
| 271 | 5amido2Nal | 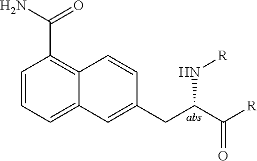 | NC(c1c(ccc(C[C @@H](C([R])═O) N[R])c2)c2ccc1)═O |
| | | C₁₄H₁₂N₂O₂R₂ | |
| 272 | 6amido2Nal | 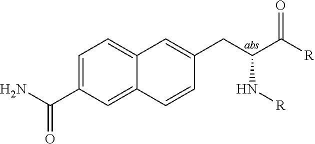 | NC(c1ccc(cc(C[C @@H](C([R])═O) N[R])cc2)c2c1)═O |
| | | C₁₄H₁₂N₂O₂R₂ | |
| 273 | 5OMe2Nal | 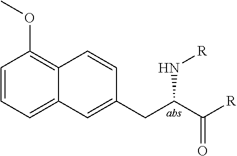 | COc1c(ccc(C[C@ @H](C([R])═O)N [R])c2)c2ccc1 |
| | | C₁₄H₁₃NO₂R₂ | |
| 274 | 6OMe2Nal | 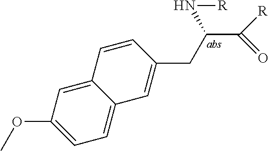 | COc1ccc(cc(C[C @@H](C([R])═O) N[R])cc2)c2c1 |
| | | C₁₄H₁₃NO₂R₂ | |
| 275 | 5Me2Nal | 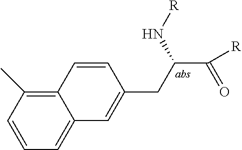 | Cc1c(ccc(C[C@@ H](C([R])═O)N[R]) c2)c2ccc1 |
| | | C₁₄H₁₃NOR₂ | |
| 276 | NMe2NAL | 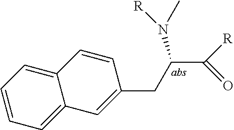 | CN([C@@H](Cc1 cc2ccccc2cc1)C ([R])═O)[R] CN[C@@H](Cc1 cc2ccccc2cc1)C═O |
| | | C₁₄H₁₃NOR₂ | |
| 277 | aMe2Nal | 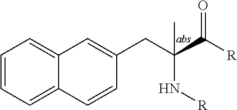 | C[C@](Cc1cc2ccc cc2cc1)(C([R])═O) N[R] |
| | | C₁₄H₁₃NOR₂ | |
| 278 | bMe2Nal(2S,3R), bMe2Nal(2S3R) | 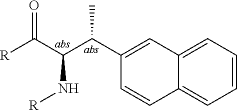 | C[C@@H]([C@ @H](C([R])═O)N [R])c1cc2ccccc2cc1 |
| | | C₁₄H₁₃NOR₂ | |
| 279 | bMe2Nal(2S3S), bMe2Nal(2S3R) | 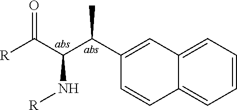 | C[C@H]([C@@H] (C([R])═O)N[R]) c1cc2ccccc2cc1 |
| | | C₁₄H₁₃NOR₂ | |
| 280 | AEF(EtCO) | 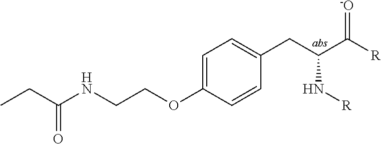 | CCC(NCCOc1ccc (C[C@@H](C([R)] ═O)N[R])cc1)═O |
| | | C₁₄H₁₈N₂O₃R₂ | |
| 281 | NMeY(tBu) | 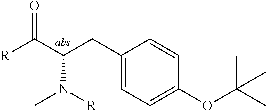 | CC(C)(C)Oc1ccc (C[C@H](C([R])═ O)N(C)[R])cc1 |
| | | C₁₄H₁₉NO₂R₂ | |
| 282 | AEF(NMe3) | 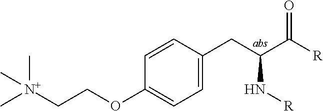 | C[N+](C)(C)CCO c1ccc(C[C@@H] (C([R])═O)N[R])cc1 |
| | | C₁₄H₂₁N₂O₂R₂⁺ | |
| 283 | 6O(COCF3)2Nal | 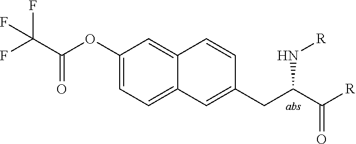 | O═C([C@H](Cc(c cc1c2)cc1ccc2OC (C(F)(F)F)═O)N[R]) [R] |
| | | C₁₅H₁₀F₃NO₃R₂ | |
| 284 | BIF | 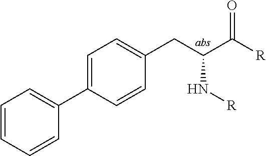 | O═C([C@H](Cc(c c1)ccc1- c1ccccc1)N[R])[R] |
| | | C₁₅H₁₃NOR₂ | |
| 285 | DiPhAla | 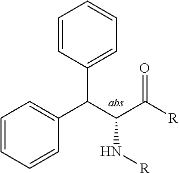 | O═C([C@H](C(c1 ccccc1)c1ccccc1) N[R])[R] |
| | | C₁₅H₁₃NOR₂ | |
| 286 | 5Et2Nal | 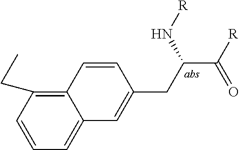 | CCc1c(ccc(C[C@ @H](C([R])═O)N [R])c2)c2ccc1 |
| | | C₁₅H₁₅NOR₂ | |
| 287 | CMF | 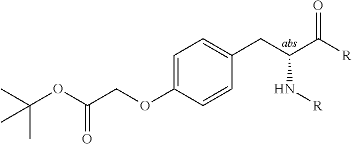 | CC(C)(C)OC(COc 1ccc(C[C@@H] (C([R])═O)N[R])cc1) ═O |
| | | C₁₅H₁₉NO₄R₂ | |
| 288 | F(4TzlTMA1) | 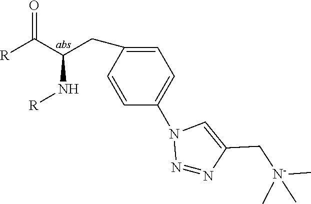 | C[N+](C)(C)Cc1c n(- c2ccc(C[C@@H] (C([R])═O)N[R])cc 2)nn1 |
| | | C₁₅H₂₀N₅OR₂⁺ | |
| 289 | PiperazinequatF | 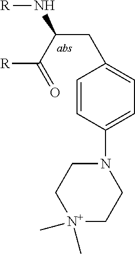 | C[N+](C)(CC1)C CNlc1ccc(C[C@ @H](C([R])═O)N [R])cc1 |
| | | C₁₅H₂₂N₃OR₂⁺ | |
| 290 | TMA3F | 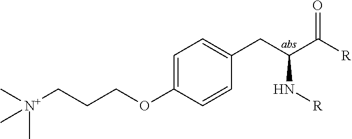 | C[N+](C)(C)CCC Oc1ccc(C[C@@H] (C([R])═O)N[R]) cc1 |
| | | C₁₅H₂₃N₂O₂R₂⁺ | |
| 291 | TMA4F | 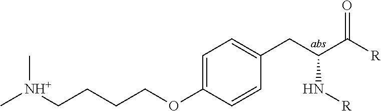 | C[NH+](C)CCCC Oc1ccc(C[C@@H] (C([R])═O)N[R]) cc1 |
| | | C₁₅H₂₃N₂O₂R₂⁺ | |
| 292 | K5cpa, K(5cpa), K(5cpaCO) | 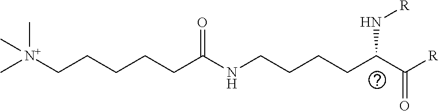 | C[N+](C)(C)CCC CCC(NCCCC[C@ @H](C([R])═O)N [R])═O |
| | | C₁₅H₃₀N₃O₂R₂⁺ | |
| 293 | dK(5cpa), k(5cpa), k(5cpaCO) | 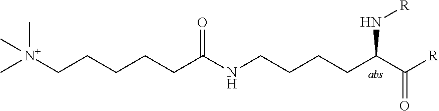 | C[N+](C)(C)CCC CCC(NCCCC[C@ H](C([R])═O)N[R]) ═O |
| | | C₁₅H₃₀N₃O₂R₂⁺ | |
| 294 | 2Nal6(3pyrazole) | 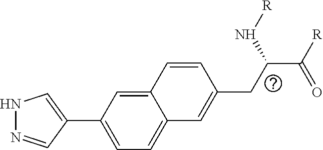 | O-C([C@H](Cc(c cc1c2)cc1ccc2- c1c[nH]nc1)N[R]) [R] |
| | | C₁₆H₁₃N₃OR₂ | |
| 295 | 7PyrTrp | 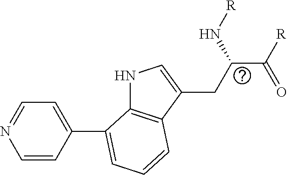 | O═C([C@H](Cc1c [nH]c2c1cccc2- c1ccncc1)N[R])[R] |
| | | C₁₆H₁₃N₃OR₂ | |
| 296 | 4BzF | 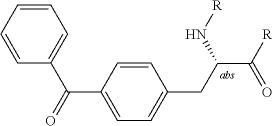 | O═C([C@H](Cc(c c1)ccc1C(c1ccccc1) ═O)N[R])[R] |
| | | C₁₆H₁₃NO₂R₂ | |
| 297 | aMeBiF | 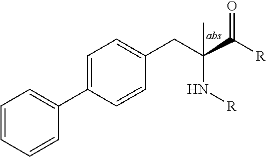 | C[C@](Cc(cc1)cc c1- c1ccccc1)(C([R])═ O)N[R] |
| | | C₁₆H₁₅NOR₂ | |
| 298 | NPyEF | 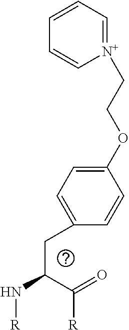 | O═C([C@H](Cc(c c1)ccc1OCC[n+]1 ccccc1)N[R])[R] |
| | | C₁₆H₁₇N₂O₂R₂⁺ | |
| 299 | 5iPr2Nal | 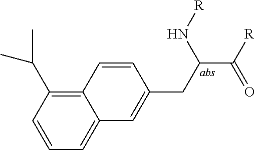 | CC(C)c1c(ccc(C [c@@H](C([R])═ O)N[R])c2)c2ccc1 |
| | | C₁₆H₁₇NOR₂ | |
| 300 | TetraFAEF(Boc) | 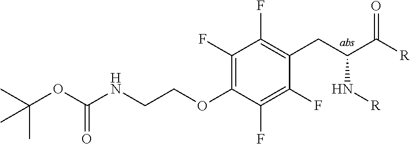 | CC(C)(C)OC(NC COc(c(F)c(c(C[C @@H](C([R])═O) N[R])c1F)F)c1F)═O |
| | | C₁₆H₁₈F₄N₂O₄R₂ | |
| 301 | 4TMABYF | 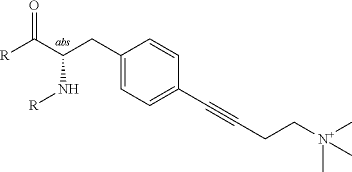 | C[N+](C)(C)CCC #Cc1ccc(C[C@@ H](C([R])═O)N[R]) cc1 |
| | | C₁₆H₂₁N₂OR₂⁺ | |
| 302 | AEF(Boc) | 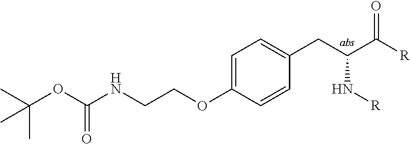 | CC(C)(C)OC(NC COc1ccc(C[C@@ H](C([R])═O)N[R 1)cc1)═O |
| | | C₁₆H₂₂N₂O₄R₂ | |
| 303 | F(4TzlTMA2) | 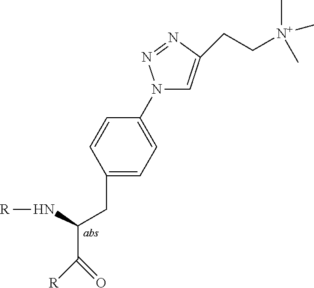 | C[N+](C)(C)CCc1 cn(- c2ccc(C[C@@H] (C([R])═O)N[R])cc 2)nn1 |
| | | C₁₆H₂₂N₅OR₂⁺ | |
| 304 | DMPMF | 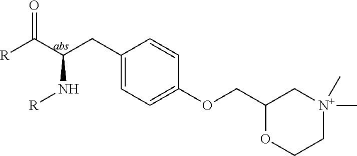 | C[N+]1(C)CC(CO c2ccc(C[C@@H] (C([R])═O)N[R])cc 2)OCC1 |
| | | C₁₆H₂₃N₂O₃R₂⁺ | |
| 305 | KDde, K(Dde) | 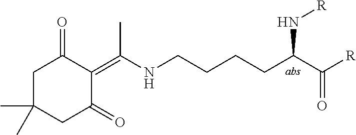 | CC(C)(CC(C1═C (C)NCCCC[C@@ H](C([R])═O)N[R D)═O)CC1═O |
| | | C₁₆H₂₄N₂O₃R₂ | |
| 306 | dKDde, k(Dde), dK(Dde) | 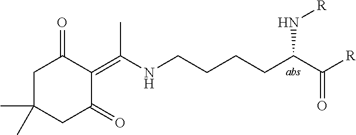 | CC(C)(CC(C1═C (C)NCCCC[C@H] (C([R])═O)N[R])═ O)CC1═O |
| | | C₁₆H₂₄N₂O₃R₂ | |
| 307 | Y(OEOXIMECh) | 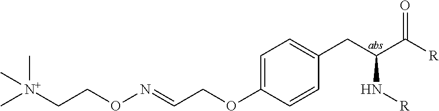 | C[N+](C)(C)CCO/ N═C/COc1ccc(C [c@@H](C([R])═ O)N[R])cc1 |
| | | C₁₆H₂₄N₃O₃R₂⁺ | |
| 308 | Y(OZOXIMECh) | 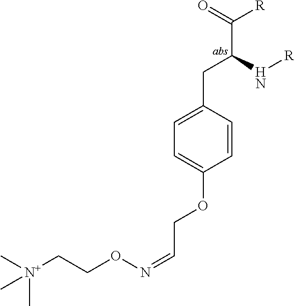 | C[N+](C)(C)CCO/ N═C\COc1ccc(C [c@@H](C([R])═ O)N[R])cc1 |
| | | C₁₆H₂₄N₃O₃R₂⁺ | |
| 309 | AEF(NHCh) | 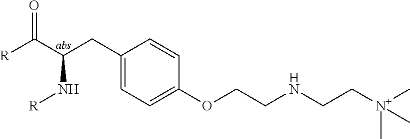 | C[N+](C)(C)CCN CCOc1ccc(C[C@ @H](C([R])═O)N [R])cc1 |
| | | C₁₆H₂₆N₃O₂R₂⁺ | |
| 310 | K(Biotina), K(Biotin) | 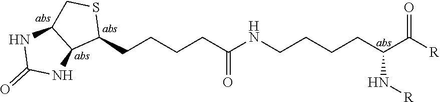 | O═C(CCCC[C@H] ([C@@H]IN2)S C[C@H]1NC2═O) NCCCC[C@@H] (C([R])═O)N[R] |
| | | C₁₆H₂₆N₄O₃SR₂ | |
| 311 | K(DAGSuc) | 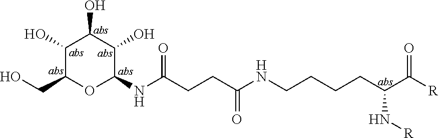 | OC[C@H]([C@H] ([C@@H]([C@H] 1O)O)O)O[C@H] 1NC(CCC(NCCC C[c@@H](C([R]) ═O)N[R])═O)═O |
| | | C₁₆H₂₇N₃O₈R₂ | |
| 312 | k(DAGSuc), dK(DAGSuc) | 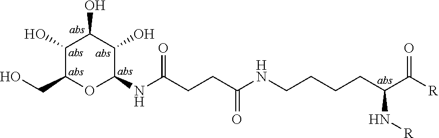 | OC[C@H]([C@H] ([C@@H]([C@H] 1O)O)O)O[C@H] 1NC(CCC(NCCC C[C@H](C([R])═ O)N[R])═O)═O |
| | | C₁₆H₂₇N₃O₈R₂ | |
| 313 | DOTA | 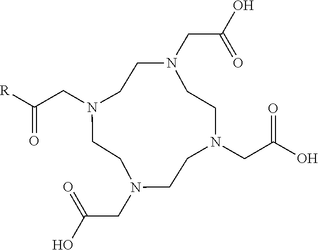 | OC(CNICCN(CC (O)═O)CCN(CC ([R])═O)CCN(CC (O)═O)CC1)═O |
| | | C₁₆H₂₇N₄O₇R | |
| 314 | Dab(NMeCarn) | 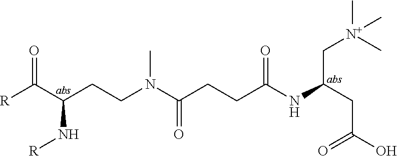 | CN(CC[C@@H] (C([R])═O)N[R])C (CCC(N[C@@H] (CC(O)═O)C[N+] (C)(C)C)═O)═O |
| | | C₁₆H₂₉N₄O₅R₂⁺ | |
| 315 | Dab(NMecarn) | 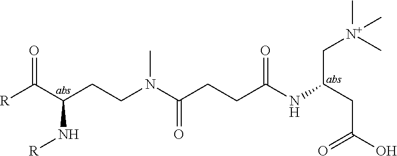 | CN(CC[C@@H] (C([R])═O)N[R])C (CCC(N[C@H](C C(O)═O)C[N+](C) (C)C)═O)═O |
| | | C₁₆H₂₉N₄O₅R₂⁺ | |
| 316 | orn(d) | 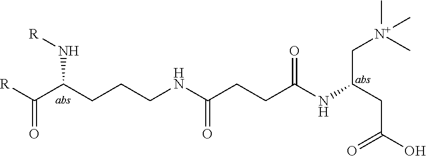 | C[N+](C)(C)C[C @@H](CC(O)═O) NC(CCC(NCCC [C@H](C([R])═O) N[R])═O)═O |
| | | C₁₆H₂₉N₄O₅R₂⁺ | |
| 317 | 2Nal6((5CF3)3 pyrazole) | 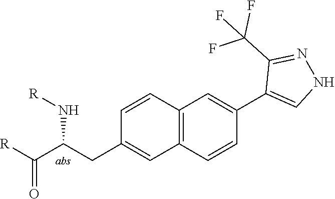 | O═C([C@H](Cc(c cc1c2)cc1ccc2- c1c[nH]nc1C(F)(F) F)N[R])[R] |
| | | C₁₇H₁₂F₃N₃OR₂ | |
| 318 | 7(2ClPh)W | 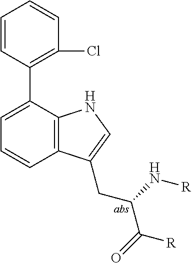 | O═C([C@H](Cc1c [nH]c2c1cccc2- c(cccc1)c1Cl)N[R]) [R] |
| | | C₁₇H₁₃ClN₂OR₂ | |
| 319 | TMAPF | 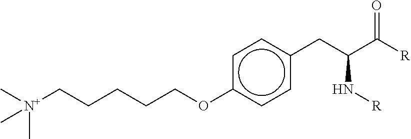 | C[N+](C)(CCCCC Oc1ccc(C[C@H] (N[R])C([R])═O)cc 1)C |
| 320 | 7(2OMe5Pyr)W | 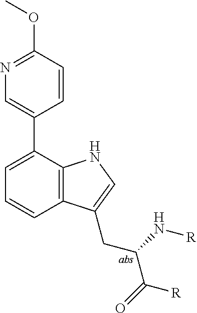 | COc(cc1)ncc1- c1cccc2c1[nH]cc2 C[c@@H](C([R]) ═O)N[R] |
| | | C₁₇H₁₅N₃O₂R₂ | |
| 321 | W-7Ph, 7-phenyl-L- tryptophan | 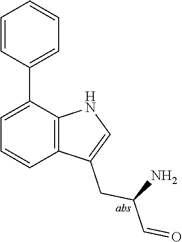 [SMILES: N[C@@H](C=O)Cc1c[nH]c2c(-c3ccccc3)cccc12] | N[C@@H](Cc1c [nH]c2c1cccc2- c1ccccc1)C═O |
| | | C₁₇H₁₆N₂O | |
| 322 | 5OH2Nal | 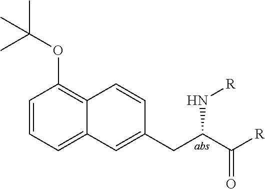 | CC(C)(C)Oc1c(cc c(C[C@@H](C ([R])═O)N[R])c2)c2 ccc 1 |
| | | C₁₇H₁₉NO₂R₂ | |
| 323 | 5tBu2Nal | 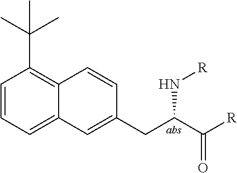 | CC(C)(C)c1c(ccc (C[C@@H](C([R]) ═O)N[R])c2)c2ccc1 |
| | | C₁₇H₁₉NOR₂ | |
| 324 | hFTMAPF | 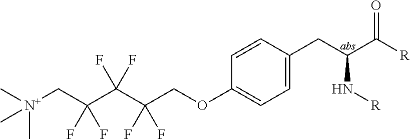 | C[N+](C)(C)CC(C (C(COcccc(C[C @@H](C([R])═O) N[R])cc1)(F)F)(F) F)(F)F |
| | | C₁₇H₂₁F₆N₂O₂R₂⁺ | |
| 325 | F(4TzlTMA3) | 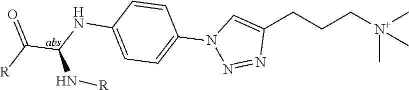 | C[N+](C)(C)CCC c1cn(- c2ccc(C[C@@H] (C([R])═O)N[R])cc 2)nn1 |
| | | C₁₇H₂₄N₅OR₂⁺ | |
| 326 | DMMMF | 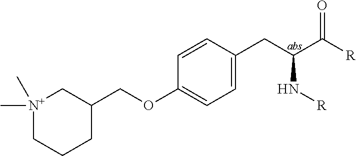 | C[N+]1(C)CC(CO c2ccc(C[C@@H] (C([R])═O)N[R])cc 2)CCC1 |
| | | C₁₇H₂₅N₂O₂R₂⁺ | |
| 327 | MMoEF | 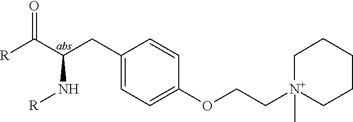 | C[N+]1(CCOc2cc c(C[C@@H](C ([R])═O)N[R])cc2) CCCCC1 |
| | | C₁₇H₂₅N₂O₂R₂⁺ | |
| 328 | MMoPF | 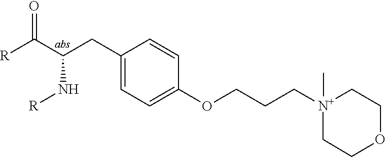 | C[N+]1(CCCOc2c cc(C[C@H](C([R]) ═O)N[R])cc2)CC OCC1 |
| | | C₁₇H₂₅N₂O₃R₂⁺ | |
| 329 | AEF(MEP) | 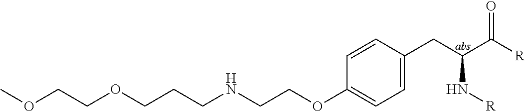 | COCCOCCCNCC Oc1ccc(C[C@@H] (C([R])═O)N[R]) cc1 |
| | | C₁₇H₂₆N₂O₄R₂ | |
| 330 | 4DMPzEF | 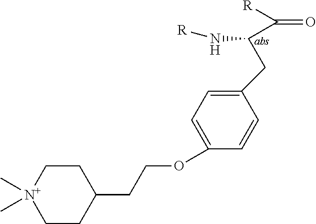 | C[N+]1(C)CCN(C COc2ccc(C[C@@ 1)cc2)CC1 C[N+]1(C)CCN(C COc2ccc(C[C@@ H](C═O)N)cc2)C C1 |
| | | C₁₇H₂₆N₃O₂R₂⁺ | |
| 331 | TMAPF | 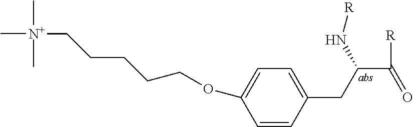 | C[N+](C)(C)CCC CCOc1ccc(C[C@ @H](C([R])═O)N [R])cc1 C[N+](C)(C)CCC CCOc1ccc(C[C@ @H](C═O)N)cc1 |
| | | C₁₇H₂₇N₂O₂R₂⁺ | |
| 332 | K(D), KCar | 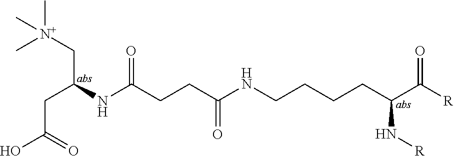 | C[N+](C)(C)C[C @H](CC(O)═O)N C(CCC(NCCCC[ C@@H](C([R])═ O)N[R])═O)═O |
| | | C₁₇H₃₁N₄O₅R₂⁺ | |
| 333 | K(d), KdCar | 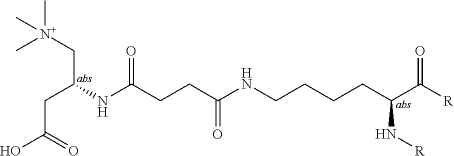 | C[N+](C)(C)C[C @@H](CC(O)═O) NC(CCC(NCCCC [C@@H](C([R])═ O)N[R])═O)═O |
| | | C₁₇H₃₁N₄O₅R₂⁺ | |
| 334 | k(D), dKCar | 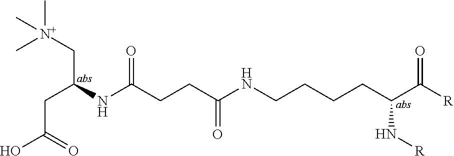 | C[N+](C)(C)C[C @H](CC(O)═O)N C(CCC(NCCCC [C@H](C([R])═O) N[R])═O)═O |
| | | C₁₇H₃₁N₄O₅R₂⁺ | |
| 335 | k(d), dKdCar | 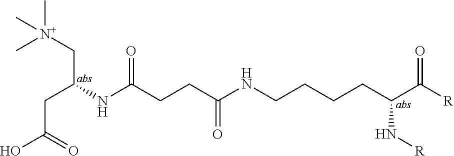 | C[N+](C)(C)C[C @@H](CC(O)═O) NC(CCC(NCCCC [C@H](C([R])═O) N[R])═O)═O |
| | | C₁₇H₃₁N₄O₅R₂+ | |
| 336 | 7(3CF3TAZP)W | 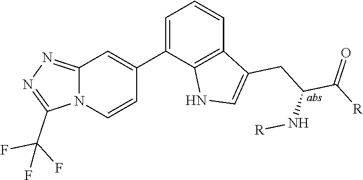 | O═C([C@H](Cc1c [nH]c2c1cccc2- c1cc2nnc(C(F)(F) F)n2cc1)N[R])[R] |
| | | C₁₈H₁₂F₃N₅OR₂ | |
| 337 | 7(40CF3Ph)W | 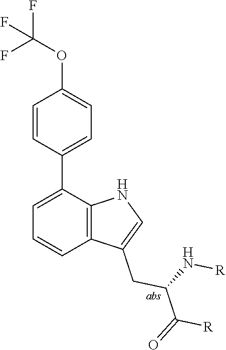 | O═C([C@H](Cc1c [nH]c2c1cccc2- c(cc1)ccc1OC(F) (F)F)N[R])[R] |
| | | C₁₈H₁₃F₃N₂O₂R₂ | |
| 338 | 7(4CF3Ph)W | 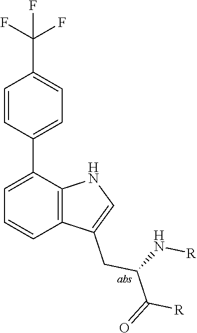 | O═C([C@H](Cc1c [nH]c2c1cccc2- c1ccc(C(F)(F)F)cc 1)N[R])[R] |
| | | C₁₈H₁₃F₃N₂OR₂ | |
| 339 | 7(7ImidPyr)W | 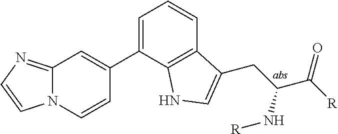 | O═C([C@H](Cc1c [nH]c2c1cccc2- c1cc2nccn2cc1)N [R])[R] |
| | | C₁₈H₁₄N₄OR₂ | |
| 340 | Y(C9OH) | 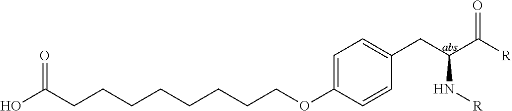 | OC(CCCCCCCC Oc1ccc(C[C@@H] (C([R])═O)N[R]) cc1)═O |
| | | C₁₈H₂₅NO₄R₂ | |
| 341 | Y(OTzlCh) | 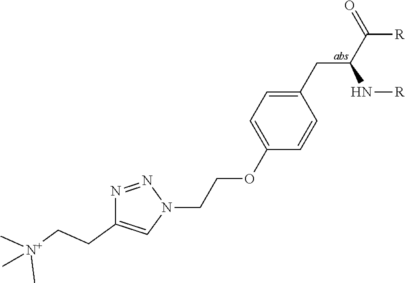 | C[N+](C)(C)CCc1 cn(CCOc2ccc(C[C @@H](C([R])═O) N[R])cc2)nn1 |
| | | C₁₈H₂₆N₅O₂R₂⁺ | |
| 342 | 4DMPEF | 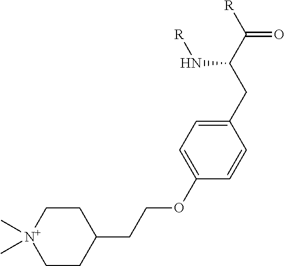 | C[N+]1(C)CCC(C COc2ccc(C[C@@ H](C([R])═O)N[R]) cc2)CC1 C[N+]1(C)CCC(C COc2ccc(C[C@@ H](C═O)N)cc2)C C1 |
| | | C₁₈H₂₇N₂O₂R₂⁺ | |
| 343 | AEF(AcCh) | 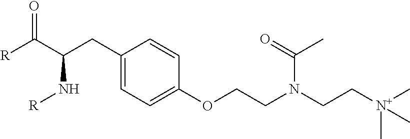 | CC(N(CC[N+](C) (C)C)CCOc1ccc (C[c@@H](C([R]) ═O)N[R])cc1)═O |
| | | C₁₈H₂₈N₃O₃R₂⁺ | |
| 344 | TMA6F | 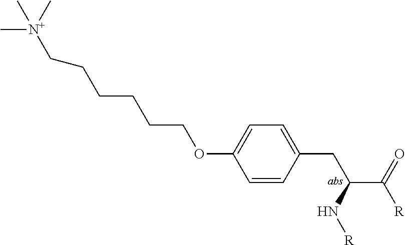 | C[N+](C)(C)CCC CCCOc1ccc(C[C @@H](C([R])═O) N[R])cc1 |
| | | C₁₈H₂₉N₂O₂R₂⁺ | |
| 345 | AEF(MePrpa) | 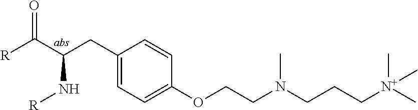 | CN(CCC[N+](C)( C)C)CCOc1ccc(C [C@@H](C([R])═ O)N[R])cc1 |
| | | C₁₈H₃₀N₃O₂R₂⁺ | |
| 346 | 2Nal6(Ph2OH) | 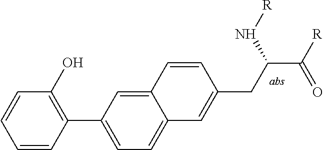 | Oc(cccc1)c1- c1ccc(cc(C[C@@ H](C([R])═O)N[R]) cc2)c2c1 |
| | | C₁₉H₁₅NO₂R₂ | |
| 347 | 7(3NAcPh)W | 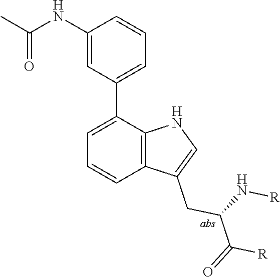 | CC(Nc1cccc(- c2cccc3c2[nH]cc3 C[C@@H](C([R]) ═O)N[R])c1═O |
| | | C₁₉H₁₇N₃O₂R₂ | |
| 348 | 7(4NAcPh)W | 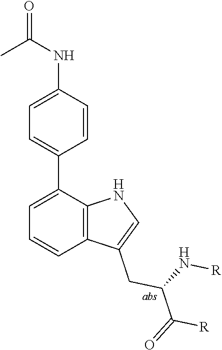 | CC(Nc(cc1)ccc1- c1cccc2c1[nH]cc2 C[c@@H](C([R]) ═O)N[R])═O |
| | | C₁₉H₁₇N₃O₂R₂ | |
| 349 | 4PipPhe | 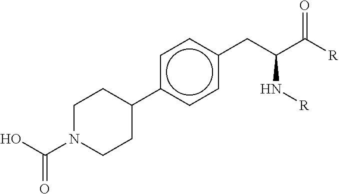 | CC(C)(C)OC(N(C C1) CCClc1ccc(C [c@@H](C([R])═ O)N[R])cc1)═O |
| | | C₁₉H₂₆N₂O₃R₂ | |
| 350 | a | 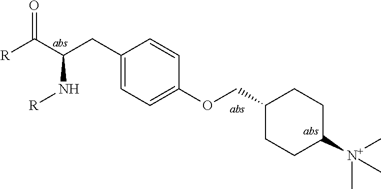 | C[N+](C)(C)[C@ H]1CC[C@H](CO c2ccc(C[C@@H] (C([R])═O)N[R])cc 2)CC1 |
| | | C₁₉H₂₉N₂O₂R₂⁺ | |

TABLE 2D
Peg Moeties and Peg Modified Monomers

| | | | Smiles |
|---|---|---|---|
| 1 | Structure | Names and Synonyms | Structure |

| 2 | 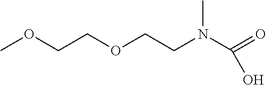 | |
|---|---|---|
| | C7H15NO3 | |
| | CON(MePEG2) | CN(CCOCCOC)C═O |
| 3 | 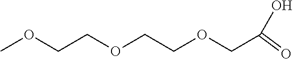 | |
| | C7H14O4 | |
| | mPEG3CO | COCCOCCOCC═O |
| 4 | 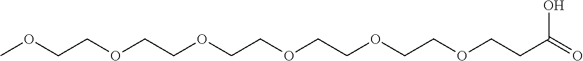 | |
| | C14H28O7 | |
| | mPEG6CO | COCCOCCOCCOCCOCCOCCC═O |
| 5 | 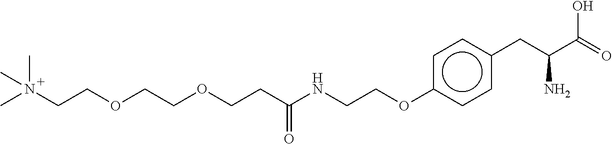 | |
| | C21H36N3O5+ | |
| | AEFNMePEG3a, AEF(NHcPEG3a) | C[N+](C)(C)CCOCCOCCC(NCCOc1ccc(C[C@ |
| | | @H(C═O)N)cc1)═O |
| 6 | 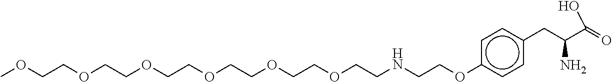 | |
| | C24H42N2O8 | |
| | AEFNmPEG6, AEF(NmPEG6) | COCCOCCOCCOCCOCCOCCNCCOc1ccc(C[C |
| | | @@H](C═O)N)cc1 |
| 7 | 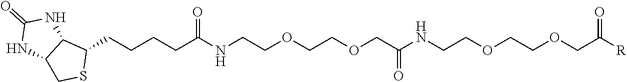 | |
| | C22H37N4O8SR | |
| | BiotinPEG2PEG2, Biotin(PEG2PEG2) | O═C(CCCC[C@@H]([C@H]1N2)SC[C@@H]1 |
| | | NC2═O)NCCOCCOCC(NCCOCCOCC |
| | | ([R])═O)═O |
| 8 | 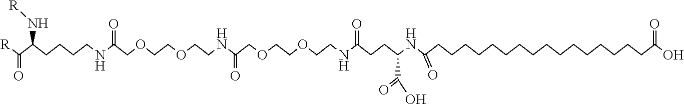 | |
| | C41H73N5O13R2 | |
| | K(PEG2PEG2gEC18OH) | OC(CCCCCCCCCCCCCCCCC(N[C@@H](CC |
| | | C(NCCOCCOCC(NCCOCCOCC(NCCCC[C@@ |
| | | H](C([R])═O)N[R])═O)═O)C(O)═O)═O)═O |
| 9 | 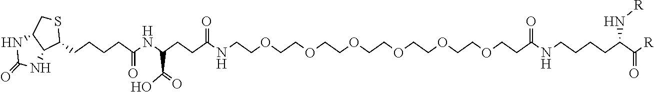 | |
| | C36H62N6O13SR2 | |
| | K(PEG6gEBiotin) | OC([C@H](CCC(NCCOCCOCCOCCOCCOCC |
| | | OCCC(NCCCC[C@@H](C([R])═O)N[R])═O)═ |
| | | O)NC(CCCC[C@H]([C@@H]IN2)SC[C@H]INC |
| | | 2═O)═O)═O |
| 10 | 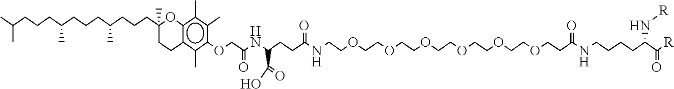 | |
| | C57H98N4O14R2 | |
| | K(PEG6gEVitE) | CC(C)CCC[C@@H](C)CCC[C@@H](C)CCC[C |
| | | @](C)(CC1)Oc(c(C)c2C)c1c(C)c2OCC(N[C@@ |
| | | H](CCC(NCCOCCOCCOCCOCCOCCOCCC(N |
| | | CCCC[C@@H](C([R])═O)N[R])═O)═O)C(O)═ |
| | | O)═O |
| 11 | 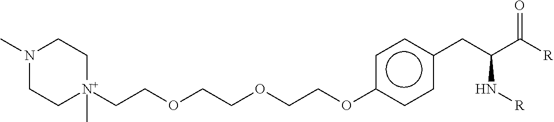 | |
| | C21H34N3O4R2+ | |
| | MPzPEG3F | CNICC[N+](C)(CCOCCOCCOc2ccc(C[C@@H] |
| | | (C([R])═O)N[R])cc2)CC1 |
| 12 | 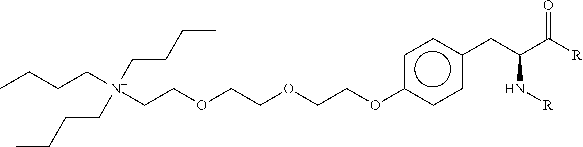 | |
| | C27H47N2O4R2+ | |
| | TBAPEG3F | CCCC[N+](CCCC)(CCCC)CCOCCOCCOc1ccc |
| | | (C[C@@H](C([R])═O)N[R])cc1 |
| 13 | 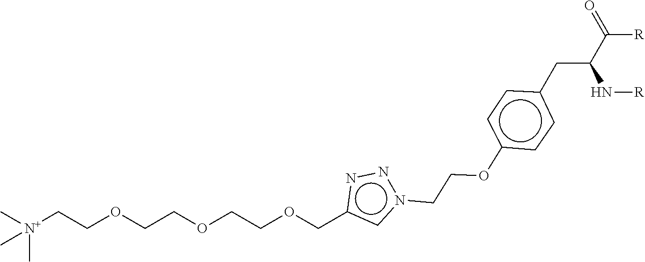 | |
| | C23H36N5O5R2+ | |
| | Y(OTzlPEG3a) | C[N+](C)(C)CCOCCOCCOCc1cn(CCOc2ccc(C |
| | | [C@@H](C([R])═O)N[R])cc2)nn1 |
| 14 | 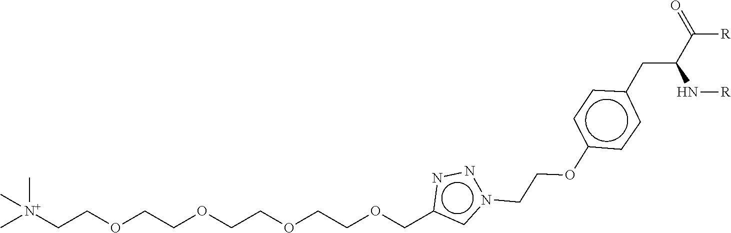 | |
| | C25H40N5O6R2+ | |
| | Y(OTzlPEG4a) | C[N+](C)(C)CCOCCOCCOCCOCc1cn(CCOc2cc |
| | | c(C[C@@H](C([R])═O)N[R])cc2)nn1 |
| 15 | 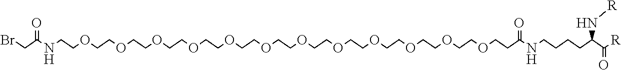 | |
| | C35H66BrN3O15R2 | |
| | k(PEG6Biotin), | O═C(CCOCCOCCOCCOCCOCCOCCOCCOCC |
| | dK(PEG6Biotin) | OCCOCCOCCOCCNC(CBr)═O)NCCCC[C@H] |
| | | (C([R])═O)N[R] |
| 16 | 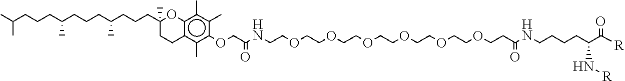 | |
| | C52H91N3O11R2 | |
| | k(dPEG12Ac), dK(dPEG12Ac) | CC(C)CCC[C@@H](C)CCC[C@@H](C)CCC[C |
| | | @](C)(CC1)Oc(c(C)c2C)c1c(C)c2OCC(NCCOC |
| | | COCCOCCOCCOCCOCCC(NCCCC[C@H](C |
| | | ([R])═O)N[R])═O)═O |
| 17 | 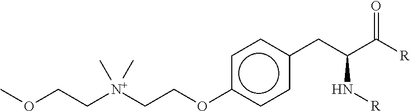 | |
| | C16H25N2O3R2+ | |
| | mPEG2TMA2F | C[N+](C)(CCOC)CCOc1ccc(C[C@@H](C([R])═ |
| | | O)N[R])cc1 |
| 18 | 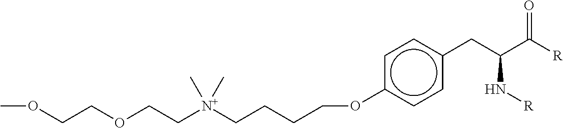 | |
| | C20H33N2O4R2+ | |
| | mPEG3TMA4F | C[N+](C)(CCCCOc1ccc(C[C@@H](C([R])═O)N |
| | | [R])cc1)CCOCCOC |
| 19 | 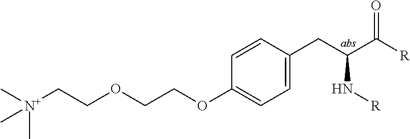 | |
| | C16H25N2O3R2+ | |
| | | C[N+](C)(C)CCOCCOc1cc |
| | | c(C[C@@H](C([R])═O)N |
| | | [R])cc1 |
| 20 | 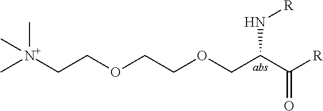 | |
| | C10H21N2O3R2 | |
| | | C[N+](C)(C)CCOCCOC[C |
| | | @@H](C([R])═O)N[R] |
| 21 | 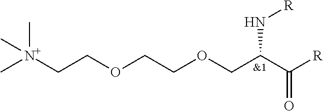 | |
| | C10H21N2O3R2+ | |
| | | C[N+](C)(C)CCOCCOCC( |
| | | C([R])═O)N[R] |
| 22 | 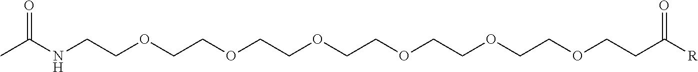 | |
| | C17H32NO8R | |
| | | CC(NCCOCCOCCOCCOC |
| | | COCCOCCC([R])═O)═O |
| 23 | 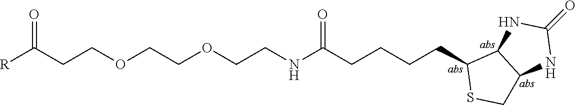 | |
| | C17H28N3O5SR | |
| | | O═C(CCCC[C@@H]([C@ |
| | | H]1N2)SC[C@@H]1NC2═ |
| | | O)NCCOCCOCCC([R])═O |
| 24 | 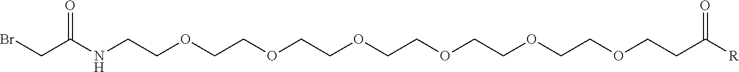 | |
| | C17H31BrNO8R | |
| | | O═C(CBr)NCCOCCOCCO |
| | | CCOCCOCCOCCC([R])═ |
| | | O |
| 25 | 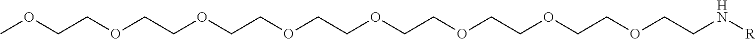 | |
| | C17H36NO8R | |
| | | COCCOCCOCCOCCOCC |
| | | OCCOCCOCCN[R] |
| 26 | 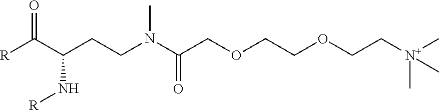 | |
| | C14H28N3O4R2+ | |
| | | CN(CC[C@@H](C([R])═O) |
| | | N[R])C(COCCOCC[N+] |
| | | (C)(C)C)═O |
| 27 | 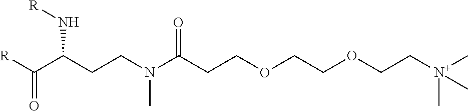 | |
| | C15H30N3O4R2+ | |
| | | CN(CC[C@@H](C([R])═O) |
| | | N[R])C(CCOCCOCC[N+] |
| | | (C)(C)C)═O |
| 28 | 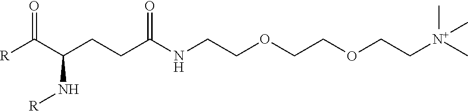 | |
| | C14H28N3O4R2+ | |
| | | C[N+](C)(C)CCOCCOCC |
| | | NC(CC[C@@H](C([R])═O) |
| | | N[R])═O |
| 29 | 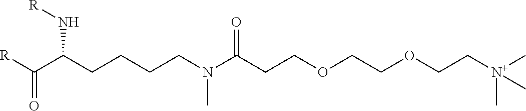 | |
| | C17H34N3O4R2+ | |
| | | CN(CCCC[C@@H](C([R])═ |
| | | O)N[R])C(CCOCCOCC |
| | | [N+](C)(C)C)═O |
| 30 | 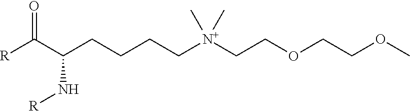 | |
| | C13H27N2O3R2+ | |
| | | C[N+](C)(CCCC[C@@H] |
| | | (C([R])═O)N[R])CCOCCO |
| | | C |
| 31 | 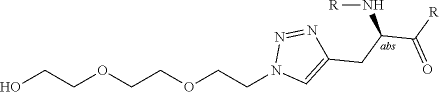 | |
| | C11H18N4O4R2 | |
| | | OCCOCCOCCn1nnc(C[C |
| | | @@H](C([R])═O)N[R])c1 |
| 32 | 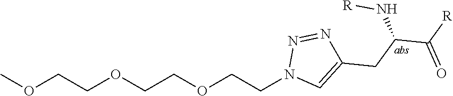 | |
| | C12H20N4O4R2 | |
| | | COCCOCCOCCn1nnc(C[C |
| | | @@H](C([R])═O)N[R])c1 |
| 33 | 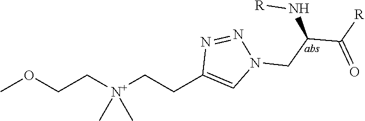 | |
| | C12H22N5O2R2+ | |
| | | C[N+](C)(CCc1cn(C[C@ |
| | | @H](C([R])═O)N[R])nn1) |
| | | CCOC |
| 34 | 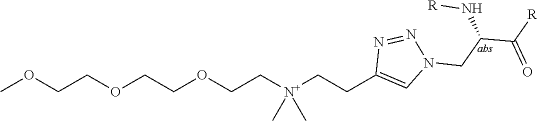 | |
| | C16H30N5O4R2+ | |
| | | C[N+](C)(CCc1cn(C[C@ |
| | | @H](C([R])═O)N[R])nn1) |
| | | CCOCCOCCOC |
| 35 | 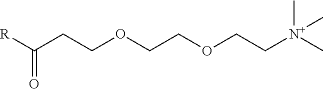 | |
| | C10H21NO3R+ | |
| | | C[N+](C)(C)CCOCCOCC |
| | | C([R])═O |
| 36 | 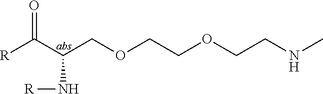 | |
| | C8H16N2O3R2 | |
| | | CNCCOCCOC[C@H](C([ |
| | | R])═O)N[R] |
| 37 | 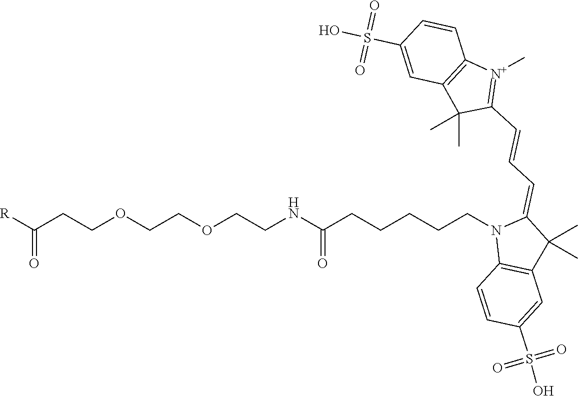 | |
| | C37H49N3O10S2R+ (SulfoCy3dPEG2) | |
| | CC1(C)c(cc(cc2)S(O)(═O)═O)c2[N+](C)═C1/C═C/C═C(/C1(C)C)\N(CCCCCC(NCCOCCOCCC( | |
| | [R])═O)═O)c(cc2)c1cc2S(O)(═O)═O | |
| 38 | 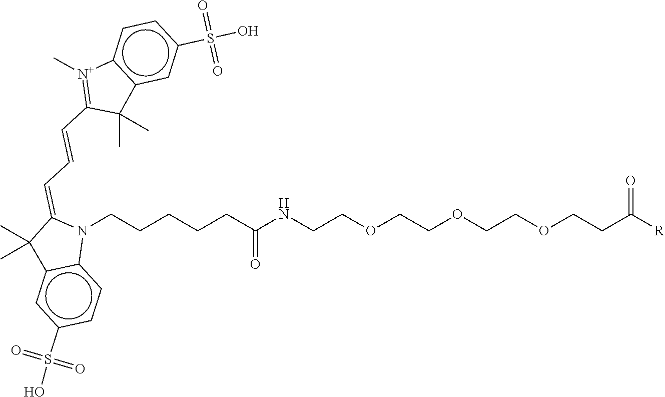 | |
| | C39H53N3O11S2R+ (SulfoCy3dPEG3) | |
| | CC1(C)c(cc(cc2)S(O)(═O)═O)c2[N+](C)═C1/C═C/C═C(/C1(C)C)\N(CCCCCC(NCCOCCOCCO | |
| | CCC([R])═O)═O)c(cc2)c1cc2S(O)(═O)═O | |
| 39 | 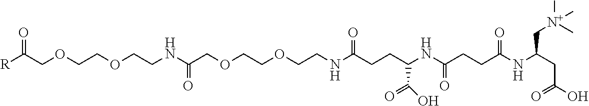 | |
| | C28H49N5O13R+ (d)gEPEG2PEG2 | |
| | | C[N+](C)(C)C[C@@H](CC(O)═O)NC(CCC(N[ |
| | | c@@H](CCC(NCCOCCOCC(NCCOCCOCC([R])═ |
| | | O)═O)═O)C(O)═O)═O)═O |
| 40 | 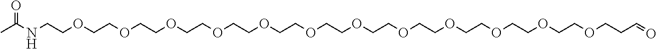 | |
| | C29H57NO14 AcdPEG12CO | |
| | | CC(NCCOCCOCCOCCOCCOCCOCCOCCOCC |
| | | OCCOCCOCCOCCC═O)═O |
| 41 | 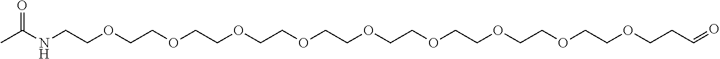 | |
| | C23H45NO11 AcdPEG9CO | |
| | | CC(NCCOCCOCCOCCOCCOCCOCCOCCOCC |
| | | OCCC═O)═O |
| 42 | 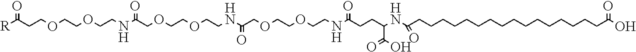 | |
| | C42H75N4O15R | |
| | AEEP(PEG2PEG2gEC18OH) | |
| | | OC(CCCCCCCCCCCCCCCCC(N[C@@H](CC |
| | | C(NCCOCCOCC(NCCOCCOCC(NCCOCCOCC |
| | | C([R])═O)═O)═O)═O)C(O)═O)═O)═O) |
| 43 | 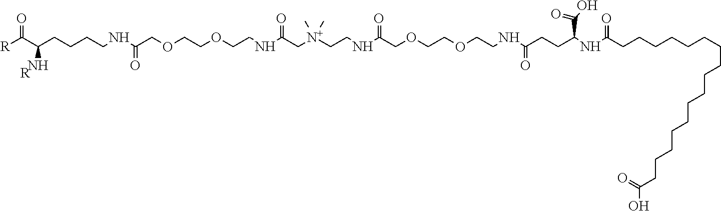 | |
| | C47H86N7O14R2+ | |
| | AEEPPEG2PEG2gEC18OH, | C[N+](C)(CCNC(COCCOCCNC(CC[C@@H](C |
| | k(PEG2Sp6PEG2gEC18OH), | (O)═O)NC(CCCCCCCCCCCCCCCCC(O)═O)═ |
| | dK(PEG2Sp6PEG2gEC18OH) | O)═O)═O)CC(NCCOCCOCC(NCCCC[C@H](C |
| | | ([R])═O)N[R])═O)═O |
| 44 | 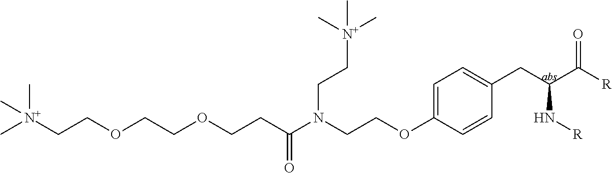 | |
| | C26H46N4O5R2 + 2 | |
| | AEF((Ch)cPEG3a) | C[N+](C)(C)CCN(CCOc1ccc(C[C@@H](C([R])═O) |
| | | N[R])cc1)C(CCOCCOCC[N+](C)(C)C)═O |
| 45 | 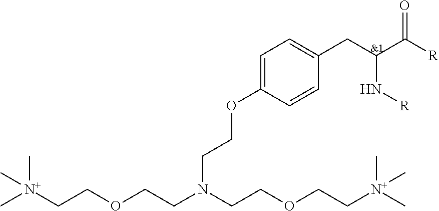 | |
| | C25H46N4O4R2 + 2 | |
| | AEF(BisPEG2a)(RS) | C[N+](C)(C)CCOCCN(CCOCC[N+](C)(C)C)CC |
| | AEF(BisPEG2a)(S*) | Oc1ccc(CC(C([R])═O)N[R])cc1 |
| | (The RS and the S* indicates the | |
| | stereochemistry) | |
| 46 | 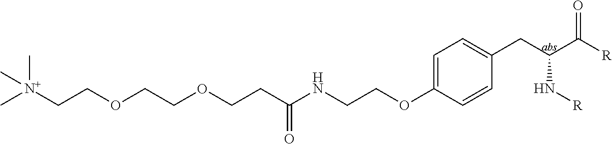 | |
| | C21H34N3O5R2+ | |
| | AEF(NMePEG3a), AEF(NMecPEG3aCO) | C[N+](C)(C)CCOCCOCCC(NCCOc1ccc(C[C@ |
| | | @H](C([R])═O)N[R])cc1)═O |
| 47 | 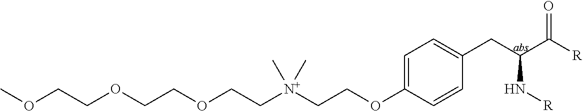 | |
| | C20H33N2O5R2+ | |
| | AEF(NMe2mPEG3) | C[N+](C)(CCOCCOCCOC)CCOc1ccc(C[C@@ |
| | | H](C([R])═O)N[R])cc1 |
| 48 | 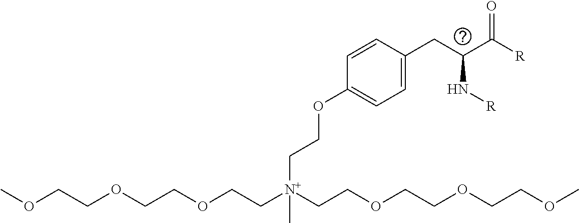 | |
| | C26H45N2O8R2+ | |
| | AEF(NMeBismPEG3) | C[N+](CCOCCOCCOC)(CCOCCOCCOC)CCOc |
| | | 1ccc(C[C@@H](C([R])═O)N[R])cc1 |
| 49 | 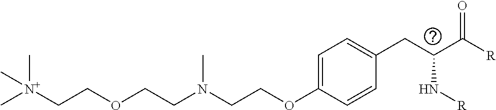 | |
| | C19H32N3O3R2+ | |
| | AEF(NMePEG2a) | CN(CCOCC[N+](C)(C)C)CCOc1ccc(C[C@H](C |
| | | ([R])═O)N[R])cc1 |
| 50 | 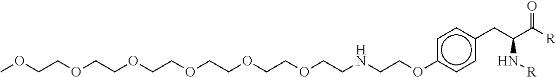 | |
| | C24H40N2O8R2 | |
| | AEF(NmPEG6) | COCCOCCOCCOCCOCCOCCNCCOc1ccc(C[C @@H](C([R])═O)N[R])cc1 |
| 51 | 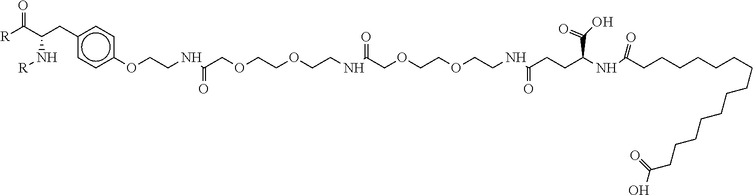 | |
| | C44H71N5O14R2 | |
| | AEF(PEG2PEG2gEC16OH) | OC(CCCCCCCCCCCCCCC(N[C@@H](CCC(N |
| | | CCOCCOCC(NCCOCCOCC(NCCOc1ccc(C[C@ |
| | | @H](C([R])═O)N[R])cc1)═O)═O)═O)C(O)═O)═ |
| | | O)═O |
| 52 | 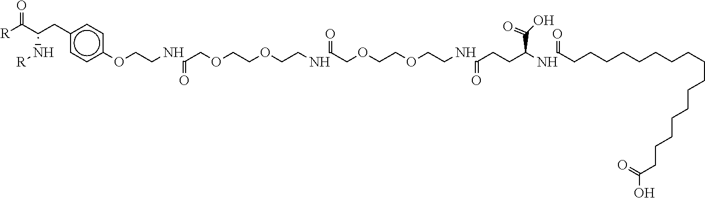 | |
| | C46H75N5O14R2 | |
| | AEF(PEG2PEG2gEC18OH) | OC(CCCCCCCCCCCCCCCCC(N[C@@H](CC |
| | | C(NCCOCCOCC(NCCOCCOCC(NCCOc1ccc(C |
| | | [C@@H](C([R])═O)N[R])cc1)═O)═O)═O)C(O)═ |
| | | O)═O)═O |

| | |
|---|---|
| 53 | 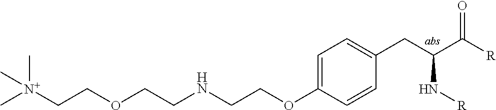 |

| | C18H30N3O3R2+ | |
|---|---|---|
| | AEF(Peg2a), AEF(PEG2a) | C[N+](C)(C)CCOCCNCCOc1ccc(C[C@@H](C |
| | | ([R])═O)N[R])cc1 |
| 54 | 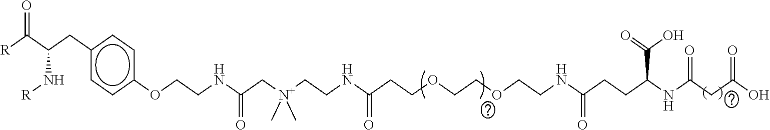 | |
| | C67H119N6O22R2+ | |
| | AEF(SP6PEG12gEC18OH) | C[N+](C)(CCNC(CCOCCOCCOCCOCCOCCO |
| | | CCOCCOCCOCCOCCOCCOCCNC(CC[C@@ |
| | | H](C(O)═O)NC(CCCCCCCCCCCCCCCCC(O)═ |
| | | O)═O)═O)═O)CC(NCCOc1ccc(C[C@@H](C([R])═ |
| | | O)N[R])cc1)═O |
| 55 | 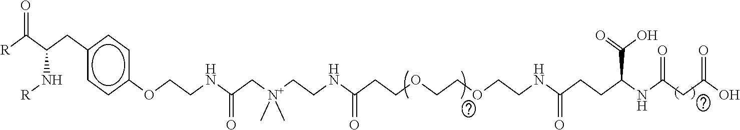 | |
| | C69H123N6O22R2+ | |
| | AEF(SP6PEG12gEC20OH) | C[N+](C)(CCNC(CCOCCOCCOCCOCCOCCO |
| | | CCOCCOCCOCCOCCOCCOCCNC(CC[C@@ |
| | | HJ(C(O)═O)NC(CCCCCCCCCCCCCCCCCCC( |
| | | O)═O)═O)═O)═O)CC(NCCOc1ccc(C[C@@H](C |
| | | ([R])═O)N[R])cc1)═O |
| 56 | 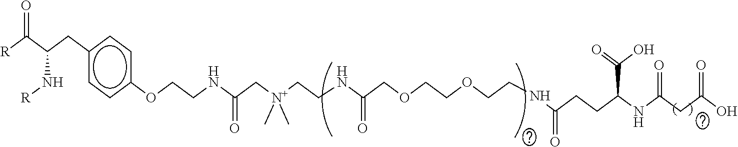 | |
| | C52H88N7O15R2+ | |
| | AEF(SP6PEG2PEG2gEC18OH) | C[N+](C)(CCNC(COCCOCCNC(COCCOCCNC |
| | | (CC[C@@H](C(O)═O)NC(CCCCCCCCCCCCC |
| | | CCCC(O)═O)═O)═O)═O)═O)CC(NCCOc1ccc(C |
| | | [C@@H(C([R])═O)N[R])cc1)═O |
| 57 | 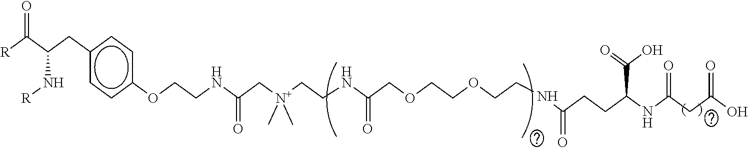 | |
| | C54H92N7O15R2+ | |
| | AEF(SP6PEG2PEG2gEC20OH) | C[N+](C)(CCNC(COCCOCCNC(COCCOCCNC |
| | | (CC[C@@H](C(O)═O)NC(CCCCCCCCCCCCC |
| | | CCCCCC(O)═O)═O)═O)═O)═O)CC(NCCOc1ccc |
| | | (C[C@@H(C([R])═O)N[R])cc1)═O |
| 58 | 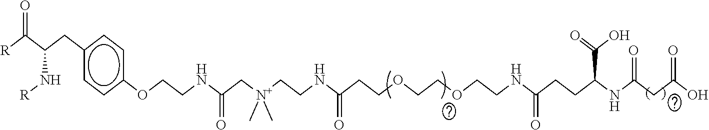 | |
| | C55H95N6O16R2+ | |
| | AEF(SP6PEG6gEC18OH) | C[N+](C)(CCNC(CCOCCOCCOCCOCCOCCO |
| | | CCNC(CC[C@@H](C(O)═O)NC(CCCCCCCCC |
| | | CCCCCCCC(O)═O)═O)═O)═O)CC(NCCOc1ccc |
| | | (C[C@@H(C([R])═O)N[R])cc1)═O |
| 59 | 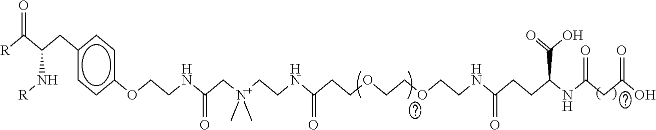 | |
| | C57H99N6O16R2+ | |
| | AEF(SP6PEG6gEC20OH) | C[N+](C)(CCNC(CCOCCOCCOCCOCCOCCO |
| | | CCNC(CC[C@@H](C(O)═O)NC(CCCCCCCCC |
| | | CCCCCCCCCC(O)═O)═O)═O)═O)CC(NCCOc1 |
| | | ccc(C[C@@H](C([R])═O)N[R])cc1)═O |

| | |
|---|---|
| 60 | 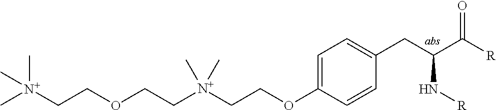 |

| C20H35N3O3R2+2 | |
|---|---|
| AEF(aPEG2a) | C[N+](C)(C)CCOCC[N+](C)(C)CCOc1ccc(C[C |
| | @@H(C([R])═O)N[R])cc1 |

| | |
|---|---|
| 61 | 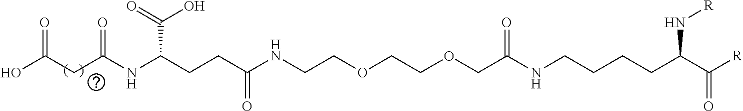 |

| C35H62N4O10R2 | |
|---|---|
| k(PEG2gEC18OH), d K(PEG2gEC18OH) | OC(CCCCCCCCCCCCCCCCC(N[C@@H](CC |
| | C(NCCOCCOCC(NCCCC[C@H](C([R])═O)N[R])═ |
| | O)═O)C(O)═O)═O)═O |

| | |
|---|---|
| 62 | 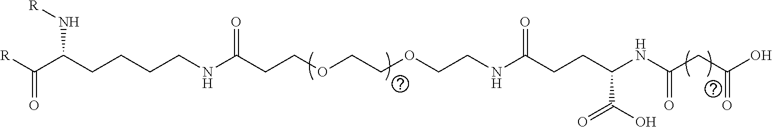 |

| | C44H80N4O14R2 | |
|---|---|---|
| | k(PEG6gEC18OH), d K(PEG6gEC18OH) | OC(CCCCCCCCCCCCCCCCC(N[C@@H](CC |
| | | C(NCCOCCOCCOCCOCCOCCOCCC(NCCCC |
| | | [C@H](C([R])═O)N[R])═O)═O)C(O)═O)═O)═O |
| 63 | 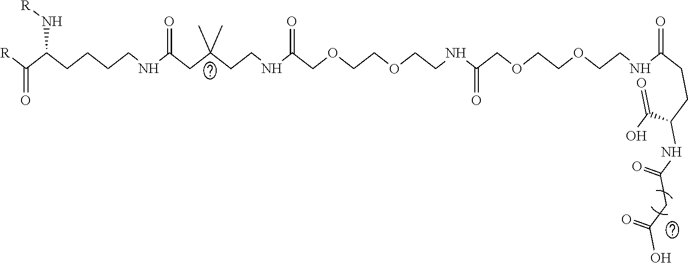 | |
| | C47H86N7O14R2+ | |
| | k(Sp6PEG2PEG2gEC18OH), | C[N+](C)(CCNC(COCCOCCNC(COCCOCCNC |
| | dK(Sp6PEG2PEG2gEC18OH) | (CC[C@@H](C(O)═O)NC(CCCCCCCCCCCCC |
| | | CCCC(O)═O)═O)═O)═O)═O)CC(NCCCC[C@H] |
| | | (C([R])═O)N[R])═O |

| | |
|---|---|
| 64 | 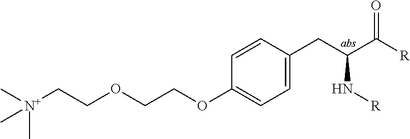 |

| C16H25N2O3R2+ | |
|---|---|
| APEG2F | C[N+](C)(C)CCOCCOc1ccc(C[C@@H](C([R])═ |
| | O)N[R])cc1 |

| | |
|---|---|
| 65 | 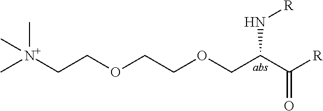 |

| C10H21N2O3R2+ | |
|---|---|
| APEG2ser | C[N+](C)(C)CCOCCOC[C@@H](C([R])═ |
| | O)N[R] |

| | |
|---|---|
| 66 | 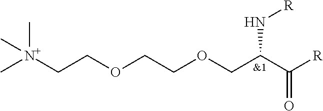 |

| C10H21N2O3R2+ | |
|---|---|
| APEG2Ser(R*) | C[N+](C)(C)CCOCCOCC(C([R])═O)N[R] |
| APEG2Ser(S*) | |

| | |
|---|---|
| 67 | 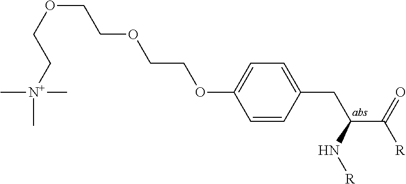 |

| C18H29N2O4R2+ | |
|---|---|
| APEG3F | C[N+](C)(C)CCOCCOCCOc1ccc(C[C@@H](C( |
| | [R])═O)N[R])cc1 |
| | C[N+](C)(C)CCOCCOCCOc1ccc(C[C@@H](C═ |
| | O)N)cc1 |

| | |
|---|---|
| 68 | 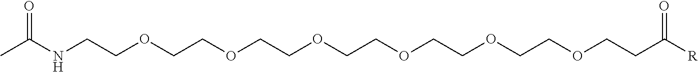 |

| | C17H32NO8R | |
|---|---|---|
| | AcdPEG6CO | CC(NCCOCCOCCOCCOCCOCCOCCC([R])═ |
| | | O)═O |
| 69 | 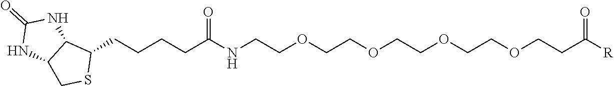 | |
| | C21H36N3O7SR | |
| | BiotinPEG4CO, Biotin(PEG4CO), | O═C(CCCC[C@@H]([C@H]1N2)SC[C@@H]1 |
| | Biotin(PEG4) | NC2═O)NCCOCCOCCOCCOCCC([R])═O |

| | |
|---|---|
| 70 | 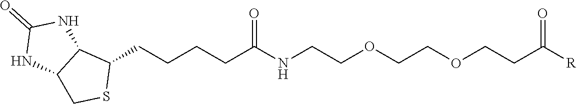 |

| | |
|---|---|
| Biotinyl(dPEG2), Biotin(dPEG2) | O═C(CCCC[C@@H]([C@H]1N2)SC[C@@H]1 |
| | NC2═O)NCCOCCOCCC([R])═O |

| | |
|---|---|
| 71 | 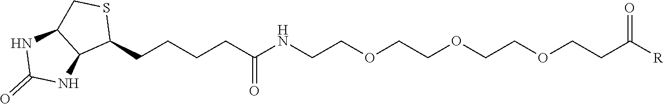 |

| C19H32N3O6SR | |
|---|---|
| Biotinyl(dPEG3), Biotin(dPEG3) | O═C(CCCC[C@@H]([C@H]1N2)SC[C@@H]1 |
| | NC2═O)NCCOCCOCCOCCC([R])═O |

| | |
|---|---|
| 72 | 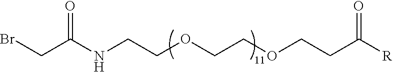 |

| C29H55BrNO14R | |
|---|---|
| BrAcdPEG12CO | O═C(CBr)NCCOCCOCCOCCOCCOCCOCCOC |
| | COCCOCCOCCOCCOCCC([R])═O |

| | |
|---|---|
| 73 | 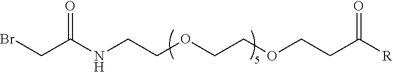 |

| C17H31BrNO8R | |
|---|---|
| BrAcdPEG6CO | O═C(CBr)NCCOCCOCCOCCOCCOCCOCCC |
| | ([R])═O |

| | |
|---|---|
| 74 | 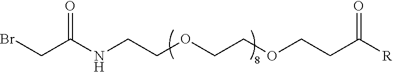 |

| | C23H43BrNO11R | |
|---|---|---|
| | BrAcdPEG9CO | O═C(CBr)NCCOCCOCCOCCOCCOCCOCCOC |
| | | COCCOCCC([R])═O |
| 75 | 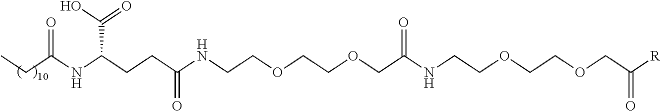 | |
| | C29H52N3O10R | |
| | C12gEPEG2PEG2, | CCCCCCCCCCCC(N[C@@H](CCC(NCCOCC |
| | C12gEPEG2PEG2CO | OCC(NCCOCCOCC([R])═O)═O)═O)C(O)═ |
| | | O)═O |
| 76 | 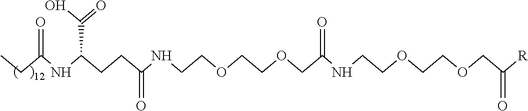 | |
| | C31H56N3O10R | |
| | C14gEPEG2PEG2, | CCCCCCCCCCCCCC(N[C@@H](CCC(NCCO |
| | C14gEPEG2PEG2CO | CCOCC(NCCOCCOCC([R])═O)═O)═O)C(O)═ |
| | | O)═O |

| | |
|---|---|
| 77 | 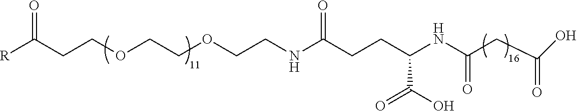 |

| | C50H93N2O19R | |
|---|---|---|
| | C18OHgEPEG12, HOC18gEPEG12 | OC(CCCCCCCCCCCCCCCCC(N[C@@H](CC |
| | | C(NCCOCCOCCOCCOCCOCCOCCOCCOCCO |
| | | CCOCCOCCOCCC([R])═O)═O)C(O)═O)═O)═O |
| 78 | 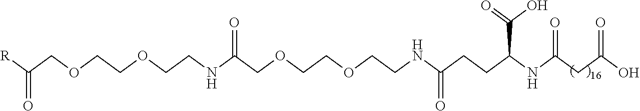 | |
| | C35H62N3O12R | |
| | C18OHgEPEG2PEG2, HOC18gEPEG2PEG2 | OC(CCCCCCCCCCCCCCCCC(N[C@@H](CC |
| | PEG2PEG2gEC18OH | C(NCCOCCOCC(NCCOCCOCC([R])═O)═O)═ |
| | | O)C(O)═O)═O)═O |
| | | OC(CCCCCCCCCCCCCCCCC(N[C@@H](CC |
| | | C(NCCOCCOCC(NCCOCCOCC([R])═O)═O)═ |
| | | O)C(O)═O)═O)═O |
| | | OC(CCCCCCCCCCCCCCCCC(N[C@@H](CC |
| | | C(NCCOCCOCC(NCCOCCOCC═O)═O)═O)C |
| | | (O)═O)═O)═O |
| 79 | 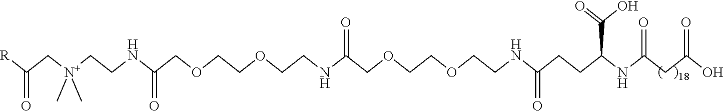 | |
| | C41H75N5O13R+ | |
| | C18OHgEPEG2PEG2SP6, | C[N+](C)(CCNC(COCCOCCNC(COCCOCCNC |
| | HOC18gEPEG2PEG2SP6 | (CC[C@@H](C(O)═O)NC(CCCCCCCCCCCCC |
| | | CCCC(O)═O)═O)═O)═O)═O)CC([R])═O |
| 80 | 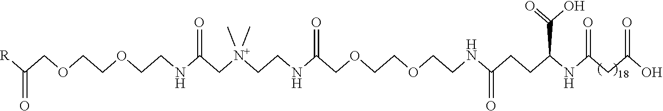 | |
| | C41H75N5O13R+ | |
| | C18OHgEPEG2SP6PEG2, | C[N+](C)(CCNC(COCCOCCNC(CC[C@@H](C |
| | HOC18gEPEG2SP6PEG2 | (O)═O)NC(CCCCCCCCCCCCCCCCC(O)═O)═ |
| | | O)═O)═O)CC(NCCOCCOCC([R])═O)═O |
| 81 | 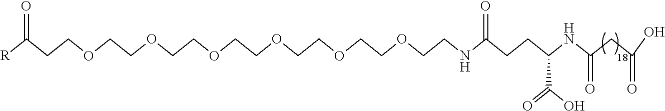 | |
| | C38H69N2O13R | |
| | C18OHgEPEG6, HOC18gEPEG6 | OC(CCCCCCCCCCCCCCCCC(N[C@@H](CC |
| | | C(NCCOCCOCCOCCOCCOCCOCCC([R])═O)═ |
| | | O)C(O)═O)═O)═O |
| 82 | 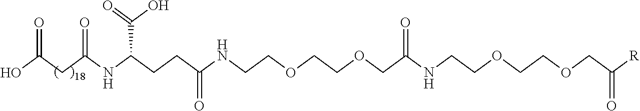 | |
| | C37H66N3O12R | |
| | C200HgEPEG2PEG2, HOC20gEPEG2PEG2 | OC(CCCCCCCCCCCCCCCCCCC(N[C@@H] |
| | | (CCC(NCCOCCOCC(NCCOCCOCC([R])═O)═ |
| | | O)═O)C(O)═O)═O)═O |
| 83 | 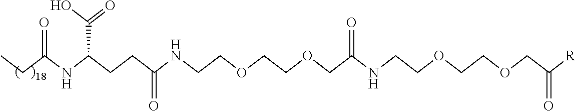 | |
| | C37H68N3O10R | |
| | C20gEPEG2PEG2 | CCCCCCCCCCCCCCCCCCCC(N[C@@H](CC |
| | | C(NCCOCCOCC(NCCOCCOCC([R])═O)═O)═ |
| | | O)C(O)═O)═O |

| | |
|---|---|
| 84 | 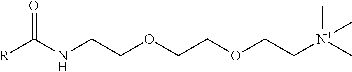 |

| C10H22N2O3R+ | |
|---|---|
| CO(NHPEG3a) | C[N+](C)(C)CCOCCOCCNC([R])═O |
| CON(PEG3a) | |
| CONHPEG3a | |

| | |
|---|---|
| 85 | 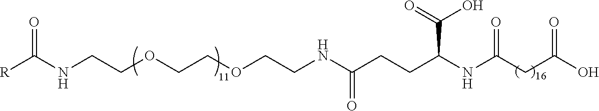 |

| | C50H94N3O19R | |
|---|---|---|
| | CO(PEG12gEC18OH) | OC(CCCCCCCCCCCCCCCCC(N[C@@H](CC |
| | | C(NCCOCCOCCOCCOCCOCCOCCOCCOCCO |
| | | CCOCCOCCOCCNC([R])═O)═O)C(O)═O)═ |
| | | O)═O |
| 86 | 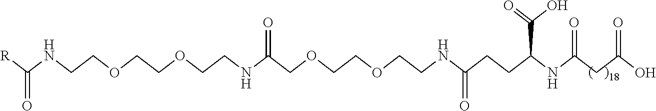 | |
| | C36H65N4O12R | |
| | CO(PEG2PEG2gEC18OH) | OC(CCCCCCCCCCCCCCCCC(N[C@@H](CC |
| | | C(NCCOCCOCC(NCCOCCOCCNC([R])═O)═ |
| | | O)═O)C(O)═O)═O)═O |
| 87 | 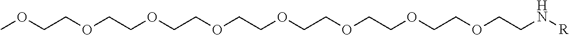 | |
| | C17H36NO8R | |
| | CO(mPEG8) | COCCOCCOCCOCCOCCOCCOCCOCCN[R] |

| | |
|---|---|
| 88 | 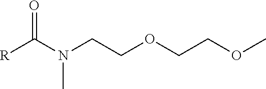 |

| C7H14NO3R | |
|---|---|
| CON(MePEG2) | CN(CCOCCOC)C([R])═O |

| | |
|---|---|
| 89 | 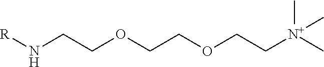 |

| C9H22N2O2R+ | |
|---|---|
| CONH(PEG3a) | C[N+](C)(C)CCOCCOCCN[R] |

| | |
|---|---|
| 90 | 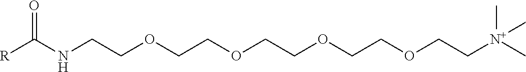 |

| C14H30N2O5R+ | |
|---|---|
| CONH(PEG5a) | C[N+](C)(C)CCOCCOCCOCCOCCNC([R])═O |

| | |
|---|---|
| 91 | 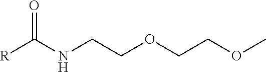 |

| C6H12NO3R | |
|---|---|
| CONH(mPEG2) | COCCOCCNC([R])═O |

| | |
|---|---|
| 92 | 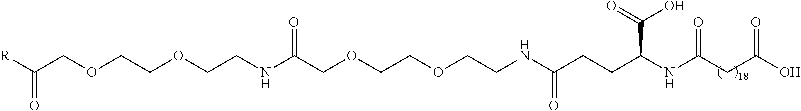 |

| C33H58N3O12R | |
|---|---|
| PEG2PEG2gEC160H | OC(CCCCCCCCCCCCCCC(N[C@@H](CCC(N |
| | CCOCCOCC(NCCOCCOCC([R])═O)═O)═O) |
| | C(O)═O)═O)═O |
| | OC(CCCCCCCCCCCCCCC(N[C@@H](CCC(N |
| | CCOCCOCC(NCCOCCOCC═O)═O)═O)C(O)═ |
| | O)═O)═O |

| | |
|---|---|
| 93 | 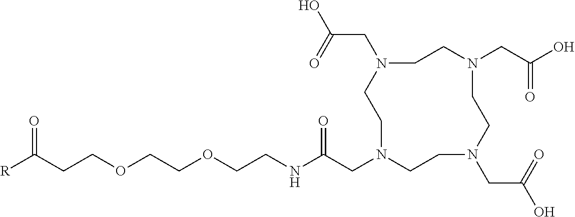 |

| C23H40N5O10R | |
|---|---|
| DOTA(dPEG2) | OC(CN1CCN(CC(O)═O)CCN(CC(O)═O)CCN(C |
| | C(NCCOCCOCCC([R])═O)═O)CC1)═O |

| | |
|---|---|
| 94 | 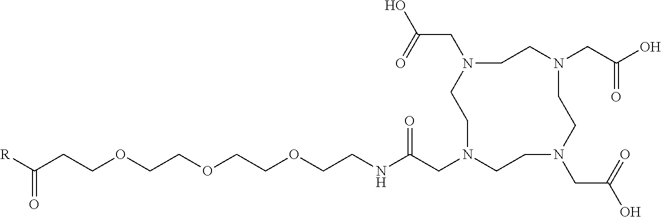 |

| | C25H44N5O11R | |
|---|---|---|
| | DOTA(dPEG3) | OC(CN1CCN(CC(O)═O)CCN(CC(O)═O)CCN(C |
| | | C(NCCOCCOCCOCCC([R])═O)═O)CC1)═O |
| 95 | 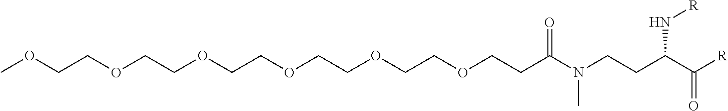 | |
| | C19H36N2O8R2 | |
| | Dab(NMeCOmPEG6) | CN(CC[C@@H](C([R])═O)N[R])C(CCOCCOC |
| | | COCCOCCOCCOC)═O |

| | |
|---|---|
| 96 | 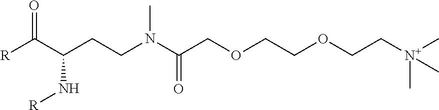 |

| C14H28N3O4R2+ | |
|---|---|
| Dab(NMecPEG2aCO), Dab(NMecPEG2a) | CN(CC[C@@H](C([R])═O)N[R])C(COCCOCC |
| | [N+](C)(C)C)═O |

| | |
|---|---|
| 97 | 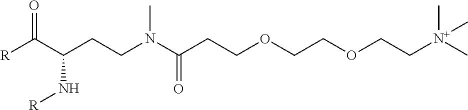 |

| | |
|---|---|
| Dab(NMecPEG3aCO), Dab(NMecPEG3a) | CN(CC[C@@H](C([R])═O)N[R])C(CCOCCOC |
| | C[N+](C)(C)C)═O |

| | |
|---|---|
| 98 | 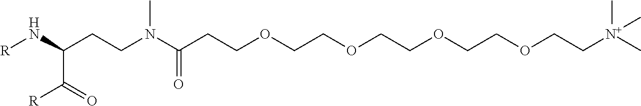 |

| C19H38N3O6R2+ | |
|---|---|
| Dab(NMecPEG5aCO), Dab(NMecPEG5a) | CN(CC[C@@H](C([R])═O)N[R])C(CCOCCOC |
| | COCCOCC[N+](C)(C)C)═O |

| 99 | 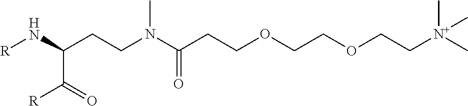 | |
|---|---|---|
| | C14H28N3O4R2+ | |
| | E(COcPEG3a)) | C[N+](C)(C)CCOCCOCCNC(CC[C@@H](C([R])═ |
| | | O)N[R])═O |
| 100 | 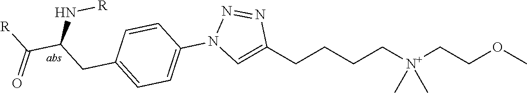 | |
| | C20H30N5O2R2+ | |
| | F(4TzlDMA4mPEG) | C[N+](C)(CCCCc1cn(- |
| | | c2ccc(C[C@@H](C([R])═O)N[R])cc2)nn1)CCO |
| | | C |
| 101 | 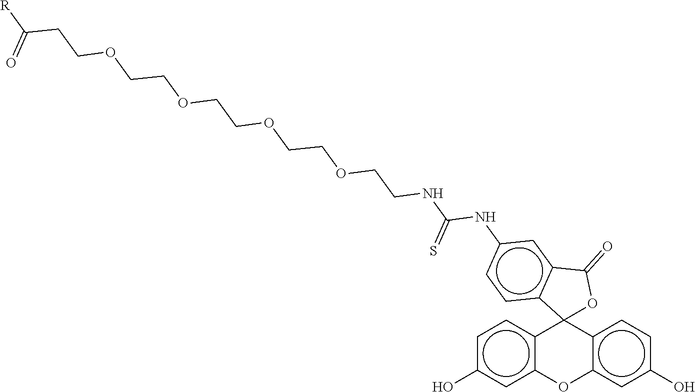 | |
| | C32H33N2O10SR | |
| | FITCPEG4CO | Oc1cc(Oc2c(C3(c(cc4)c5cc4NC(NCCOCCOCCO |
| | | CCOCCC([R])═O)═S)OC5═O)ccc(O)c2)c3cc1 |
| 102 | 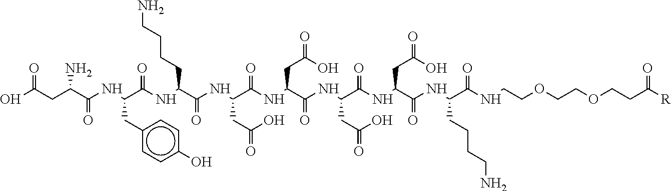 | |
| | C48H72N11O22R | |
| | Flag Tag(dPEG2) | NCCCC[C@@H](C(NCCOCCOCCC([R])═O)═ |
| | | O)NC([C@H](CC(O)═O)NC([C@H](CC(O)═O) |
| | | NC([C@H](CC(O)═O)NC([C@H](CC(O)═O)NC |
| | | ([C@H](CCCCN)NC([C@H](Cc(cc1)ccc1O)NC |
| | | ([C@H](CC(O)═O)N)═O)═O)═O)═O)═O)═O)═O |
| 103 | 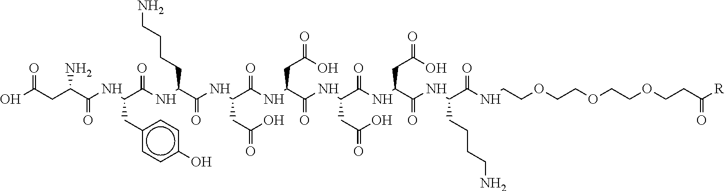 | |
| | Flag Tag(dPEG3) | NCCCC[C@@H](C(NCCOCCOCCOCCC([R])═ |
| | | O)═O)NC([C@H](CC(O)═O)NC([C@H](CC |
| | | (O)═O)NC([C@H](CC(O)═O)NC([C@H](CC(O)═ |
| | | O)NC([C@H](CCCCN)NC([C@H](Cc(cc1)ccc1O) |
| | | NC(C@H](CC(O)═O)N)═O)═O)═O)═O)═O)═O)═O |
| 104 | 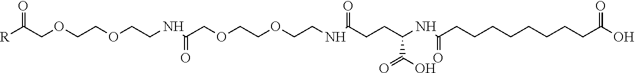 | |
| | C27H46N3O12R | |
| | HOC10gEPEG2PEG2, | OC(CCCCCCCCC(N[C@@H](CCC(NCCOCCO |
| | HOC10gEPEG2PEG2CO | CC(NCCOCCOCC([R])═O)═O)═O)C(O)═O)═ |
| | | O)═O |
| | | OC(CCCCCCCCC(N[C@@H](CCC(NCCOCCO |
| | | CC(NCCOCCOCC═O)═O)═O)C(O)═O)═O)═O |
| 105 | 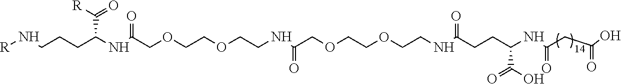 | |
| | C38H67N5O13R2 | |
| | HOC16gEPEG2PEG2orn, | OC(CCCCCCCCCCCCCCC(N[C@@H](CCC(N |
| | HOC160HgEPEG2PEG2om(2) | CCOCCOCC(NCCOCCOCC(N[C@H](CCCN[R |
| | | NCCC[C@H](C═O)NC(COCCOCCNC(COCCO |
| | | CCNC(CC[C@@H](C(O)═O)NC(CCCCCCCCC |
| | | CCCCCC(O)═O)═O)═O)═O)═O |
| 106 | 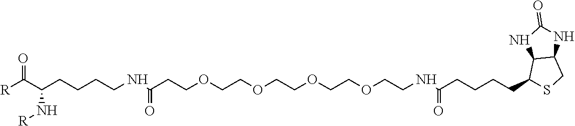 | |
| | C27H47N5O8SR2 | |
| | K(BiotinPEG4) | O═C(CCCC[C@@H]([C@H]1N2)SC[C@@H]1 |
| | | NC2═O)NCCOCCOCCOCCOCCC |
| | | (NCCCC[C@@H](C([R])═O)N[R])═O |
| 107 | 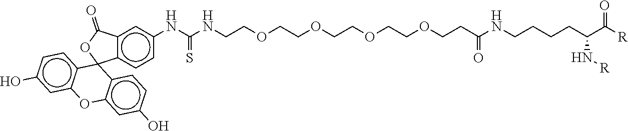 | |
| | C38H44N4O11SR2 | |
| | K(FITCPEG4) | Oc1cc(Oc2c(C3(c(cc4)c5cc4NC(NCCOCCOCCO |
| | | CCOCCC(NCCCC[C@@H](C([R])═O)N[R])═ |
| | | O)═S)OC5═O)ccc(O)c2)c3cc1 |
| 108 | 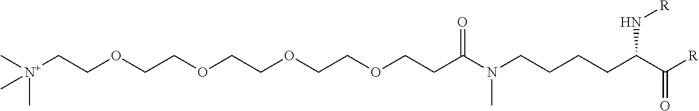 | |
| | C21H42N3O6R2+ | |
| | K(NMeCOPEG4N+Me3) | CN(CCCC[C@@H](C([R])═O)N[R])C(CCOCC |
| | | OCCOCCOCC[N+](C)(C)C)═O |
| 109 | 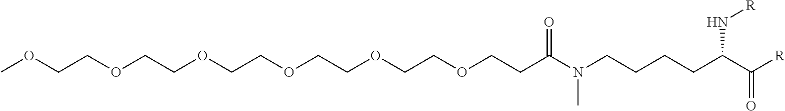 | |
| | C21H40N2O8R2 | |
| | K(NMeCOmPEG6) | CN(CCCC[C@@H](C([R])═O)N[R])C(CCOCC |
| | | OCCOCCOCCOCCOC)═O |
| 110 | 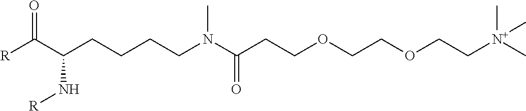 | |
| | C17H34N3O4R2+ | |
| | K(NMePEG3a), K(NMecPEG3a), | CN(CCCC[C@@H](C([R])═O)N[R])C(CCOCC |
| | K(NMecPEG3aCO) | OCC[N+](C)(C)C)═O |
| 111 | 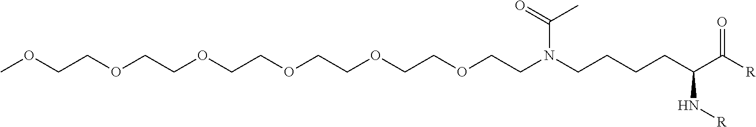 | |
| | C21H40N2O8R2 | |
| | K(NmPEG6Ac) | CC(N(CCCC[C@@H](C([R])═O)N[R])CCOCC |
| | | OCCOCCOCCOCCOC)═O |
| 112 | 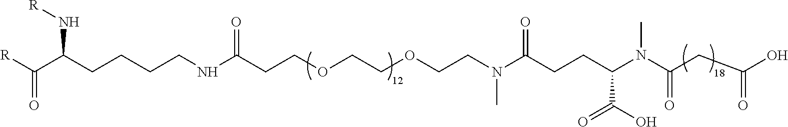 | |
| | C58H108N4O20R2 | |
| | K(PEG12NMegENMeC18OH) | CN(CCOCCOCCOCCOCCOCCOCCOCCOCCO |
| | | CCOCCOCCOCCC(NCCCC[C@@H](C([R])═O |
| | | CCCCCCCCCCCCCC(O)═O)═O)═O |
| 113 | 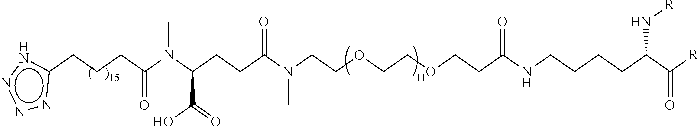 | |
| | C59H110N8O18R2 | |
| | K(PEG12NMegENMeC18Tetrazole) | CN(CCOCCOCCOCCOCCOCCOCCOCCOCCO |
| | | CCOCCOCCOCCC(NCCCC[C@@H](C([R])═O) |
| | | N[R])═O)C(CC[C@@H](C(O)═O)N(C)C(CCC |
| | | CCCCCCCCCCCCCCc1nnn[nH]1)═O)═O |
| 114 | 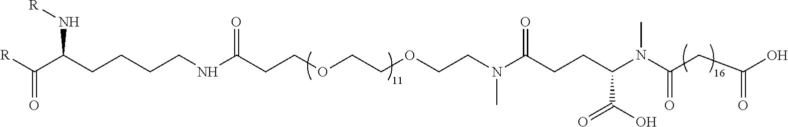 | |
| | C56H104N4O20R2 | |
| | K(PEG12gEC18OH) | OC(CCCCCCCCCCCCCCCCC(N[C@@H](CC |
| | | C(NCCOCCOCCOCCOCCOCCOCCOCCOCCO |
| | | CCOCCOCCOCCC(NCCCC[C@@H](C([R])═ |
| | | O)N[R])═O)═O)C(O)═O)═O)═O |
| 115 | 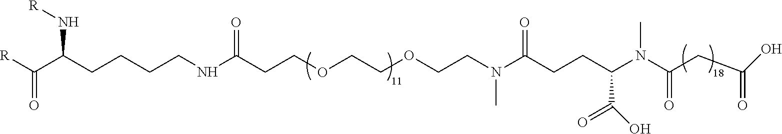 | |
| | C58H108N4O20R2 | |
| | K(PEG12gEC20OH) | OC(CCCCCCCCCCCCCCCCCCC(N[C@@H] |
| | | (CCC(NCCOCCOCCOCCOCCOCCOCCOCCOC |
| | | COCCOCCOCCOCCC(NCCCC[C@@H](C |
| | | ([R])═O)N[R]═O)═O)C(O)═O)═O)═O |
| 116 | 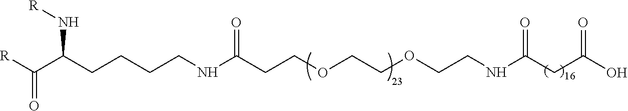 | |
| | C75H145N3O29R2 | |
| | K(PEG24C18OH) | OC(CCCCCCCCCCCCCCCCC(NCCOCCOCCO |
| | | CCOCCOCCOCCOCCOCCOCCOCCOCCOCC |
| | | OCCOCCOCCOCCOCCOCCOCCOCCOCCOC |
| | | COCCOCCC(NCCCC[C@@H](C([R])═O) |
| | | N[R])═O)═O)═O |
| 117 | 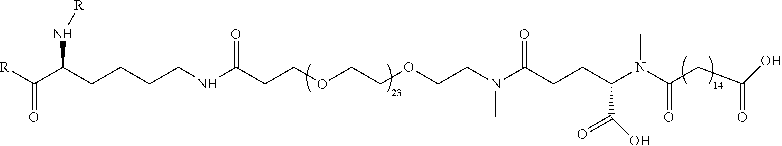 | |
| | C78H148N4O32R2 | |
| | K(PEG24gEC16OH) | OC(CCCCCCCCCCCCCCC(N[C@@H](CCC(N |
| | | CCOCCOCCOCCOCCOCCOCCOCCOCCOCC |
| | | OCCOCCOCCOCCOCCOCCOCCOCCOCCOC |
| | | COCCOCCOCCOCCOCCC(NCCCC[C@@H](C |
| | | ([R])═O)N[R]═O)═O)C(O)═O)═O)═O |
| 118 | 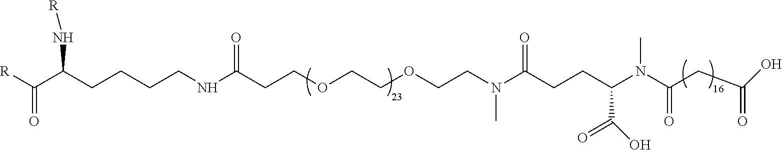 | |
| | C81H154N4O32R2 | |
| | K(PEG24gEC18OH) | C[C@](CCCCNC(CCOCCOCCOCCOCCOCCO |
| | | CCOCCOCCOCCOCCOCCOCCOCCOCCOCC |
| | | OCCOCCOCCOCCOCCOCCOCCOCCOCCNC |
| | | (CC[C@@H](C(O)═O)NC(CCCCCCCCCCCCC |
| | | CCCC(O)═O)═O)═O)═O)(C([R])═O)N[R] |
| 119 | 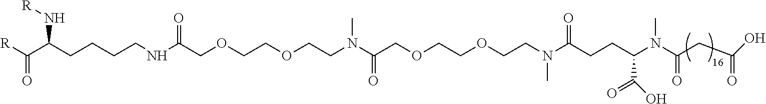 | |
| | C44H79N5O13R2 | |
| | K(PEG2NMePEG2NMegENMeC18OH) | CN(CCOCCOCC(N(C)CCOCCOCC(NCCCC[C |
| | | @@H](C([R])═O)N[R])═O)═O)C(CC[C@@H] |
| | | (C(O)═O)N(C)C(CCCCCCCCCCCCCCCCC(O)═ |
| | | O)═O)═O |
| 120 | 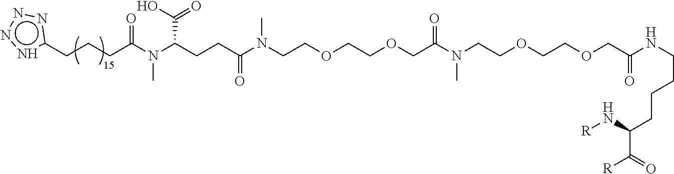 | |
| | C45H81N9O11R2 | |
| | K(PEG2NMePEG2NMegENMeC18Tetrazole | CN(CCOCCOCC(N(C)CCOCCOCC(NCCCC[C |
| | | @@H](C([R])═O)N[R])═O)═O)C(CC[C@@H] |
| | | (C(O)═O)N(C)C(CCCCCCCCCCCCCCCCCc1nn |
| | | n[nH]1)═O)═O |
| 121 | 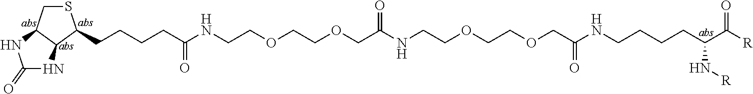 | |
| | C28H48N6O9SR2 | |
| | K(PEG2PEG2Biotin) | O═C(CCCC[C@@H]([C@H]1N2)SC[C@@H]1 |
| | | NC2═O)NCCOCCOCC(NCCOCCOCC(NCCCC |
| | | [C@@H](C([R])═O)N[R])═O)═O |
| 122 | 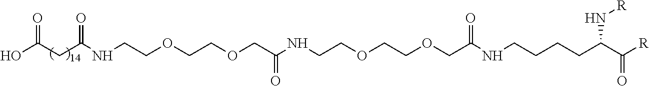 | |
| | C34H62N4O10R2 | |
| | K(PEG2PEG2C16OH) | OC(CCCCCCCCCCCCCCC(NCCOCCOCC(NC |
| | | COCCOCC(NCCCC[C@@H](C([R])═O)N[R])═ |
| | | O)═O)═O)═O |
| 123 | 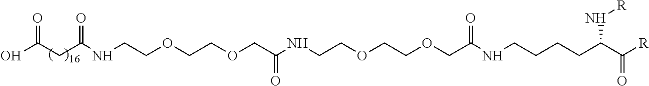 | |
| | C36H66N4O10R2 | |
| | K(PEG2PEG2C18OH) | OC(CCCCCCCCCCCCCCCCC(NCCOCCOCC |
| | | (NCCOCCOCC(NCCCC[C@@H](C([R])═O) |
| | | N[R]))═O)═O)═O)═O |
| 124 | 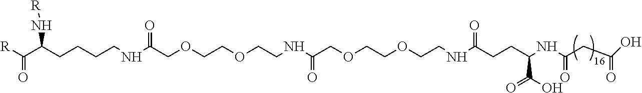 | |
| | C41H73N5O13R2 | |
| | K(PEG2PEG2DgEC18OH) | OC(CCCCCCCCCCCCCCCCC(N[C@H](CCC |
| | | (NCCOCCOCC(NCCOCCOCC(NCCCC[C@@H] |
| | | (C([R])═O)N[R])═O)═O)═O)C(O)═O)═O) |
| 125 | 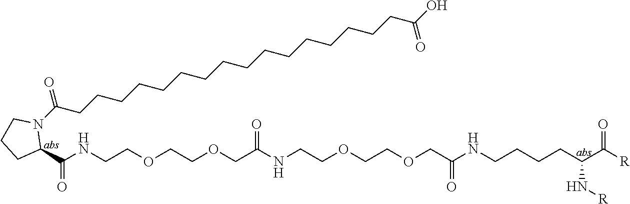 | |
| | C41H73N5O11R2 | |
| | K(PEG2PEG2PC18OH) | OC(CCCCCCCCCCCCCCCCC(N(CCC1)[C@@ |
| | | H1C(NCCOCCOCC(NCCOCCOCC(NCCCC[C |
| | | @@H](C([R])═O)N[R])═O)═O)═O)═O)═O) |
| 126 | 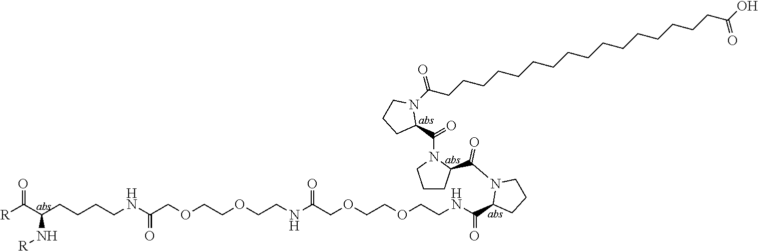 | |
| | C51H87N7O13R2 | |
| | K(PEG2PEG2PPPC18OH) | OC(CCCCCCCCCCCCCCCCC(N(CCC1)[C@@ |
| | | H]1C(N(CCC1)[C@@H]1C(N(CCC1)[C@@H]1 |
| | | C(NCCOCCOCC(NCCOCCOCC(NCCCC[C@@ |
| | | H](C([R])═O)N[R])═O)═O)═O)═O)═O)═O)═O |
| 127 | 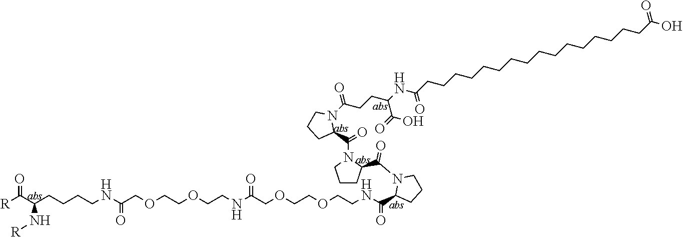 | |
| | C56H94N8O16R2 | |
| | K(PEG2PEG2PPPgEC18OH) | OC(CCCCCCCCCCCCCCCCC(N[C@@H](CC |
| | | C(N(CCC1)[C@@H]IC(N(CCC1)[C@@H]1C |
| | | (N(CCC1)[C@@H]1C(NCCOCCOCC(NCCOCC |
| | | OCC(NCCCC[C@@H](C([R])═O)N[R])═O)═ |
| | | O)═O)═O)═O)═O)C(O)═O)═O)═O |
| 128 | 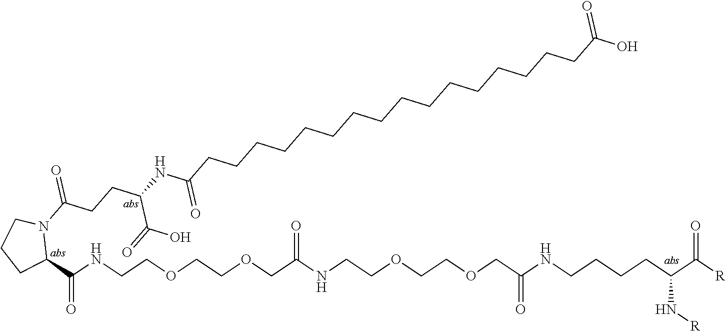 | |
| | C46H80N6O14R2 | |
| | K(PEG2PEG2PgEC18OH) | OC(CCCCCCCCCCCCCCCCC(N[C@@H](CC |
| | | C(N(CCC1)[C@@H]1C(NCCOCCOCC(NCCOC |
| | | COCC(NCCCC[C@@H](C([R])═O)N[R])═O)═ |
| | | O)═O)═O)C(O)═O)═O)═O |
| 129 | 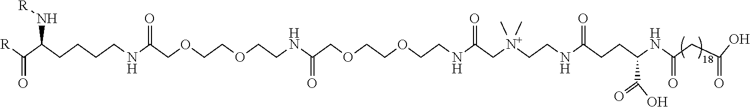 | |
| | C47H86N7O14R2+ | |
| | K(PEG2PEG2Sp6gEC18OH) | C[N+](C)(CCNC(CC[C@@H](C(O)═O)NC(CC |
| | | CCCCCCCCCCCCCCC(O)═O)═O)═O)CC(NCC |
| | | OCCOCC(NCCOCCOCC(NCCCC[C@@H](C |
| | | ([R])═O)N[R])═O)═O)═O |
| 130 | 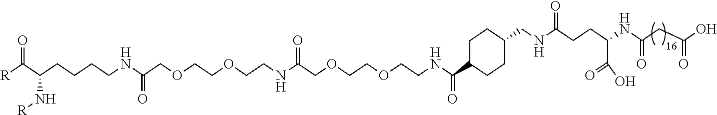 | |
| | C49H86N6O14R2 | |
| | K(PEG2PEG2TrxgEC18OH) | OC(CCCCCCCCCCCCCCCCC(N[C@@H](CC |
| | | C(NC[C@H](CC1)CC[C@@H]1C(NCCOCCOC |
| | | C(NCCOCCOCC(NCCCC[C@@H](C([R])═O)N |
| | | [R])═O)═O)═O)═O)C(O)═O)═O)═O |
| 131 | 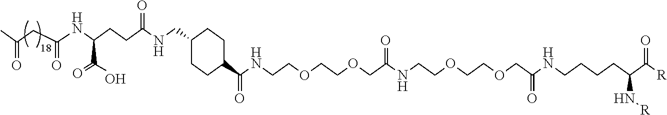 | |
| | C51H90N6O14R2 | |
| | K(PEG2PEG2TrxgEC20OH) | OC(CCCCCCCCCCCCCCCCCCC(N[C@@H]( |
| | | CCC(NC[C@H](CC1)CC[C@@H]1C(NCCOCC |
| | | OCC(NCCOCCOCC(NCCCC[C@@H](C([R])═ |
| | | O)N[R])═O)═O)═O)═O)C(O)═O)═O)═O |
| 132 | 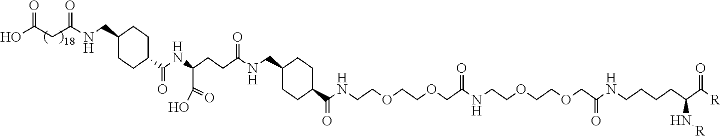 | |
| | C59H103N7O15R2 | |
| | K(PEG2PEG2TrxgETrxC20OH) | |
| 133 | 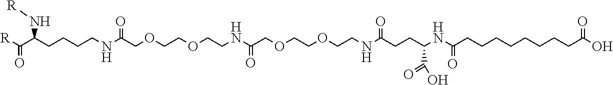 | |
| | C33H57N5O13R2 | |
| | K(PEG2PEG2gEC10OH) | OC(CCCCCCCCC(N[C@@H](CCC(NCCOCCO |
| | | CC(NCCOCCOCC(NCCCC[C@@H](C([R])═O) |
| | | N[R])═O)═O)═O)C(O)═O)═O)═O) |
| 134 | 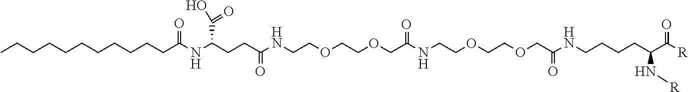 | |
| | C35H63N5O11R2 | |
| | K(PEG2PEG2gEC12) | CCCCCCCCCCCC(N[C@@H](CCC(NCCOCC |
| | | OCC(NCCOCCOCC(NCCCC[C@@H](C([R])═ |
| | | O)N[R])═O)═O)═O)C(O)═O)═O |
| 135 | 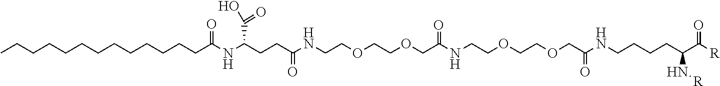 | |
| | C37H67N5O11R2 | |
| | K(PEG2PEG2gEC14) | CCCCCCCCCCCCCC(N[C@@H](CCC(NCCO |
| | NMeK(PEG2PEG2gEC14) | CCOCC(NCCOCCOCC(NCCCC[C@@H](C |
| | | ([R]═O)N[R])═O)═O)═O)C(O)═O)═O) |
| 136 | 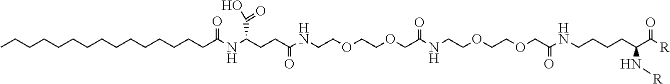 | |
| | C39H71N5O11R2 | |
| | K(PEG2PEG2gEC16) | CCCCCCCCCCCCCCCC(N[C@@H](CCC(NC |
| | | COCCOCC(NCCOCCOCC(NCCCC[C@@H](C |
| | | ([R])═O)N[R])═O)═O)═O)C(O)═O)═O) |
| 137 | 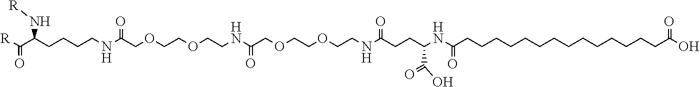 | |
| | C39H69N5O13R2 | |
| | K(PEG2PEG2gEC16OH) | OC(CCCCCCCCCCCCCCC(N[C@@H](CCC(N |
| | | CCOCCOCC(NCCOCCOCC(NCCCC[C@@H] |
| | | (C([R])═O)N[R])═O)═O)═O)C(O)═O)═O)═O |
| 138 | 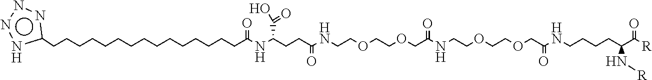 | |
| | C40H71N9O11R2 | |
| | K(PEG2PEG2gEC16tetrazole) | OC([C@H](CCC(NCCOCCOCC(NCCOCCOCC |
| | | (NCCCC[C@@H](C([R])═O)N[R])═O)═O)═O)N |
| | | C(CCCCCCCCCCCCCCCc1nnn[nH]1)═O)═O |
| 139 | 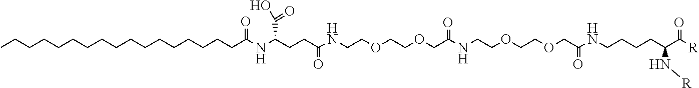 | |
| | C41H75N5O11R2 | |
| | K(PEG2PEG2gEC18) | CCCCCCCCCCCCCCCCCC(N[C@@H](CCC |
| | | (NCCOCCOCC(NCCOCCOCC(NCCCC[C@@H] |
| | | (C([R])═O)N[R])═O)═O)═O)C(O)═O)═O |
| 140 | 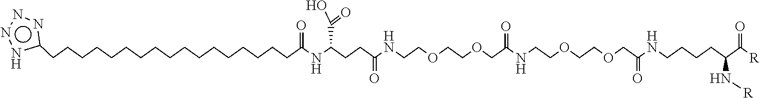 | |
| | C42H75N9O11R2 | |
| | K(PEG2PEG2gEC18tetrazole) | OC([C@H](CCC(NCCOCCOCC(NCCOCCOCC |
| | | (NCCCC[C@@H](C([R])═O)N[R])═O)═O)═O)N |
| | | C(CCCCCCCCCCCCCCCCCc1nnn[nH]1)═O)═O |
| 141 | 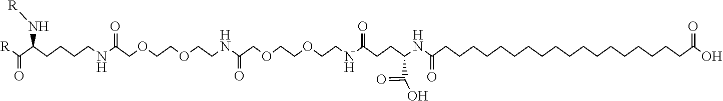 | |
| | C43H77N5O13R2 | |
| | K(PEG2PEG2gEC20OH) | OC(CCCCCCCCCCCCCCCCCCC(N[C@@H]( |
| | | CCC(NCCOCCOCC(NCCOCCOCC(NCCCC[C |
| | | @@H](C([R])═O)N[R])═O)═O)═O)C(O)═O)═ |
| | | O)═O |
| 142 | 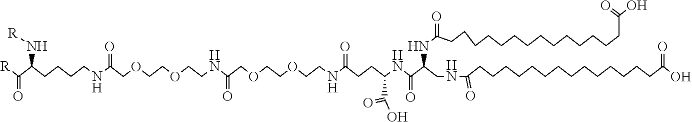 | |
| | C58H103N7O17R2 | |
| | KPEG2PEG2gEDap(C16OH)2, | OC(CCCCCCCCCCCCCCC(NC[C@@H](C(N[ |
| | K(PEG2PEG2gEDAP(C16OH)2) | C@@H](CCC(NCCOCCOCC(NCCOCCOCC(N |
| | | CCCC[C@@H](C([R])═O)N[R])═O)═O)═O)C |
| | | (O)═O)═O)NC(CCCCCCCCCCCCCCC(O)═ |
| | | O)═O)═O)═O |
| 143 | 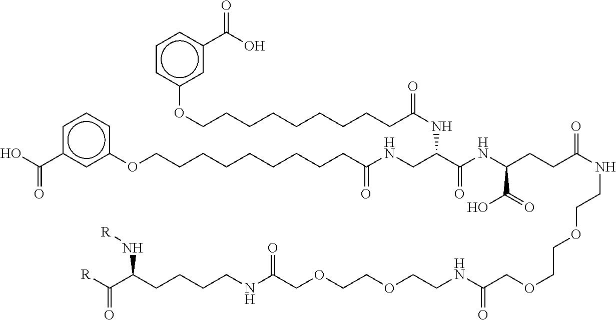 | |
| | C60H91N7O19R2 | |
| | K(PEG2PEG2gEDAP(mXOH)2) | OC([C@H](CCC(NCCOCCOCC(NCCOCCOCC |
| | KPEG2PEG2gEDAP(mXOH)2 | (NCCCC[C@@H](C([R])═O)N[R])═O)═O)═O)N |
| | | C([C@H](CNC(CCCCCCCCCOc1cc(C(O)═O)cc |
| | | c1)═O)NC(CCCCCCCCCOc1cc(C(O)═O)ccc1)═ |
| | | O)═O)═O |
| 144 | 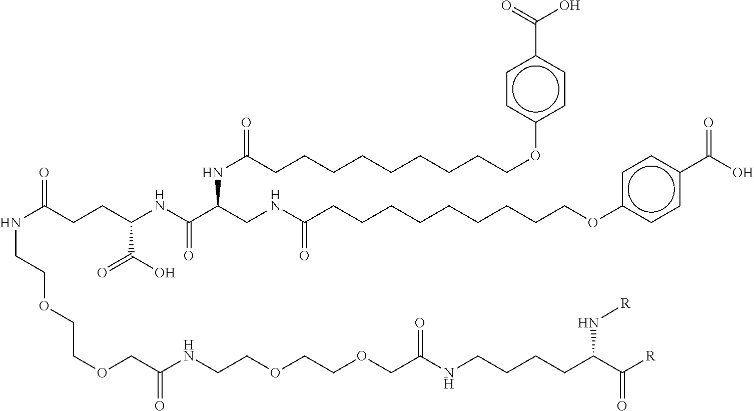 | |
| | C60H91N7O19R2 | |
| | K(PEG2PEG2gEDAP(pXOH)2) | OC([C@H](CCC(NCCOCCOCC(NCCOCCOCC |
| | KPEG2PEG2gEDAP(pXOH)2 | (NCCCC[C@@H](C([R])═O)N([R])═O)═O)═O)N |
| | | C([C@H](CNC(CCCCCCCCCOc(cc1)ccc1C(O)═ |
| | | O)═O)NC(CCCCCCCCCOc(cc1)ccc1C(O)═O)═ |
| | | O)═O)═O |
| 145 | 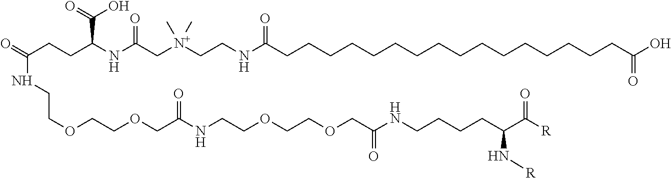 | |
| | C47H86N7O14R2+ | |
| | K(PEG2PEG2gESp6C18OH) | C[N+](C)(CCNC(CCCCCCCCCCCCCCCCC(O)═ |
| | | O)═O)CC(N[C@@H](CCC(NCCOCCOCC(NC |
| | | COCCOCC(NCCCC[C@@H](C([R])═O)N[R])═ |
| | | O)═O)═O)C(O)═O)═O |
| 146 | 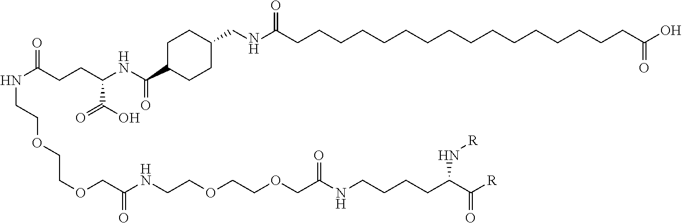 | |
| | C49H86N6O14R2 | |
| | K(PEG2PEG2gETrxC18OH) | OC(CCCCCCCCCCCCCCCCC(NC[C@H](CC1) |
| | | CC[C@@H]1C(N[C@@H](CCC(NCCOCCOCC |
| | | (NCCOCCOCC(NCCCC[C@@H](C([R])═O)N |
| | | [R])═O)═O)═O)C(O)═O)═O)═O)═O |
| 147 | 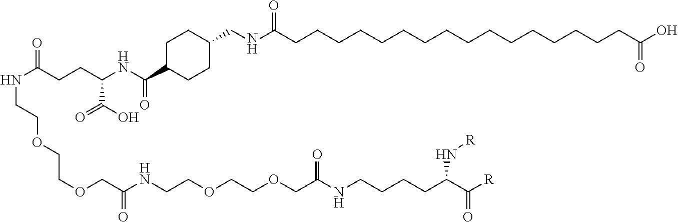 | |
| | C51H90N6O14R2 | |
| | K(PEG2PEG2gETrxC20OH) | OC(CCCCCCCCCCCCCCCCCCC(NC[C@H](C |
| | | C1)CC[C@@H]1C(N[C@@H](CCC(NCCOCC |
| | | OCC(NCCOCCOCC(NCCCC[C@@H](C([R])═ |
| | | O)N[R])═O)═O)═O)C(O)═O)═O)═O)═O |
| 148 | 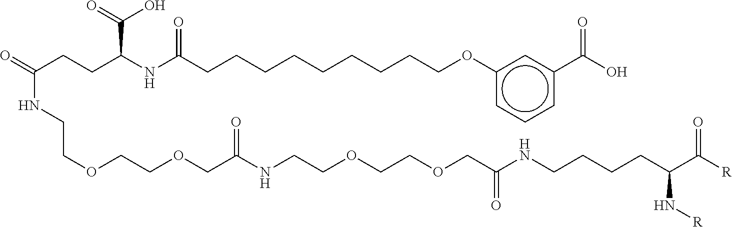 | |
| | C40H63N5O14R2 | |
| | K(PEG2PEG2gEmXOH) | OC([C@@H](NC(CCCCCCCCCOc1cccc(C(O)═ |
| | | O)c1)═O)CCC(NCCOCCOCC(NCCOCCOCC(N |
| | | CCCC[C@H](N[R])C([R])═O)═O)═O)═O)═O |
| 149 | 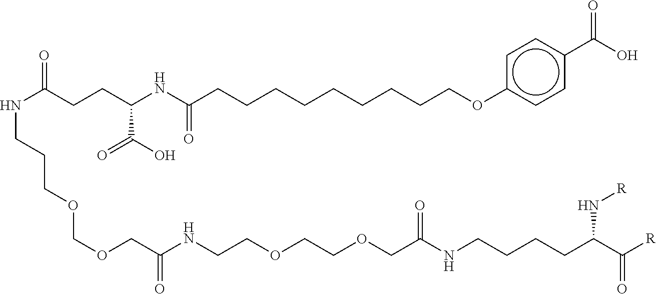 | |
| | C40H63N5O14R2 | |
| | K(PEG2PEG2gEpXOH) | OC([C@@H](NC(CCCCCCCCCOc1ccc(C(O)═ |
| | | O)cc1)═O)CCC(NCCOCCOCC(NCCOCCOCC |
| | | (NCCCC[C@H](N[R])C([R])═O)═O)═O)═O)═O |
| 150 | 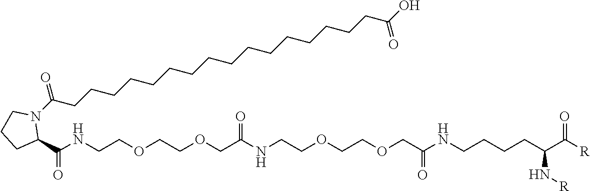 | |
| | C41H73N5O11R2 | |
| | K(PEG2PEG2pC18OH) | OC(CCCCCCCCCCCCCCCCC(N1CCC[C@@H] |
| | | 1C(NCCOCCOCC(NCCOCCOCC(NCCCC[C@ |
| | | H](N[R])C([R])═O)═O)═O)═O)═O)═O |
| 151 | 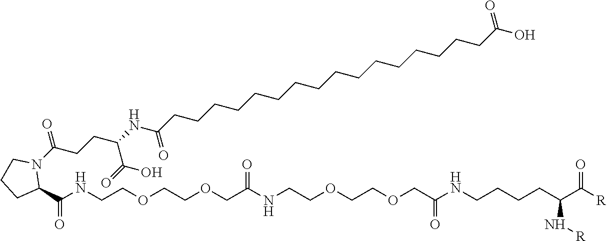 | |
| | C46H80N6O14R2 | |
| | K(PEG2PEG2pgEC18OH) | OC(CCCCCCCCCCCCCCCCC(N[C@@H](CC |
| | | C(N(CCC1)[C@H]1C(NCCOCCOCC(NCCOCC |
| | | OCC(NCCCC[C@@H](C([R])═O)N[R])═O)═ |
| | | O)═O)═O)C(O)═O)═O)═O |
| 152 | 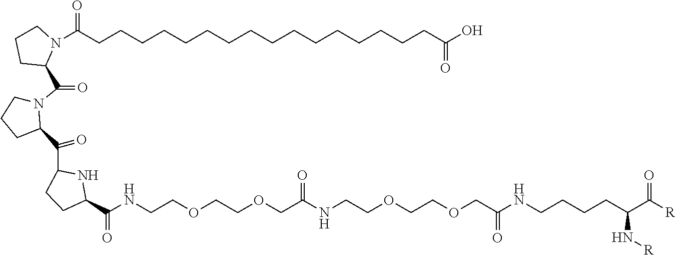 | |
| | C51H87N7O13R2 | |
| | K(PEG2PEG2pppC18OH) | OC(CCCCCCCCCCCCCCCCC(N(CCC1)[C@H] |
| | | 1C(N(CCC1)[C@H]1C(N(CCC1)[C@H]1C(NCC |
| | | OCCOCC(NCCOCCOCC(NCCCC[C@@H](C |
| | | R])═O)N[R])═O)═O)═O)═O)═O)═O)═O) |
| 153 | 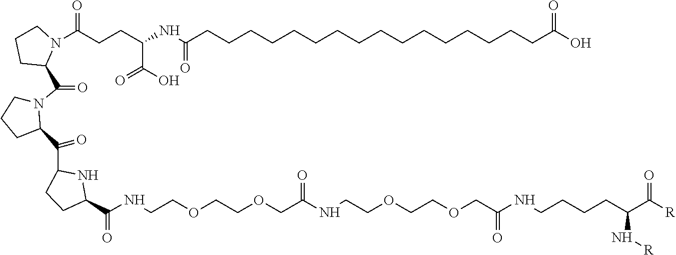 | |
| | C56H94N8O16R2 | |
| | K(PEG2PEG2pppgEC18OH) | OC(CCCCCCCCCCCCCCCCC(N[C@@H](CC |
| | | C(N(CCC1)[C@H]1C(N(CCC1)[C@H]1C(N(CC |
| | | C1)[C@H]1C(NCCOCCOCC(NCCOCCOCC(N |
| | | CCCC[C@@H](C([R])═O)N[R])═O)═O)═O)═ |
| | | O)═O)═O)C(O)═O)═O)═O |
| 154 | 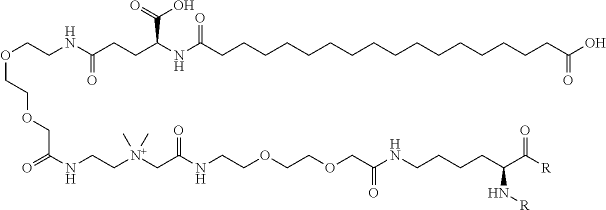 | |
| | C48H87N5O17R2 | |
| | K(PEG2PEG6gEC16OH) | OC(CCCCCCCCCCCCCCC(N[C@@H](CCC(N |
| | | CCOCCOCCOCCOCCOCCOCCC(NCCOCCOC |
| | | C(NCCCC[C@@H](C([R])═O)N[R])═O)═O)═O) |
| | | C(O)═O)═O)═O |
| 155 | 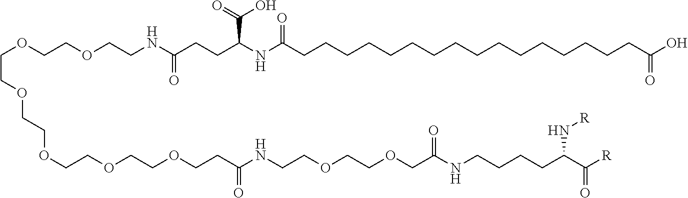 | |
| | C50H91N5O17R2 | |
| | K(PEG2PEG6gEC18OH) | OC(CCCCCCCCCCCCCCCCC(N[C@@H](CC |
| | | C(NCCOCCOCCOCCOCCOCCOCCC(NCCOC |
| | | COCC(NCCCC[C@@H](C([R])═O)N[R])═O)═ |
| | | O)═O)C(O)═O)═O)═O |
| 156 | 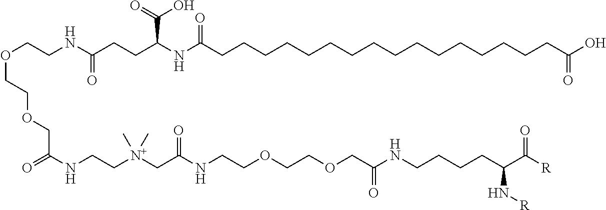 | |
| | C47H86N7O14R2+ | |
| | K(PEG2Sp6PEG2gEC18OH) | C[N+](C)(CCNC(COCCOCCNC(CC[C@@H](C |
| | | (O)═O)NC(CCCCCCCCCCCCCCCCC(O)═O)═ |
| | | O)═O)═O)CC(NCCOCCOCC(NCCCC[C@@H]( |
| | | C([R])═O)N[R])═O)═O |
| 157 | 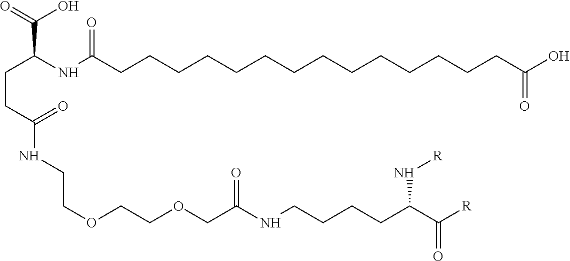 | |
| | C33H58N4O10R2 | |
| | K(PEG2gEC16OH) | OC(CCCCCCCCCCCCCCC(N[C@@H](CCC(N |
| | | CCOCCOCC(NCCCC[C@@H](C([R])═O) |
| | | N[R])═O)═O)C(O)═O)═O)═O |
| 158 | 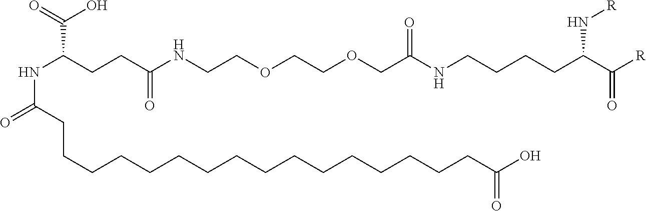 | |
| | C35H62N4O10R2 | |
| | K(PEG2gEC18OH) | OC(CCCCCCCCCCCCCCCCC(N[C@@H](CC |
| | | C(NCCOCCOCC(NCCCC[C@@H](C([R])═O)N |
| | | [R])═O)═O)C(O)═O)═O)═O |
| 159 | 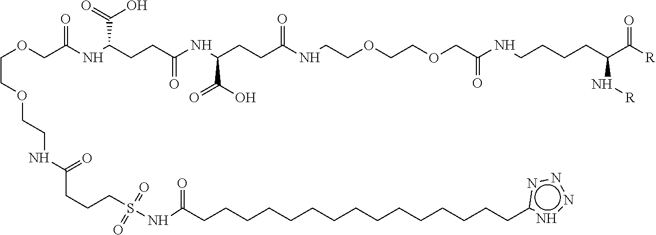 | |
| | C49H85N11O17SR2 | |
| | K(PEG2gEgEPEG24SBC16Tetrazole) | OC([C@H](CCC(NCCOCCOCC(NCCCC[C@@ |
| | | H](C([R])═O)N[R])═O)═O)NC(CC[C@@H](C |
| | | (O)═O)NC(COCCOCCNC(CCCS(NC(CCCCCCC |
| | | CCCCCCCCc1nnn[nH]1)═O)(═O)═O)═O)═O)═ |
| | | O)═O |
| 160 | 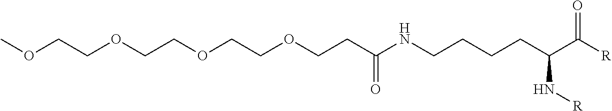 | |
| | C16H30N2O6R2 | |
| | K(PEG3OMe) | COCCOCCOCCOCCC(NCCCC[C@@H](C |
| | K(mPEG4) | ([R])═O)N[R])═O |
| 161 | 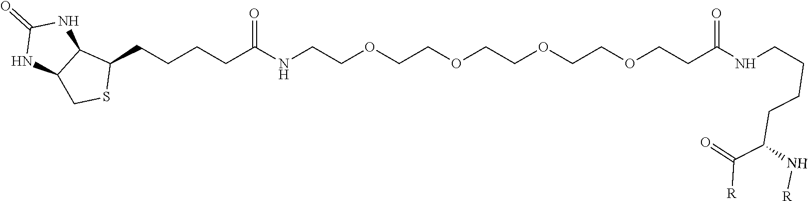 | |
| | C27H47N5O8SR2 | |
| | K(PEG4Biotina), K(PEG4Biotin) | O═C(CCCC[C@H]([C@@H]IN2)SC[C@H]1N |
| | | C2═O)NCCOCCOCCOCCOCCC(NCCCC[C@ |
| | | @H](C([R])═O)N[R])═O |
| 162 | 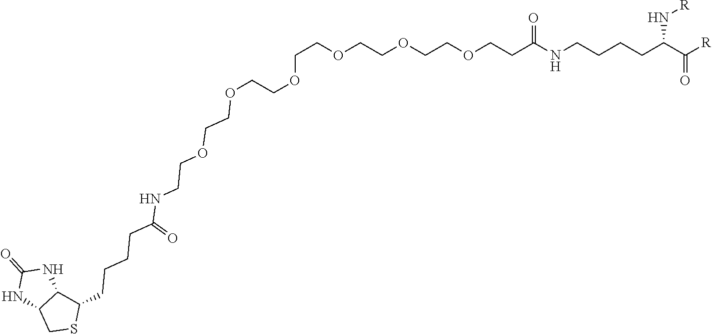 | |
| | C31H55N5O10SR2 | |
| | K(PEG6Biotin) | O═C(CCCC[C@@H]([C@H]IN2)SC[C@@H]1 |
| | | NC2═O)NCCOCCOCCOCCOCCOCCOCCC(NC |
| | | CCC[C@@H](C([R])═O)N[R])═O |
| 163 | 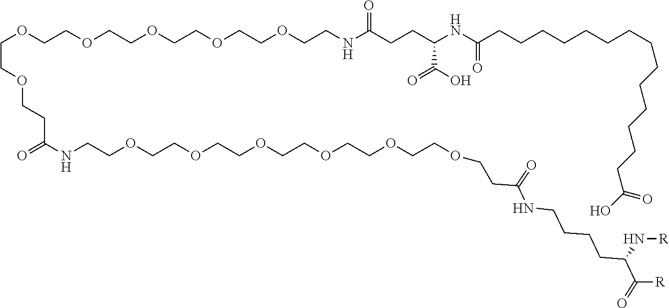 | |
| | C57H105N5O21R2 | |
| | K(PEG6PEG6gEC16OH) | OC(CCCCCCCCCCCCCCC(N[C@@H](CCC(N |
| | | CCOCCOCCOCCOCCOCCOCCC(NCCOCCOC |
| | | COCCOCCOCCOCCC(NCCCC[C@@H](C([R])═ |
| | | O)N[R])═O)═O)═O)C(O)═O)═O)═O |
| 164 | 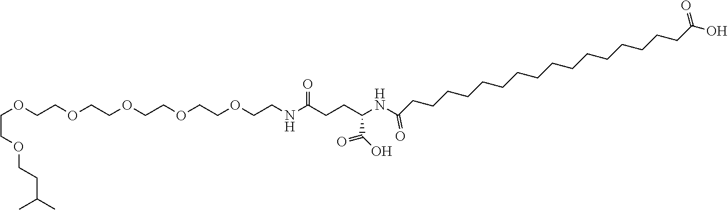 [SMILES: CC(C)CCOCCOCCOCCOCCOCCOCCNC(=O)CC[C@H](NC(=O)CCCCCCCCCCCCCCCCC(=O)O)C(=O)O] | |
| | C59H109N5O21R2 | |
| | K(PEG6PEG6gEC18OH) | OC(CCCCCCCCCCCCCCCCC(N[C@@H](CC |
| | | C(NCCOCCOCCOCCOCCOCCOCCC(NCCOC |
| | | COCCOCCOCCOCCOCCC(NCCCC[C@@H](C |
| | | ([R])═O)N([R])═O)═O)═O)C(O)═O)═O)═O |
| 165 | 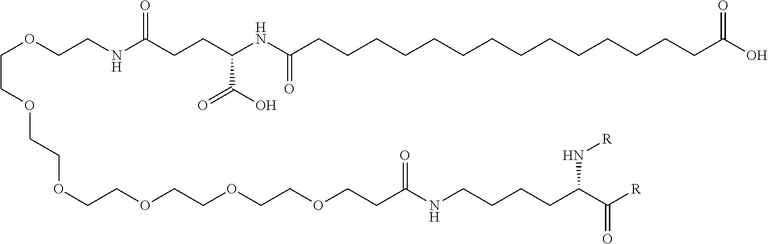 | |
| | C42H76N4O14R2 | |
| | K(PEG6gEC16OH) | OC(CCCCCCCCCCCCCCC(N[C@@H](CCC(N |
| | | CCOCCOCCOCCOCCOCCOCCC(NCCCC[C@ |
| | | @H](C([R])═O)N[R])═O)═O)C(O)═O)═O)═O |
| 166 | 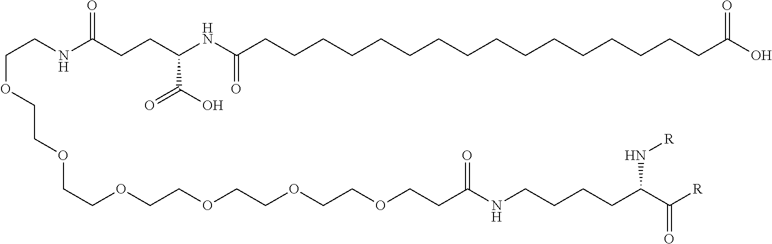 | |
| | C44H80N4O14R2 | |
| | K(PEG6gEC18OH) | OC(CCCCCCCCCCCCCCCCC(N[C@@H](CC |
| | | C(NCCOCCOCCOCCOCCOCCOCCC(NCCCC |
| | | [C@@H](C([R])═O)N[R])═O)═O)C(O)═O)═O)═O |
| 167 | 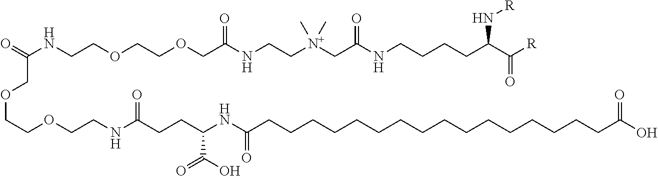 | |
| | C47H86N7O14R2+ | |
| | K(Sp6PEG2PEG2gEC18OH) | C[N+](C)(CCNC(COCCOCCNC(COCCOCCNC |
| | | (CC[C@@H](C(O)═O)NC(CCCCCCCCCCCCC |
| | | CCCC(O)═O)═O)═O)═O)═O)CC(NCCCC[C@@ |
| | | H](C([R])═O)N[R])═O) |
| 168 | 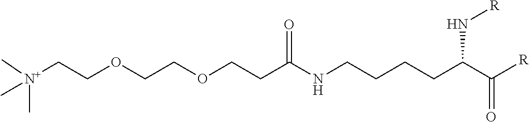 | |
| | C16H32N3O4R2+ | |
| | K(cPEG3a), K(cPEG3aCO) | C[N+](C)(C)CCOCCOCCC(NCCCC[C@@H](C |
| | | ([R])═O)N[R])═O |
| 169 | 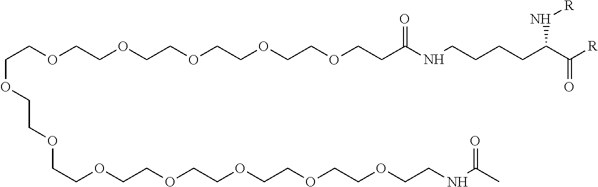 | |
| | C35H67N3O15R2 | |
| | K(dPEG12Ac) | CC(NCCOCCOCCOCCOCCOCCOCCOCCOCC |
| | | OCCOCCOCCOCCC(NCCCC[C@@H](C([R])═ |
| | | O)N[R])═O)═O |
| 170 | 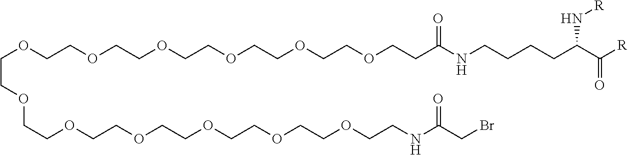 | |
| | C35H66BrN3O15R2 | |
| | K(dPEG12AcBr) | O═C(CCOCCOCCOCCOCCOCCOCCOCCOCC |
| | | OCCOCCOCCOCCNC(CBr)═O)NCCCC[C@@ |
| | | H](C([R])═O)N[R] |
| 171 | 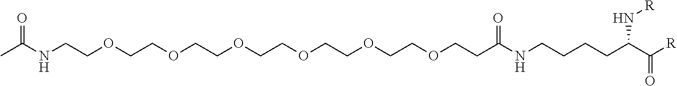 | |
| | C23H43N3O9R2 | |
| | K(dPEG6Ac) | CC(NCCOCCOCCOCCOCCOCCOCCC(NCCC |
| | | C[C@@H](C([R])═O)N[R])═O)═O) |
| 172 | 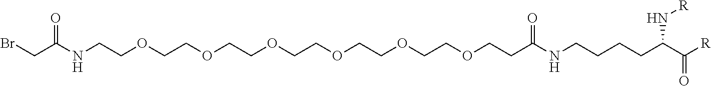 | |
| | C23H42BrN3O9R2 | |
| | K(dPEG6AcBr) | O═C(CCOCCOCCOCCOCCOCCOCCNC(CBr)═ |
| | | O)NCCCC[C@@H](C([R])═O)N[R] |
| 173 | 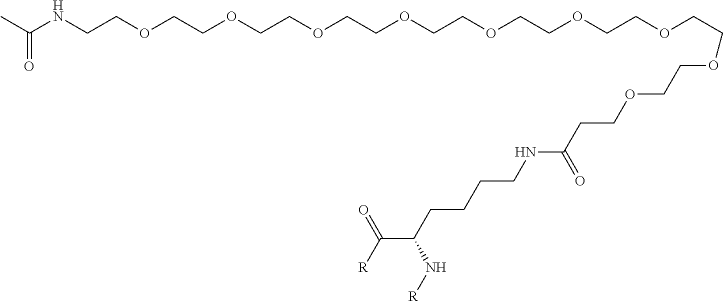 | |
| | C29H55N3O12R2 | |
| | K(dPEG9Ac) | CC(NCCOCCOCCOCCOCCOCCOCCOCCOCC |
| | | OCCC(NCCCC[C@@H](C([R])═O)N[R])═O)═O |
| 174 | 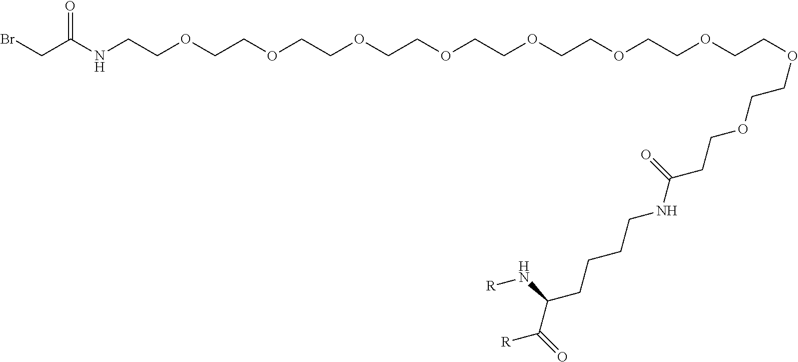 | |
| | C29H54BrN3O12R2 | |
| | dPEG9AcBr | O═C(CCOCCOCCOCCOCCOCCOCCOCCOCC |
| | | OCCNC(CBr)═O)NCCCC[C@@H](C([R])═O)N[R] |
| 175 | 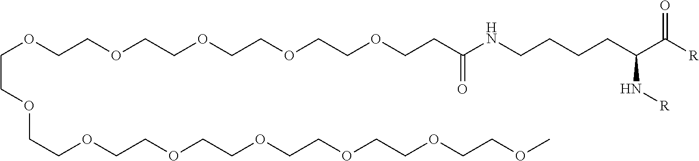 | |
| | C32H62N2O14R2 | |
| | K(mPEG12) | COCCOCCOCCOCCOCCOCCOCCOCCOCCO |
| | | CCOCCOCCC(NCCCC[C@@H](C([R])═O)N[R])═O |
| 176 | 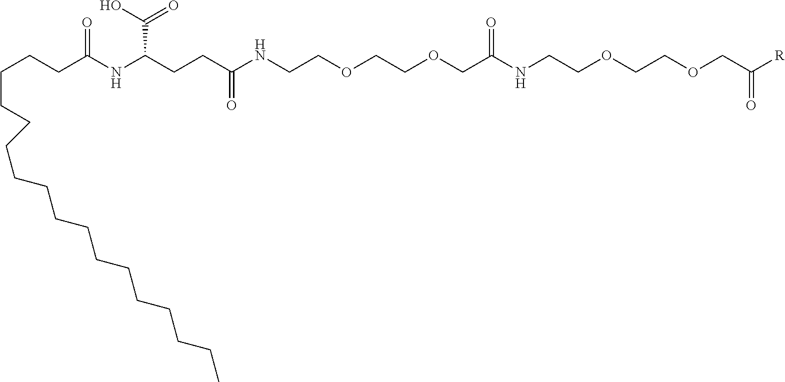 | |
| | C35H64N3O10R | |
| | PEG2PEG2gEC18 | CCCCCCCCCCCCCCCCCC(N[C@@H](CCC( |
| | | NCCOCCOCC(NCCOCCOCC([R])═O)═O)═O)C |
| | | (O)═O)═O |
| | | CCCCCCCCCCCCCCCCCC(N[C@@H](CCC( |
| | | NCCOCCOCC(NCCOCCOCC═O)═O)═O)C(O)═ |
| | | O)═O |
| 177 | 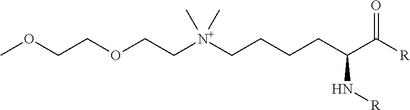 | |
| | C13H27N2O3R2+ | |
| | Lys(N+Me2mPEG3) | C[N+](C)(CCCC[C@@H](C([R])═O)N[R])CCO |
| | | CCOC |
| 178 | 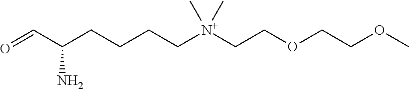 [SMILES: COCCOCC[N+](C)(C)CCCC[C@H](N)C=O] | |
| | C13H29N2O3+ | |
| | LysQuatMe2mPEG3, | C[N+](C)(CCCC[C@@H](C═O)N)CCOCCOC |
| | Lys(N+(Me)2mPEG3) | |
| 180 | 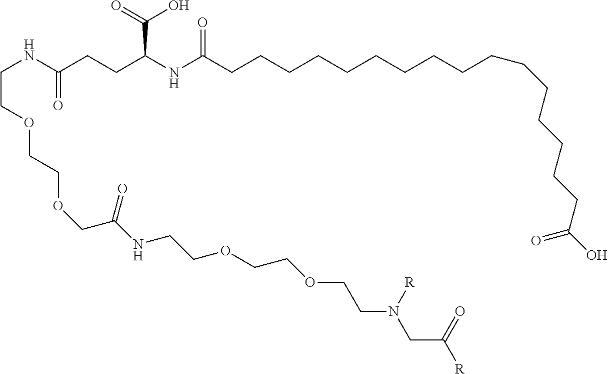 | |
| | C37H66N4O12R2 | |
| | N(PEG2PEG2gEC18OH)Gly | OC(CCCCCCCCCCCCCCCCC(N[C@@H](CC |
| | | C(NCCOCCOCC(NCCOCCOCCN(CC([R])═O) |
| | | [R])═O)═O)C(O)═O)═O)═O |
| 181 | 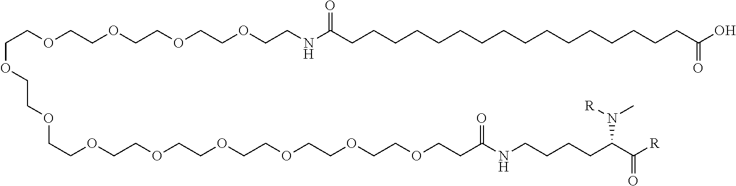 | |
| | C52H99N3O17R2 | |
| | NMeK(PEG12C18OH) | CN([C@@H](CCCCNC(CCOCCOCCOCCOCC |
| | | OCCOCCOCCOCCOCCOCCOCCOCCNC(CCC |
| | | CCCCCCCCCCCCCC(O)═O)═O)═O)C([R])═O)[R] |
| 182 | 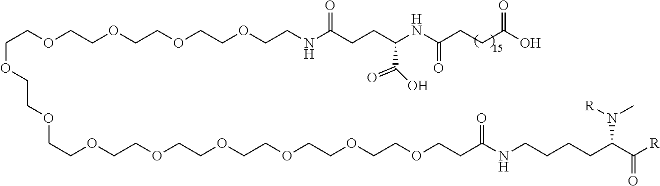 | |
| | C57H106N4O20R2 | |
| | NMeK(PEG12gEC18OH) | CN([C@@H](CCCCNC(CCOCCOCCOCCOCC |
| | | OCCOCCOCCOCCOCCOCCOCCOCCNC(CC[ |
| | | C@@H](C(O)═O)NC(CCCCCCCCCCCCCCCC |
| | | C(O)═O)═O)═O)═O)C([R])═O)[R] |
| 183 | 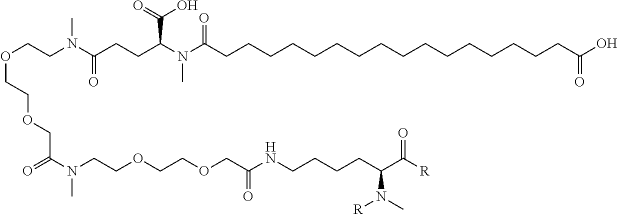 | |
| | C45H81N5O13R2 | |
| | NMeK(PEG2NMePEG2NMegENMeC18OH) | CN(CCOCCOCC(N(C)CCOCCOCC(NCCCC[C |
| | | @@H](C([R])═O)N(C)[R])═O)═O)C(CC[C@@ |
| | | H](C((O)═O)N(C)C(CCCCCCCCCCCCCCCCC |
| | | (O)═O)═O)═O |
| 184 | 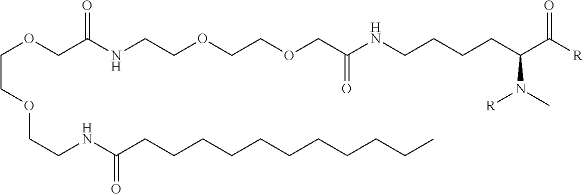 | |
| | C31H58N4O8R2 | |
| | NMeK(PEG2PEG2C12) | CCCCCCCCCCCC(NCCOCCOCC(NCCOCCO |
| | | CC(NCCCC[C@@H](C([R])═O)N(C)[R])═O)═ |
| | | O)═O |
| 185 | 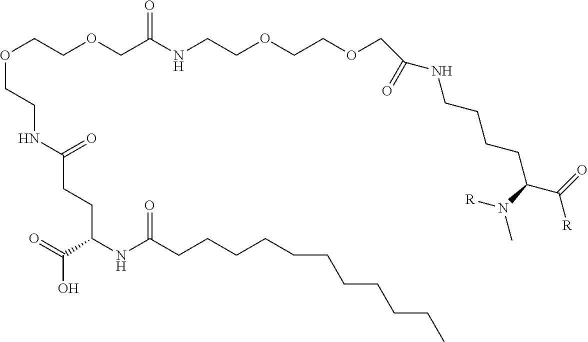 | |
| | C36H65N5O11R2 | |
| | NMeK(PEG2PEG2gEC12) | CCCCCCCCCCCC(N[C@@H](CCC(NCCOCC |
| | | OCC(NCCOCCOCC(NCCCC[C@@H](C([R])═ |
| | | O)N(C)[R])═O)═O)═O)C(O)═O)═O |
| 186 | 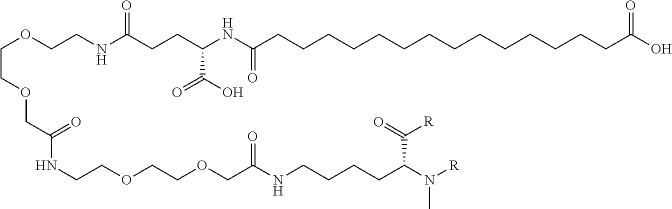 | |
| | C40H71N5O13R2 | |
| | NMeK(PEG2PEG2gEC160H) | CN([C@@H](CCCCNC(COCCOCCNC(COCCO |
| | | CCNC(CC[C@@H](C(O)═O)NC(CCCCCCCCC |
| | | CCCCCC(O)═O)═O)═O)═O)═O)C([R])═O)[R] |
| 187 | 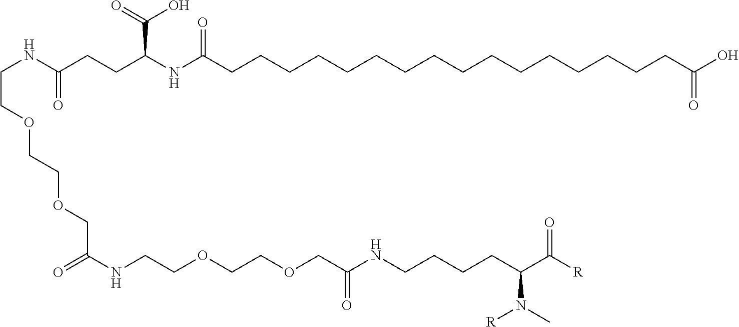 | |
| | C42H75N5O13R2 | |
| | NMeK(PEG2PEG2gEC18OH) | CN([C@@H](CCCCNC(COCCOCCNC(COCCO |
| | | CCNC(CC[C@@H](C(O)═O)NC(CCCCCCCCC |
| | | CCCCCCCC(O)═O)═O)═O)═O)═O)C([R])═O)[R] |
| 188 | 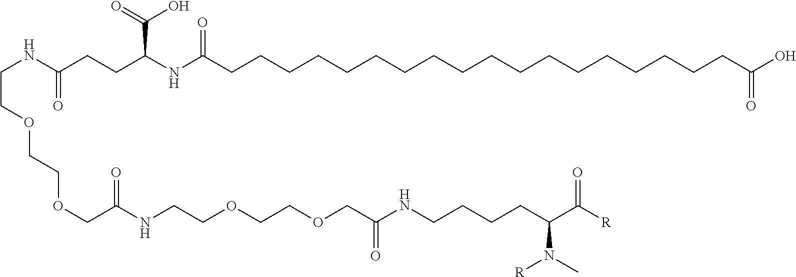 | |
| | C44H79N5O13R2 | |
| | NMeK(PEG2PEG2gEC20OH) | CN([C@@H](CCCCNC(COCCOCCNC(COCCO |
| | | CCNC(CC[C@@H](C(O)═O)NC(CCCCCCCCC |
| | | CCCCCCCCCC(O)═O)═O)═O)═O)═O)C([R])═ |
| | | O)[R] |
| 189 | 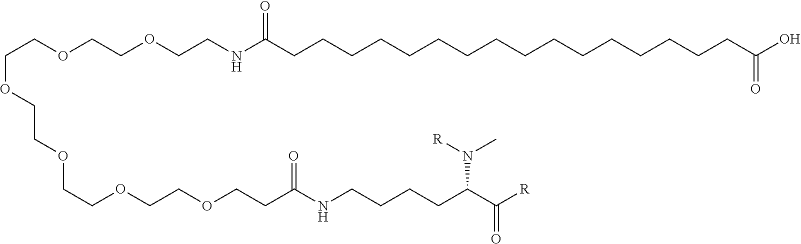 | |
| | C39H74N3O11R3 | |
| | NMeK(PEG6C18OH) | CN([R])[R](CCCCNC(CCOCCOCCOCCOCCO |
| | | CCOCCNC(CCCCCCCCCCCCCCCCC(O)═O)═ |
| | | O)═O)C([R])═O |
| 190 | 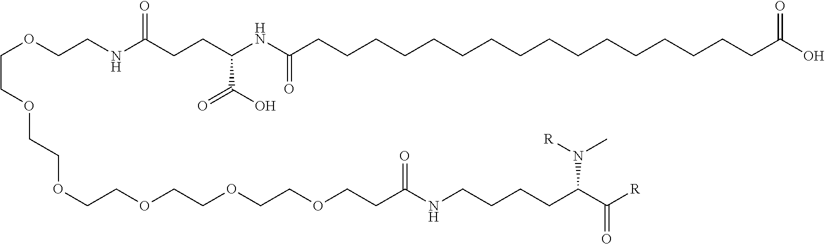 | |
| | C45H82N4O14R2 | |
| | NMeK(PEG6gEC18OH) | CN([C@@H](CCCCNC(CCOCCOCCOCCOCC |
| | | OCCOCCNC(CC[C@@H](C(O)═O)NC(CCCCC |
| | | CCCCCCCCCCCC(O)═O)═O)═O)═O)C([R])═O) |
| | | [R] |
| 191 | 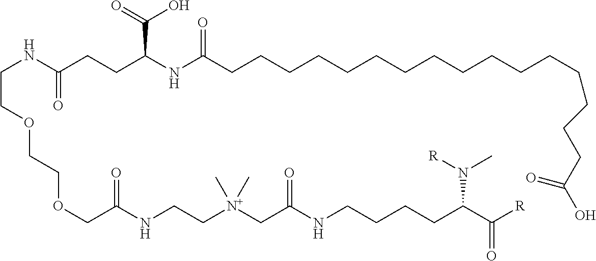 | |
| | C42H77N6O11R2+ | |
| | NMeK(SP6PEG2gEC18OH) | CN([C@@H](CCCCNC(C[N+](C)(C)CCNC(CO |
| | | CCOCCNC(CC[C@@H](C(O)═O)NC(CCCCCC |
| | | CCCCCCCCCCC(O)═O)═O)═O)═O)═O)C([R])═ |
| | | O)[R] |
| 192 | 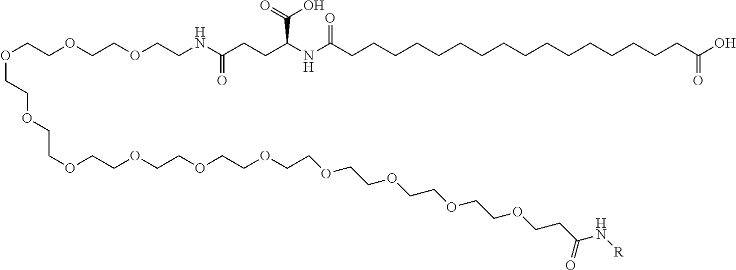 | |
| | C50H94N3O19R | |
| | PEG12gEC18OH | OC(CCCCCCCCCCCCCCCCC(N[C@@H](CC |
| | | C(NCCOCCOCCOCCOCCOCCOCCOCCOCCO |
| | | CCOCCOCCOCCC(N[R])═O)═O)C(O)═O)═O)═O |
| 193 | 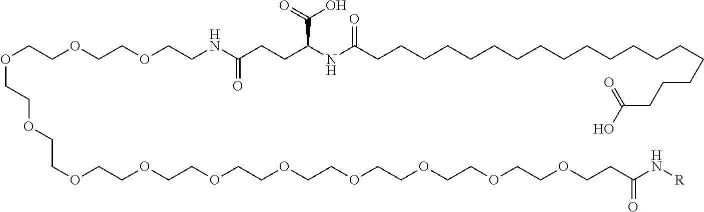 | |
| | C52H98N3O19R | |
| | PEG12gEC20OH | OC(CCCCCCCCCCCCCCCCCCC(N[C@@H] |
| | | (CCC(NCCOCCOCCOCCOCCOCCOCCOCCOC |
| | | COCCOCCOCCOCCC(N[R])═O)═O)C(O)═O)═ |
| | | O)═O |
| 194 | 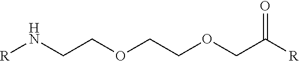 | |
| | C6H11NO3R2 | |
| | PEG2, PEG2(2) | O═C(COCCOCCN[R])[R] |
| 195 | 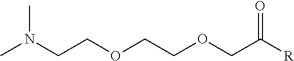 | |
| | C7H13NO3R2 | |
| | PEG2(NMe(2)) | CN(CCOCCOCC([R])═O)[R] |
| | PEG2NMe | |
| 196 | 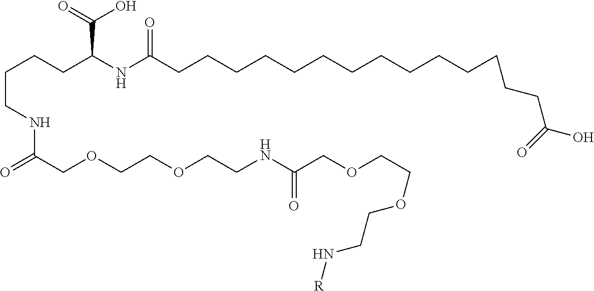 | |
| | C34H63N4O11R | |
| | PEG2PEG2eKC16OH | OC(CCCCCCCCCCCCCCC(N[C@@H](CCCC |
| | | NC(COCCOCCNC(COCCOCCN[R])═O)═O)C( |
| | | O)═O)═O)═O |
| | | NCCOCCOCC(NCCOCCOCC(NCCCC[C@@H] |
| | | (C(O)═O)NC(CCCCCCCCCCCCCCC(O)═O)═O |
| | | O)═O)═O |
| 197 | 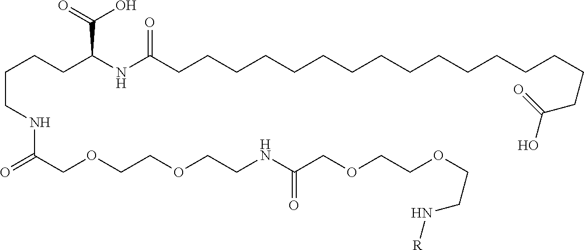 | |
| | C36H67N4O11R | |
| | PEG2PEG2eKC18OH | OC(CCCCCCCCCCCCCCCCC(N[C@@H](CC |
| | | CCNC(COCCOCCNC(COCCOCCN[R])═O)═O) |
| | | C(O)═O)═O)═O |
| | | NCCOCCOCC(NCCOCCOCC(NCCCC[C@@H] |
| | | (C(O)═O)NC(CCCCCCCCCCCCCCCCC(O)═ |
| | | O)═O)═O)═O |
| 198 | 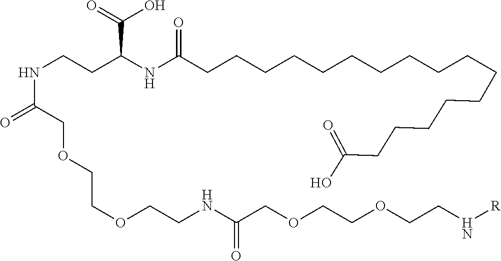 | |
| | C34H63N4O11R | |
| | PEG2PEG2gDabC18OH | OC(CCCCCCCCCCCCCCCCC(N[C@@H](CC |
| | | NC(COCCOCCNC(COCCOCCN[R])═O)═O)C( |
| | | O)═O)═O)═O |
| | | NCCOCCOCC(NCCOCCOCC(NCC[C@@H](C( |
| | | O)═O)NC(CCCCCCCCCCCCCCCCC(O)═O)═ |
| | | O)═O)═O |
| 199 | 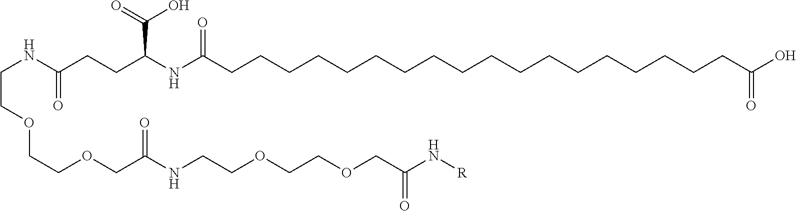 | |
| | C37H67N4O12R | |
| | PEG2PEG2gEC20OH | OC(CCCCCCCCCCCCCCCCCCC(N[C@@H]( |
| | | CCC(NCCOCCOCC(NCCOCCOCC(N[R])═O)═ |
| | | O)═O)C(O)═O)═O)═O |
| 200 | 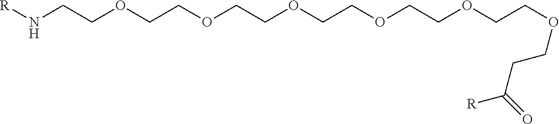 | |
| | C15H29NO7R2 | |
| | PEG6 | O═C(CCOCCOCCOCCOCCOCCOCCN[R])[R] |
| 201 | 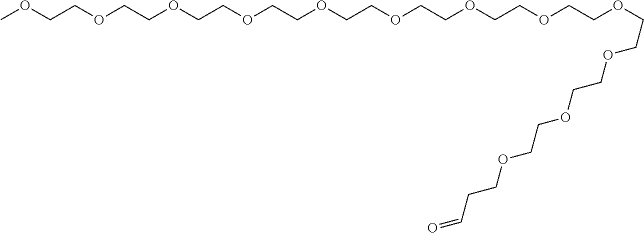 [SMILES: COCCOCCOCCOCCOCCOCCOCCOCCOCCOCCOCCOCCC=O] | |
| | C26H52O13 | |
| | Peg 12-Ome | COCCOCCOCCOCCOCCOCCOCCOCCOCCO |
| | Peg 12OMe, Polyethylene 12-O-Methyl | CCOCCOCCC═O |
| | Peg 12-O methyl | |
| 202 | 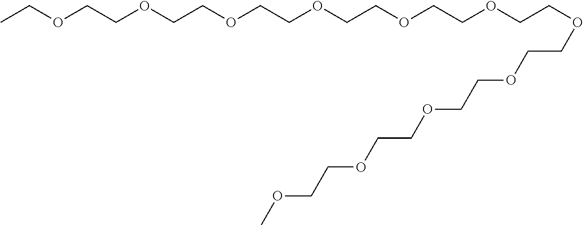 [SMILES: CCOCCOCCOCCOCCOCCOCCOCCOCCOCCOCCOC] | |
| | C23H48O11 | |
| | Peg12OMe, Peg 12-Omethyl | CCOCCOCCOCCOCCOCCOCCOCCOCCOCC |
| | | OCCOC |
| 203 | 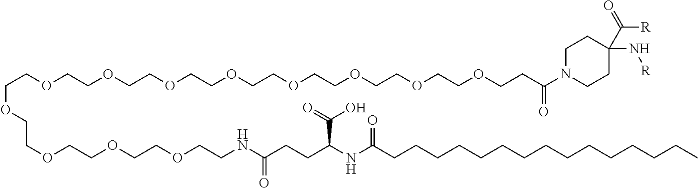 | |
| | C54H100N4O18R2 | |
| | Pip(PEG12gEC16), | CCCCCCCCCCCCCCCC(N[C@@H](CCC(NC |
| | Spiral_Pip_PEG12_IsoGlu_Palm | COCCOCCOCCOCCOCCOCCOCCOCCOCCO |
| | | CCOCCOCCC(N(CC1)CCC1(C([R])═O)N[R])═ |
| | | O)═O)C(O)═O)═O |
| 204 | 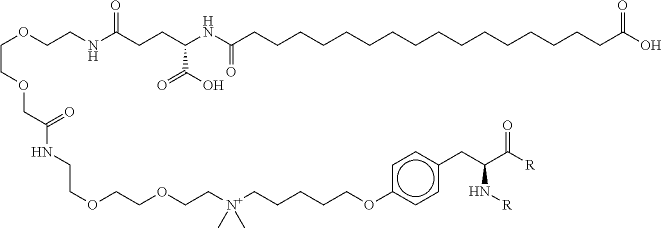 | |
| | C51H88N5O13R2+ | |
| | TMAPF(PEG2PEG2gEC18OH) | C[N+](C)(CCCCCOc1ccc(C[C@@H](C([R])═O) |
| | | N[R])cc1)CCOCCOCCNC(COCCOCCNC(CC[C |
| | | @@H](C(O)═O)NC(CCCCCCCCCCCCCCCCC |
| | | (O)═O)═O)═O)═O |
| 205 | 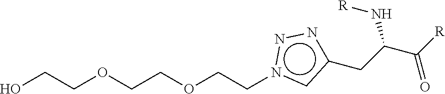 | |
| | C11H18N4O4R2 | |
| | Tzl(PEG3OH) | OCCOCCOCCn1nnc(C[C@@H](C([R])═O)N[R])c1 |
| 206 | 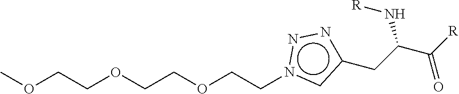 | |
| | C12H20N4O4R2 | |
| | Tzl(mPEG3) | COCCOCCOCCn1nnc(C[C@@H](C([R])═O)N |
| | | [R])c1 |
| 207 | 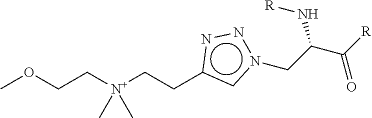 | |
| | C12H22N5O2R2+ | |
| | TzlChmPEG | C[N+](C)(CCc1cn(C[C@@H](C([R])═O)N[R])n |
| | | n1)CCOC |
| 208 | 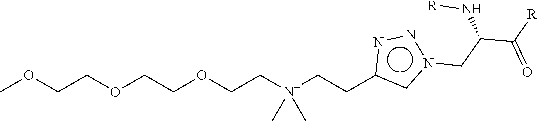 | |
| | C16H30N5O4R2+ | |
| | TzlChmPEG3 | C[N+](C)(CCc1cn(C[C@@H](C([R])═O)N[R])n |
| | | n1)CCOCCOCCOC |
| 209 | 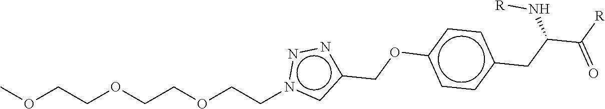 | |
| | C19H26N4O5R2 | |
| | Y(OTzl(mPEG3)) | COCCOCCOCCn1nnc(COc2ccc(C[C@@H](C |
| | | ([R])═O)N[R])cc2)c1 |
| 210 | 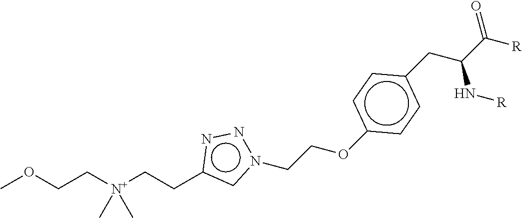 | |
| | C20H30N5O3R2+ | |
| | Y(OTzlChmPEG) | C[N+](C)(CCc1cn(CCOc2ccc(C[C@@H](C([R])═O) |
| | | N[R])cc2)nn1)CCOC |
| 211 | 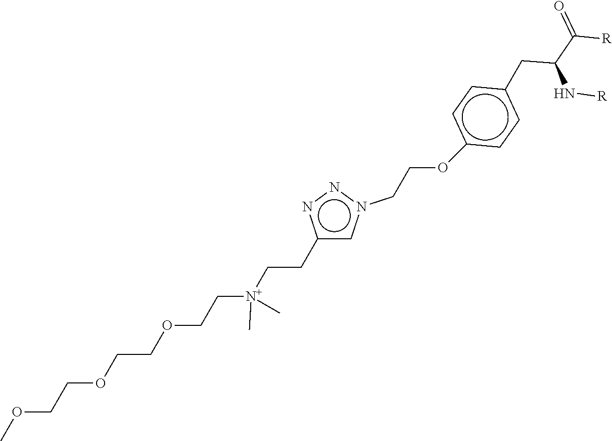 | |
| | C24H38N5O5R2+ | |
| | Y(OTzlChmPEG3) | C[N+](C)(CCc1cn(CCOc2ccc(C[C@@H](C ([R])═O)N[R])cc2)nn1)CCOCCOCCOC |
| 212 | 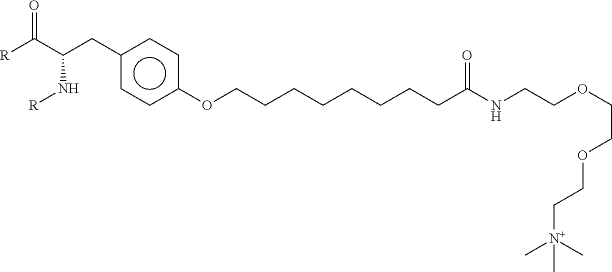 | |
| | C27H46N3O5R2+ | |
| | YC8CO(NHPEG3a) | C[N+](C)(C)CCOCCOCCNC(CCCCCCCCOc1c |
| | | cc(C[C@@H](C([R])═O)N[R])cc1)═O |
| 213 | 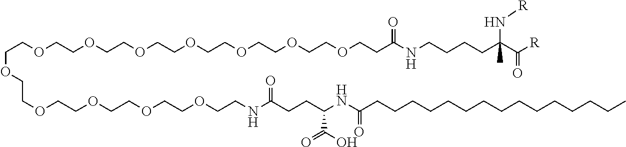 | |
| | C55H104N4O18R2 | |
| | aMeK(PEG12gEC16) | CCCCCCCCCCCCCCCC(N[C@@H](CCC(NC |
| | | COCCOCCOCCOCCOCCOCCOCCOCCOCCO |
| | | CCOCCOCCC(NCCCC[C@@](C)(C([R])═O)N[ |
| | | R])═O)═O)C(O)═O)═O |
| 214 | 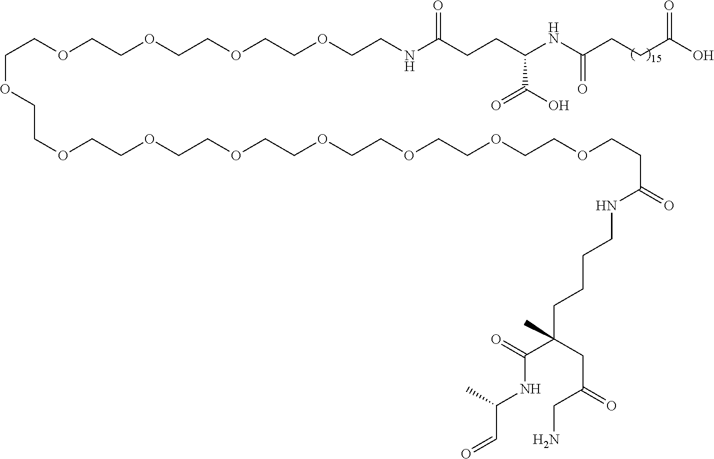 | |
| | C62H116N6O22 | |
| | aMeK(PEG12gEC18OH) | C[C@@H](C═O)NC([C@](C)(CCCCNC(CCOC |
| | | COCCOCCOCCOCCOCCOCCOCCOCCOCCO |
| | | CCOCCNC(CC[C@@H](C(O)═O)NC(CCCCCC |
| | | CCCCCCCCCCC(O)═O)═O)═O)═O)NC(CN)═O)═O |
| 215 | 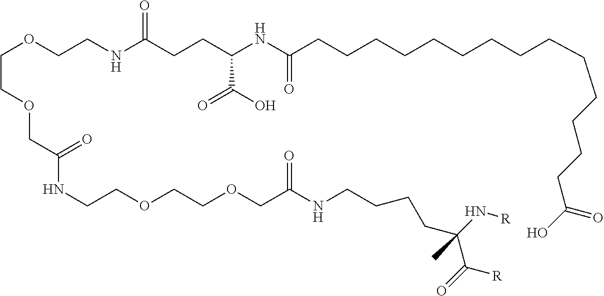 | |
| | C40H71N5O13R2 | |
| | aMeK(PEG2PEG2gEC16OH) | C[C@](CCCCNC(COCCOCCNC(COCCOCCN |
| | | C(CC[C@@H](C(O)═O)NC(CCCCCCCCCCCC |
| | | CCC(O)═O)═O)═O)═O)═O)(C([R])═O)N[R] |
| 216 | 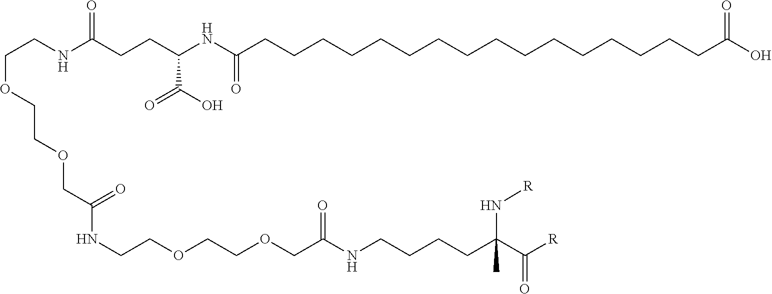 | |
| | C42H75N5O13R2 | |
| | aMeK(PEG2PEG2gEC18OH) | C[C@](CCCCNC(COCCOCCNC(COCCOCCN |
| | | C(CC[C@@H](C(O)═O)NC(CCCCCCCCCCCC |
| | | CCCCC(O)═O)═O)═O)═O)═O)(C([R])═O)N[R] |
| 217 | 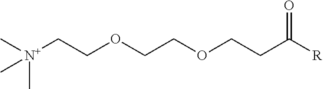 | |
| | C10H21NO3R+ | |
| | cPEG3aCO, cPEG3a | C[N+](C)(C)CCOCCOCCC([R])═O |
| 218 | 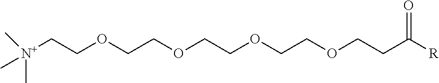 | |
| | C14H29NO5R+ | |
| | cPEG5aCO, cPEG5a | C[N+](C)(C)CCOCCOCCOCCOCCC([R])═O |
| 219 | 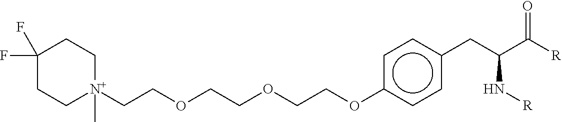 | |
| | C21H31F2N2O4R2+ | |
| | dFPPEG3F | C[N+](CCOCCOCCOc1ccc(C[C@@H](C([R])═ |
| | | O)N[R])cc1)(CC1)CCC1(F)F |
| 220 | 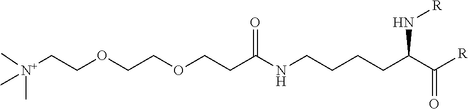 | |
| | C16H32N3O4R2+ | |
| | dK(cPEG3a), k(cPEG3a), dK(cPEG3aCO), | C[N+](C)(C)CCOCCOCCC(NCCCC[C@H](C([ |
| | k(cPEG3aCO) | R])═O)N[R])═O |
| 221 | 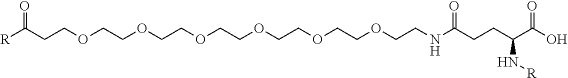 | |
| | C20H36N2O10R2 | |
| | gEPEG6 | OC([C@H](CCC(NCCOCCOCCOCCOCCOCC |
| | | OCCC([R])═O)═O)N[R])═O |
| 222 | 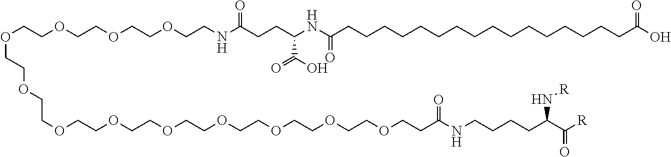 | |
| | C56H104N4O20R2 | |
| | k(PEG12gEC18OH), dK(PEG12gEC18OH) | OC(CCCCCCCCCCCCCCCCC(N[C@@H](CC |
| | | C(NCCOCCOCCOCCOCCOCCOCCOCCOCCO |
| | | CCOCCOCCOCCC(NCCCC[C@H](C([R])═O)N |
| | | [R])═O)═O)C(O)═O)═O)═O |
| 223 | 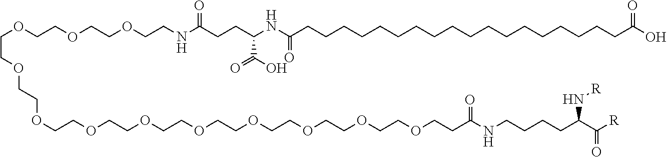 | |
| | C58H108N4O20R2 | |
| | k(PEG12gEC20OH) | OC(CCCCCCCCCCCCCCCCCCC(N[C@@H]( |
| | dK(PEG12gEC20OH) | CCC(NCCOCCOCCOCCOCCOCCOCCOCCOC |
| | | COCCOCCOCCOCCC(NCCCC[C@H](C([R])═ |
| | | O)N[R])═O)═O)C(O)═O)═O)═O |
| 224 | 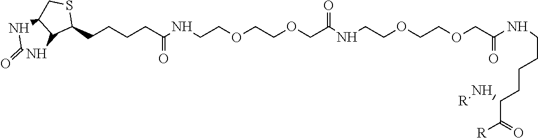 | |
| | C28H48N6O9SR2 | |
| | dK(PEG2PEG2Biotin), | O═C(CCCC[C@@H]([C@H]IN2)SC[C@@H]1 |
| | k(PEG2PEG2Biotin) | NC2═O)NCCOCCOCC(NCCOCCOCC(NCCCC[ |
| | | C@H](C([R])═O)N[R])═O)═O |
| 225 | 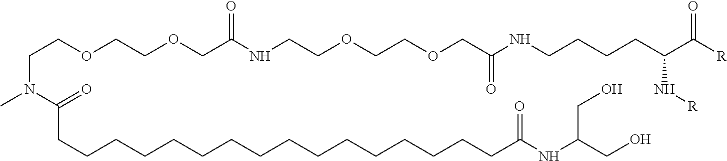 | |
| | C40H75N5O11R2 | |
| | k(PEG2PEG2C18GolB), | CN(CCOCCOCC(NCCOCCOCC(NCCCC[C@H] |
| | dK(PEG2PEG2C18GolB) | (C([R])═O)N[R])═O)═O)C(CCCCCCCCCCCCC |
| | | CCCC(NC(CO)CO)═O)═O |
| 226 | 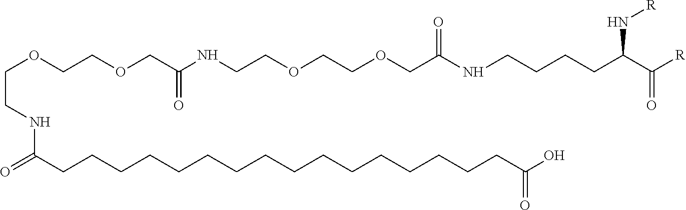 | |
| | C36H66N4O10R2 | |
| | k(PEG2PEG2C18OH), | OC(CCCCCCCCCCCCCCCCC(NCCOCCOCC( |
| | dK(PEG2PEG2C18OH) | NCCOCCOCC(NCCCC[C@H](C([R])═O) |
| | | N[R])═O)═O)═O)═O |
| 227 | 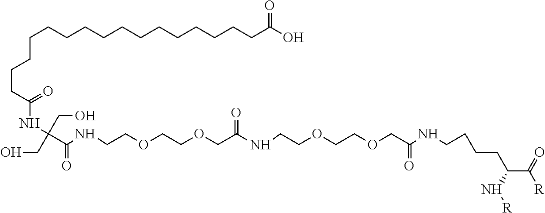 | |
| | C40H73N5O13R2 | |
| | k(PEG2PEG2GolAC18OH), | OCC(CO)(C(NCCOCCOCC(NCCOCCOCC(NC |
| | dK(PEG2PEG2GolAC18OH) | CCC[C@H](C([R])═O)═O)═O)NC(CC |
| | | CCCCCCCCCCCCCCC(O)═O)═O |
| 228 | 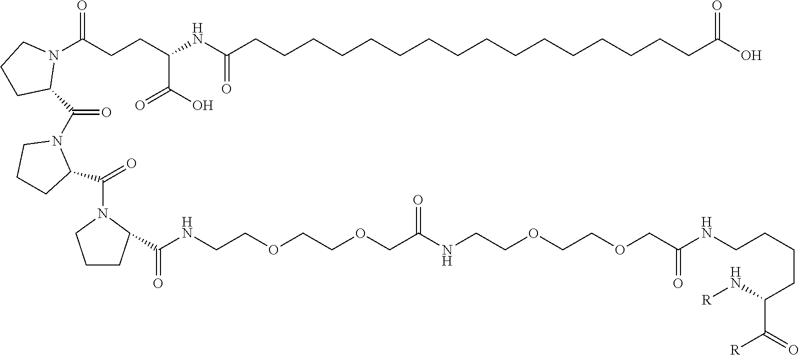 | |
| | C56H94N8O16R2 | |
| | k(PEG2PEG2PPPgEC18OH) | OC(CCCCCCCCCCCCCCCCC(N[C@@H](CC |
| | dK(PEG2PEG2PPPgEC18OH) | C(N(CCC1)[C@@H]1C(N(CCC1)[C@@H]IC( |
| | | N(CCC1)[C@@H]1C(NCCOCCOCC(NCCOCC)═ |
| | | OCC(NCCCC[C@H](C([R])═O)N[R])═O)═O)═O |
| | | O)═O)═O)C(O)═O)═O)═O |
| 229 | 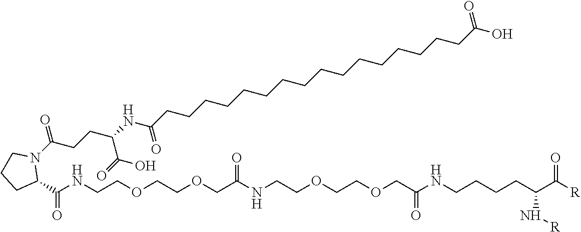 | |
| | C46H80N6O14R2 | |
| | k(PEG2PEG2PgEC18OH), | OC(CCCCCCCCCCCCCCCCC(N[C@@H](CC |
| | dK(PEG2PEG2PgEC18OH) | C(N(CCC1)[C@@H]1C(NCCOCCOCC(NCCOC |
| | | COCC(NCCCC[C@H](C([R])═O)N[R])═O)═O)═ |
| | | O)═O)C(O)═O)═O)═O |
| 230 | 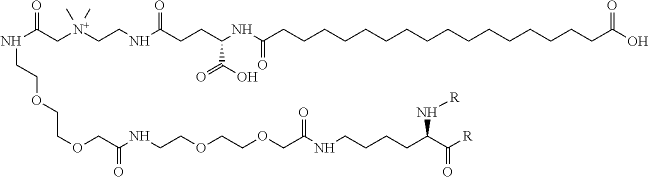 | |
| | C47H86N7O14R2+ | |
| | k(PEG2PEG2Sp6gEC18OH), | C[N+](C)(CCNC(CC[C@@H](C(O)═O)NC(CC |
| | dK(PEG2PEG2Sp6gEC18OH) | CCCCCCCCCCCCCCC(O)═O)═O)═O)CC(NCC |
| | | OCCOCC(NCCOCCOCC(NCCCC[C@H](C([R])═ |
| | | O)N[R])═O)═O)═O |
| 231 | 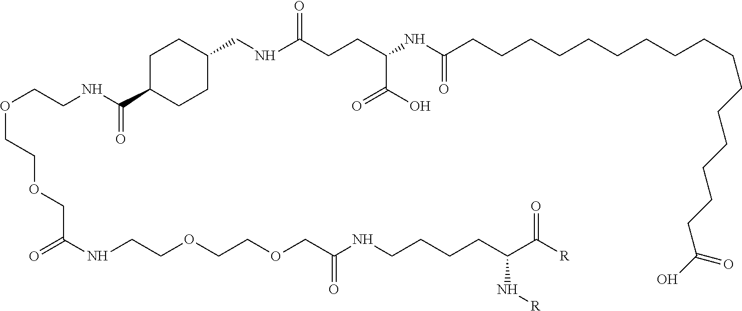 | |
| | C49H86N6O14R2 | |
| | k(PEG2PEG2TrxgEC18OH), | OC(CCCCCCCCCCCCCCCCC(N[C@@H](CC |
| | dK(PEG2PEG2TrxgEC18OH) | C(NC[C@H](CC1)CC[C@@H]1C(NCCOCCOC |
| | | C(NCCOCCOCC(NCCCC[C@H](C([R])═O)N[R])═ |
| | | O)═O)═O)═O)C(O)═O)═O)═O |
| 232 | 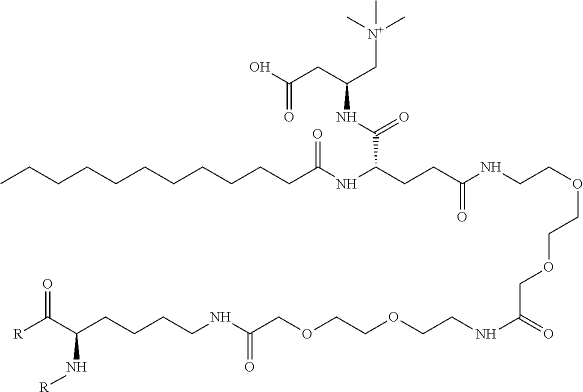 | |
| | C42H78N7O12R2+ | |
| | k(PEG2PEG2gE(C)C12, | CCCCCCCCCCCC(N[C@@H](CCC(NCCOCC |
| | dK(PEG2PEG2gE(C)C12 | OCC(NCCOCCOCC(NCCCC[C@H](C([R])═O) |
| | | N[R])═O)═O)═O)C(N[C@@H](CC(O)═O)C[N+] |
| | | (C)(C)C)═O)═O |
| 233 | 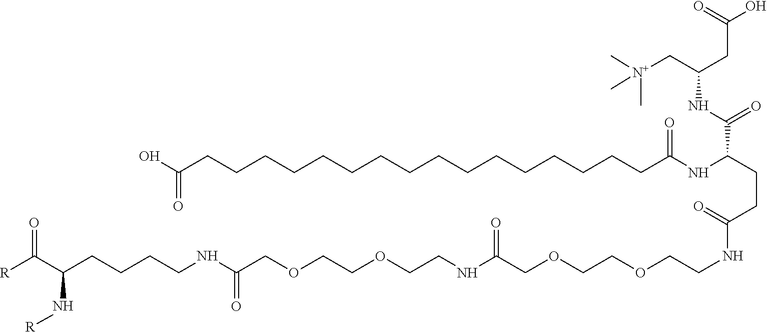 | |
| | C48H88N7O14R2+ | |
| | k(PEG2PEG2gE(C)C18OH, | C[N+](C)(C)C[C@H](CC(O)═O)NC([C@H](CC |
| | dK(PEG2PEG2gE(C)C18OH | C(NCCOCCOCC(NCCOCCOCC(NCCCC[C@H] |
| | | (C([R])═O)N[R])═O)═O)═O)NC(CCCCCCCCC |
| | | CCCCCCCC(O)═O)═O)═O |
| 234 | 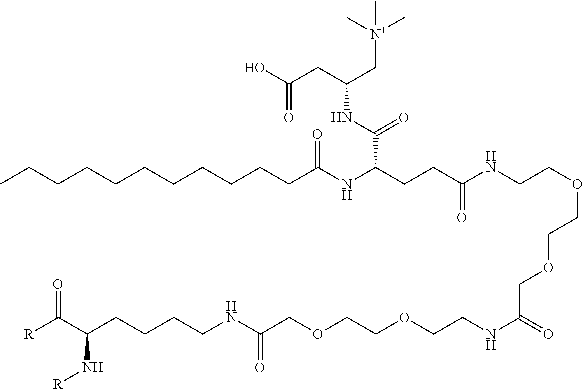 | |
| | C42H78N7O12R2+ | |
| | k(PEG2PEG2gE(c)C12, | CCCCCCCCCCCC(N[C@@H](CCC(NCCOCC |
| | dK(PEG2PEG2gE(c)C12 | OCC(NCCOCCOCC(NCCCC[C@H](C([R])═O) |
| | | N[R])═O)═O)═O)C(N[C@H](CC(O)═O)C[N+](C) |
| | | (C)C)═O)═O |
| 235 | 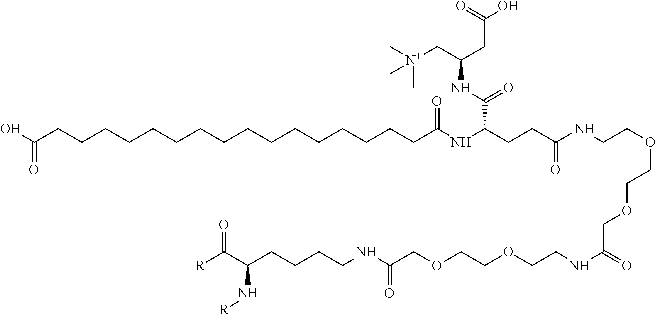 | |
| | C48H88N7O14R2+ | |
| | k(PEG2PEG2gE(c)C18OH, | C[N+](C)(C)C[C@@H](CC(O)═O)NC([C@H](C |
| | dK(PEG2PEG2gE(c)C18OH | CC(NCCOCCOCC(NCCOCCOCC(NCCCC[C@ |
| | | H](C([R])═O)N[R])═O)═O)═O)NC(CCCCCCCC |
| | | CCCCCCCCC(O)═O)═O)═O |
| 236 | 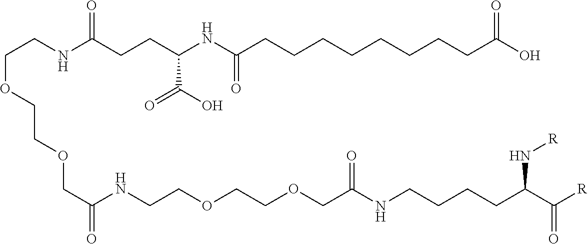 | |
| | C33H57N5O13R2 | |
| | k(PEG2PEG2gEC10OH), | OC(CCCCCCCCC(N[C@@H](CCC(NCCOCCO |
| | dK(PEG2PEG2gEC10OH) | CC(NCCOCCOCC(NCCCC[C@H](C([R])═O)N[ |
| | | R])═O)═O)═O)C(O)═O)═O)═O |
| 237 | 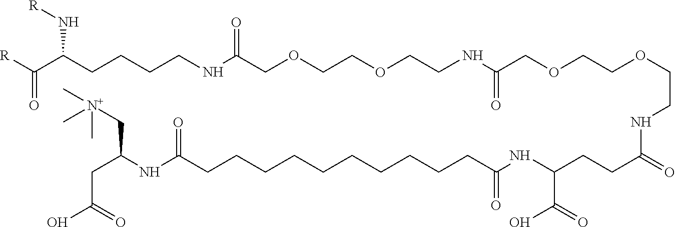 | |
| | C42H76N7O14R2+ | |
| | k(PEG2PEG2gEC12OH(C), | C[N+](C)(C)C[C@H](CC(O)═O)NC(CCCCCCC |
| | dK(PEG2PEG2gEC12OH(C) | CCCC(N[C@@H](CCC(NCCOCCOCC(NCCOC |
| | | COCC(NCCCC[C@H](C([R])═O)N[R])═O)═O)═ |
| | | O)C(O)═O)═O)═O |
| 238 | 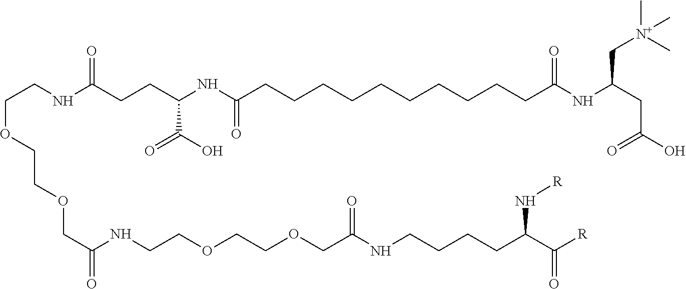 | |
| | C42H76N7O14R2+ | |
| | k(PEG2PEG2gEC12OH(c), | C[N+](C)(C)C[C@@H](CC(O)═O)NC(CCCCCC |
| | dK(PEG2PEG2gEC12OH(c) | CCCCC(N[C@@H](CCC(NCCOCCOCC(NCCO |
| | | CCOCC(NCCCC[C@H](C([R])═O)N[R])═O)═ |
| | | O)═O)C(O)═O)═O)═O |
| 239 | 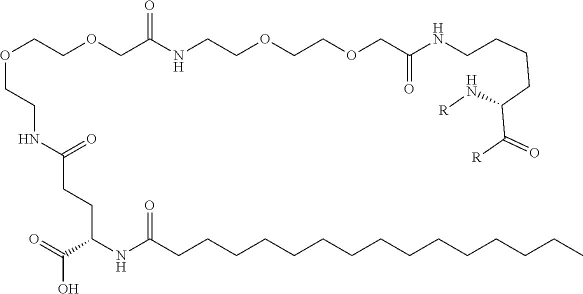 | |
| | C39H71N5O11R2 | |
| | k(PEG2PEG2gEC16), | CCCCCCCCCCCCCCCC(N[C@@H](CCC(NC |
| | dK(PEG2PEG2gEC16) | COCCOCC(NCCOCCOCC(NCCCC[C@H](C([ |
| | | R])═O)N[R])═O)═O)═O)C(O)═O)═O |
| 240 | 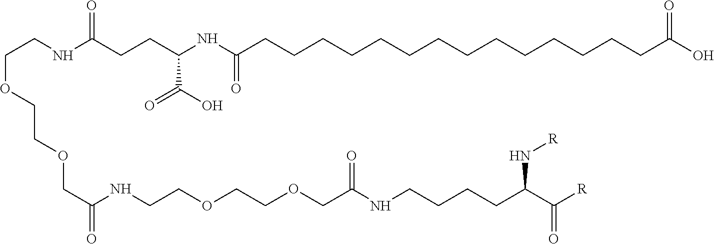 | |
| | C39H69N5O13R2 | |
| | k(PEG2PEG2gEC160H), | OC(CCCCCCCCCCCCCCC(N[C@@H](CCC(N |
| | dK(PEG2PEG2gEC160H) | CCOCCOCC(NCCOCCOCC(NCCCC[C@H](C( |
| | | [R])═O)N[R])═O)═O)═O)C(O)═O)═O)═O |
| 241 | 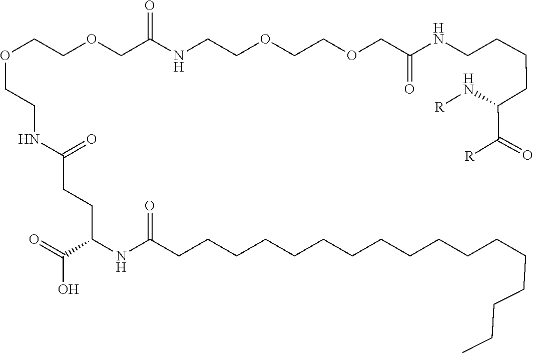 | |
| | C41H75N5O11R2 | |
| | k(PEG2PEG2gEC18), | CCCCCCCCCCCCCCCCCC(N[C@@H](CCC( |
| | dK(PEG2PEG2gEC18) | NCCOCCOCC(NCCOCCOCC(NCCCC[C@H]( |
| | | C([R])═O)N[R])═O)═O)═O)C(O)═O)═O |
| 242 | 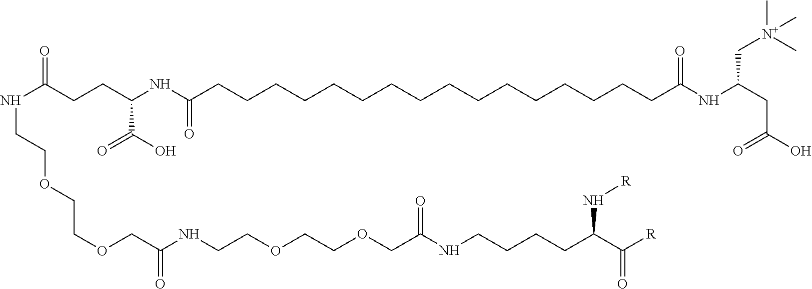 | |
| | C48H88N7O14R2+ | |
| | k(PEG2PEG2gEC18OH(C), | C[N+](C)(C)C[C@H](CC(O)═O)NC(CCCCCCC |
| | dK(PEG2PEG2gEC18OH(C) | CCCCCCCCCC(N[C@@H](CCC(NCCOCCOC |
| | | C(NCCOCCOCC(NCCCC[C@H](C([R])═O)N[R])═ |
| | | O)═O)═O)C(O)═O)═O)═O |
| 243 | 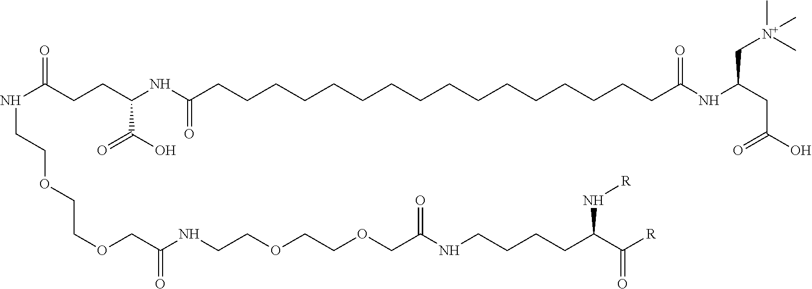 | |
| | C48H88N7O14R2+ | |
| | k(PEG2PEG2gEC18OH(c), | C[N+](C)(C)C[C@@H](CC(O)═O)NC(CCCCCC |
| | dK(PEG2PEG2gEC18OH(c) | CCCCCCCCCCC(N[C@@H](CCC(NCCOCCO |
| | | CC(NCCOCCOCC(NCCCC[C@H](C([R])═O)N[ |
| | | R])═O)═O)═O)C(O)═O)═O)═O |
| 244 | 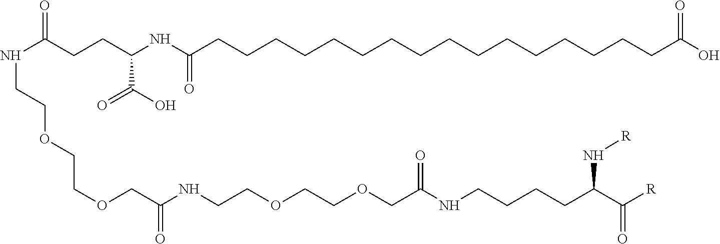 | |
| | C41H73N5O13R2 | |
| | k(PEG2PEG2gEC18OH), | OC(CCCCCCCCCCCCCCCCC(N[C@@H](CC |
| | dK(PEG2PEG2gEC18OH) | C(NCCOCCOCC(NCCOCCOCC(NCCCC[C@H] |
| | | (C([R])═O)N[R])═O)═O)═O)C(O)═O)═O)═O |
| 245 | 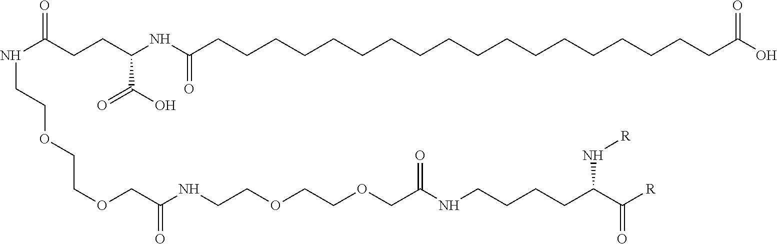 | |
| | C43H77N5O13R2 | |
| | k(PEG2PEG2gEC20OH), | OC(CCCCCCCCCCCCCCCCCCC(N[C@@H] |
| | dK(PEG2PEG2gEC20OH) | (CCC(NCCOCCOCC(NCCOCCOCC(NCCCC[C |
| | | @H](C([R])═O)N[R])═O)═O)═O)C(O)═O)═O)═O |
| 246 | 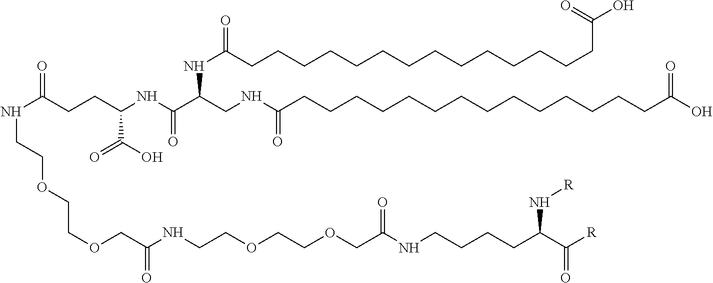 | |
| | C58H103N7O17R2 | |
| | k(PEG2PEG2gEDAP(C16OH)2), | OC(CCCCCCCCCCCCCCC(NC[C@@H](C(N |
| | dK(PEG2PEG2gEDAP(C16OH)2) | [C@@H](CCC(NCCOCCOCC(NCCOCCOCC(N |
| | | CCCC[C@H](C([R])═O)N[R])═O)═O)═O)C(O)═ |
| | | O)═O)NC(CCCCCCCCCCCCCCC(O)═O)═O)═ |
| | | O)═O |
| 247 | 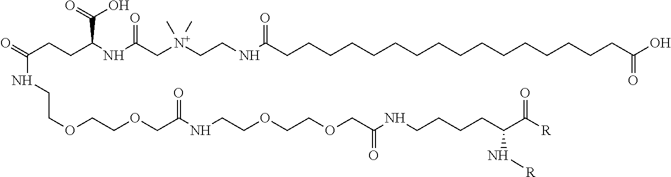 | |
| | C47H86N7O14R2+ | |
| | kPEG2PEG2gEDAP(C16OH)2;kPEG2PEG2 | C[N+](C)(CCNC(CCCCCCCCCCCCCCCCC(O)═ |
| | gEDap(C16OH)2, | O)═O)CC(N[C@@H](CCC(NCCOCCOCC(NC |
| | k(PEG2PEG2gEDAP(C16OH)2), | COCCOCC(NCCCC[C@H](C([R])═O)N[R])═O)═ |
| | dKPEG2PEG2gEDAP(C16OH)2;dKPEG2PE | O)═O)C(O)═O)═O |
| | G2gEDap(C16OH)2, | |
| | dK(PEG2PEG2gEDAP(C16OH)2) | |
| 248 | 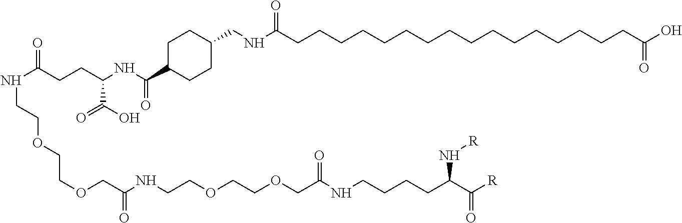 | |
| | C49H86N6O14R2 | |
| | kPEG2PEG2gEDAP(C16OH)2, | OC(CCCCCCCCCCCCCCCCC(NC[C@H](CC1) |
| | dKPEG2PEG2gEDAP(C16OH)2 | CC[C@@H]1C(N[C@@H](CCC(NCCOCCOCC |
| | | (NCCOCCOCC(NCCCC[C@H](C([R])═O)N[R])═ |
| | | O)═O)═O)C(O)═O)═O)═O)═O |
| 249 | 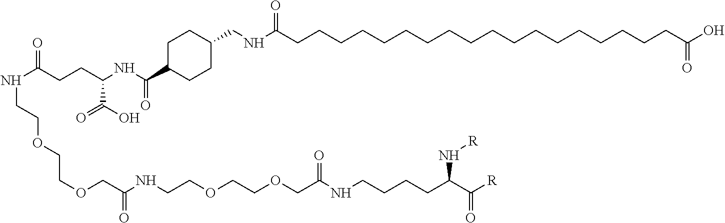 | |
| | c | |
| | C51H90N6O14R2 | |
| | k(PEG2PEG2gESp6C18OH), | OC(CCCCCCCCCCCCCCCCCCC(NC[C@H](C |
| | dK(PEG2PEG2gESp6C18OH) | C1)CC[C@@H]1C(N[C@@H](CCC(NCCOCC |
| | | OCC(NCCOCCOCC(NCCCC[C@H](C([R])═O) |
| | | N[R])═O)═O)═O)C(O)═O)═O)═O)═O |
| 250 | 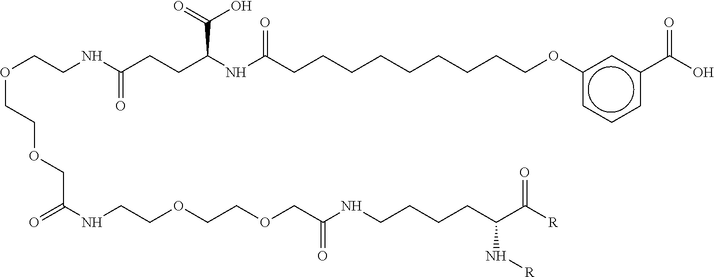 | |
| | C40H63N5O14R2 | |
| | k(PEG2PEG2gETrxC18OH), | OC([C@H](CCC(NCCOCCOCC(NCCOCCOCC |
| | dK(PEG2PEG2gETrxC18OH) | (NCCCC[C@H](C([R])═O)N[R])═O)═O)═O)NC( |
| | | CCCCCCCCCOc1cc(C(O)═O)ccc1)═O)═O |
| 251 | 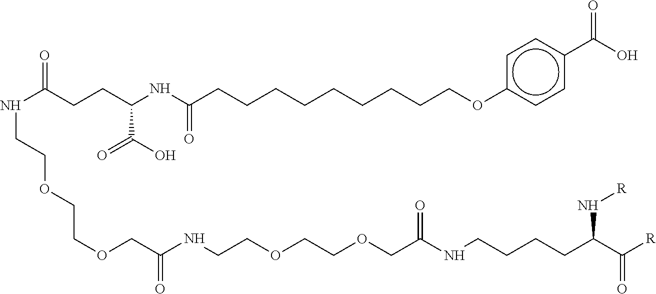 | |
| | C40H63N5O14R2 | |
| | k(PEG2PEG2gETrxC20OH), | OC([C@H](CCC(NCCOCCOCC(NCCOCCOCC |
| | dK(PEG2PEG2gETrxC20OH) | (NCCCC[C@H](C([R])═O)N[R])═O)═O)═O)NC( |
| | | CCCCCCCCCOc(cc1)ccc1C(O)═O)═O)═O |
| 252 | 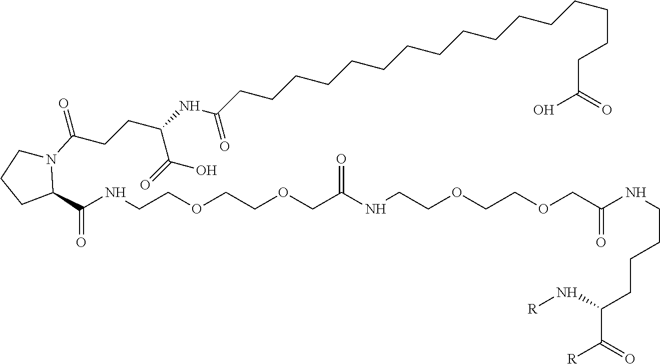 | |
| | C46H80N6O14R2 | |
| | k(PEG2PEG2gEmXOH), | OC(CCCCCCCCCCCCCCCCC(N[C@@H](CC |
| | dK(PEG2PEG2gEmXOH) | C(N(CCC1)[C@H]1C(NCCOCCOCC(NCCOCC |
| | | OCC(NCCCC[C@H](C([R])═O)N[R])═O)═O)═ |
| | | O)═O)C(O)═O)═O)═O |
| 253 | 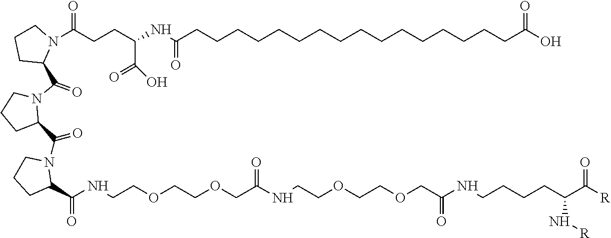 | |
| | C56H94N8O16R2 | |
| | k(PEG2PEG2gEpXOH), | OC(CCCCCCCCCCCCCCCCC(N[C@@H](CC |
| | dK(PEG2PEG2gEpXOH) | C(N(CCC1)[C@H]1C(N(CCC1)[C@H]1C(N(CC |
| | | C1)[C@H]IC(NCCOCCOCC(NCCOCCOCC(N |
| | | CCCC[C@H](C([R])═O)N[R])═O)═O)═O)═O)═ |
| | | O)═O)C(O)═O)═O)═O |
| 254 | 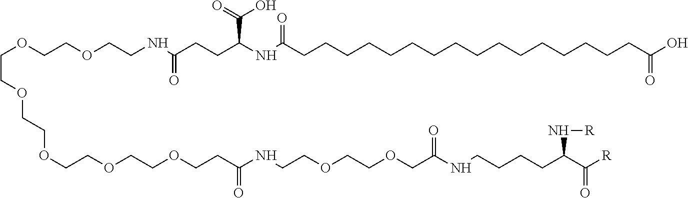 | |
| | C50H91N5O17R2 | |
| | k(PEG2PEG2pgEC18OH), | OC(CCCCCCCCCCCCCCCCC(N[C@@H](CC |
| | dK(PEG2PEG2pgEC18OH) | C(NCCOCCOCCOCCOCCOCCOCCC(NCCOC |
| | | COCC(NCCCC[C@H](C([R])═O)N[R])═O)═O)═ |
| | | O)C(O)═O)═O)═O |
| 255 | 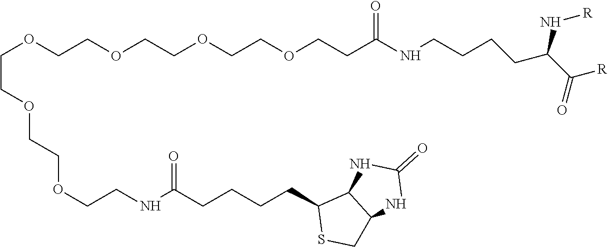 | |
| | C31H55N5O10SR2 | |
| | k(PEG2PEG2pppgEC18OH), | O═C(CCCC[C@@H]([C@H]IN2)SC[C@@H]1 |
| | dK(PEG2PEG2pppgEC18OH) | NC2═O)NCCOCCOCCOCCOCCOCCOCCC(NC |
| | | CCC[C@H](C([R])═O)N[R])═O) |
| 256 | 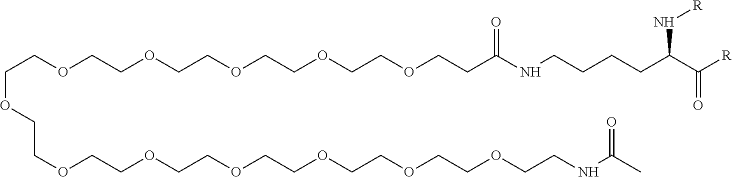 | |
| | C35H67N3O15R2 | |
| | k(PEG2PEG6gEC18OH), | CC(NCCOCCOCCOCCOCCOCCOCCOCCOCC |
| | dK(PEG2PEG6gEC18OH) | OCCOCCOCCOCCC(NCCCC[C@H](C([R])═O) |
| | | N[R])═O)═O |
| 257 | 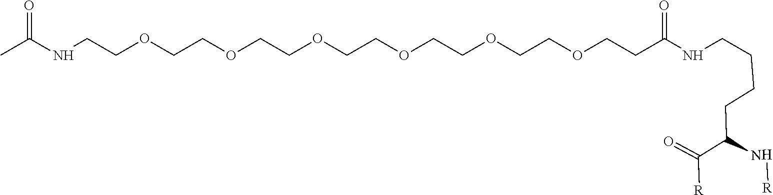 | |
| | C23H43N3O9R2 | |
| | k(dPEG12AcBr), dK(dPEG12AcBr) | CC(NCCOCCOCCOCCOCCOCCOCCC(NCCC |
| | | C[C@H](C([R])═O)N[R])═O)═O |
| 258 | 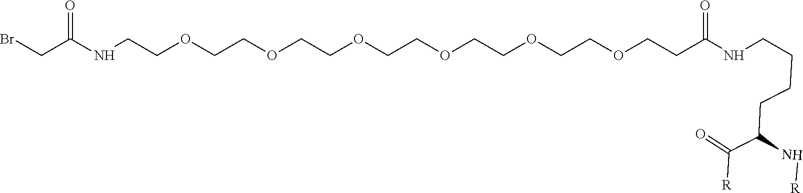 | |
| | C23H42BrN3O9R2 | |
| | k(dPEG12AcVitE), dK(dPEG12AcVitE) | O═C(CCOCCOCCOCCOCCOCCOCCNC(CBr)═ |
| | | O)NCCCC[C@H](C([R])═O)N[R] |
| 259 | 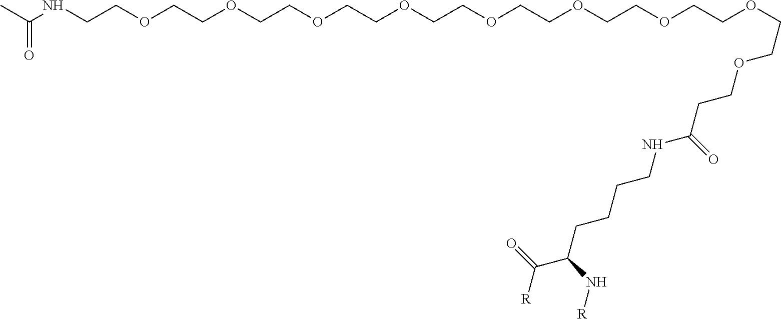 | |
| | C29H55N3O12R2 | |
| | k(dPEG6Ac), dK(dPEG6Ac) | CC(NCCOCCOCCOCCOCCOCCOCCOCCOCC |
| | | OCCC(NCCCC[C@H](C([R])═O)N[R])═O)═O |
| 260 | 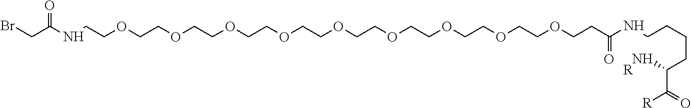 | |
| | C29H54BrN3O12R2 | |
| | k(dPEG6AcBr), dK(dPEG6AcBr) | O═C(CCOCCOCCOCCOCCOCCOCCOCCOCC |
| | | OCCNC(CBr)═O)NCCCC[C@H](C([R])═O)N[R] |
| 261 | 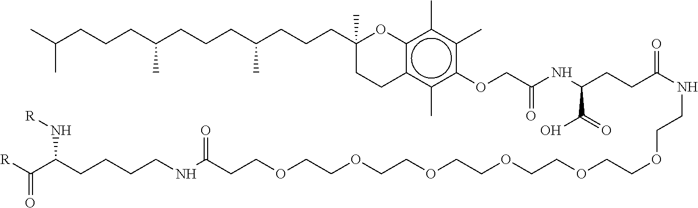 | |
| | C57H98N4O14R2 | |
| | k(dPEG9Ac), dK(dPEG9Ac) | CC(C)CCC[C@@H](C)CCC[C@@H](C)CCC[C |
| | | @](C)(CC1)Oc(c(C)c2C)c1c(C)c20CC(N[C@@ |
| | | H](CCC(NCCOCCOCCOCCOCCOCCOCCC(N |
| | | CCCC[C@H](C([R])═O)N[R])═O)═O)C(O)═O)═O |
| 262 | 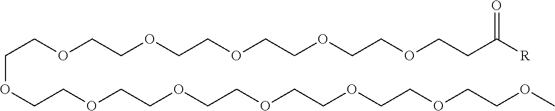 | |
| | C26H51O13R | |
| | mPEG12CO | COCCOCCOCCOCCOCCOCCOCCOCCOCCO |
| | | CCOCCOCCC([R])═O |
| 263 | 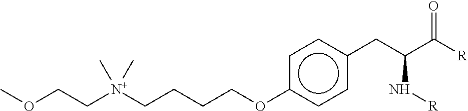 | |
| | C18H29N2O3R2+ | |
| | mPEG2TMA4F | C[N+](C)(CCCCOc1ccc(C[C@@H](C([R])═O)N |
| | | [R])cc1)CCOC |
| 264 | 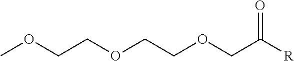 | |
| | C7H13O4R | |
| | mPEG3CO | COCCOCCOCC([R])═O |
| 265 | 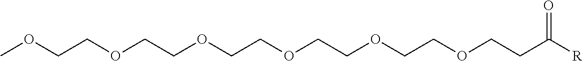 | |
| | C14H27O7R | |
| | mPEG6CO | COCCOCCOCCOCCOCCOCCC([R])═O |

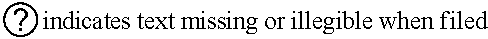

### General Peptide Synthetic Procedure 1

IL-23R inhibitor compounds described herein were synthesized from amino acids monomers using Merrifield solid phase synthesis techniques on Protein Technology's Symphony multiple channel synthesizer. The peptides were assembled using HBTU (O-Benzotriazole-N,N,N′,N′-tetramethyl-uronium-hexafluoro-phosphate), Diisopropylethyl amine(DIEA) coupling conditions. For some amino acid couplings PyAOP(7-Azabenzotriazol-1-yloxy)tripyrrolidinophosphonium hexafluorophosphate) and DIEA conditions were used. Rink Amide MBHA resin (100-200 mesh, 0.57 mmol/g) was used for peptide with C-terminal amides and pre-loaded Wang Resin with N-α-Fmoc protected amino acid was used for peptide with C-terminal acids. The coupling reagents (HBTU and DIEA premixed) were prepared at 100 mmol concentration. Similarly, amino acids solutions were prepared at 100 mmol concentration. Peptide inhibitors of the present invention were identified based on medical chemistry optimization and/or phage display and screened to identify those having superior binding and/or inhibitory properties.

### Preparation of Certain Modified Amino Acids

Certain modified amino acids appear in the sequences of the IL-23R inhibitors described herein. Those modified amino acids, and their precursors suitable for synthesizing the inhibitors described herein may be obtained from commercial sources, synthesized as described in the art, or by any suitable route. For example, substituted tryptophans may be prepared by any suitable route. Preparation of certain substituted tryptophans including those substituted at the seven position, such as 7-alkyl-tryptophans (e.g., 7-ethyl-L-tryptophans), which along with other substituted tryptophans, are described in, for example WO 2021/146441 A1. The synthesis of certain additional modified amino acids are described herein below.

### a. Synthesis of (S)-5-(4-(2-((((9H-fluoren-9-yl)methoxy)carbonyl)amino)-2-carboxyethyl)phenoxy)-N,N,N-trimethylpentan-1-aminium (TMAPF)

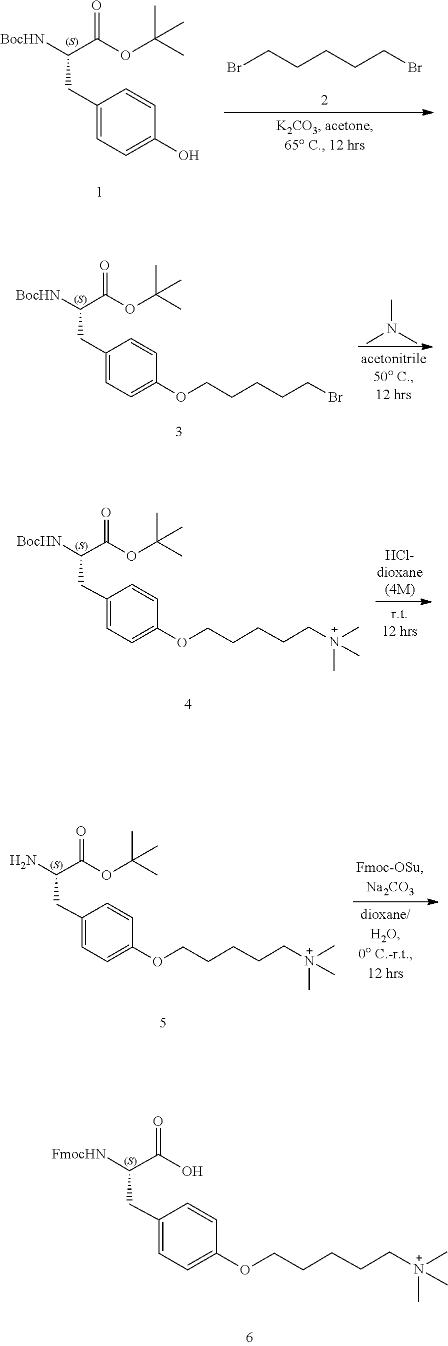

To a mixture of 1 (6.60 g, 19.7 mmol), K₂CO₃ (4.09 g, 29.6 mmol) and acetone (50 mL) was added 2 (4.99 g, 21.7 mmol). The reaction mixture was heated to refluxed and stirred for 12 hours. The reaction mixture was poured into water (500 mL) and extracted with ethyl acetate (500 mL×3). The combined organic extracts were washed with brine (500 mL), dried over anhydrous Na₂SO₄, filtered and concentrated under reduced pressure to afford the crude product, which was purified by FCC (eluent: petroleum ether:ethyl acetate=1:0 to 5:1) to afford crude product 3 (5.26 g, yield: 54.8%) as pale colourless oil. MS (ESI): mass calculated for C₂₃H₃₆BrNO₅, 486.44, m/z found 509.9 [M+23]⁺. ¹H NMR (400 MHz, CDCl₃): δ ppm 7.07 (d, J=8.4 Hz, 2H), 6.81 (d, J=8.6 Hz, 2H), 4.97 (br d, J=8.2 Hz, 1H), 4.36-4.48 (m, 1H), 3.95 (t, J=6.3 Hz, 2H), 3.45 (t, J=6.8 Hz, 2H), 3.00 (br d, J=3.7 Hz, 2H), 1.87-2.01 (m, 2H), 1.76-1.86 (m, 2H), 1.62-1.69 (m, 2H), 1.42 (d, J=2.8 Hz, 18H).

To a mixture of 3 (5.26 g, 10.8 mmol) in acetonitrile (50 mL) was added trimethylamine in acetonitrile (2 M, 8.11 mL). The reaction mixture was stirred for 12 hours at 50° C. The reaction mixture was concentrated under reduced pressure to obtain the product 4 (5.0 g, yield: 99.3%) as pale-yellow solid.

MS (ESI): mass calculated for C₂₆H₄₅N₂O₅, 465.646, m/z found 465.2 [M]⁺. The mixture of 4 (4.00 g, 8.59 mmol) in 4M HCl-dioxane (43.0 mL, 172 mmol) was stirred for 12 hours at room temperature. The solvent was removed under reduced pressure to obtain the product 5 (3.00 g, yield: crude) as a white solid, which was used to next step directly. MS (ESI): mass calculated For C₁₇H₂₉N₂O₃, 309.424, m/z found 309.1 [M+H]⁺.

Compound 5 (3.00 g, 8.67 mmol) was dissolved in dioxane (20 mL) and water (20 mL) in a round-bottom flask. Na₂CO₃ (1.38 g, 13.0 mol) was added, and the solution cooled to 0° C. in an ice bath. Then Fmoc-OSu (3.22 g, 9.54 mol) was dissolved in dioxane (20 mL) and added in portions to the solution at 0° C. The reaction was stirred for 2 hours at 0° C. The reaction was allowed to warm to room temperature overnight. The reaction was acidized with 2N HCl (50 mL). The reaction mixture was purified by preparative HPLC using a Xtimate C18 150*40 mm*5 um (eluent: 20% to 50% (v/v) CH₃CN and H₂O with 0.05% HCl) to afford product. The product was suspended in water (40 mL), the mixture frozen using dry ice/ethanol, and then lyophilized to dryness to afford the title compound 6 (TMAPF, 3.57 g, yield: 61.9%, purity: 99.2%) as pale-yellow solid. MS (ESI): mass calculated For C₃₂H₃₉N₂O₅, 531.662, m/z found 531.4 [M+H]⁺. 1H NMR (400 MHz, DMSO-d₆) δ ppm 7.89 (d, J=7.6 Hz, 2H), 7.73 (d, J=8.2 Hz, 1H), 7.65 (t, J=7.2 Hz, 2H), 7.39-7.43 (m, 2H), 7.27-7.34 (m, 2H), 7.19 (d, J=8.2 Hz, 2H), 6.78-6.89 (m, 2H), 4.06-4.25 (m, 4H), 3.84-3.99 (m, 2H), 3.25-3.37 (m, 2H), 3.05 (s, 9H), 3.00 (d, J=4.0 Hz, 1H), 2.70-2.84 (m, 1H), 1.63-1.82 (m, 4H), 1.30-1.46 (m, 2H)

### b. Synthesis of (S)-2-((((9H-fluoren-9-yl)methoxy)carbonyl)amino)-3-(7-(3-acetamidophenyl)-1H-indol-3-yl)propanoic acid (7-(3-Nacetyl-phenyl)-tryptophan or 7(3NAcPh)W)

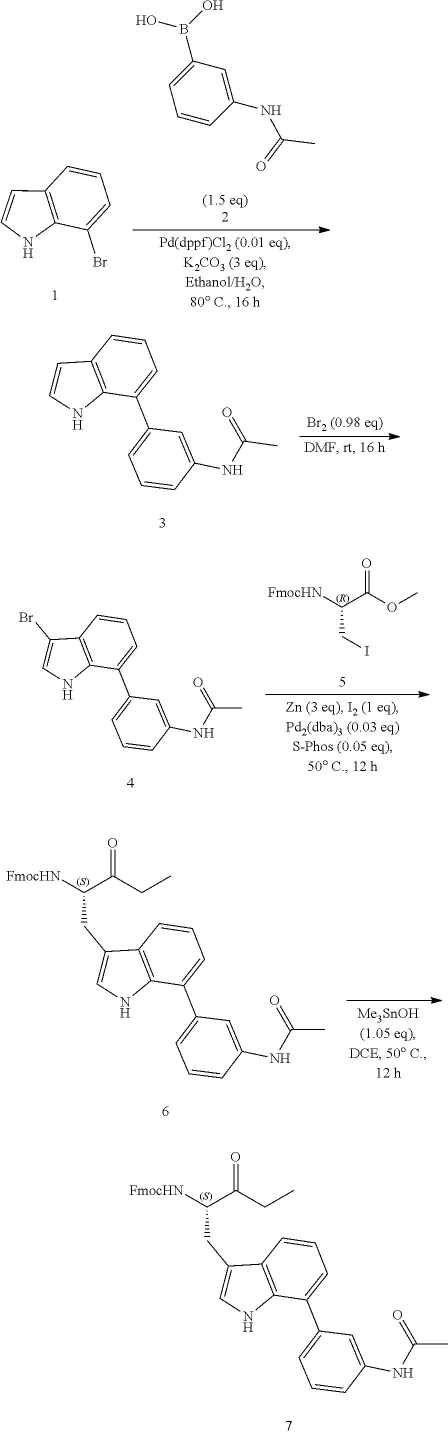

To a solution of 1 (30.0 g, 153 mmol), compound 2 (41.1 g, 230 mmol) and K₃PO₄ (97.4 g, 459 mmol) in H₂O/ethanol (500 mL) and, Pd(dppf)Cl₂ (1.12 g, 1.53 mmol) was added under an N₂ atmosphere. The mixture was stirred at 80° C. for 16 h. The mixture was filtered. The mixture was concentrated, then extracted with ethyl acetate (500 mL×2), dried with anhydrous Na₂SO₄. The organic layer was concentrated and purified by FCC (eluent: petroleum ether/ethyl acetate=1:0 to 55:45) to give 3 (25.0 g, yield: 62.5%) as yellow oil MS (ESI): mass calculated for C₁₆H₁₄N₂O, 250.295, m/z found 251.0 [M+].

To a 1 L round-bottomed flask containing a solution of 3 (12.0 g, 47.9 mmol) in DMF (300 mL) bromine (Br₂, 2.422 mL, 47.0 mmol) was slowly added. The mixture was stirred at 25° C. for 16 hours. The solution was added to aqueous sodium sulfite (500 mL), the mixture was stirred at 25° C. for 2 hours. The mixture was filtered, the filter cake was mixed with H₂O (400 mL) and stirred at 25° C. for 1 h. The mixture was filtered, the solid was collected to give 4 as a crude product, which was purified by preparative high-performance liquid chromatography (Column: Phenomenex C18 250×50 mm×10 um, Condition: water (FA)-CAN (20%-60%)). The mixture was concentrated, extracted with CH₂Cl₂ (1 L×2), washed with brine, dried with anhydrous Na₂SO₄. The organic layers were filtered and concentrated to give 4 (9.70 g, yield: 60.8%) as a pale white. MS (ESI): mass calculated For C₁₆H₁₃BrN₂O, 329.191, m/z found 328.8 [M].

A 250 mL three neck round-bottomed flask was charged with activated Zn powder (5.84 g, 89.3 mmol), DMF (120 mL) and I₂ (382 mg, 1.50 mmol) was added under an N₂ atmosphere at room temperature. After stirring for 20 min, a solution of 5 (13.6 g, 30.1 mmol) in DMF (30 mL) was added to the mixture. The reaction mixture was stirred for 30 min. at room temperature, after which 4 (9.70 g, 29.5 mmol), tris(dibenzylideneacetone)palladium (826 mg, 0.902 mmol), 2-dicyclohexylphosphino-2′,6′-dimethoxybiphenyl (617 mg, 1.50 mmol) were added under an N₂ atmosphere. The reaction mixture was stirred at 50° C. for 12 hours, after which solvent was removed under reduced pressure to give crude product 6. The crude product was extracted with ethyl acetate (1500 mL). The extract was washed with H₂O (500 mL×2), followed by brine (500 mL), after which it was dried over anhydrous Na₂SO₄, filtered, and concentrated to dryness in vacuo to give crude intermediate 6, which was purified by silica gel chromatography (0-100% ethyl acetate/petroleum ether (EtOAc/PE)) to afford 6 (11.0 g, yield: 63.8%) as a brown-yellow oil. MS (ESI): mass calculated for C₃₅H₃₁N₃O₅, 573.638, m/z found 574.1 [M+1].

Intermediate 6 (11.0 g, 19.2 mmol), a stir bar, Me₃SnOH (3.64 g, 20.1 mmol) and DCE (150 mL) were added to a 250 mL round-bottomed flask and stirred at 50° C. for 12 hours. To the reaction mixture 2 N HCl was added to adjust the to pH to 6. A second reaction series starting with a solution of 1 was prepared and the combined reaction mixtures were concentrated under reduced pressure to give the crude product 7, which was purified by preparative HPLC using a Xtimate C18 150×40 mm×5 um (eluent: 38% to 68% (v/v) CH₃CN and H₂O with 0.05% HCl) to afford product 7. The product was suspended in water (100 mL), the mixture frozen using dry ice/ethanol, and then lyophilized to dryness to afford 7 (7(3NAcPh)W, 11.8 g, yield: 66.8%) as a white solid. MS (ESI): mass calculated For C₃₄H₂₉N₃O₅, 559.611, m/z found 560.0 [M+1]. ¹H NMR DMSO-d₆ (400 MHz) δ 10.73 (s, 1H), 10.10 (s, 1H), 7.52-8.02 (m, 7H), 6.96-7.52 (m, 9H), 4.03-4.44 (m, 3H), 3.25 (d, J=13.2 Hz, 2H), 3.01-3.15 (m, 1H), 2.08 (s, 3H).

### c. Synthesis of (S)-2-((((9H-fluoren-9-yl)methoxy)carbonyl)amino)-3-(6-(tert-butoxy)naphthalen-2-yl)propanoic acid (5-methyl-pyridyl-alanine or 5MePyridinAla)

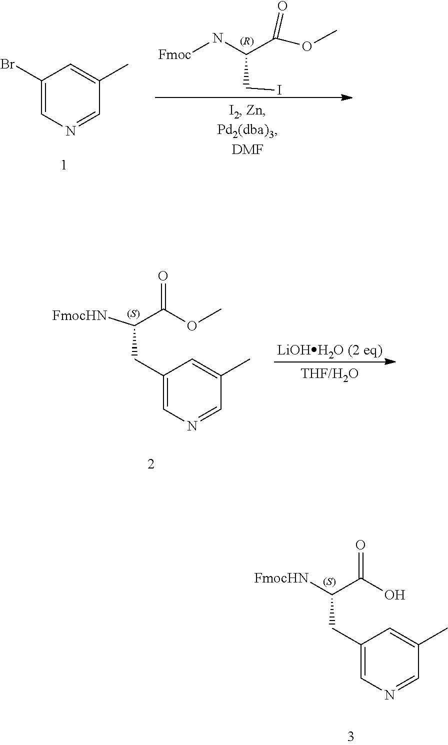

Activated Zn powder (8.18 g, 125 mmol), DMF (150 mL) and I₂ (0.534 g, 2.11 mmol) were stirred under an N₂ atmosphere at room temperature for 20 min, after which (R)-methyl 2-((((9H-fluoren-9-yl)methoxy)carbonyl)amino)-3-iodopropanoate (19.0 g, 42.1 mmol) in DMF (25 mL) was added. The reaction mixture was stirred for 30 min at room temperature, after which a mixture of 1 (7.97 g, 46.3 mmol), tris(dibenzylideneacetone)palladium (1.16 g, 1.26 mmol) and 2-dicyclohexylphosphino-2′,6′-dimethoxybiphenyl (0.864 g, 2.11 mmol) in DMF (25 mL) was added under an N₂ atmosphere. The resulting reaction mixture was stirred at 50° C. for 12 h. The solvent was removed under reduced pressure to give the crude, which was purified by FCC (eluent: petroleum ether:ethyl acetate=1:0 to 0:1 and ethyl acetate:methanol=1:0 to 2:1) to afford the product 2 (10.00 g, 57.0% yield) as pale-yellow liquid. MS (ESI): mass calculated for C₂₅H₂₄N₂O₄, 416.469, m/z found 417.1 [M+H]⁺.

To a mixture of 2 (9.50 g, 22.8 mmol) in THE (100 mL) was added LiOH·H₂O (1.91 g, 45.6 mmol) in H₂O (10 mL). The mixture was stirred for 1 h at 0° C. TLC showed most SM were consumed. To the reaction mixture was added HCl (1 N) dropwise at ice bath to pH=5. The reaction mixture was concentrated under reduced pressure, then poured into water (200 mL) the mixture was extracted with THE (200 mL×3). The organic layers were combined, washed with brine (100 mL), dried over anhydrous Na₂SO₄. After filtering the organic layers were concentrated under reduced pressure to afford crude product 3, which was purified by FCC (eluent: ethyl acetate:methanol=1:0 to 2:1) to obtain 3 (5MePyridinAla, 6.716 g, yield: 72.3%) as a white powder. MS (ESI): mass calculated For C₂₄H₂₂N₂O₄, 402.442, m/z found 403.1 [M+H]⁺. 1H NMR DMSO-d₆ (Bruker_400 MHz): δ 8.18 (s, 2H), 7.88 (d, J=7.6 Hz, 2H), 7.63 (d, J=7.2 Hz, 2H), 7.45-7.26 (m, 5H), 6.81 (s, 1H), 4.33-4.21 (m, 1H), 4.20-4.09 (m, 2H), 3.95 (s, 1H), 3.06-3.05 (m, 1H), 2.92-2.89 (m, 1H), 2.18 (s, 3H).

### d. Synthesis of (S)-2-((((9H-fluoren-9-yl)methoxy)carbonyl)amino)-3-(4-(2-(3-((2,2,4,6,7-pentamethyl-2,3-dihydrobenzofuran-5-yl)sulfonyl)guanidino)ethoxy) phenyl)propanoic acid (AEF(G)

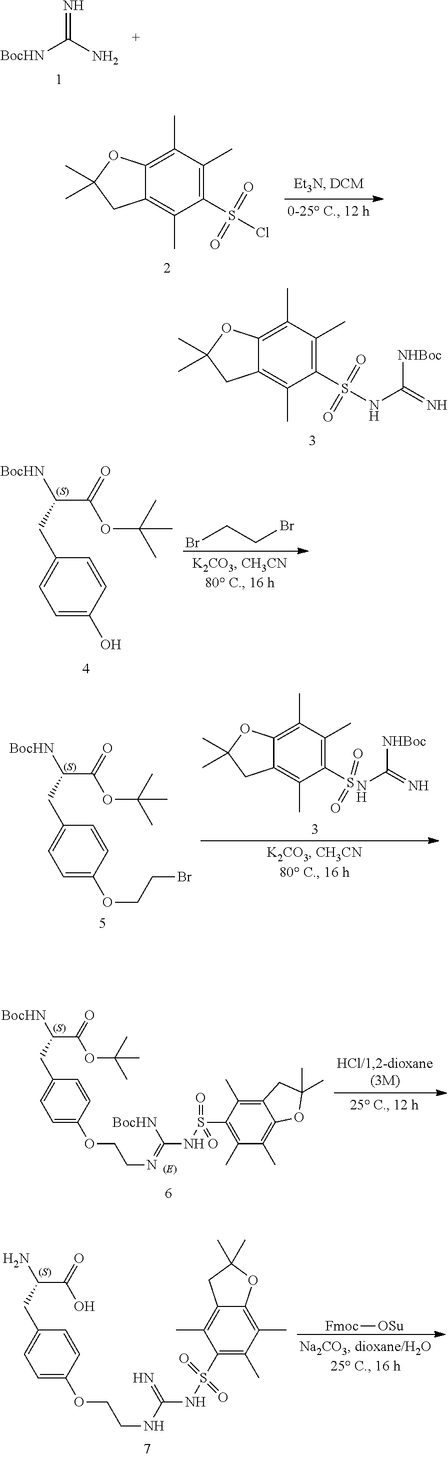

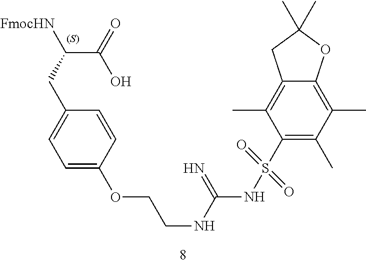

Starting material 1 (9.9 g, 62.2 mmol), a stir bar, Et₃N (14 mL, 101 mmol), and dichloromethane (DCM, 250 mL) were added to a 500 mL round-bottomed flask. The resulting mixture was treated with 2 (10 g, 34.6 mmol) in portions under ice-water bath. Then the reaction mixture was stirred at 25° C. for 12 hours. The reaction mixture was diluted with H₂O (800 mL), extracted with DCM (400 mL×2). The organic phase extracts were combined, washed with brine (800 mL), and concentrated to give the crude intermediate 3 as a yellow solid. The crude intermediate was triturated with ethyl acetate (50 mL) and the suspension isolated via filtration. The filter cake was washed with ethyl acetate (20 mL×3) before drying under reduced pressure to give the 3 (7.12 g, 49%) as a white solid. MS (ESI): mass calculated for C₁₉H₂₉N₃O₅S6, 411.5, m/z found 412.1 [M+H]⁺.

Starting material 4 (50.0 g, 148 mmol), a stir bar, DMF (300 mL), and K₂CO₃ (102 g, 739 mmol) were added to a nitrogen-purged 1000 mL round-bottomed flask. The flask was subsequently evacuated and refilled with nitrogen (×3), after which 1,2-dibromoethane (154 mL, 1.78 mol) was added, and the resulting mixture was stirred at 80° C. for 16 h under a N₂ atmosphere. The reaction mixture was filtered and concentrated to dryness under reduced pressure to give the crude product, which was subjected to silica gel chromatography (eluent: EtOAc:pet ether=0-60%) to give the 5 (64 g, 96%) as a light-yellow oil. MS (ESI): mass calculated for C₂₀H₃₀BrNO₅, 444.36, m/z found 466.1 [M+Na]⁺.

Intermediate 5 (6.1 g, 13.7 mmol), 3 (6.2 g, 15.1 mmol), K₂CO₃ (7.6 g, 55.0 mmol), a stir bar, and CH₃CN (100 mL) were charged into a 250 mL round-bottomed flask. The reaction mixture was stirred at 80° C. for 16 h under a N₂ atmosphere. The reaction mixture was cooled to room temperature, diluted with H₂O (200 mL), extracted with ethyl acetate (100 mL×2). The organic phases were combined and washed with brine (300 mL) and concentrated to give the crude intermediate 6. The crude intermediate was purified by flash column chromatography (FCC, eluent: ethyl acetate/petroleum ether=0:1 to 2:1) to give the 6 (6.62 g, 44.2%) as a white solid. MS (ESI): mass calculated for C₃₉H₅₈N₄O₁₀S, 774.9, m/z found 775.5 [M+H]⁺.

Intermediate 6 (6.6 g, 8.52 mmol), HCl/1, 4-dioxane (90 mL, 4M), a stir bar, and 1, 4-dixoane (30 mL) were charged into a 250 mL round bottomed flask. The resulting mixture was stirred at 25° C. for 12 hr. The solvent was removed under reduced pressure to give intermediate 7 (7.8 g, crude product) as a colourless oil, which was directly used to next step. MS (ESI): mass calculated for C₂₅H₃₄N₄O₆S, 518.6, m/z found 519.2 [M+H]⁺.

Intermediate 7 (7.80 g, 15.0 mmol), a stir bar, Na₂CO₃ (3.19 g, 30.1 mmol), Fmoc-OSu (5.58 g, 16.5 mmol), 1, 4-dioxane (50 mL), and H₂O (50 mL) were added into a 250 mL round-bottomed flask at 25° C. The reaction mixture was stirred at 25° C. for 16 hours, after which it was adjusted to pH=5-6 with HCl (2M) and the resulting reaction mixture was extracted with EtOAc (150 mL×3). The organic phases from the extraction were combined and washed with brine (200 mL) and concentrated to give the crude intermediate 7. The crude intermediate was purified by preparative HPLC with a Column: Phenomenex C18 150×40 mm×5 um, (eluent: 42% to 72% (v/v) CH₃CN and H₂O with 0.1% HCl) to afford pure product. The product was suspended in water (100 mL), the mixture frozen using dry ice/ethanol, and then lyophilized to dryness to afford desired product 8 (AEF(G), 4 g, 36%) as a white solid. MS (ESI): mass calculated for C₄₀H₄₄N₄O₈S, 740.9, m/z found 741.3 [M+H]⁺. 1H NMR (400 MHz, DMSO-d₆): 7.87 (d, J=7.2 Hz, 2H), 7.71-7.62 (m, 2H), 7.39 (td, J=4.0, 7.2 Hz, 2H), 7.29 (td, J=7.6, 12.0 Hz, 2H), 7.14 (br d, J=8.0 Hz, 2H), 6.99-6.85 (m, 1H), 6.77 (br d, J=8.4 Hz, 2H), 6.59-6.50 (m, 1H), 4.21-4.06 (m, 4H), 3.88 (br s, 2H), 3.42-3.36 (m, 4H), 2.99 (br dd, J=4.4, 14.0 Hz, 1H), 2.92 (s, 2H), 2.78 (br dd, J=10.8, 13.6 Hz, 1H), 2.47 (br s, 3H), 2.41 (s, 3H), 1.97 (s, 3H), 1.38 (s, 6H).

### e. Synthesis of 2-(2-(2-carboxyethoxy)ethoxy)-N,N,N-trimethylethan-1-aminium (cPEG3a)

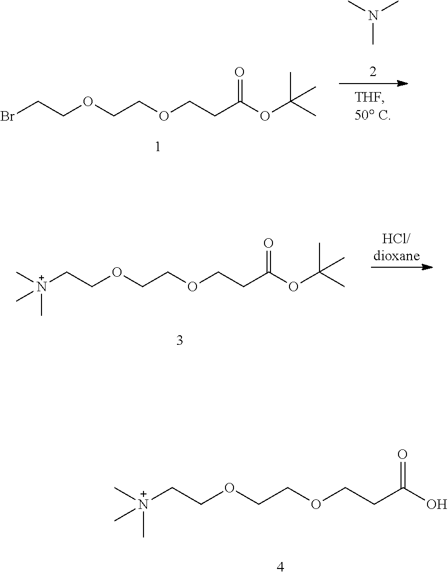

A mixture 1 (5.00 g, 16.8 mmol) and trimethylamine 2 (25 mL, 50 mmol, in THF) in dry THE (10 mL) was stirred for 16 hours at 50° C. under N2. The mixture was concentrated to give the product 3 (6.0 g, yield: 99.8%) as yellow oil. ¹H NMR (DMSO-d₆, 400 MHz): δ3.88-3.79 (m, 2H), 3.64-3.48 (m, 8H), 3.12 (s, 9H), 2.42 (t, J=6.4 Hz, 2H), 1.39 (s, 9H). A mixture of 3 (6.00 g, 16.8 mmol) and HCl/dioxane (60 mL, 240 mmol) was stirred for 16 hours at 25° C. under N₂. The mixture was concentrated to give the product 4 (cPEG3a, 4.3 g, yield: 99.8%) as yellow oil. ¹H NMR (D₂O, 400 MHz): δ3.96-3.87 (m, 2H), 3.74 (t, J=5.6 Hz, 2H), 3.64 (s, 4H), 3.57-3.49 (m, 2H), 3.12 (s, 9H), 2.60 (t, J=5.6 Hz, 2H).

### f. Synthesis of (S)-2-(2-(2-(4-(2-((((9H-fluoren-9-yl)methoxy)carbonyl) amino)-2-carboxyethyl)phenoxy)ethoxy)ethoxy)-N,N,N-trimethylethan-1-aminium (APEG3F)

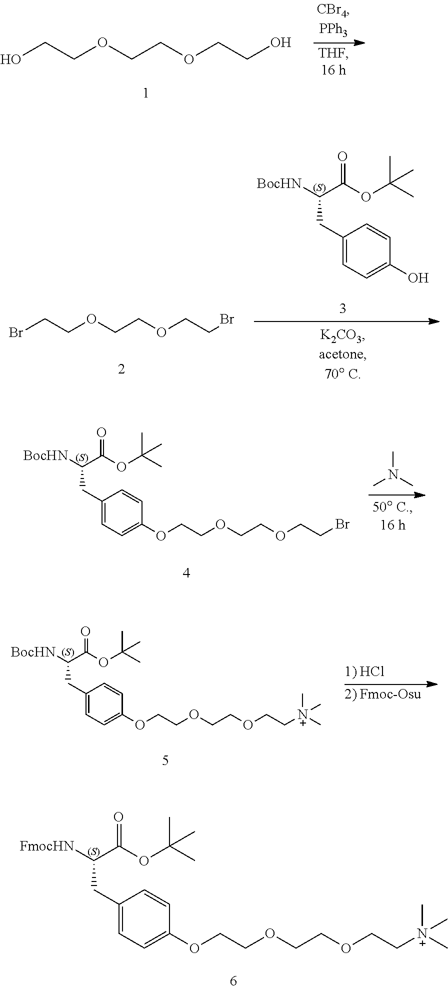

To a mixture of 1 (50.0 g, 333 mmol) in THF (1.3 L) was added PPh3 (188 g, 716 mmol), after which CBr4 (243 g, 732 mmol) was very slowly added to the mixture at 0° C. The mixture was stirred at room temperature overnight (16 h) and then concentrated under reduced pressure to give the crude intermediate 2. Petroleum ether (2.0 L) and ethyl acetate (200 mL) were added to the mixture and stirred at 25° C. for 0.5 h. The mixture was filtered, concentrated under reduced pressure, and purified by FCC (eluent: petroleum ether:ethyl acetate=1:0 to 1:9) to give intermediate 2 (52 g, yield: 56.6%) as colorless oil. ¹H NMR (400 MHz, Chloroform-d): 3.91-3.81 (m, 4H), 3.75-3.68 (m, 4H), 3.55-3.46 (m, 4H).

To a solution of 3 (45.9 g, 136 mmol) and K2CO3 (56.3 g, 408 mmol) in acetone (1 L) was added 2 (75.0 g, 272 mmol) under a nitrogen atmosphere. The mixture was stirred at 70° C. for 16 h. The mixture was filtered and evaporated, and the residue was purified by flash column chromatography FCC (eluent: petroleum ether:ethyl acetate=1:0 to 1:9) to give the intermediate 4 (45 g, yield: 61.6%) as a pale-yellow oil. MS (ESI): mass calculated for C₂₄H₃₈BrNO₇, 532.47, m/z found 433.8 [M−100]⁺.

A solution of 4 (51 g, 96 mmol) in trimethylamine (239 mL, 2 M, in THF), was stirred at 50° C. for 16 h. The mixture was concentrated under reduced pressure to give the crude intermediate 5 (56 g, crude) as pale-yellow oil, which was used in the next step without purification. MS (ESI): mass calculated for C₂₇H₄₇N₂O₇+, 511.67, m/z found 511.4 [M]+

A mixture of 5 (56.0 g, 94.7 mmol) in HCl/dioxane (592 mL, 4 M) was stirred at 25° C. for 16 h, after which it was concentrated under reduced pressure, dissolved in H₂O (200 mL), and quenched with an aqueous solution of Na₂CO₃ at 0° C. to adjust pH=7. Then Na₂CO₃ (15.0 g, 142 mmol) and Fmoc-OSu (31.9 g, 94.4 mmol) in acetone (150 mL) were added under a nitrogen atmosphere and stirred at 25° C. for 3 h. The mixture was acidified with 2 M HCl, adjusted to pH=4 and concentrated under reduced pressure. The mixture was extracted with ethyl acetate (300 mL×2). The aqueous phase was concentrated under reduced pressure to give crude product 6 (H₂O solution), which was purified by preparative HPLC using a Phenomenex Gemini Xtimate C18 150*40 mm*5 um, 100A (eluent: 53% to 83% (v/v) water (0.225% FA)-ACN) to afford the title compound 6 (APEG3F, 43 g, yield: 78.8%) as an off-white solid. MS (ESI): mass calculated for C₁₈H31N₂O₅⁺, 355.45, m/z found 355.1 [M]⁺. ¹H NMR (400 MHz, DMSO-d₆) δ 8.40 (s, 1H), 7.88 (d, J=7.6 Hz, 2H), 7.66 (d, J=7.2 Hz, 2H), 7.44-7.36 (m, 2H), 7.31 (q, J=7.2 Hz, 2H), 7.18-7.04 (m, 3H), 6.77 (d, J=8.4 Hz, 2H), 4.24-4.13 (m, 3H), 4.00 (d, J=3.6 Hz, 3H), 3.81 (s, 2H), 3.73-3.67 (m, 2H), 3.58 (s, 4H), 3.54-3.48 (m, 2H), 3.07 (s, 9H), 3.05-2.98 (m, 1H), 2.85-2.76 (m, 1H).

### f. Synthesis of N2-(((9H-fluoren-9-yl)methoxy)carbonyl)-N4,N4-dimethyl-L-asparagine (N(N(Me)2)

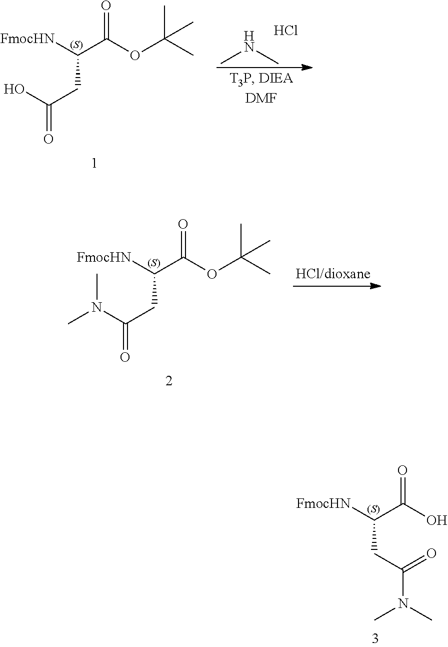

To a solution of starting material 1 (50 g, 122 mmol), dimethylamine (10.9 mg, 134 mmol), and diisopropyl ethyl amine (DIEA, 62.0 g, 365 mmol) in DMF (200 mL) at 0° C. was degassed with N₂ three times and propylphosphonic anhydride (T₃P®, 109 g, 182 mmol) was added via syringe. The mixture was stirred at 20° C. for 12 hours after which it was poured into ice water (500 mL) and extracted with ethyl acetate (500 mL×3). The combined organic extracts were washed with brine, dried over anhydrous Na₂SO₄, filtered, and concentrated under reduced pressure to afford the crude intermediate 2, which was purified by fast column chromatography (FCC, eluent: petroleum ether:ethyl acetate=1:0 to 1:2) to afford 2 (45 g, yield: 84.4%) as pale-yellow solid. MS (ESI): mass calculated for C₂₅H₃₀N₂O₅, 438.52, m/z found 439.2 [M+H]⁺.

Intermediate 2 (45 g, 103 mmol) was stirred in HCl/dioxane (1L, 4 M) at 20° C. for 16 h. The reaction mixture was filtered and concentrated. EtOAc (200 mL) was added to the concentrated material after which petroleum ether (200 mL) was added dropwise. The mixture was stirred at 20° C. for 3 h resulting in a solid that was filtered to afford 3 (N(N(Me)2), 25 g, yield: 62.3%) as white solid. MS (ESI): mass calculated for C₂₁H₂₂N₂O₅, 382.41, m/z found 383.1 [M+H]⁺. 1H NMR (DMSO-d₆, 400 MHz): δ ppm 12.59 (s, 1H), 7.86 (d, J=7.6 Hz, 2H), 7.67 (d, J=7.2 Hz, 2H), 7.43-7.21 (m, 5H), 4.39-4.31 (m, 1H), 4.29-4.23 (m, 2H), 4.21-4.15 (m, 1H), 2.90 (s, 3H), 2.78 (s, 3H), 2.75-2.62 (m, 2H).

### g. Synthesis of N2-(((9H-fluoren-9-yl)methoxy)carbonyl)-N6-acetyl-N6-methyl-L-lysine (Lysine N-(MeAc) or K(NMeAc))

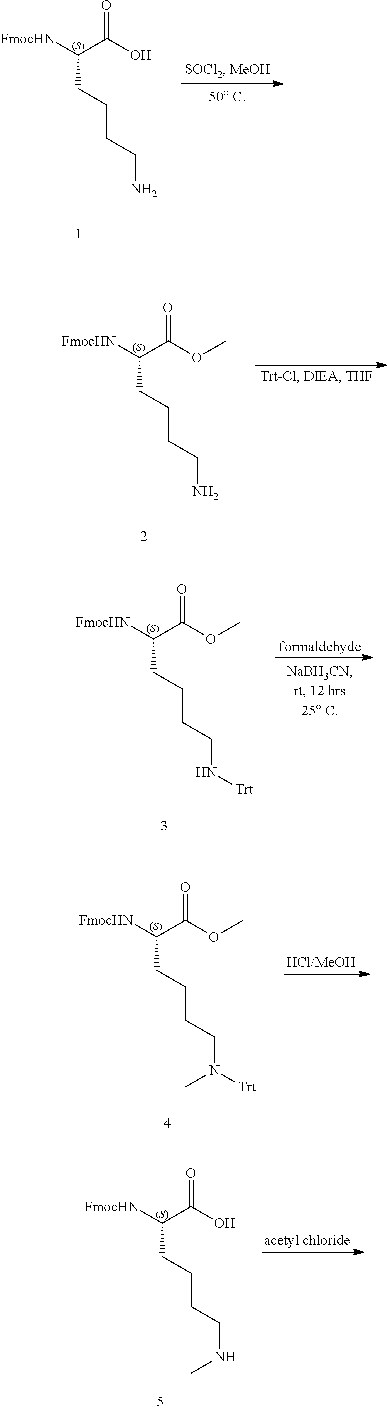

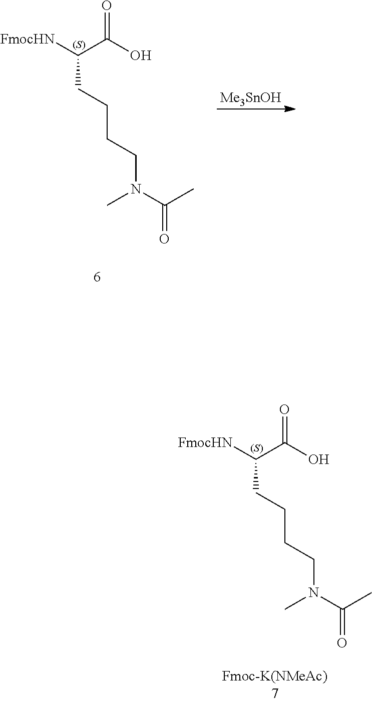

Starting material 1 (21 g, 57.0 mmol) and MeOH (300 mL) were combined in a flask under a N₂ atmosphere. Thionyl chloride (8.14 g, 68.4 mmol) was added to the flask dropwise over 15 minutes at a temperature of 25° C. resulting in a pale-yellow mixture. The mixture was heated at reflux for 4 h. The resulting yellow solution was concentrated in vacuo. Ethyl acetate (50 mL) was added to the concentrated material and the mixture was stirred at 25° C. for 1 h. The solid was filtered to afford crude intermediate 2 (23 g, crude) as white solid. MS (ESI): mass calculated for C₂₂H₂₆N₂O₄, 382.45, m/z found 383.5 [M+H]⁺.

To a solution of 2 (6.1 g, 14.6 mmol) and TEA (4.41, 43.7 mmol) in 100 mL of anhydrous CH₂Cl₂/THF (100 mL) was added trityl chloride (Trt-Cl, 4.47 g, 16.0 mmol). The reaction mixture was stirred at 20° C. for 2 h. The reaction mixture was diluted with water (80 mL), extracted with ethyl acetate (100 mL×2), washed with brine (20 mL) and dried over Na₂SO₄. The combined organic extracts were filtered and concentrated under reduced pressure to afford the crude intermediate 3, which was purified by FCC (eluent: petroleum ether:ethyl acetate=1:0 to 1:2) to afford 3 (7 g, yield: 76.7%) as pale-yellow solid. MS (ESI): mass calculated for C₄₁H₄₀N₂O₄, 624.77, m/z found 647.3 [M+Na]⁺. ¹H NMR (DMSO-d₆, 400 MHz): δ ppm 7.84 (d, J=7.5 Hz, 2H), 7.71 (d, J=7.7 Hz, 1H), 7.66 (d, J=6.8 Hz, 2H), 7.36 (d, J=7.3 Hz, 9H), 7.29-7.20 (m, 8H), 7.17-7.08 (m, 3H), 4.29-4.22 (m, 2H), 4.21-4.11 (m, 1H), 3.97-3.91 (m, 1H), 3.56 (s, 3H), 2.56-2.50 (m, 1H), 1.91 (d, J=6.2 Hz, 2H), 1.55 (m, 2H), 1.46-1.31 (m, 2H), 1.26 (d, J=7.5 Hz, 2H).

A solution of 3 (5.20 g, 8.32 mmol), formaldehyde (20.3 g, 250 mmol) and NaBH₃CN (2.62 g, 41.6 mmol) in methanol (100 mL) was stirred at 25° C. for 16 hours. The mixture was quenched with water (100 mL), extracted with dichloromethane (200 mL×3), the organic layer was dried over anhydrous Na₂SO₄, filtered, and concentrated under reduced pressure. The residue was purified by flash chromatography (FCC, eluent: petroleum ether:ethyl acetate=1:0 to 1:9) to afford 4 (2.7 g, yield: 41.2%) as pale-yellow solid. MS (ESI): mass calculated For C₄₂H₄₂N₂O₄, 638.79, m/z found 661.1[M+Na]⁺.

Intermediate 4 (80 g, 125 mmol) was dissolved in HCl/MeOH (800 mL) and stirred at 20° C. for 1 h. The reaction mixture was concentrated under reduced pressure to afford the crude product. Ethyl acetate (100 mL) and petroleum ether (200 mL) were added, and the reaction mixture was stirred at 20° C. for 4 h. The solid was filtered to afford intermediate 5 (60 g, crude) as pale-yellow solid. MS (ESI): mass calculated for C₂₃H₂₈N₂O₄, 396.48, m/z found 397.1 [M+H]⁺.

To a solution of 5 (120 g, 277 mmol) in CH₂Cl₂ (1200 mL) was added TEA (107 g, 832 mmol) at 0° C. Acetyl chloride (26.1 g, 333 mmol) was added, and the reaction mixture was stirred at 20° C. for 2 h. The reaction mixture was diluted with water (300 mL), extracted with CH₂Cl₂(500 mL×2), washed with brine, and dried over Na₂SO₄. The combined organic extracts were filtered and concentrated under reduced pressure to afford crude intermediate 6, which was purified by FCC (eluent: petroleum ether:ethyl acetate=1:0 to 1:2) to afford 6 (67 g, yield: 38.0%) as pale yellow oil. MS (ESI): mass calculated For C₂₅H₃₀N₂O₅, 438.52, m/z found 439.6 [M+H]⁺.

To a solution 6 (2.6 g, 5.93 mmol) in DCE (50 mL) was added Me3SnOH (1.61 g, 8.90 mmol) and stirred at 20° C. for 16 h. 1 M HCl (5 mL) was added dropwise at 0° C. The mixture was stirred at room temperature for 0.5 h, dried over Na₂SO₄, and filtered. The filtrate was concentrated, and the residue was purified by FCC (eluent: CH₂Cl₂:MeOH=1:0 to 95:5) to afford 7 (K(NMeAc), 2.02 g, yield: 80.51%) as pale-yellow solid. MS (ESI): mass calculated for C₂₄H₂₈N₂O₅, 424.49, m/z found 425.1 [M+H]⁺. ¹H NMR (DMSO-d₆, 400 MHz): δ 7.89 (d, J=7.6 Hz, 2H), 7.73 (d, J=7.2 Hz, 2H), 7.62 (m, 1H), 7.46-7.38 (m, 2H), 7.36-7.28 (m, 2H), 4.33-4.16 (m, 3H), 3.89 (s, 1H), 3.22 (m, 2H), 2.93-2.73 (m, 3H), 1.94 (d, J=7.2 Hz, 3H), 1.77-1.55 (m, 2H), 1.55-1.36 (m, 2H), 1.28 (m, 2H).

### h. Synthesis of (S)-2-amino-N-(2-(dimethylamino)-2-oxoethyl)-N-methyl-3-(pyridin-3-yl)propanamide (NH2-3Pya-Sar-CON(Me)2)

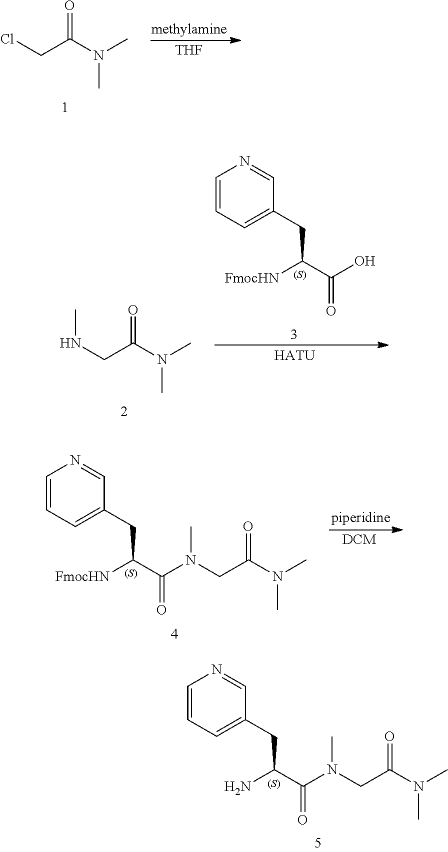

A 100-mL vial was charged with starting material 1 (10 g, 82.3 mmol) and a solution of methylamine (51.1 g, 494 mmol, 30% in ethanol) was added. The reaction mixture was stirred for 16 h at 25° C., after which the mixture was concentrated to give crude intermediate 2. To the crude intermediate, petroleum ether (30 mL) was added and the mixture was stirred at 25° C. for 0.5 h to yield a solid. The resulting solid was filtered to give 2 (10 g, crude) as a light-yellow solid. 1H NMR (DMSO-d₆, 400 MHz): δ ppm 9.09-8.02 (m, 2H), 3.97 (s, 2H), 2.92 (s, 3H), 2.87 (s, 3H), 2.52 (s, 3H).

To a stirred solution of compound 3 (9 g, 23.2 mmol), intermediate 2 (3.23 g, 27.81 mmol), and DIEA (7.03 g, 69.5 mmol) was added in DMF (90 mL) HATU (10.6 g, 27.8 mmol). The reaction mixture was stirred at 25° C. for 2 h then poured into ice water (100 mL) and extracted with ethyl acetate (200 mL×4). The combined organic extracts were washed with brine (100 mL), dried over anhydrous Na₂SO₄, filtered, and concentrated under reduced pressure to afford the crude intermediate 4, which was purified by FCC (eluent: CH₂Cl₂:MeOH=1:0 to 95:5) to afford 4 (11 g, yield: 96.5%) as pale-yellow solid. MS (ESI): mass calculated for C₂₈H₃₀N₄O₄, 486.56, m/z found 487.2 [M+H]⁺.

To a solution of 4 (10.5 g, 21.6 mmol) in DCM (400 mL) was added piperidine (5 mL, 50.5 mmol). The reaction mixture was stirred at room temperature for 16 h under a nitrogen atmosphere, and then it was concentrated under vacuum. The residue was purified by FCC (eluent: CH₂Cl₂:MeOH=1:0 to 95:5) to afford crude product 5 (5.5 g, impure) as pale-yellow solid. Then crude product was purified by preparative HPLC using a Phenomenex Gemini NX C18 (150*40 mm*5 um) (eluent: 1% to 25% (v/v) water (0.04% NH₃H₂O+10 mM NH₄HCO₃)-MeCN to afford pure product. The pure fractions were collected and lyophilized to dryness to give 5 (NH2-3Pya-Sar-CON(Me)2, 3.6 g, yield: 62.7%) as a gummy liquid. MS (ESI): mass calculated for C₁₃H₂₀N₄O₂, 264.32, m/z found 265.1 [M+H]⁺. ¹H NMR (400 MHz, D₂O) δ ppm 8.44-8.22 (m, 2H), 7.76-7.54 (m, 1H), 7.34 (m, 1H), 4.31-4.19 (m, 1H), 4.18-3.96 (m, 2H), 2.95 (m, 3H), 2.92-2.85 (m, 6H), 2.77 (m, 2H).

### i. Synthesis of (2-((((9H-fluoren-9-yl)methoxy)carbonyl)amino)-N-(carboxymethyl)-N,N-dimethylethan-1-aminium) chloride (Fmoc-SP6)

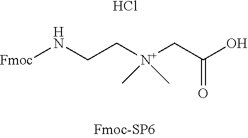

Tert-butyl (2-((((9H-fluoren-9-yl)methoxy)carbonyl)amino)ethyl)glycinate was dissolved in H₂O/ACN and Na₂CO₃ (3 Eq) was added, followed by CH₃I (10 Eq). The mixture was stirred at RT. After 1 h, ACN was evaporated in vacuum and the mixture was extracted with EtOAc, then washed with water and brine. The organic extracts were dried on Na₂SO₄, filtered, concentrated to dryness. The crude mixture was dissolved in HCl 6M in dioxane and stirred for 6 hr at RT to remove the tButyl group. Solvent was evaporated, stripped several times with Et₂O and lyophilized to afford intermediate compound Fmoc-SP6 ((2-((((9H-fluoren-9-yl)methoxy)carbonyl)amino)-N-(carboxymethyl)-N,N-dimethylethan-1-aminium)) chloride: LCMS anal. calc. For C₂₁H₂₅N₂O₄+: 369.44; found: 369.4; ¹H NMR (400 MHz, DMSO-d₆) δ 3.20 (s, 6H) 3.39-3.48 (m, 2H) 3.57 (s, 2H) 4.20-4.27 (m, 1H) 4.38 (s, 1H) 4.33 (s, 2H) 4.31-4.36 (m, 1H) 7.30-7.38 (m, 2H) 7.40-7.46 (m, 2H) 7.59-7.64 (m, 1H) 7.65-7.71 (m, 2H) 7.90 (d, J=7.45 Hz, 2H).

### Assembly

The peptides were assembled using standard Symphony protocols. The peptide sequences were assembled as follows: Resin (250 mg, 0.14 mmol) in each reaction vial was washed twice with 4 ml of DMF followed by treatment with 2.5 ml of 20% 4-methyl piperidine (Fmoc de-protection) for 10 min. The resin was then filtered and washed two times with DMF (4 ml) and re-treated with N-methyl piperifine for additional 30 minute. The resin was again washed three times with DMF (4 ml) followed by addition 2.5 ml of amino acid and 2.5 ml of HBTU-DIEA mixture. After 45 min of frequent agitations, the resin was filtered and washed three timed with DMF (4 ml each). For a typical peptide of the present invention, double couplings were performed. After completing the coupling reaction, the resin was washed three times with DMF (4 ml each) before proceeding to the next amino acid coupling.

### Cleavage

Following completion of the peptide assembly, the peptide was cleaved from the resin by treatment with cleavage reagent, such as reagent K (82.5% trigluoroacetic acid, 5% water, 5% thioanisole, 5% phenol, 2.5% 1,2-ethanedithiol). The cleavage reagent was able to successfully cleave the peptide from the resin, as well as all remaining side chain protecting groups.

The cleaved peptides were precipitated in cold diethyl ether followed by two washings with ethyl ether. The filtrate was poured off and a second aliquot of cold ether was added, and the procedure repeated. The crude peptide was dissolved in a solution of acetonitrile:water (7:3 with 1% TFA) and filtered. The quality of linear peptide was verified using electrospray ionization mass spectrometry (ESI-MS) (Micromass/Waters ZQ) before being purified.

### Disulfide Bond Formation Via Oxidation

The peptide containing the free thiol (for example diPen) was assembled on a Rink Amide-MBHA resin following general Fmoc-SPPS procedure. The peptide was cleaved from the resin by treatment with cleavage reagent 90% trifluoroacetic acid, 5% water, 2.5% 1,2-ethanedithiol, 2.5% tri-isopropylsilane). The cleaved peptides were precipitated in cold diethyl ether followed by two washings with ethyl ether. The filtrate was poured off and a second aliquot of cold ether was added, and the procedure repeated. The crude peptide was dissolved in a solution of acetonitrile:water (7:3 with 1% TFA) and filtered giving the wanted unoxidized peptide crude peptide.

The crude, cleaved peptide with positions X4 and X9, for example, possessing either Cys, Pen, hCys, (D)Pen, (D)Cys or (D)hCys, was dissolved in 20 ml of water: acetonitrile. Saturated Iodine in acetic acid was then added drop wise with stirring until yellow color persisted. The solution was stirred for 15 minutes, and the reaction was monitored with analytic HPLC and LCMS. When the reaction was completed, solid ascorbic acid was added until the solution became clear. The solvent mixture was then purified by first being diluted with water and then loaded onto a reverse phase HPLC machine (Luna C18 support, 10u, 100 A, Mobile phase A: water containing 0.1% TFA, mobile phase B: Acetonitrile (ACN) containing 0.1% TFA, gradient began with 5% B, and changed to 50% B over 60 minutes at a flow rate of 15 ml/min). Fractions containing pure product were then freeze-dried on a lyophilizer.

### Purification

Analytical reverse-phase, high performance liquid chromatography (HPLC) was performed on a Gemini C18 column (4.6 mm×250 mm) (Phenomenex). Semi-Preparative reverse phase HPLC was performed on a Gemini 10 m C18 column (22 mm×250 mm) (Phenomenex) or Jupiter 10 m, 300 angstrom (A) C18 column (21.2 mm×250 mm) (Phenomenex). Separations were achieved using linear gradients of buffer B in A (Mobile phase A: water containing 0.15% TFA, mobile phase B: Acetonitrile (ACN) containing 0.1% TFA), at a flow rate of 1 mL/min (analytical) and 15 mL/min (preparative). Separations were achieved using linear gradients of buffer B in A (Mobile phase A: water containing 0.15% TFA, mobile phase B: Acetonitrile (ACN) containing 0.1% TFA), at a flow rate of 1 mL/min (analytical) and 15 mL/min (preparative).

### General Procedure 1A:

IL-23R inhibitor compounds described herein were synthesized from amino acids monomers using standard Fmoc solid phase synthesis techniques on a CEM Liberty Blue™ microwave peptide synthesizer. The peptides were assembled using Oxyma/DIC (ethyl cyanohydroxyiminoacetate/diisopropyl-carbodiimide) with microwave heating. Rink Amide-MBHA resin (100-200 mesh, 0.66 mmol/g) was used for peptides with C-terminal amides and pre-loaded Wang Resin with N-α-Fmoc protected amino acid was used for peptide with C-terminal acids. Oxyma was prepared as a 1M solution in DMF with 0.1M DIEA. DIC was prepared as 0.5M solution in DMF. The Amino acids were prepared at 200 mM. Peptide inhibitors of the present invention were identified based on medicinal chemistry optimization and/or phage display and screened to identify those having superior binding and/or inhibitory properties.

### Assembly

The peptides were made using standard CEM Liberty Blue™ protocols. The peptide sequences were assembled as follows: Resin (400 mg, 0.25 mmol) was suspended in 10 ml of 50/50 DMF/DCM. The resin was then transferred to the reaction vessel in the microwave cavity. The peptide was assembled using repeated Fmoc deprotection and Oxyma/DIC coupling cycles. For deprotection, 20% 4-methylpiperidine in DMF was added to the reaction vessel and heated to 90° C. for 65 seconds. The deprotection solution was drained and the resin washed three times with DMF. For most amino acids, 5 equivalents of amino acid, Oxyma and DIC were then added to the reaction vessel and microwave irradiation rapidly heated the mixing reaction to 90° C. for 4 min. For Arginine and Histidine residues, milder conditions using respective temperatures of 75 and 50° C. for 10 min were used to prevent racemization. Rare and expensive amino acids were often coupled manually overnight at room temperature using only 1.5-2 eq of reagents. Difficult couplings were often double coupled 2×4 min at 90° C. After coupling the resin was washed with DMF and the whole cycle was repeated until the desired peptide assembly was completed.

### Cleavage

Following completion of the peptide assembly, the peptide was then cleaved from the resin by treatment with a standard cleavage cocktail of 91:5:2:2 TFA/H2O/TIPS/DODT for 2 hrs. If more than one Arg(pbf) residue was present the cleavage was allowed to go for an additional hour.

The cleaved peptides were precipitated in cold diethyl ether. The filtrate was decanted off and a second aliquot of cold ether was added, and the procedure was repeated. The quality of linear peptide was then verified using electrospray ionization mass spectrometry (ESI-MS) (Waters® Micromass® ZQ™) before being purified.

### Disulfide Bond Formation Via Oxidation

The peptide containing the free thiol (for example diPen) was assembled on a Rink Amide-MBHA resin following general Fmoc solid phase synthesis, cleavage and isolation as described above.

The crude cleaved peptide comprising two thiol containing amino acids selected independently from Cys, Pen, hCys, (D)Pen, (D)Cys and (D)hCys was dissolved ˜2 mg/ml in 50/50 acetonitrile/water. Saturated iodine in acetic acid was then added dropwise with stirring until yellow color persisted. The solution was stirred for a few minutes, and the reaction was monitored with analytic HPLC and LCMS. When the reaction was completed, solid ascorbic acid was added until the solution became clear. The solvent mixture was then purified by first being diluted with water and then loaded onto a reverse phase HPLC Column (Luna® C18 support, 10u, 100 A, Mobile phase A: water containing 0.1% TFA, mobile phase B: acetonitrile (ACN) containing 0.1% TFA, gradient began with 15% B, and changed to 50% B over 60 minutes at a flow rate of 15 ml/min). Fractions containing pure product were then freeze-dried on a lyophilizer.

### Purification

Analytical reverse-phase, high performance liquid chromatography (HPLC) was performed on a Gemini® C18 column (4.6 mm×250 mm) (Phenomenex). Semi-Preparative reverse phase HPLC was performed on a Gemini® 10 m C18 column (22 mm×250 mm) (Phenomenex) or Jupiter® 10 m, 300 angstrom (A) C18 column (21.2 mm×250 mm) (Phenomenex). Separations were achieved using linear gradients of buffer B in A (Mobile phase A: water containing 0.15% TFA, mobile phase B: Acetonitrile (ACN) containing 0.1% TFA), at a flow rate of 1 mL/min (analytical) and 20 mL/min (preparative).

### Example 1. Preparation of Peptide of SEQ ID NO.:1

Ac-[Pen]*—N-T-[W(7-Me)]-[Lys(Ac)]-[Pen]*-Phe[4-(2-aminoethoxy)]-[2-Nal]-[THP]-E-N-[3-Pal]-Sarc-NH₂(*Pen-Pen form disulfide bond) (SEQ ID NO.:1)

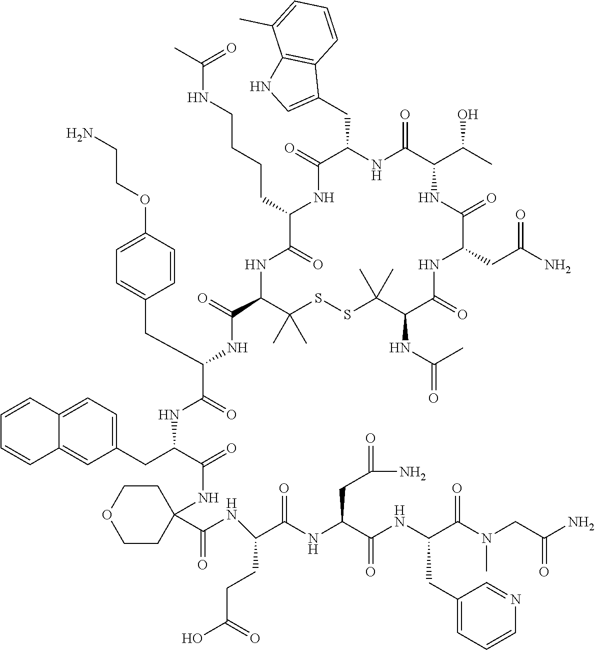 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]

The synthesis of SEQ ID NO.:1 is prepared using FMOC solid phase peptide synthesis techniques.

The peptide is constructed on Rink Amide MBHA resin using standard FMOC protection synthesis conditions reported in the literature. The constructed peptide is isolated from the resin and protecting groups by cleavage with strong acid followed by precipitation. Oxidation to form the disulfide bond is performed followed by purification by reverse phase HPLC (RP-HPLC) and counterion exchange. Lyophilization of pure fractions gives the final product.

Swell Resin: 10 g of Rink Amide MBHA solid phase resin (0.66 mmol/g loading) is transferred to a 250 ml peptide vessel with filter frit, ground glass joint and vacuum side arm. The resin is washed 3× with DMF.

Step 1: Coupling of FMOC-Sarc-OH: Deprotection of the resin bound FMOC group is realized by adding 2 resin-bed volumes of 20% 4-methyl-piperidine in DMF to the swollen resin and shaking for 3-5 min prior to draining and adding a second, 2-resin-bed volume of the 4-methyl piperidine solution and shaking for an additional 20-30 min. After deprotection the resin is washed 3× DMF with shaking. FMOC-Sarc-OH (3 eq, 6.2 g) is dissolved in 100 ml DMF along with Oxyma (4.5 eq, 4.22 g). Preactivation of the acid is accomplished by addition of DIC (3.9 eq, 4 ml) with shaking for 15 min prior to addition to the deprotected resin. An additional aliquot of DIC (2.6 eq, 2.65 ml) is then added after ˜15 min of coupling. The progress of the coupling reaction is monitored by the colorimetric Kaiser test. Once the reaction is judged complete the resin is washed 3× DMF with shaking prior to starting the next deprotection/coupling cycle.

Step 2: Coupling of FMOC-3Pal-OH: FMOC deprotection is again accomplished by adding two sequential, 2-resin-bed volumes of 20% 4-methyl-piperidine in DMF, one times 3-5 minutes, and one times 20-30 minutes, draining in between treatments. The resin is then washed 3 times prior to coupling with protected 3-pyridyl alanine (3Pal). FMOC-3Pal-OH (3 eq, 7.8 g) is dissolved in DMF along with Oxyma (4.5 eq, 4.22 g). Preactivation with DIC (3.9 eq, 4 ml) for 15 minutes is done prior to addition to the Sarc-Amide resin. After 15 minutes, an additional aliquot of DIC (2.6 eq, 2.65 ml) is added to the reaction. Once the reaction is complete as determined by the Kaiser test, the resin is again washed 3× with DMF prior to starting the next deprotection/coupling cycle.

Step 3: Coupling of FMOC-Asn(Trt)-OH: The FMOC is removed from the N-terminus of the resin bound 3Pal and washed as previously described. FMOC-Asn(Trt)-OH (2 eq, 8 g) is dissolved in 100 ml of DMF along with Oxyma (3 eq, 2.81 g). DIC (2.6 eq, 2.65 ml) is added for preactivation of the acid for ˜15 minutes prior to addition to the 3Pal-Sarc-Amide resin. After ˜15 minutes, an additional aliquot of DIC (1.4 eq, 1.43 ml) is added to the reaction. Once the reaction is complete as determined by the Kaiser test, the resin is washed 3× with DMF prior to starting the next deprotection/coupling cycle.

Step 4: Coupling of FMOC-Glu(OtBu)-OH: The FMOC is removed from the N-terminus of the resin bound Asparagine and the resin washed with DMF as previously described. FMOC-Glu(OtBu)-OH (2 eq, 5.91 g) is dissolved in 100 ml of DMF along with Oxyma (3 eq, 2.81 g). DIC (2.6 eq, 2.65 ml) is added for preactivation of the acid ˜15 minutes prior to addition to the Asn(Trt)-3Pal-Sarc-Amide resin. After ˜15 minutes, an additional aliquot of DIC (1.4 eq, 1.43 ml) is added to the reaction. Once the reaction is complete as determined by the Kaiser test the resin is washed 3× with DMF prior to starting the next deprotection/coupling cycle.

Step 5: Coupling of FMOC-THP-OH: The FMOC is removed from the N-terminus of the resin bound peptide and the resin is washed as previously described. FMOC-THP-OH (3 eq, 7.36 g) is dissolved in 100 ml of DMF along with Oxyma (4.5 eq, 4.22 g). DIC (3.9 eq, 4 ml) is added for preactivation of the acid ˜15 minutes prior to addition to the Glu(OtBu)-Asn(Trt)-3Pal-Sarc-Amide resin. After ˜15 minutes, an additional aliquot of DIC (2.6 eq, 2.65 ml) is added to the reaction. Once the reaction is complete as determined by the Kaiser test the resin is washed 3× with DMF prior to starting the next deprotection/coupling cycle.

Step 6: Coupling of FMOC-L-Ala(2-Naphthyl)-OH (Nal): The FMOC is removed from the N-terminus of the resin bound peptide and the resin washed as previously described. FMOC-L-Ala(2-Naphthyl)-OH (3 eq, 8.66 g) is dissolved in 100 ml of DMF along with Oxyma (4.5 eq, 4.22 g). DIC (3.9 eq, 4 ml) is added for preactivation of the acid ˜15 minutes prior to addition to the THP-Glu(OtBu)-Asn(Trt)-3Pal-Sarc-Amide resin. After ˜15 minutes, an additional aliquot of DIC (2.6 eq, 2.65 ml) is added. Once the reaction is complete as determined by the Kaiser test the resin was again washed 3× with DMF prior to starting the next deprotection/coupling cycle.

Step 7: Coupling of FMOC-4-[2-(Boc-amino-ethoxy)]-L-Phenylalanine (FMOC-AEF): The FMOC is removed from the N-terminus of the resin bound peptide and the resin washed as previously described. FMOC-4-[2-(Boc-amino-ethoxy)]-L-Phenylalanine (3 eq, 10.8 g) is dissolved in 100 ml of DMF along with Oxyma (4.5 eq, 4.22 g). DIC (3.9 eq, 4 ml) is added for preactivation of the acid ˜15 minutes prior to addition to the Nal-THP-Glu(OtBu)-Asn(Trt)-3Pal-Sarc-Amide resin. After ˜15 minutes, an additional aliquot of DIC (2.6 eq, 2.65 ml) is added to the reaction. Once the reaction is complete as determined by the Kaiser test the resin is washed 3× with DMF prior to starting the next deprotection/coupling cycle.

Step 8: Coupling of FMOC-Pen(Trt)-OH: The FMOC is removed from the N-terminus of the resin bound peptide and the resin washed as previously described. FMOC-Pen(Trt)-OH (3 eq, 12.14 g) is dissolved in 100 ml of DMF along with Oxyma (4.5 eq, 4.22 g). DIC (3.9 eq, 4 ml) is added for preactivation of the acid ˜15 minutes prior to addition to the AEF-Nal-THP-Glu(OtBu)-Asn(Trt)-3Pal-Sarc-Amide resin. After ˜15 minutes, an additional aliquot of DIC (2.6 eq, 2.65 ml) is added to the reaction. Once the reaction is complete as determined by the Kaiser test, the resin is again washed 3× with DMF prior to starting the next deprotection/coupling cycle.

Step 9: Coupling of FMOC-Lys(Ac)—OH: The FMOC is removed from the N-terminus of the resin bound peptide and the resin washed as previously described. FMOC-Lys(Ac)—OH (2 eq, 5.4 g) is dissolved in 100 ml of DMF along with Oxyma (3 eq, 2.81 g). DIC (2.6 eq, 2.65 ml) is added for preactivation of the acid ˜15 minutes prior to addition to the Pen(Trt)-AEF-Nal-THP-Glu(OtBu)-Asn(Trt)-3Pal-Sarc-Amide resin. After ˜15 minutes, an additional aliquot of DIC (1.4 eq, 1.43 ml) is added to the reaction. Once the reaction was complete as determined by the Kaiser test, the resin is again washed 3× with DMF prior to starting the next deprotection/coupling cycle.

Step 10: Coupling of FMOC-7-Me-Trp-OH: The FMOC is removed from the N-terminus of the resin bound peptide and the resin washed as previously described. FMOC-7-Me-Trp-OH (2 eq, 5.81 g) is dissolved in 100 ml of DMF along with Oxyma (3 eq, 2.81 g). DIC (2.6 eq, 2.65 ml) is added for preactivation of the acid ˜15 minutes prior to addition to the Lys(Ac)-Pen(Trt)-AEF-Nal-THP-Glu(OtBu)-Asn(Trt)-3Pal-Sarc-Amide resin. After ˜15 minutes, an additional aliquot of DIC (1.4 eq, 1.43 ml) is added to the reaction. Once the reaction is complete as determined by the Kaiser test, the resin is again washed 3× with DMF prior to starting the next deprotection/coupling cycle.

Step 11: Coupling of FMOC-Thr(tBu)-OH: The FMOC is removed from the N-terminus of the resin bound peptide and the resin washed as previously described. FMOC-Thr(tBu)-OH (4 eq, 10.5 g) is dissolved in 100 ml of DMF along with Oxyma (6 eq, 5.62 g). DIC (5.2 eq, 5.3 ml) is added for preactivation of the acid ˜15 minutes prior to addition to the 7MeTrp-Lys(Ac)-Pen(Trt)-AEF-Nal-THP-Glu(OtBu)-Asn(Trt)-3Pal-Sarc-Amide resin. After −15 minutes, an additional aliquot of DIC (2.6 eq, 2.65 ml) is added to the reaction. Once the reaction is complete as determined by the Kaiser test, the resin is again washed 3× with DMF prior to starting the next deprotection/coupling cycle.

Step 12: Coupling of FMOC-Asn(Trt)-OH: The FMOC is removed from the N-terminus of the resin bound peptide and the resin washed as previously described. FMOC-Asn(Trt)-OH (4 eq, 15.8 g) is dissolved in 100 ml of DMF along with Oxyma (6 eq, 5.62 g). DIC (5.2 eq, 5.3 ml) is added for preactivation of the acid ˜15 minutes prior to addition to the Thr(tBu)-7MeTrp-Lys(Ac)-Pen(Trt)-AEF-Nal-THP-Glu(OtBu)-Asn(Trt)-3Pal-Sarc-Amide resin. After ˜15 minutes, an additional aliquot of DIC (2.6 eq, 2.65 ml) is added to the reaction. Once the reaction is complete as determined by the Kaiser test, the resin is again washed 3× with DMF prior to starting the next deprotection/coupling cycle.

Step 13: Coupling of FMOC-Pen(Trt)-OH: The FMOC is removed from the N-terminus of the resin bound peptide and the resin washed as previously described. FMOC-Pen(Trt)-OH (2 eq, 8.1 g) is dissolved in 100 ml of DMF along with Oxyma (3 eq, 2.81 g). DIC (2.6 eq, 2.65 ml) is added for preactivation of the acid ˜15 minutes prior to addition to the Asn(Trt)-Thr(tBu)-7MeTrp-Lys(Ac)-Pen(Trt)-AEF-Nal-THP-Glu(OtBu)-Asn(Trt)-3Pal-Sarc-Amide resin. After −15 minutes, an additional aliquot of DIC (2.6 eq, 2.65 ml) is added to the reaction. Once the reaction is complete as determined by the Kaiser test, the resin is again washed 3× with DMF prior to the final deprotection and acetic acid capping of the constructed peptide.

Step 14: Acetyl Capping: The FMOC is removed from the N-terminus of the resin bound peptide and the resin washed as previously described. 150 ml of Capping Reagent A (THF/Acetic anhydride/Pyridine, 80:10:10) is added to the constructed Pen(Trt)-Asn(Trt)-Thr(tBu)-7MeTrp-Lys(Ac)-Pen(Trt)-AEF-Nal-THP-Glu(OtBu)-Asn(Trt)-3Pal-Sarc-Amide resin and shaken for 30 min. The resin is washed 3× with DMF followed by 5× with DCM. The resin is divided into 5-50 ml centrifuge tubes and placed under vacuum for 1.5 hrs prior to cleavage with TFA.

Step 15: TFA Cleavage and Ether precipitation: 200 ml of the TFA cleavage cocktail (90/5/2.5/2.5 TFA/water/TIPS/DODT) is prepared. 40 ml of the cleavage cocktail is added to each of the 5 tubes containing the protected resin bound peptide and shaken for two hours. The spent resin is filtered away and the filtrate divided evenly into 18-50 ml centrifuge tubes for precipitation. Cold diethyl ether is added to each forming a white precipitate that is then centrifuged. The ether is decanted to waste and 2 more ether washes of the precipitate are performed. The resulting white precipitate cake is dried overnight in the hood to give the crude reduced peptide.

Step 16: Disulfide Oxidation: The crude peptide is oxidized and purified in four 1L batches. ˜2.5 g of crude peptide is dissolved in 1 L 20% ACN/water. With stirring, a saturated solution of iodine in acetic acid/methanol is added dropwise to the 1 L peptide solution until the yellow/brown color of the I₂ remains and does not fade away. The light-yellow solution is allowed to sit for 5 min prior to quenching the excess I₂ with a pinch of ascorbic acid.

Step 17: RP-HPLC purification: The RP-HPLC purification is performed s immediately following each I₂ oxidation. A preparative purification column (Phenomenex, Luna, C18(2), 100 Å, 250×50 mm) is equilibrated at 70 ml/min with 20% MPB in MPA (MPA=0.1% TFA/water, MPB=0.1% TFA in ACN). The 1 L of quenched oxidized peptide is loaded onto the equilibrated column at 70 ml/min. After the solvent front elutes, a gradient of 25-45% MPB at 70 ml/min is run over 60 min. The desired material is isolated in fractions, and each are analyzed by analytical RP-HPLC. Pure fractions are combined from all four purifications and lyophilized to give purified TFA salt ready for counterion exchange.

Step 18: Counterion Exchange to Acetate: The same preparative RP-HPLC column is equilibrated with 5% MPB in MPA at 70 ml/min (MPA=0.3% AcOH in Water, MPB=0.3% AcOH in ACN, MPC=0.5M NH₄OAc in Water.) The purified peptide TFA salt is dissolved in 50/50 ACN/water and diluted to 15% ACN. The solution is loaded onto the equilibrated column at 70 ml/min and the solvent front is eluted. The captured peptide is washed with 5% MPB in MPA for 5 min. The captured peptide is then washed with 5% MPB in MPC for 40 min at 70 ml/min to exchange the counterions to Acetate. The captured peptide is washed with 5% MPB in MPA at 70 ml/min for 10 min to clear all NH₄OAc from the system. Finally, the peptide is eluted with a gradient of 5-70% MPB in MPA over 60 minutes and collected in fractions.

Step 19: Final Lyophilization and Analysis: The collected fractions are analyzed by analytical RP-HPLC, and all fractions>95% purity are combined. Lyophilization of the combined fractions gives SEQ ID NO.:1 as a white powder with a purity>95% as determined by RP-HPLC. Peptide identity is confirmed with LC/MS of the purified Peptide of SEQ ID NO.:1, giving 2 charged states of the peptide, M*2/2 of 950 amu and the molecular ion of 1899 amu.

### Example 2. Synthesis of MeCO-r-Pen*—N-T-7MeW—K(Ac)-Pen*-AEF-2Nal-THP-E-N-3Pya-Sar-K(PEG2PEG2gEC18OH)—CONH2 (*Pen-Pen form disulfide bond)

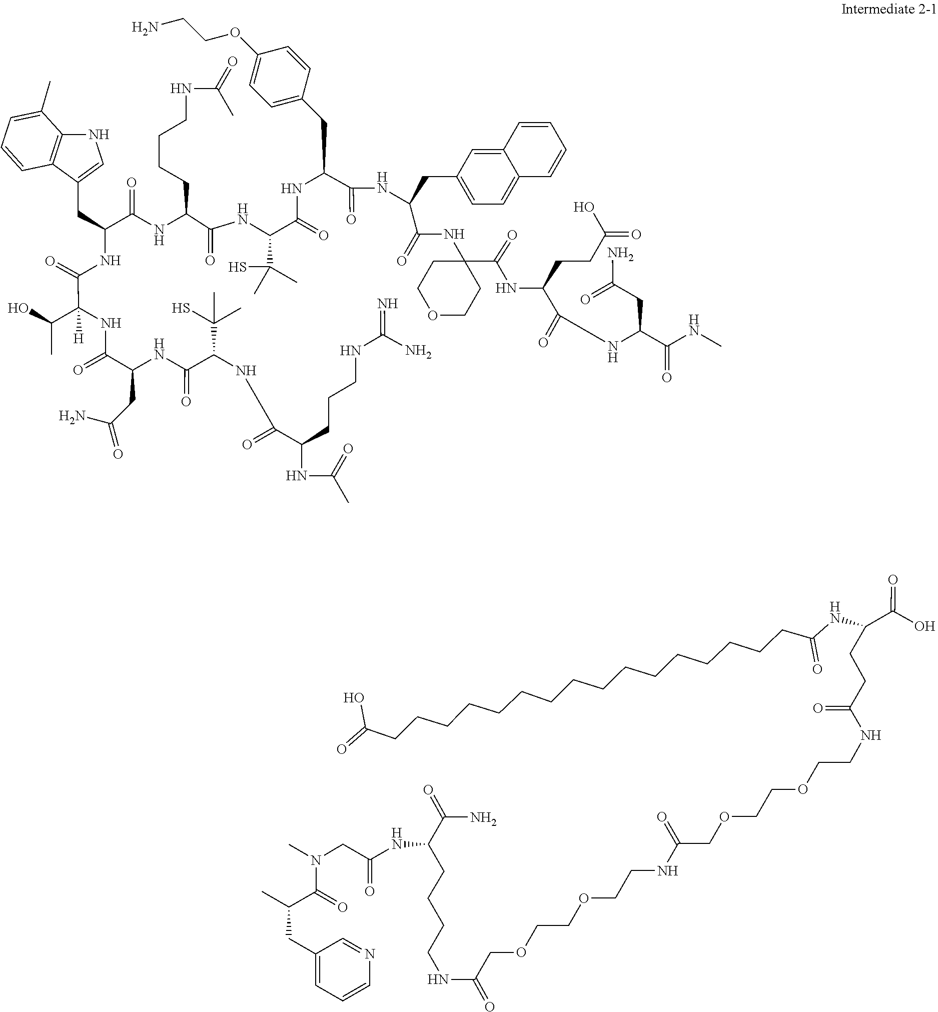

### Synthesis of Intermediate 2-1

Intermediate 2-1 was synthesized by standard Solid-phase Peptide Synthesis (SPPS) using Fmoc/t-Bu chemistry. The assembly was performed on a Rink-amide AM resin (110 mol, 100-200Mesh; loading 0.33 mmol/g) on the Cem Liberty Blue microwave peptide synthesizer (CEM Inc.). During peptide assembly on solid phase, the side chain protecting groups were: tert-butyl for Thr and Glu; trityl for Pen and Asn; tert-butoxy-carbonyl for AEF, Pbf (2,2,4,6,7-pentamethyldihydrobenzofuran-5-sulfonyl) for Arg. The C-terminal Lys was protected by the orthogonal DDe protecting group.

All the amino acids were dissolved at a 0.4 M concentration in DMF. The acylation reactions were performed for 3 min at 90° C. under microwave (MW) irradiation with 5 folds excess of activated amino acids over the resin free amino groups. The amino acids were activated with equimolar amounts of 0.5M solution of DIC in DMF and Oxyma solution 1M in DMF. Double acylation reactions were performed for 3Pya and 2Nal. Fmoc deprotections were performed using 20% (V/V) piperidine in DMF. Capping of the free amino group was performed manually using 10 eq of acetic anhydride in DMF. At the end of the peptide assembly on solid phase, the resin was treated with 100 ml of 3% hydrazine solution in DMF. The solution was drained, and the resin washed with DCM (3×5 mL). The deprotection step was repeated, and then the resin was washed with DCM (5×5 mL) and DMF (5×5 mL). Further side chain derivatization was performed on Cem Liberty Blue microwave peptide synthesizer using standard coupling conditions with 5 folds excess of activated building blocks (Fmoc-PEG2, Fmoc-PEG2 and the Fmoc-gE (Fmoc-Glu-OtBu) and equimolar amounts of 0.5M solution of DIC in DMF and Oxyma solution 1M in DMF. C18OH (18-(tert-butoxy)-18-oxooctadecanoic acid) was coupled manually using DIC-HOAT (3 Eq, 1:1:1) at room temperature and complete acylation was monitored by ninhydrin test.

At the end of the assembly the resin was washed with DMF, MeOH, DCM, and Et₂O. The peptide was cleaved from solid support using 15 ml of TFA solution (v/v) (87.5% TFA, 5% H₂O, 2.5% TIPS, 5% Phenol) for approximately 1.5 hours, at room temperature. The resin was then filtered and precipitated in cold MTBE (135 mL). After centrifugation, the peptide pellets were washed with fresh cold diethyl-ether to remove the organic scavengers. The process was repeated twice. Final pellets were dried, re-suspended in H₂O and acetonitrile 1:1+0.1% TFA and stirred overnight. The mixture was then lyophilized to afford the desired Intermediate 2-1 (50% yield). LCMS anal. calc. for C₁₃₇H₂₀₇N₂₉O₃₆S₂: 2900.45; found: 967.8 (M+3)³⁺.

### Synthesis of MeCO-r-Pen*—N-T-7MeW—K(Ac)-Pen*-AEF-2Nal-THP-E-N-3Pya-Sar-K(PEG2PEG2gEC18OH)—CONH₂ (*Pen-Pen form disulfide bond)

Intermediate 2-1 was dissolved in ACN/H₂O (1 mg/ml). Saturated iodine in acetic acid was then added dropwise under stirring until yellow color persisted. Reaction was completed in 30 min (monitored by UPLC-MS). Solid ascorbic acid was added until the solution became clear. After lyophilization the cyclized peptide was purified by reverse-phase HPLC using preparative Waters DeltaPak C4 (200×40 mm, 300 Å, 15 μm). Mobile phase A: +0.1% TFA, mobile phase B: Acetonitrile (ACN)+0.1% TFA. The following gradient of eluent B was used: 25% B to 25% B over 5 min, to 40% B over 25 min, flow rate 80 mL/min, wavelength 214 nm. Collected fractions were lyophilized to afford the desired compound (5% yield). LCMS anal. calc. for C₁₃₇H₂₀₅N₂₉O₃₆S₂: 2898.4; found; 1450.0 (M+2)²⁺.

### Example 3. Synthesis of MeCO-r-Pen*—N-T-7MeW—K(Ac)-Pen*-AEF-2Nal-THP-E-N-3Pya-NMeK(PEG2PEG2gEC18OH)—CONH2 (*Pen-Pen form disulfide bond)

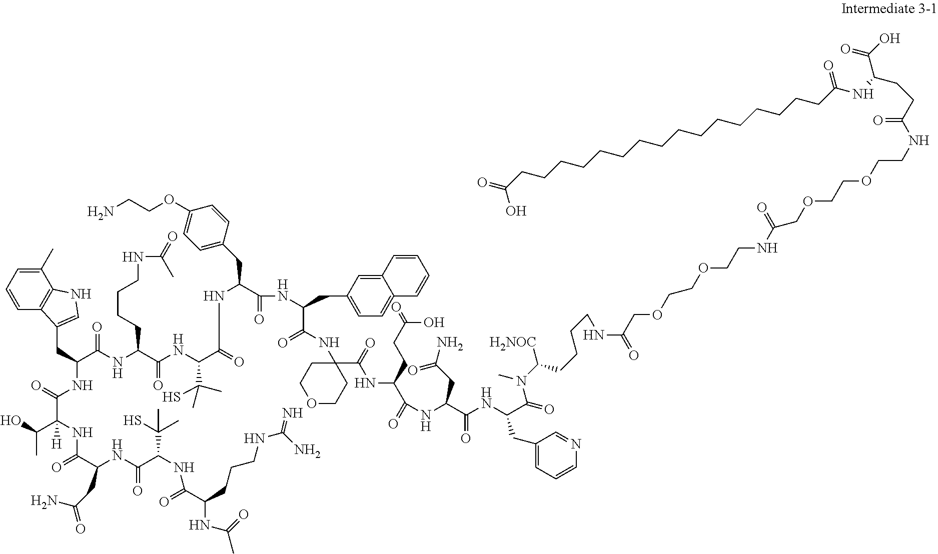 [SMILES: CC(=O)NCCCC[C@H](NC(=O)[C@H](Cc1c[nH]c2c(C)cccc12)NC(=O)[C@@H](NC(=O)[C@H](CC(N)=O)NC(=O)[C@@H](NC(=O)[C@@H](CCCNC(=N)N)NC(C)=O)C(C)(C)S)[C@@H](C)O)C(=O)N[C@H](C(=O)N[C@@H](Cc1ccc(OCCN)cc1)C(=O)N[C@@H](Cc1ccc2ccccc2c1)C(=O)NC1(C(=O)N[C@@H](CCC(=O)O)C(=O)N[C@@H](CC(N)=O)C(=O)N[C@@H](Cc2cccnc2)C(=O)N(C)[C@@H](CCCCNC(=O)COCCOCCNC(=O)COCCOCCNC(=O)CC[C@H](NC(=O)CCCCCCCCCCCCCCCCC(=O)O)C(=O)O)C(N)=O)CCOCC1)C(C)(C)S]

### Synthesis of Intermediate 3-1

The peptide was synthesized by standard Solid-phase Peptide Synthesis (SPPS) using Fmoc/t-Bu chemistry. The assembly was performed on a Rink-amide AM resin (110 mol, 100-200Mesh; loading 0.33 mmol/g) on the Cem Liberty Blue microwave peptide synthesizer (CEM Inc.). During peptide assembly on solid phase, the side chain protecting groups were: tert-butyl for Thr and Glu; trityl for Pen and Asn; tert-butoxy-carbonyl for AEF, Pbf (2,2,4,6,7-pentamethyldihydrobenzofuran-5-sulfonyl) for Arg. The C-terminal NMeLys was protected by the orthogonal DDe protecting group. All the amino acids were dissolved at a 0.4 M concentration in DMF. The acylation reactions were performed for 3 min at 90° C. under MW irradiation with 5 folds excess of activated amino acids over the resin free amino groups. The amino acids were activated with equimolar amounts of 0.5M solution of DIC in DMF and Oxyma solution 1M in DMF.

Double acylation reactions were performed for 3Pya and 2Nal. Fmoc deprotections were performed using 20%(V/V) piperidine in DMF. Capping of the free amino group was performed manually using 10 eq of acetic anhydride in DMF. At the end of the peptide assembly on solid phase, the resin was treated with 100 ml of 3% hydrazine solution in DMF. The solution was drained, and the resin washed with DCM (3×5 mL). The deprotection step was repeated, and then the resin was washed with DCM (5×5 mL), DMF (5×5 mL). Further side chain derivatization was performed manually (PEG2, PEG2 and the gE (Fmoc-Glu-OtBu) residues) using DIC-HOAT (3 Eq, 1:1:1) at room temperature. C18OH (18-(tert-butoxy)-18-oxooctadecanoic acid) was coupled using DIC-HOAT (3 Eq, 1:1:1) at room temperature and complete acylation was monitored by ninhydrin test.

At the end of the assembly the resin was washed with DMF, MeOH, DCM, Et₂O. The peptide was cleaved from solid support using 15 ml of TFA solution (v/v) (87.5% TFA, 5% H₂O, 2.5% TIPS, 5% Phenol) for approximately 1.5 hours, at room temperature. The resin was then filtered and precipitated in cold MTBE (135 mL). After centrifugation, the peptide pellets were washed with fresh cold diethyl-ether to remove the organic scavengers. The process was repeated twice. Final pellets were dried, re-suspended in H₂O and acetonitrile 1:1+0.1% TFA and stirred overnight. The mixture was then lyophilized to afford the desired Intermediate 3-1 (59.2% yield). LCMS anal. calc. For C₁₃₅H₂₀₄N₂₈O₃₅S₂: 2843.38; found: 948.8 (M+3)³⁺.

### Synthesis of MeCO-r-Pen*—N-T-7MeW—K(Ac)-Pen*-AEF-2Nal-THP-E-N-3Pya-NMeK(PEG2PEG2gEC18OH)—CONH₂ (*Pen-Pen form disulfide bond)

Intermediate 3-1 was dissolved in ACN/H₂O (5 mg/ml). Saturated iodine in acetic acid was then added dropwise under stirring until yellow color persisted. Reaction was completed in 30 min (monitored by UPLC-MS). Solid ascorbic acid was added until the solution became clear. After lyophilization the cyclized peptide was purified by reverse-phase HPLC using preparative Waters DeltaPak C4 (200×40 mm, 300 Å, 15 μm). Mobile phase A: +0.1% TFA, mobile phase B: Acetonitrile (ACN)+0.1% TFA. The following gradient of eluent B was used: 25% B to 25% B over 5 min, to 40% B over 25 min, flow rate 80 mL/min, wavelength 214 nm. Collected fractions were lyophilized to afford the desired compound (11% yield). LCMS anal. calc. For C₁₃₅H₂₀₂N₂₈O₃₅S₂: 2841.38; found: 1421.7 (M+2)²⁺.

### Example 4. Synthesis of MeCO—Pen*—N-T-7MeW—K(Ac)-Pen*-AEF-2Nal-THP-K(PEG2PEG2gEC18OH)—N-3Pya-Sar-CONH2 (*Pen-Pen form disulfide bond)

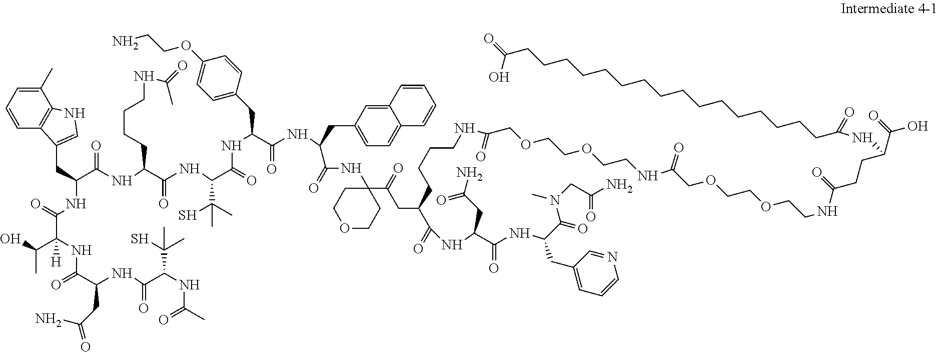 [SMILES: CC(=O)NCCCC[C@H](NC(=O)[C@H](Cc1c[nH]c2c(C)cccc12)NC(=O)[C@@H](NC(=O)[C@H](CC(N)=O)NC(=O)[C@@H](NC(C)=O)C(C)(C)S)[C@@H](C)O)C(=O)N[C@H](C(=O)N[C@@H](Cc1ccc(OCCN)cc1)C(=O)N[C@@H](Cc1ccc2ccccc2c1)C(=O)NC1(C(=O)C[C@@H](CCCCNC(=O)COCCOCCNC(=O)COCCOCCNC(=O)CC[C@@H](NC(=O)CCCCCCCCCCCCCCCCC(=O)O)C(=O)O)C(=O)N[C@@H](CC(N)=O)C(=O)N[C@@H](Cc2cccnc2)C(=O)N(C)CC(N)=O)CCOCC1)C(C)(C)S]

### Synthesis of Intermediate 4-1

The peptide was synthesized by standard Solid-phase Peptide Synthesis (SPPS) using Fmoc/t-Bu chemistry. The assembly was performed on a Rink-amide AM resin (110 mol, 100-200Mesh; loading 0.33 mmol/g) on the Cem Liberty Blue microwave peptide synthesizer (CEM Inc.). During peptide assembly on solid phase, the side chain protecting groups were: tert-butyl for Thr; trityl for Pen and Asn; tert-butoxy-carbonyl for AEF. The Lys to be lipidated was protected by the orthogonal DDe protecting group.

All the amino acids were dissolved at a 0.4 M concentration in DMF. The acylation reactions were performed for 3 min at 90° C. under MW irradiation with 5 folds excess of activated amino acids over the resin free amino groups. The amino acids were activated with equimolar amounts of 0.5M solution of DIC in DMF and Oxyma solution 1M in DMF. Double acylation reactions were performed for 3Pya and 2Nal. Fmoc deprotections were performed using 20%(V/V) piperidine in DMF. Capping of the free amino group was performed manually using 10 eq of acetic anhydride in DMF. At the end of the peptide assembly on solid phase, the resin was treated with 100 ml of 3% hydrazine solution in DMF. The solution was drained, and the resin washed with DCM (3×5 mL). The deprotection step was repeated, and then the resin was washed with DCM (5×5 mL), DMF (5×5 mL). Further side chain derivatization was performed manually (PEG2, PEG2 and the gE (Fmoc-Glu-OtBu) residues) using DIC-HOAT (3 Eq, 1:1:1) at room temperature. C18OH (18-(tert-butoxy)-18-oxooctadecanoic acid) was coupled using DIC-HOAT (6 Eq, 1:1:1) at room temperature and complete acylation was monitored by ninhydrin test.

At the end of the assembly the resin was washed with DMF, MeOH, DCM, Et₂O. The peptide was cleaved from solid support using 15 ml of TFA solution (v/v) (87.5% TFA, 5% H₂O, 2.5% TIPS, 5% Phenol) for approximately 1.5 hours, at room temperature. The resin was then filtered and precipitated in cold MTBE (135 mL). After centrifugation, the peptide pellets were washed with fresh cold diethyl-ether to remove the organic scavengers. The process was repeated twice. Final pellets were dried, re-suspended in H₂O and acetonitrile 1:1+0.1% TFA and stirred overnight. The mixture was then lyophilized to afford the desired Intermediate 4-1 (70% yield). LCMS anal. calc C₁₂₆H₁₈₈N₂₄O₃₂S₂: 2615.13; found: 1308.5 (M+2)²⁺.

### Synthesis of MeCO—Pen*—N-T-7MeW—K(Ac)-Pen*AEF-2Nal-THP-K(PEG2PEG2gEC18OH)—N-3Pya-Sar-CONH₂ (*Pen-Pen form disulfide bond)

Intermediate 4-1 was dissolved in ACN/H₂O (1 mg/ml). Saturated iodine in acetic acid was then added dropwise under stirring until yellow color persisted. Reaction was completed in 30 min (monitored by UPLC-MS). Solid ascorbic acid was added until the solution became clear. After lyophilization the cyclized peptide was purified by reverse-phase HPLC using preparative Waters DeltaPak C4 (200×40 mm, 300 Å, 15 μm). Mobile phase A: +0.1% TFA, mobile phase B: Acetonitrile (ACN)+0.1% TFA. The following gradient of eluent B was used: 25% B to 25% B over 5 min, to 45% B over 25 min, flow rate 80 mL/min, wavelength 214 nm. Collected fractions were lyophilized to afford the desired compound (24% yield): LCMS anal. calc. For C₁₂₆H₁₈₆N₂₄O₃₂S₂: 2613.13; found: 1307.4 (M+2)²⁺.

### Example 5. Synthesis of MeCO-k(PEG2PEG2gEC18OH)-Pen*—N-T-7MeW—K(Ac)-Pen*-AEF-2Nal-THP-K(Ac)—N-3Pya-Sar-CONH2 (*Pen-Pen form disulfide bond)

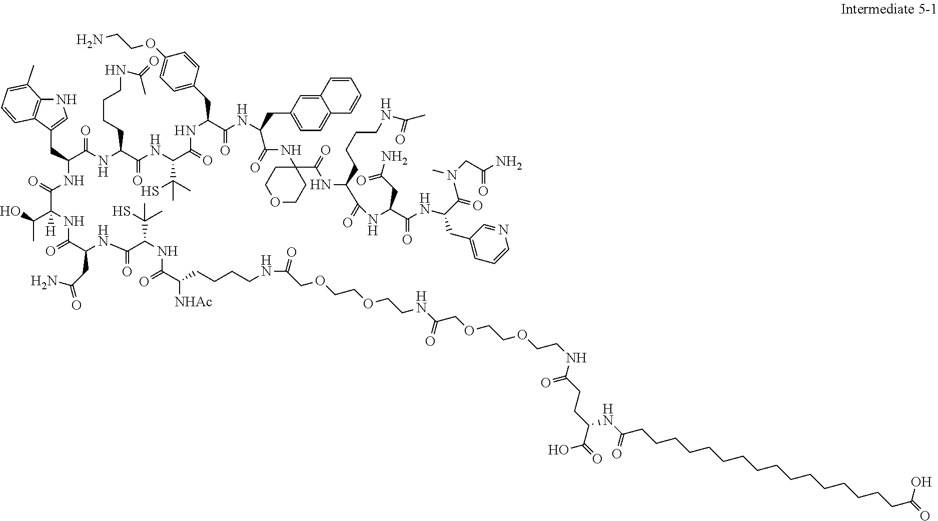 [SMILES: CC(=O)NCCCC[C@H](NC(=O)[C@H](Cc1c[nH]c2c(C)cccc12)NC(=O)[C@@H](NC(=O)[C@H](CC(N)=O)NC(=O)[C@@H](NC(=O)[C@H](CCCCNC(=O)COCCOCCNC(=O)COCCOCCNC(=O)CC[C@H](NC(=O)CCCCCCCCCCCCCCCCC(=O)O)C(=O)O)NC(C)=O)C(C)(C)S)[C@@H](C)O)C(=O)N[C@H](C(=O)N[C@@H](Cc1ccc(OCCN)cc1)C(=O)N[C@@H](Cc1ccc2ccccc2c1)C(=O)NC1(C(=O)N[C@@H](CCCCNC(C)=O)C(=O)N[C@@H](CC(N)=O)C(=O)N[C@@H](Cc2cccnc2)C(=O)N(C)CC(N)=O)CCOCC1)C(C)(C)S]

### Synthesis of Intermediate 5-1

The peptide was synthesized by standard Solid-phase Peptide Synthesis (SPPS) using Fmoc/t-Bu chemistry. The assembly was performed on a Rink-amide AM resin (110 mol, 100-200Mesh; loading 0.33 mmol/g) on the Cem Liberty Blue microwave peptide synthesizer (CEM Inc.). During peptide assembly on solid phase, the side chain protecting groups were: tert-butyl for Thr; trityl for Pen and Asn; tert-butoxy-carbonyl for AEF. The N-terminal D-Lys was protected by the orthogonal DDe protecting group.

All the amino acids were dissolved at a 0.4 M concentration in DMF. The acylation reactions were performed for 3 min at 90° C. under MW irradiation with 5 folds excess of activated amino acids over the resin free amino groups. The amino acids were activated with equimolar amounts of 0.5M solution of DIC in DMF and Oxyma solution 1M in DMF. Double acylation reactions were performed for 3Pya and 2Nal. Fmoc deprotections were performed using 20%(V/V) piperidine in DMF. Capping of the free amino group was performed manually using 10 eq of acetic anhydride in DMF. At the end of the peptide assembly on solid phase, the resin was treated with 100 ml of 3% hydrazine solution in DMF. The solution was drained, and the resin washed with DCM (3×5 mL). The deprotection step was repeated, and then the resin was washed with DCM (5×5 mL), DMF (5×5 mL). Further side chain derivatization was performed manually (PEG2, PEG2 and the gE (Fmoc-Glu-OtBu) residues) using DIC-HOAT (3 Eq, 1:1:1) at room temperature. C18OH (18-(tert-butoxy)-18-oxooctadecanoic acid) was coupled using DIC-HOAT (6 Eq, 1:1:1) at room temperature and complete acylation was monitored by ninhydrin test.

At the end of the assembly the resin was washed with DMF, MeOH, DCM, and Et₂O. The peptide was cleaved from solid support using 15 ml of TFA solution (v/v) (87.5% TFA, 5% H₂O, 2.5% TIPS, 5% Phenol) for approximately 1.5 hours, at room temperature. The resin was then filtered and precipitated in cold MTBE (135 mL). After centrifugation, the peptide pellets were washed with fresh cold diethyl-ether to remove the organic scavengers. The process was repeated twice. Final pellets were dried, re-suspended in H₂O and acetonitrile 1:1+0.1% TFA and stirred overnight. The mixture was then lyophilized to afford the desired Intermediate 5-1 (89% yield). LCMS anal. calc. For C₁₃₄H₂₀₂N₂₆O₃₄S₂: 2785.3; found: 1393.4 (M+2)²⁺.

### Synthesis of MeCO-k(PEG2PEG2gEC18OH)-Pen*—N-T-7MeW—K(Ac)-Pen* AEF-2Nal-THP-K(Ac)—N-3Pya-Sar-CONH₂ (*Pen-Pen form disulfide bond)

Intermediate 5-1 was dissolved in ACN/H₂O (5 mg/ml). Saturated iodine in acetic acid was then added dropwise under stirring until yellow color persisted. Reaction was completed in 30 min (monitored by UPLC-MS). Solid ascorbic acid was added until the solution became clear. After lyophilization the cyclized peptide was purified by reverse-phase HPLC using preparative Waters DeltaPak C4 (200×40 mm, 300 Å, 15 μm). Mobile phase A: +0.1% TFA, mobile phase B: Acetonitrile (ACN)+0.1% TFA. The following gradient of eluent B was used: 25% B to 25% B over 5 min, to 40% B over 25 min, flow rate 80 mL/min, wavelength 214 nm. Collected fractions were lyophilized to afford desired compound (28% yield): LCMS anal. calc. For C₁₃₄H₂₀₀N₂₆O₃₄S₂: 2783.34; found: 1392.4 (M+2)₂₊.

### Example 6. Synthesis of MeCO-r-Pen*—N-T-7MeW—K(Ac)-Pen*-AEF(PEG2PEG2gEC18OH)-2Nal-THP-K(Ac)—N-3Pya-Sar-CONH2 (*Pen-Pen form disulfide bond)

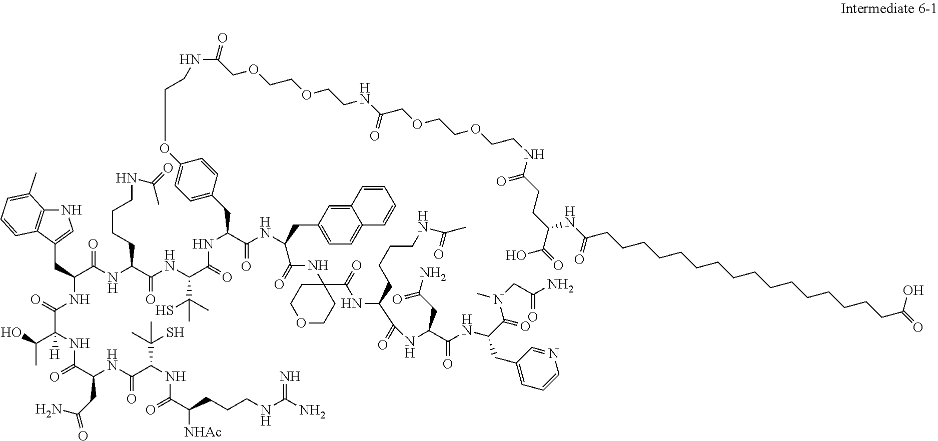 [SMILES: CC(=O)NCCCC[C@H](NC(=O)[C@H](Cc1c[nH]c2c(C)cccc12)NC(=O)[C@@H](NC(=O)[C@H](CC(N)=O)NC(=O)[C@@H](NC(=O)[C@@H](CCCNC(=N)N)NC(C)=O)C(C)(C)S)[C@@H](C)O)C(=O)N[C@H](C(=O)N[C@@H](Cc1ccc(OCCNC(=O)COCCOCCNC(=O)COCCOCCNC(=O)CC[C@H](NC(=O)CCCCCCCCCCCCCCCCC(=O)O)C(=O)O)cc1)C(=O)N[C@@H](Cc1ccc2ccccc2c1)C(=O)NC1(C(=O)N[C@@H](CCCCNC(C)=O)C(=O)N[C@@H](CC(N)=O)C(=O)N[C@@H](Cc2cccnc2)C(=O)N(C)CC(N)=O)CCOCC1)C(C)(C)S]

### Synthesis of Intermediate 6-1

The peptide was synthesized by standard Solid-phase Peptide Synthesis (SPPS) using Fmoc/t-Bu chemistry. The assembly was performed on a Rink-amide AM resin (110 mol, 100-200Mesh; loading 0.33 mmol/g) on the Cem Liberty Blue microwave peptide synthesizer (CEM Inc.). During peptide assembly on solid phase, the side chain protecting groups were: tert-butyl for Thr; trityl for Pen and Asn; Pbf (2,2,4,6,7-pentamethyldihydrobenzofuran-5-sulfonyl) for Arg. The AEF was protected by the orthogonal DDe protecting group. All the amino acids were dissolved at a 0.4 M concentration in DMF. The acylation reactions were performed for 3 min at 90° C. under MW irradiation with 5 folds excess of activated amino acids over the resin free amino groups. The amino acids were activated with equimolar amounts of 0.5M solution of DIC in DMF and Oxyma solution 1M in DMF. Double acylation reactions were performed for 3Pya and 2Nal. Fmoc deprotections were performed using 20%(V/V) piperidine in DMF. Capping of the free amino group was performed manually using 10 eq of acetic anhydride in DMF.

At the end of the peptide assembly on solid phase, the resin was treated with 100 ml of 3% hydrazine solution in DMF. The solution was drained, and the resin washed with DCM (3×5 mL) and DMF (5×5 mL). Further side chain derivatization was performed manually (PEG2, PEG2 and the gE (Fmoc-Glu-OtBu) residues) using DIC-HOAT (3 Eq, 1:1:1) at room temperature. C18OH (18-(tert-butoxy)-18-oxooctadecanoic acid) was coupled using DIC-HOAT (6 Eq, 1:1:1) in NMP at room temperature and complete acylation was monitored by ninhydrin test.

At the end of the assembly the resin was washed with DMF, MeOH, DCM, Et₂O. The peptide was cleaved from solid support using 15 ml of TFA solution (v/v) (87.5% TFA, 5% H₂O, 2.5% TIPS, 5% Phenol) for approximately 1.5 hours, at room temperature. The resin was then filtered and precipitated in cold MTBE (135 mL). After centrifugation, the peptide pellets were washed with fresh cold diethyl-ether to remove the organic scavengers. The process was repeated twice. Final pellets were dried, re-suspended in H₂O and acetonitrile 1:1+0.1% TFA and stirred overnight. The mixture was then lyophilized to afford the desired Intermediate 6-1 (78.5% yield). LCMS anal. calc. For C₁₃₄H₂₀₂N₂₈O₃₄S₂: 2813.37; found: 938.5 (M+3)³⁺.

### Synthesis of MeCO-r-Pen*—N-T-7MeW—K(Ac)-Pen* AEF(PEG2PEG2gEC18OH)-2Nal-THP-K(Ac)—N-3Pya-Sar-CONH₂ (*Pen-Pen form disulfide bond)

Intermediate 6-1 was dissolved in ACN/H₂O (1 mg/ml). Saturated iodine in acetic acid was then added dropwise under stirring until yellow color persisted. Reaction was completed in 30 min (monitored by UPLC-MS). Solid ascorbic acid was added until the solution became clear. After lyophilization the cyclized peptide was purified by reverse-phase HPLC using preparative Waters DeltaPak C4 (200×40 mm, 300 Å, 15 μm). Mobile phase A: +0.1% TFA, mobile phase B: Acetonitrile (ACN)+0.1% TFA. The following gradient of eluent B was used: 25% B to 25% B over 5 min, to 40% B over 25 min, flow rate 80 mL/min, wavelength 214 nm. Collected fractions were lyophilized to afford the desired compound (10% yield): LCMS anal. calc. For C134H200N28O34S2: 2811.36; found: 1406.2 (M+2)²⁺.

### Example 7. Synthesis of MeCO-k(PEG2PEG2SP6gEC18OH)-Pen*—N-T-7MeW—K(Ac)-Pen*-AEF-2Nal-THP-K(Ac)—N-3Pya-Sar-CONH2 (*Pen-Pen form disulfide bond)

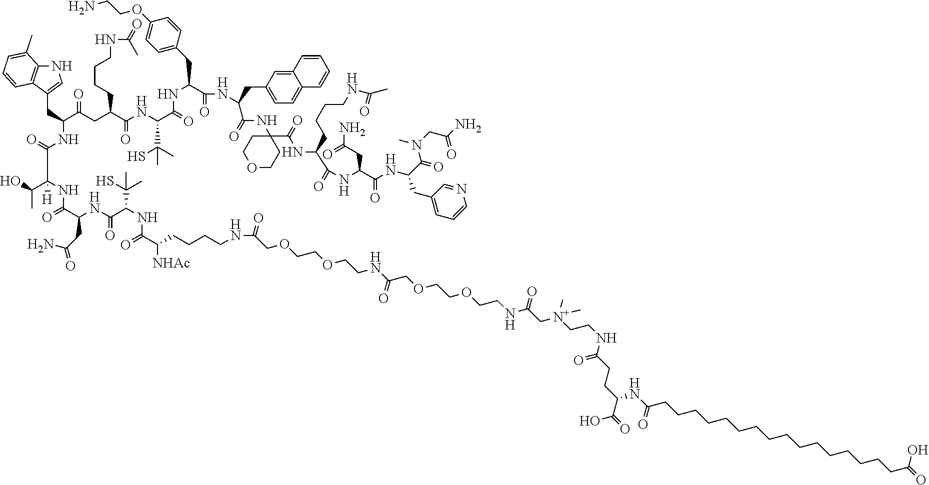 [SMILES: CC(=O)NCCCC[C@H](CC(=O)[C@H](Cc1c[nH]c2c(C)cccc12)NC(=O)[C@@H](NC(=O)[C@H](CC(N)=O)NC(=O)[C@@H](NC(=O)[C@H](CCCCNC(=O)COCCOCCNC(=O)COCCOCCNC(=O)C[N+](C)(C)CCNC(=O)CC[C@H](NC(=O)CCCCCCCCCCCCCCCCC(=O)O)C(=O)O)NC(C)=O)C(C)(C)S)[C@@H](C)O)C(=O)N[C@H](C(=O)N[C@@H](Cc1ccc(OCCN)cc1)C(=O)N[C@@H](Cc1ccc2ccccc2c1)C(=O)NC1(C(=O)N[C@@H](CCCCNC(C)=O)C(=O)N[C@@H](CC(N)=O)C(=O)N[C@@H](Cc2cccnc2)C(=O)N(C)CC(N)=O)CCOCC1)C(C)(C)S]

### Synthesis of Intermediate 7-1

The peptide was synthesized by standard Solid-phase Peptide Synthesis (SPPS) using Fmoc/t-Bu chemistry. The assembly was performed on a Rink-amide AM resin (110 mol, 100-200Mesh; loading 0.34 mmol/g) on the Cem Liberty Blue microwave peptide synthesizer (CEM Inc.). During peptide assembly on solid phase, the side chain protecting groups were: tert-butyl for Thr and Glu; trityl for Pen and Asn; tert-butoxy-carbonyl for AEF. The N-terminal D-Lys was protected by the orthogonal DDe protecting group.

All the amino acids were dissolved at a 0.4 M concentration in DMF. The acylation reactions were performed for 3 min at 90° C. under MW irradiation with 5 folds excess of activated amino acids over the resin free amino groups. The amino acids were activated with equimolar amounts of 0.5M solution of DIC in DMF and Oxyma solution 1M in DMF. Double acylation reactions were performed for 3Pya and 2Nal. Fmoc deprotections were performed using 20%(V/V) piperidine in DMF. Capping of the free amino group was performed manually using 10 eq of acetic anhydride in DMF.

At the end of the peptide assembly on solid phase, the resin was treated with 100 ml of 3% hydrazine solution in DMF. The solution was drained, and the resin washed with DCM (3×5 mL) and DMF (5×5 mL). Further side chain derivatization was performed manually (PEG2, PEG2, Fmoc-SP6 ((2-((((9H-fluoren-9-yl)methoxy)carbonyl)amino)-N-(carboxymethyl)-N,N-dimethylethan-1-aminium) and the gE (Fmoc-Glu-OtBu) residues) using DIC-HOAT (3 Eq, 1:1:1) at room temperature. C18OH (18-(tert-butoxy)-18-oxooctadecanoic acid) was coupled using DIC-HOAT (6 Eq, 1:1:1) at room temperature and complete acylation was monitored by ninhydrin test.

At the end of the assembly the resin was washed with DMF, MeOH, DCM, Et₂O. The peptide was cleaved from solid support using 15 ml of TFA solution (v/v) (87.5% TFA, 5% H₂O, 2.5% TIPS, 5% Phenol) for approximately 1.5 hours, at room temperature. The resin was then filtered and precipitated in cold MTBE (135 mL). After centrifugation, the peptide pellets were washed with fresh cold diethyl-ether to remove the organic scavengers. The process was repeated twice. Final pellets were dried, re-suspended in H₂O and acetonitrile 1:1+0.1% TFA and stirred overnight. The mixture was then lyophilized to afford the desired Intermediate 7-1 (80% yield). LCMS anal. calc. C140H215N28035S2+: 2914.52; found: 972.5 (M+3)³⁺.

### Synthesis of MeCO-k(PEG2PEG2SP6gEC18OH)-Pen*—N-T-7MeW—K(Ac)-Pen* AEF-2Nal-THP-K(Ac)—N-3Pya-Sar-CONH₂

Intermediate 7-1 was dissolved in ACN/H₂O (1 mg/ml). Saturated iodine in acetic acid was then added dropwise under stirring until yellow color persisted. Reaction was completed in 30 min (monitored by UPLC-MS). Solid ascorbic acid was added until the solution became clear. After lyophilization the cyclized peptide was purified by reverse-phase HPLC using preparative Waters DeltaPak C4 (200×40 mm, 300 Å, 15 μm). Mobile phase A: +0.1% TFA, mobile phase B: Acetonitrile (ACN)+0.1% TFA. The following gradient of eluent B was used: 25% B to 25% B over 5 min, to 40% B over 25 min, flow rate 80 mL/min, wavelength 214 nm. Collected fractions were lyophilized to afford the desired compound (35% yield). LCMS anal. calc. For C₁₄₀H₂₁₃N₂₈O₃₅S₂⁺: 2912.52; found: 1456.6 (M+2)²⁺.

### Example 8. Synthesis of MeCO-Pen*—N-T-7MeW—K(Ac)-Pen*-AEF-2Nal-THP-K(Ac)—N-3Pya-Sar-PEG2-PEG2-eK(C16OH)—COOH (*Pen-Pen form disulfide bond)

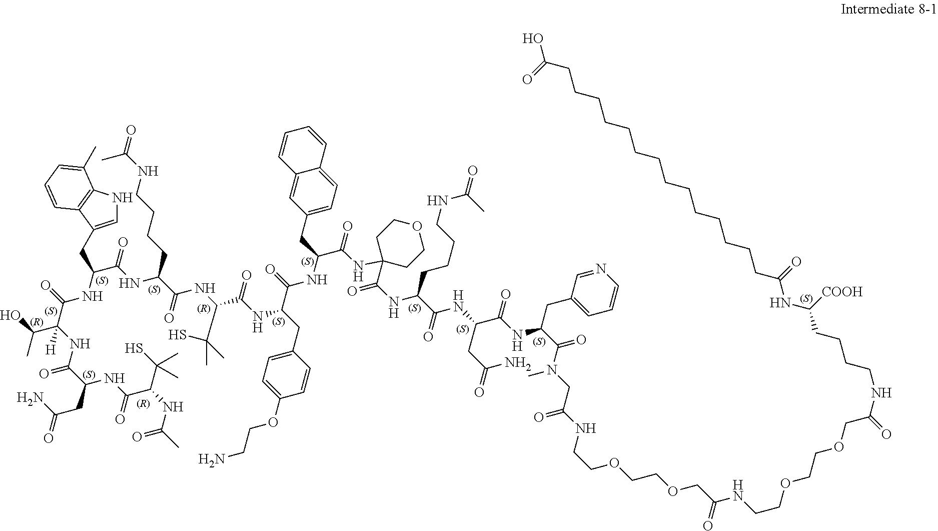

### Synthesis of Intermediate 8-1

The peptide was synthesized by standard Solid-phase Peptide Synthesis (SPPS) using Fmoc/t-Bu chemistry. The assembly was performed on a Wang resin (75 mol, 100-200Mesh; loading 0.33 mmol/g). First amino acids were incorporated manually: Dde-Lys(Fmoc)-OH (10 eq) was dissolved in 7 ml of a solution of dry DCM/dry DMF (10:1) under N₂ and DIC (5 eq) was added at 0° C., Reaction mixture was left under stirring at 0° C. for 20 min, then concentrated to dryness. The residue was dissolved in dry DMF and added to Wang resin (Novabiochem, 100-200 mesh, 0.83 mmol/g), under N₂ atmosphere. DMAP (4-Dimethylaminopyridine, 0.1 eq) was added. The mixture was stirred at RT for 1 h, then the cycle was repeated. After Fmoc removal, assembly was continued on a CEM Liberty Blue microwave peptide synthesizer (CEM Inc.). During peptide synthesis on solid phase, the side chain protecting groups were: tert-butyl for Thr; trityl for Pen and Asn; tert-butoxy-carbonyl for AEF. Lys source was N6-(((9H-fluoren-9-yl)methoxy)carbonyl)-N2-(1-(4,4-dimethyl-3,5-dioxocyclohexylidene)ethyl)-L-lysine. All the amino acids and building blocks were dissolved at a 0.4 M concentration in DMF. The acylation reactions were performed for 3 min at 90° C. under MW irradiation with 5 folds excess of activated amino acids over the resin free amino groups. The amino acids were activated with equimolar amounts of 0.5 M solution of DIC in DMF and Oxyma solution 1 M in DMF. Double acylation reactions were performed for 3Pya and 2Nal. Fmoc deprotections were performed using 20% (V/V) piperidine in DMF. Capping of the free amino group was performed manually using 10 equiv. of acetic anhydride in DMF. At the end of the peptide assembly on solid phase, the resin was treated with 100 ml of 3% hydrazine solution in DMF. The solution was drained, and the resin washed with DCM (3×5 mL) and DMF (5×5 mL). Further side chain derivatization with C16OH (hexadecandioic acid) was performed manually using DIC-HOAT (6 Eq, 1:1:1) at room temperature and complete acylation was monitored by ninhydrin test.

At the end of the assembly, the resin was washed with NMP, DMF, MeOH, DCM, and Et₂O. The peptide was cleaved from solid support using 15 ml of TFA solution (v/v) (87.5% TFA, 5% H₂O, 2.5% TIPS, 5% Phenol) for approximately 1.5 hours, at room temperature. The resin was then filtered and precipitated in cold MTBE (135 mL). After centrifugation, the peptide pellets were washed with fresh cold diethyl-ether to remove the organic scavengers. The process was repeated twice. Final pellets were dried, re-suspended in H₂O and acetonitrile 1:1+0.1% TFA and stirred overnight. The mixture was then lyophilized to afford the desired Intermediate 8-1 (60% yield). LCMS anal. calc. For C₁₂₇H₁₉₀N₂₄O₃₂S₂ 2629.16; found: 1315.7 (M+2)²⁺.

### Synthesis of MeCO-Pen*—N-T-7MeW—K(Ac)-Pen* AEF-2Nal-THP-K(Ac)—N-3Pya-Sar-PEG2-PEG2-eK(C16OH)—COOH

Intermediate 8-1 was dissolved in ACN/H₂O (5 mg/ml). Saturated iodine in acetic acid was then added dropwise under stirring until yellow color persisted. Reaction was completed in 30 min (monitored by UPLC-MS). Solid ascorbic acid was added until the solution became clear. After lyophilization the cyclized peptide was purified by reverse-phase HPLC using preparative Reprosyl C4 (200×40 mm, 300 Å, 15 μm). Mobile phase A: +0.1% TFA, mobile phase B: Acetonitrile (ACN)+0.1% TFA. The following gradient of eluent B was used: 25% B to 25% B over 5 min, to 40% B over 25 min, flow rate 60 mL/min, wavelength 214 nm. Collected fractions were lyophilized to afford the desired compound (7% yield): LCMS anal. calc. For C₁₂₇H₁₈₈N₂₄O₃₂S₂ 2627.16; found: 1314.7 (M+2)²⁺.

### Example 9. Synthesis of MeCO-k(PEG2PEG2gEmXOH)-Pen*—N-T-7MeW—K(Ac)-Pen*-AEF-2Nal-THP-K(PEG2PEG2gEmXOH)—N-3Pya-Sar-CONH2 (*Pen-Pen form disulfide bond)

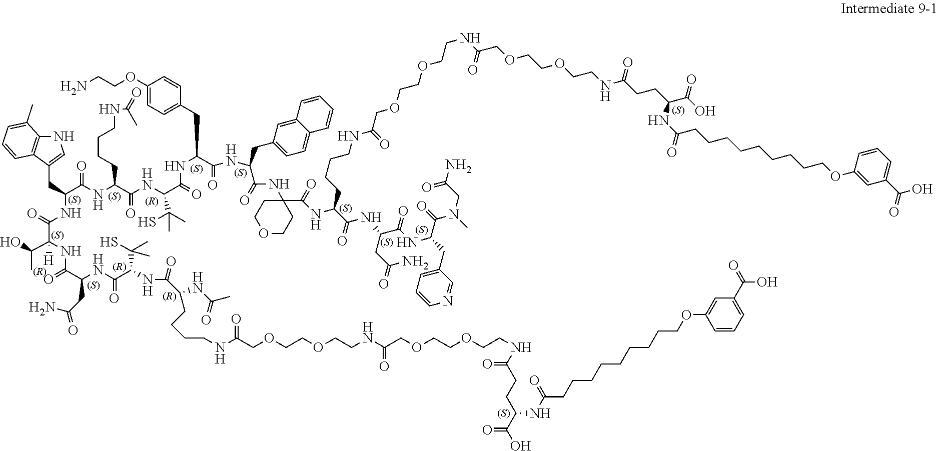

### Synthesis of Intermediate 9-1

The peptide was synthesized by standard Solid-phase Peptide Synthesis (SPPS) using Fmoc/t-Bu chemistry. The assembly was performed on a Rink-amide AM resin (110 mol, 100-200Mesh; loading 0.33 mmol/g) on the Cem Liberty Blue microwave peptide synthesizer (CEM Inc.). During peptide assembly on solid phase, the side chain protecting groups were: tert-butyl for Thr; trityl for Pen and Asn; tert-butoxy-carbonyl for AEF. The Lys to be attached to the THP and the N-terminal D-Lys were protected by the orthogonal DDe protecting group.

All the amino acids were dissolved at a 0.4 M concentration in DMF. The acylation reactions were performed for 3 min at 90° C. under microwave (MW) irradiation with 5 folds excess of activated amino acids over the resin free amino groups. The amino acids were activated with equimolar amounts of 0.5M solution of DIC in DMF and Oxyma solution 1M in DMF. Double acylation reactions were performed for 3Pya and 2Nal. Fmoc deprotections were performed using 20%(V/V) piperidine in DMF. Capping of the free amino group was performed manually using 10 eq of acetic anhydride in DMF. At the end of the peptide assembly on solid phase, the resin was treated with 100 ml of 3% hydrazine solution in DMF to remove the DDe protecting group from Lys/D-Lys. The solution was drained, and the resin washed with DCM (3×5 mL). The deprotection step was repeated, and then the resin was washed with DCM (5×5 mL), DMF (5×5 mL). Further side chain derivatization was performed manually (PEG2, PEG2 and the gE (Fmoc-Glu-OtBu) residues) using DIC-HOAT (6 Eq, 1:1:1) at room temperature. mXOH (10-(3-(tert-butoxycarbonyl)phenoxy)decanoic acid) was coupled using DIC-HOAT (4 Eq, 1:1:1) at room temperature and complete acylation was monitored by ninhydrin test.

At the end of the assembly the resin was washed with DMF, MeOH, DCM, Et₂O. The peptide was cleaved from solid support using 15 ml of TFA solution (v/v) (87.5% TFA, 5% H₂O, 2.5% TIPS, 5% Phenol) for approximately 1.5 hours, at room temperature. The resin was then filtered and precipitated in cold MTBE (135 mL). After centrifugation, the peptide pellets were washed with fresh cold diethyl-ether to remove the organic scavengers. The process was repeated twice. Final pellets were dried, re-suspended in H₂O and acetonitrile 1:1+0.1% TFA and stirred overnight. The mixture was then lyophilized to afford the desired Intermediate 9-1 (70% yield). LCMS anal. calc. For C₁₆₅H₂₄₁N₂₉O₄₇S₂ 3447; found: 1150 (M+2)²⁺.

### Synthesis of MeCO-k(PEG2PEG2gEmXOH)-Pen*—N-T-7MeW—K(Ac)-Pen*-AEF-2Nal-THP-K(PEG2PEG2gEmXOH)—N-3Pya-Sar-CONH₂

Intermediate 9-1 was dissolved in ACN/H₂O (1 mg/ml). Saturated Iodine in acetic acid was then added dropwise under stirring until yellow color persisted. Reaction was completed in 30 min (monitored by UPLC-MS). Solid ascorbic acid was added until the solution became clear. After lyophilization the cyclized peptide was purified by reverse-phase HPLC using preparative Waters DeltaPak C4 (200×40 mm, 300 Å, 15 μm). Mobile phase A: +0.1% TFA, mobile phase B: Acetonitrile (ACN)+0.1% TFA. The following gradient of eluent B was used: 25% B to 25% B over 5 min, to 40% B over 25 min, flow rate 80 mL/min, wavelength 214 nm. Collected fractions were lyophilized to afford the desired compound (13% yield): LCMS anal. calc. For C₁₆₅H₂₃₉N₂₉O₄₇S₂ 3445; found: 1149.1 (M+3)³⁺.

### Example 10. Synthesis of MeCO-k(PEG2PEG2gEC16OH)-Pen*—N-T-7MeW—K(Ac)-Pen*-AEF-2Nal-THP-K(gEC16)-N-3Pya-Sar-CONH2 (*Pen-Pen form disulfide bond)

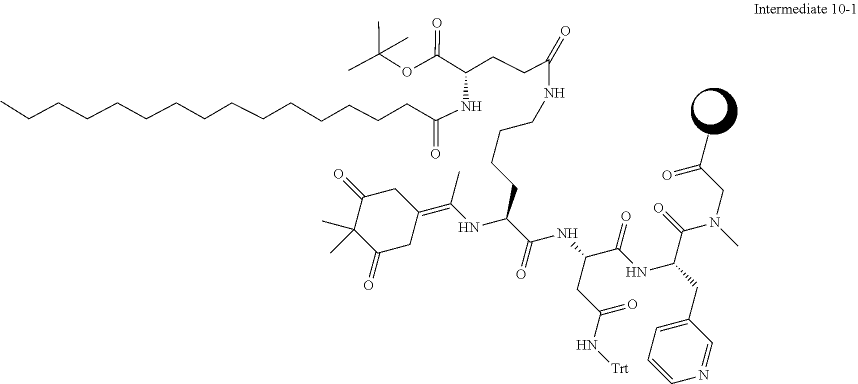

### Synthesis of Intermediate 10-1

The peptide was synthesized by standard Solid-phase Peptide Synthesis (SPPS) using Fmoc/t-Bu chemistry. The assembly was performed on a Rink-amide AM resin (75 μmol, 100-200Mesh; loading 0.33 mmol/g) on the Cem Liberty Blue microwave peptide synthesizer (CEM Inc.). During peptide assembly on solid phase, the side chain protecting groups were: tert-butyl for gE; trityl for Asn. Lys starting material was DDe-Lys(Fmoc)-OH. All the amino acids were dissolved at a 0.4 M concentration in DMF. The acylation reactions were performed for 3 min at 90° C. under MW irradiation with 5 folds excess of activated amino acids over the resin free amino groups. Double acylation reactions were performed for 3Pya15. The amino acids were activated with equimolar amounts of 0.5M solution of DIC in DMF and Oxyma solution 1M in DMF.

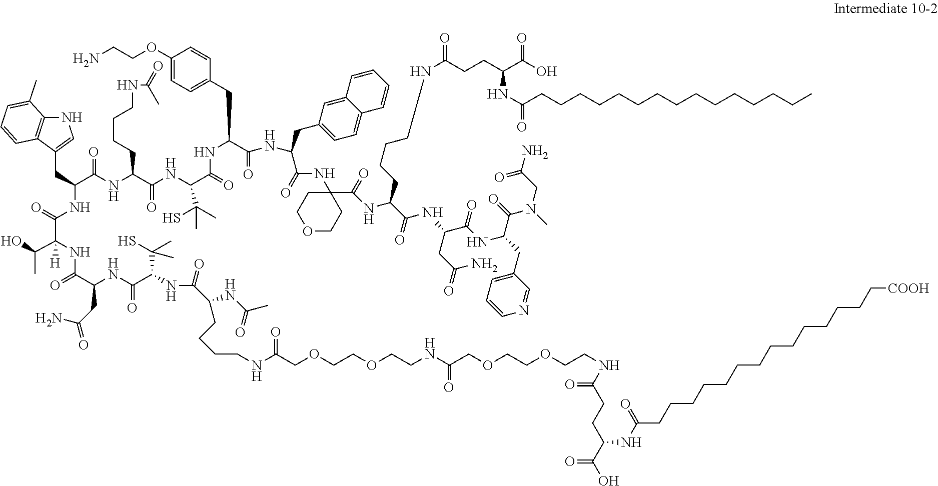 [SMILES: CCCCCCCCCCCCCCCC(=O)N[C@@H](CCC(=O)NCCCC[C@H](NC(=O)C1(NC(=O)[C@H](Cc2ccc3ccccc3c2)NC(=O)[C@H](Cc2ccc(OCCN)cc2)NC(=O)[C@@H](NC(=O)[C@H](CCCCNC(C)=O)NC(=O)[C@H](Cc2c[nH]c3c(C)cccc23)NC(=O)[C@@H](NC(=O)[C@H](CC(N)=O)NC(=O)[C@@H](NC(=O)[C@@H](CCCCNC(=O)COCCOCCNC(=O)COCCOCCNC(=O)CC[C@H](NC(=O)CCCCCCCCCCCCCCC(=O)O)C(=O)O)NC(C)=O)C(C)(C)S)[C@@H](C)O)C(C)(C)S)CCOCC1)C(=O)N[C@@H](CC(N)=O)C(=O)N[C@@H](Cc1cccnc1)C(=O)N(C)CC(N)=O)C(=O)O]

### Synthesis of Intermediate 10-2

Intermediate 10-1 was treated with 100 ml of 3% hydrazine solution in DMF to remove the Dde protecting group from Lys. The solution was drained, and the resin washed with DCM (3×5 mL) and DMF (5×5 mL). Assembly was then continued on the Cem Liberty Blue microwave peptide synthesizer using standard coupling conditions. The side chain protecting groups were: tert-butyl for Thr, trityl for Pen and Asn; tert-butoxy-carbonyl for AEF. N-terminal D-Lys residue was protected by the orthogonal DDe protecting group. Double acylation reactions were performed for 2Nal. Fmoc deprotections were performed using 20%(V/V) piperidine in DMF. Capping of the free amino group was performed manually using 10 eq of acetic anhydride in DMF. At the end of the peptide assembly on solid phase, the resin was treated with 100 ml of 3% hydrazine solution in DMF to remove the Dde protecting group from D-Lys. The solution was drained, and the resin washed with DCM (3×5 mL). The deprotection step was repeated, and then the resin was washed with DCM (5×5 mL), DMF (5×5 mL). Further side chain derivatization was performed manually (PEG2, PEG2 and the gE (Fmoc-Glu-OtBu) residues) using DIC-HOAT (6 Eq, 1:1:1) at room temperature. C16OH (Hexadecandioic acid) was coupled using DIC-HOAT (10 Eq, 1:1:1) at room temperature and complete acylation was monitored by ninhydrin test.

At the end of the assembly the resin was washed with DMF, MeOH, DCM, Et₂O. The peptide was cleaved from solid support using 15 ml of TFA solution (v/v) (87.5% TFA, 5% H₂O, 2.5% TIPS, 5% Phenol) for approximately 1.5 hours, at room temperature. The resin was then filtered and precipitated in cold MTBE (135 mL). After centrifugation, the peptide pellets were washed with fresh cold diethyl-ether to remove the organic scavengers. The process was repeated twice. Final pellets were dried, re-suspended in H₂O and acetonitrile 1:1+0.1% TFA and stirred overnight. Then lyophilized to afford the Intermediate 109-2 (94% yield). LCMS anal. calc. For C₁₅₁H₂₃₃N₂₇O₃₇S₂ 3082.80; found: 1542.2 (M+2)²⁺

### Synthesis of MeCO-k(PEG2PEG2, gEC16OH)-Pen*—N-T-7MeW—K(Ac)-Pen*-AEF-2Nal-THP-K(gEC16)—N-3Pya-Sar-CONH₂

Intermediate 10-2 was dissolved in ACN/H₂O (5 mg/ml). Saturated Iodine in acetic acid was then added dropwise under stirring until yellow color persisted. Reaction was completed in 30 min (monitored by UPLC-MS). Solid ascorbic acid was added until the solution became clear. After lyophilization the cyclized peptide was purified by reverse-phase HPLC using preparative Waters DeltaPak C4 (200×40 mm, 300 Å, 15 μm). Mobile phase A: +0.1% TFA, mobile phase B: Acetonitrile (ACN)+0.1% TFA. The following gradient of eluent B was used: 30% B to 30% B over 5 min, to 45% B over 20 min, flow rate 80 mL/min, wavelength 214 nm. Collected fractions were lyophilized to afford the desired compound (13% yield). LCMS anal. calc. For C₁₅₁H₂₃₁N₂₇O₃₇S₂ 3080.2; found: 1541.2 (M+2)²⁺.

### Example 11. Synthesis of HOC18gEPEG2PEG2-r-Pen*—N-T-7MeW—K(Ac)-Pen*-AEF-2Nal-THP-K(Ac)—N-3Pya-Sar-CONH2 (*Pen-Pen form disulfide bond)

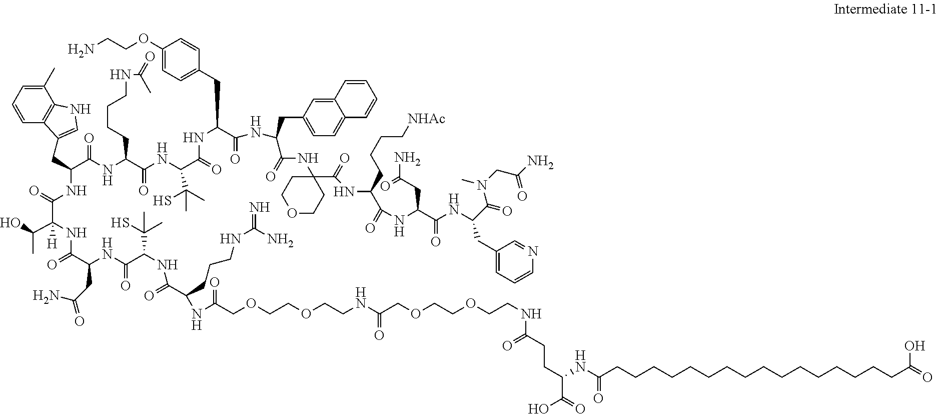 [SMILES: CC(=O)NCCCC[C@H](NC(=O)[C@H](Cc1c[nH]c2c(C)cccc12)NC(=O)[C@@H](NC(=O)[C@H](CC(N)=O)NC(=O)[C@@H](NC(=O)[C@@H](CCCNC(=N)N)NC(=O)COCCOCCNC(=O)COCCOCCNC(=O)CC[C@H](NC(=O)CCCCCCCCCCCCCCCCC(=O)O)C(=O)O)C(C)(C)S)[C@@H](C)O)C(=O)N[C@H](C(=O)N[C@@H](Cc1ccc(OCCN)cc1)C(=O)N[C@@H](Cc1ccc2ccccc2c1)C(=O)NC1(C(=O)N[C@@H](CCCCNC(C)=O)C(=O)N[C@@H](CC(N)=O)C(=O)N[C@@H](Cc2cccnc2)C(=O)N(C)CC(N)=O)CCOCC1)C(C)(C)S]

### Synthesis of Intermediate 11-1

The peptide was synthesized by standard Solid-phase Peptide Synthesis (SPPS) using Fmoc/t-Bu chemistry. The assembly was performed on a Rink-amide AM resin (110 mol, 100-200Mesh; loading 0.33 mmol/g) on the Cem Liberty Blue microwave peptide synthesizer (CEM Inc.). During peptide assembly on solid phase, the side chain protecting groups were: tert-butyl for Thr and Glu; trityl for Pen and Asn; tert-butoxy-carbonyl for AEF, Pbf (2,2,4,6,7-pentamethyldihydrobenzofuran-5-sulfonyl) for Arg. All the amino acids were dissolved at a 0.4 M concentration in DMF. The acylation reactions were performed for 3 min at 90° C. under MW irradiation with 5 folds excess of activated amino acids over the resin free amino groups. The amino acids were activated with equimolar amounts of 0.5M solution of DIC in DMF and Oxyma solution 1M in DMF. Double acylation reactions were performed for 3Pya and 2Nal. Fmoc deprotections were performed using 20%(V/V) piperidine in DMF. Capping of the free amino group was performed manually (PEG2, PEG2 and the gE (Fmoc-Glu-OtBu) residues) using DIC-HOAT (6 Eq, 1:1:1) at room temperature. C18OH (18-(tert-butoxy)-18-oxooctadecanoic acid) was coupled using DIC-HOAT (6 Eq, 1:1:1) at room temperature and complete acylation was monitored by ninhydrin test.

At the end of the assembly the resin was washed with DMF, MeOH, DCM, Et₂O. The peptide was cleaved from solid support using 15 ml of TFA solution (v/v) (87.5% TFA, 5% H₂O, 2.5% TIPS, 5% Phenol) for approximately 1.5 hours, at room temperature. The resin was then filtered and precipitated in cold MTBE (135 mL). After centrifugation, the peptide pellets were washed with fresh cold diethyl-ether to remove the organic scavengers. The process was repeated twice. Final pellets were dried, re-suspended in H₂O and acetonitrile 1:1+0.1% TFA and stirred overnight. The mixture was then lyophilized to afford the desired Intermediate 11-1 (78.1% yield). LCMS anal. calc. For C₁₃₂H₂₀₀N₂₈O₃₃S₂ 2771.32; found: 924.7 (M+3)³⁺.

### Synthesis of HOC18gEPEG2PEG2-r-Pen*—N-T-7MeW—K(Ac)-Pen*-AEF-2Nal-THP-K(Ac)—N-3Pya-Sar-CONH₂

Intermediate 12-1 was dissolved in ACN/H₂O (1 mg/ml). Saturated Iodine in acetic acid was then added dropwise under stirring until yellow color persisted. Reaction was completed in 30 min (monitored by UPLC-MS). Solid ascorbic acid was added until the solution became clear. After lyophilization the cyclized peptide was purified by reverse-phase HPLC using preparative Waters DeltaPak C4 (200×40 mm, 300 Å, 15 μm). Mobile phase A: +0.1% TFA, mobile phase B: Acetonitrile (ACN)+0.1% TFA. The following gradient of eluent B was used: 25% B to 25% B over 5 min, to 40% B over 25 min, flow rate 80 mL/min, wavelength 214 nm. Collected fractions were lyophilized to afford the desired compound (22% yield). LCMS anal. calc. For C₁₃₂H₁₉₈N₂₈O₃₃S₂ 2769.32; found: 1386.1 (M+2)²⁺.

### Example 12. Synthesis of HOC18gEPEG2PEG2-r-Pen*—N-T-7MeW—K(Ac)-Pen*-AEF-2Nal-THP-K(PEG2PEG2gEC18OH)—N-3Pya-Sar-CONH2 (*Pen-Pen form disulfide bond)

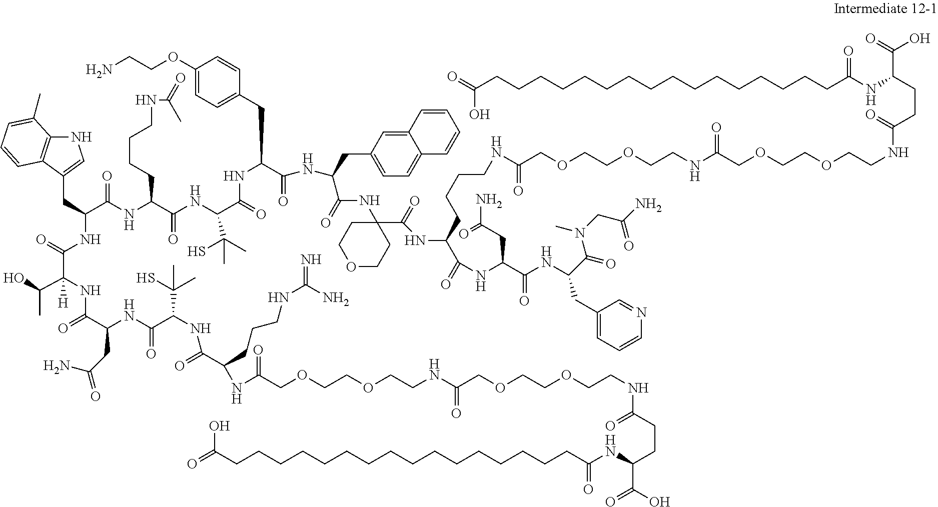 [SMILES: CC(=O)NCCCC[C@H](NC(=O)[C@H](Cc1c[nH]c2c(C)cccc12)NC(=O)[C@@H](NC(=O)[C@H](CC(N)=O)NC(=O)[C@@H](NC(=O)[C@@H](CCCNC(=N)N)NC(=O)COCCOCCNC(=O)COCCOCCNC(=O)CC[C@H](NC(=O)CCCCCCCCCCCCCCCCC(=O)O)C(=O)O)C(C)(C)S)[C@@H](C)O)C(=O)N[C@H](C(=O)N[C@@H](Cc1ccc(OCCN)cc1)C(=O)N[C@@H](Cc1ccc2ccccc2c1)C(=O)NC1(C(=O)N[C@@H](CCCCNC(=O)COCCOCCNC(=O)COCCOCCNC(=O)CC[C@H](NC(=O)CCCCCCCCCCCCCCCCC(=O)O)C(=O)O)C(=O)N[C@@H](CC(N)=O)C(=O)N[C@@H](Cc2cccnc2)C(=O)N(C)CC(N)=O)CCOCC1)C(C)(C)S]

### Synthesis of Intermediate 12-1

The peptide was synthesized by standard Solid-phase Peptide Synthesis (SPPS) using Fmoc/t-Bu chemistry. The assembly was performed on a Rink-amide AM resin (73 μmol, 100-200Mesh; loading 0.33 mmol/g) on the Cem Liberty Blue microwave peptide synthesizer (CEM Inc.). During peptide assembly on solid phase, the side chain protecting groups were: tert-butyl for Thr; trityl for Pen and Asn; tert-butoxy-carbonyl for AEF, Pbf (2,2,4,6,7-pentamethyldihydrobenzofuran-5-sulfonyl) for Arg. The Lys was protected by the orthogonal DDe protecting group.

All the amino acids were dissolved at a 0.4 M concentration in DMF. The acylation reactions were performed for 3 min at 90° C. under MW irradiation with 5 folds excess of activated amino acids over the resin free amino groups. The amino acids were activated with equimolar amounts of 0.5M solution of DIC in DMF and Oxyma solution 1M in DMF. Double acylation reactions were performed for 3Pya and 2Nal. Fmoc deprotections were performed using 20%(V/V) piperidine in DMF. At the end of the peptide assembly on solid phase, the resin was treated with 100 ml of 3% hydrazine solution in DMF. The solution was drained, and the resin washed with DCM (5×5 mL) and DMF (5×5 mL). Capping of the free amino group at the N-terminus and Lys13 side chain derivatization was performed manually (PEG2, PEG2 and the gE (Fmoc-Glu-OtBu) residues) using DIC-HOAT (5 Eq, 1:1:1) at room temperature. C18OH (18-(tert-butoxy)-18-oxooctadecanoic acid) was coupled using DIC-HOAT (5 Eq, 1:1:1) at room temperature and complete acylation was monitored by ninhydrin test.

At the end of the assembly the resin was washed with DMF, MeOH, DCM, Et₂O. The peptide was cleaved from solid support using 15 ml of TFA solution (v/v) (87.5% TFA, 5% H₂O, 2.5% TIPS, 5% Phenol) for approximately 1.5 hours, at room temperature. The resin was then filtered and precipitated in cold MTBE (135 mL). After centrifugation, the peptide pellets were washed with fresh cold diethyl-ether to remove the organic scavengers. The process was repeated twice. Final pellets were dried, re-suspended in H₂O and acetonitrile 1:1+0.1% TFA and stirred overnight. Then lyophilized to afford the desired Intermediate 12-1 (80.3% yield). LCMS anal. calc. For C₁₆₅H₂₅₉N₃₁O₄₄S₂: 3445.16; found: 1149.3 (M+3)³⁺.

### Synthesis of HOC18gEPEG2PEG2-r³-Pen*—N-T-7MeW—K(Ac)-Pen*-AEF-2Nal-THP-K(PEG2PEG2gEC18OH)—N-3Pya-Sar-CONH₂

Intermediate 12-1 was dissolved in ACN/H₂O (5 mg/ml). Saturated Iodine in acetic acid was then added dropwise under stirring until yellow color persisted. Reaction was completed in 30 min (monitored by UPLC-MS). Solid ascorbic acid was added until the solution became clear. After lyophilization the cyclized peptide was purified by reverse-phase HPLC using preparative Waters DeltaPak C4 (200×40 mm, 300 Å, 15 μm). Mobile phase A: +0.1% TFA, mobile phase B: Acetonitrile (ACN)+0.1% TFA. The following gradient of eluent B was used: 30% B to 30% B over 5 min, to 45% B over 25 min, flow rate 80 mL/min, wavelength 214 nm. Collected fractions were lyophilized to afford the desired compound (11.3% yield). LCMS anal. calc. For C₁₆₅H₂₅₇N₃₁O₄₄S₂: 3443.16; found: 1148.5 (M+3)³⁺.

### Example 13. Synthesis of MeCO-k(PEG2PEG2gEC18OH)-Pen*—N-T-7MeW—K(Ac)-Pen*-AEF-2Nal-THP-K(Ac)—N-3Pya-N(4AmBenzyl)Gly-CONH2 (*Pen-Pen form disulfide bond)

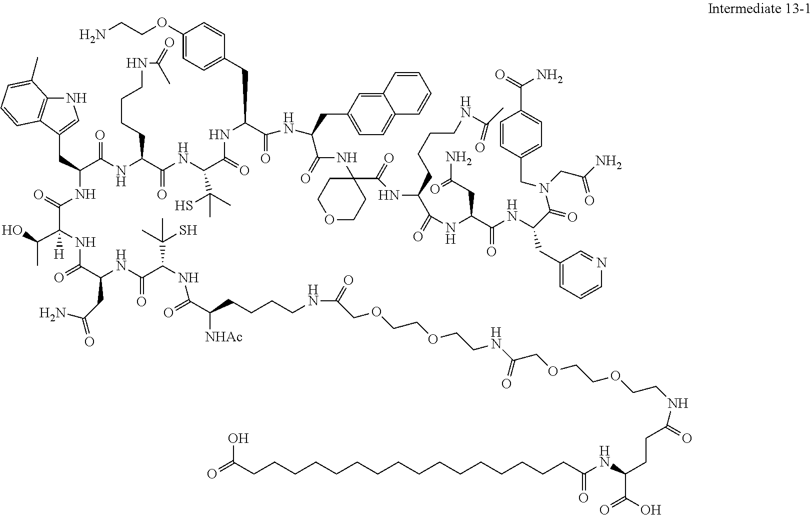 [SMILES: CC(=O)NCCCC[C@H](NC(=O)[C@H](Cc1c[nH]c2c(C)cccc12)NC(=O)[C@@H](NC(=O)[C@H](CC(N)=O)NC(=O)[C@@H](NC(=O)[C@@H](CCCCNC(=O)COCCOCCNC(=O)COCCOCCNC(=O)CC[C@H](NC(=O)CCCCCCCCCCCCCCCCC(=O)O)C(=O)O)NC(C)=O)C(C)(C)S)[C@@H](C)O)C(=O)N[C@H](C(=O)N[C@@H](Cc1ccc(OCCN)cc1)C(=O)N[C@@H](Cc1ccc2ccccc2c1)C(=O)NC1(C(=O)N[C@@H](CCCCNC(C)=O)C(=O)N[C@@H](CC(N)=O)C(=O)N[C@@H](Cc2cccnc2)C(=O)N(CC(N)=O)Cc2ccc(C(N)=O)cc2)CCOCC1)C(C)(C)S]

### Synthesis of Intermediate 13-1

Peptide assembly was performed on a rink amide MBHA resin (Novabiochem, 73 mol, 100-200Mesh; loading 0.34 mmol/g), by standard Solid-phase Peptide Synthesis (SPPS) using Fmoc/t-Bu chemistry. The resin, after FMOC deprotection was treated with a solution of 4 bromoacetic anhydride (4 eq) in DMF (5 mL) for 30 min at RT. Then, a suspension of 4-amidobenzylamine (7 eq) and DIPEA (7.5 eq) in dry NMP (5 mL) was added to the resin and stirred at RT overnight. The solution was drained, and the resin washed with DCM (3×5 mL) and DMF (3×5 mL). Peptide assembly was continued on the Cem Liberty Blue microwave peptide synthesizer (CEM Inc.), The side chain protecting groups were: tert-butyl for Thr; trityl for Pen and Asn; tert-butoxy-carbonyl for AEF. The D-Lys was protected by the orthogonal DDe protecting group. All the amino acids were dissolved at a 0.4 M concentration in DMF. The acylation reactions were performed for 3 min at 90° C. under MW irradiation with 5 folds excess of activated amino acids over the resin free amino groups. The amino acids were activated with equimolar amounts of 0.5M solution of DIC in DMF and Oxyma solution 1M in DMF. Double acylation reactions were performed for 3Pya and 2Nal. Fmoc deprotections were performed using 20%(V/V) piperidine in DMF. Capping of the free amino group was performed manually using 10 eq of acetic anhydride in DMF. At the end of the peptide assembly on solid phase, the resin was treated with 100 ml of 3% hydrazine solution in DMF to remove the Dde protecting group from D-Lys3. The solution was drained, and the resin washed with DCM (5×5 mL) and DMF (5×5 mL). Further side chain derivatization was performed manually (PEG2, PEG2 and the gE (Fmoc-Glu-OtBu) residues) using DIC-HOAT (3 Eq, 1:1:1) at room temperature. C18OH (18-(tert-butoxy)-18-oxooctadecanoic acid) was coupled using DIC-HOAT (6 Eq, 1:1:1) at room temperature and complete acylation was monitored by ninhydrin test.

At the end of the assembly the resin was washed with DMF, MeOH, DCM, Et₂O. The peptide was cleaved from solid support using 15 ml of TFA solution (v/v) (87.5% TFA, 5% H₂O, 2.5% TIPS, 5% Phenol) for approximately 1.5 hours, at room temperature. The resin was then filtered and precipitated in cold MTBE (135 mL). After centrifugation, the peptide pellets were washed with fresh cold diethyl-ether to remove the organic scavengers. The process was repeated twice. Final pellets were dried, re-suspended in H₂O and acetonitrile 1:1+0.1% TFA and stirred overnight. Then lyophilized to afford the desired Intermediate 13-1 (38% yield). LCMS anal. calc. For C₁₄₁H₂₀₇N₂₇O₃₅S₂: 2904.47; found 1452.6 (M+2)²⁺.

### Synthesis of MeCO-k(PEG2PEG2gEC18OH)-Pen*—N-T-7MeW—K(Ac)-Pen* AEF-2Nal-THP-K(Ac)—N-3Pya-N(4AmBenzyl)GLy-CONH₂

Intermediate 13-1 was dissolved in ACN/H₂O (5 mg/ml). Saturated Iodine in acetic acid was then added dropwise under stirring until yellow color persisted. Reaction was completed in 30 min (monitored by UPLC-MS). Solid ascorbic acid was added until the solution became clear. After lyophilization the cyclized peptide was purified by reverse-phase HPLC using preparative Waters DeltaPak C4 (200×25 mm, 300 Å, 15 μm). Mobile phase A: +0.1% TFA, mobile phase B: Acetonitrile (ACN)+0.1% TFA. The following gradient of eluent B was used: 25% B to 25% B over 5 min, to 40% B over 25 min, flow rate 30 mL/min, wavelength 214 nm. Collected fractions were lyophilized to afford the desired compound (26.8% yield). LCMS anal. calc. For C₁₄₁H₂₀₅N₂₇O₃₅S₂: 2902.47; found: 1451.9 (M+2)²⁺.

### Example 14. Synthesis of MeCO-r-Pen*—N-T-7MeW—K(Ac)-Pen*-AEF-2Nal-THP-K(Ac)—N-3Pya-N(PEG2PEG2gEC18OH)Gly-CONH2 (*Pen-Pen form disulfide bond)

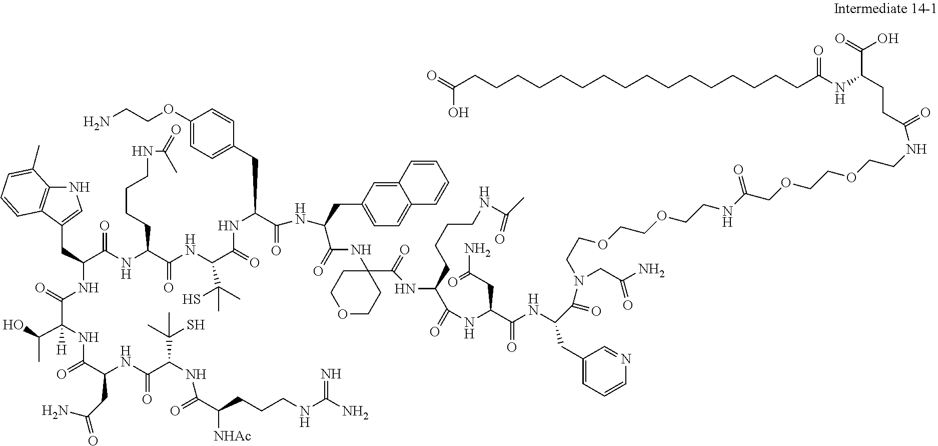 [SMILES: CC(=O)NCCCC[C@H](NC(=O)[C@H](Cc1c[nH]c2c(C)cccc12)NC(=O)[C@@H](NC(=O)[C@H](CC(N)=O)NC(=O)[C@@H](NC(=O)[C@@H](CCCNC(=N)N)NC(C)=O)C(C)(C)S)[C@@H](C)O)C(=O)N[C@H](C(=O)N[C@@H](Cc1ccc(OCCN)cc1)C(=O)N[C@@H](Cc1ccc2ccccc2c1)C(=O)NC1(C(=O)N[C@@H](CCCCNC(C)=O)C(=O)N[C@@H](CC(N)=O)C(=O)N[C@@H](Cc2cccnc2)C(=O)N(CCOCCOCCNC(=O)COCCOCCNC(=O)CC[C@H](NC(=O)CCCCCCCCCCCCCCCCC(=O)O)C(=O)O)CC(N)=O)CCOCC1)C(C)(C)S]

### Synthesis of Intermediate 14-1

Peptide assembly was performed on a rink amide MBHA resin (Novabiochem, 73 mol, 100-200Mesh; loading 0.34 mmol/g), by standard Solid-phase Peptide Synthesis (SPPS) using Fmoc/t-Bu chemistry. The resin, after FMOC deprotection was treated with a solution of 4 bromoacetic anhydride (4 eq) in DMF (5 mL) for 30 min at RT. Then, a solution of Bis-amino-PEG2 (7 eq) in dry NMP (5 mL) was added to the resin and stirred at RT overnight. The solution was drained, and the resin was treated with a solution of Dde-OH (3 eq) in DMF (5 mL) for 1 h at RT. The solution was drained, and the resin washed with DCM (3×5 mL) and DMF (3×5 mL). Peptide assembly was continued on the Cem Liberty Blue microwave peptide synthesizer (CEM Inc.), by standard Solid-phase Peptide Synthesis (SPPS) using Fmoc/t-Bu chemistry. The side chain protecting groups were: tert-butyl for Thr; trityl for Pen and Asn; tert-butoxy-carbonyl for AEF, Pbf (2,2,4,6,7-pentamethyldihydrobenzofuran-5-sulfonyl) for Arg. All the amino acids were dissolved at a 0.4 M concentration in DMF. The acylation reactions were performed for 3 min at 90° C. under MW irradiation with 5 folds excess of activated amino acids over the resin free amino groups. The amino acids were activated with equimolar amounts of 0.5M solution of DIC in DMF and Oxyma solution 1M in DMF. Double acylation reactions were performed for 3Pya and 2Nal. Fmoc deprotections were performed using 20%(V/V) piperidine in DMF. Capping of the free amino group was performed manually using 10 eq of acetic anhydride in DMF.

At the end of the peptide assembly on solid phase, the resin was treated with 100 ml of 3% hydrazine solution in DMF. The solution was drained, and the resin washed with DCM (5×5 mL) and DMF (5×5 mL). Further side chain derivatization was performed manually (gE (Fmoc-Glu-OtBu) and C18OH (18-(tert-butoxy)-18-oxooctadecanoic acid) residues) using DIC-HOAT (3 Eq, 1:1:1) at room temperature and complete acylation was monitored by ninhydrin test.

At the end of the assembly the resin was washed with DMF, MeOH, DCM, Et₂O. The peptide was cleaved from solid support using 15 ml of TFA solution (v/v) (87.5% TFA, 5% H₂O, 2.5% TIPS, 5% Phenol) for approximately 1.5 hours, at room temperature. The resin was then filtered and precipitated in cold MTBE (135 mL). After centrifugation, the peptide pellets were washed with fresh cold diethyl-ether to remove the organic scavengers. The process was repeated twice. Final pellets were dried, re-suspended in H₂O and acetonitrile 1:1+0.1% TFA and stirred overnight. Then lyophilized to afford the desired Intermediate 14-1 (76.7% yield). LCMS anal. calc. For C₁₃₃H₂₀₂N₂₈O₃₃S₂ 2785.35; found: 1393.4 (M+2)²⁺.

### Synthesis of MeCO-r-Pen*—N-T-7MeW—K(Ac)-Pen*-AEF-2Nal-THP-K(Ac)—N-3Pya-N(PEG2PEG2gEC18OH)Gly-CONH₂

Intermediate 14-1 was dissolved in ACN/H₂O (5 mg/ml). Saturated Iodine in acetic acid was then added dropwise under stirring until yellow color persisted. Reaction was completed in 30 min (monitored by UPLC-MS). Solid ascorbic acid was added until the solution became clear. After lyophilization the cyclized peptide was purified by reverse-phase HPLC using preparative Waters DeltaPak C4 (200×40 mm, 300 Å, 15 μm). Mobile phase A: +0.1% TFA, mobile phase B: Acetonitrile (ACN)+0.1% TFA. The following gradient of eluent B was used: 30% B to 30% B over 5 min, to 45% B over 25 min, flow rate 80 mL/min, wavelength 214 nm. Collected fractions were lyophilized to afford the desired compound (19.6% yield). LCMS anal. calc. For C₁₃₃H₂₀₀N₂₈O₃₃S₂: 2783.35; found: 1392.1 (M+2)²⁺.

### Example 15. Synthesis of MeCO-k(PEG2PEG2gEDab(mXOH)2)-Pen*—N-T-7MeW—K(Ac)-Pen*-AEF-2Nal-THP-K(Ac)—N-3Pya-Sar-CONH2 (*Pen-Pen form disulfide bond)

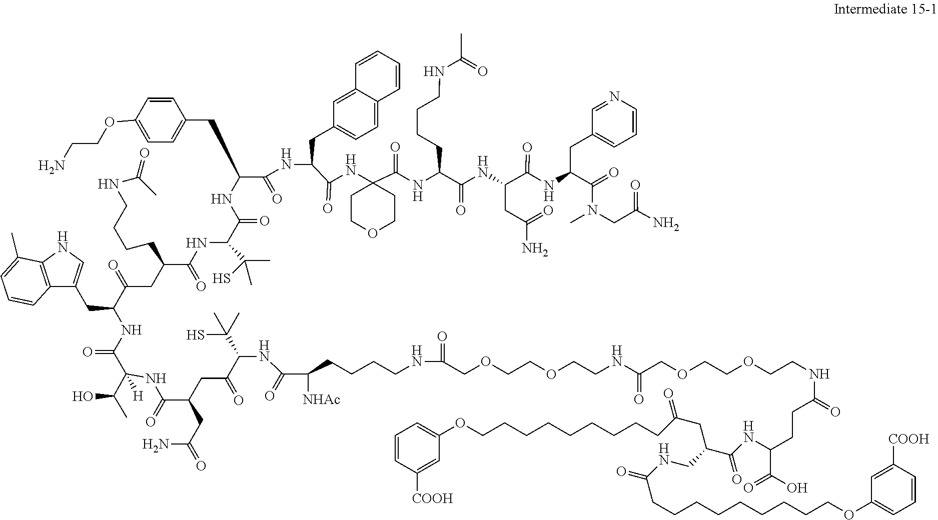 [SMILES: CC(=O)NCCCC[C@H](CC(=O)[C@H](Cc1c[nH]c2c(C)cccc12)NC(=O)[C@@H](NC(=O)[C@H](CC(N)=O)CC(=O)[C@@H](NC(=O)[C@@H](CCCCNC(=O)COCCOCCNC(=O)COCCOCCNC(=O)CCC(NC(=O)[C@H](CNC(=O)CCCCCCCCCOc1cccc(C(=O)O)c1)CC(=O)CCCCCCCCCOc1cccc(C(=O)O)c1)C(=O)O)NC(C)=O)C(C)(C)S)[C@@H](C)O)C(=O)N[C@H](C(=O)N[C@@H](Cc1ccc(OCCN)cc1)C(=O)N[C@@H](Cc1ccc2ccccc2c1)C(=O)NC1(C(=O)N[C@@H](CCCCNC(C)=O)C(=O)N[C@@H](CC(N)=O)C(=O)N[C@@H](Cc2cccnc2)C(=O)N(C)CC(N)=O)CCOCC1)C(C)(C)S]

### Synthesis of Intermediate 15-1

The peptide was synthesized by standard Solid-phase Peptide Synthesis (SPPS) using Fmoc/t-Bu chemistry. The assembly was performed on a Rink-amide AM resin (220 mol, 100-200Mesh; loading 0.33 mmol/g) on the Cem Liberty Blue microwave peptide synthesizer (CEM Inc.). During peptide assembly on solid phase, the side chain protecting groups were: tert-butyl for Thr; trityl for Pen and Asn; tert-butoxy-carbonyl for AEF, Pbf (2,2,4,6,7-pentamethyldihydrobenzofuran-5-sulfonyl) for Arg. The D-Lys was protected by the orthogonal DDe protecting group. All the amino acids were dissolved at a 0.4 M concentration in DMF. The acylation reactions were performed for 3 min at 90° C. under MW irradiation with 5 folds excess of activated amino acids over the resin free amino groups. The amino acids were activated with equimolar amounts of 0.5M solution of DIC in DMF and Oxyma solution 1M in DMF. Double acylation reactions were performed for 3Pya and 2Nal. Fmoc deprotections were performed using 20%(V/V) piperidine in DMF. Capping of the free amino group was performed manually using 10 eq of acetic anhydride in DMF. At the end of the peptide assembly on solid phase, the resin was treated with 100 ml of 3% hydrazine solution in DMF. The solution was drained, and the resin washed with DCM (5×5 mL) and DMF (5×5 mL). Further side chain derivatization was performed manually (PEG2, PEG2, gE (Fmoc-Glu-OtBu) and Dap (Fmoc-Dap(DDe)-OH)) using DIC-HOAt (5 Eq, 1:1:1) at room temperature. At the end of the peptide assembly on solid phase, the resin was treated with 100 ml of 3% hydrazine solution in DMF to remove the Dde protecting group from Dap. The solution was drained, and the resin washed with DCM (5×5 mL) and DMF (5×5 mL). Further side chain derivatization was performed manually using mXOH (10-(3-(tert-butoxycarbonyl)phenoxy)decanoic acid), DIC, HOAt (4 Eq, 1:1:1) at room temperature and complete acylation was monitored by ninhydrin test.

At the end of the assembly the resin was washed with DMF, MeOH, DCM, Et₂O. The peptide was cleaved from solid support using 30 ml of TFA solution (v/v) (87.5% TFA, 5% H₂O, 2.5% TIPS, 5% Phenol) for approximately 1.5 hours, at room temperature. The resin was then filtered and precipitated in cold MTBE (135 mL). After centrifugation, the peptide pellets were washed with fresh cold diethyl-ether to remove the organic scavengers. The process was repeated twice. Final pellets were dried, re-suspended in H₂O and acetonitrile 1:1+0.1% TFA and stirred overnight. The mixture was then lyophilized to afford the desired Intermediate 131-1 (75.6% yield). LCMS anal. calc. LCMS anal. calc. For C₁₄₁H₂₁₇N₂₈O₃₅S₂⁺: 3155.72; found: 1053.1 (M+3)³⁺.

### Synthesis of MeCO-k(PEG2PEG2gEDab(mXOH)2)-Pen*—N-T-7MeW—K(Ac)-Pen* AEF-2Nal-THP-K(Ac)—N-3Pya-Sar-CONH₂

Intermediate 15-1 was dissolved in ACN/H₂O (1 mg/ml). Saturated Iodine in acetic acid was then added dropwise under stirring until yellow color persisted. Reaction was completed in 30 min (monitored by UPLC-MS). Solid ascorbic acid was added until the solution became clear. After lyophilization the cyclized peptide was purified by reverse-phase HPLC using preparative Waters DeltaPak C4 (200×40 mm, 300 Å, 15 μm). Mobile phase A: +0.1% TFA, mobile phase B: Acetonitrile (ACN)+0.1% TFA. The following gradient of eluent B was used: 30% B to 30% B over 5 min, to 45% B over 25 min, flow rate 80 mL/min, wavelength 214 nm. Collected fractions were lyophilized to afford the desired compound (14% yield). LCMS anal. calc. For C₁₅₃H₂₁₈N₂₈O₄₀S₂: 3153.7; found: 1578 (M+2)²⁺.

### Example 16. Synthesis of MeCO-k(PEG2PEG2gE(c)C18OH)-Pen*—N-T-7MeW—K(Ac)-Pen*-AEF-2Nal-THP-K(Ac)—N-3Pya-Sar-CONH2 (*Pen-Pen form disulfide bond)

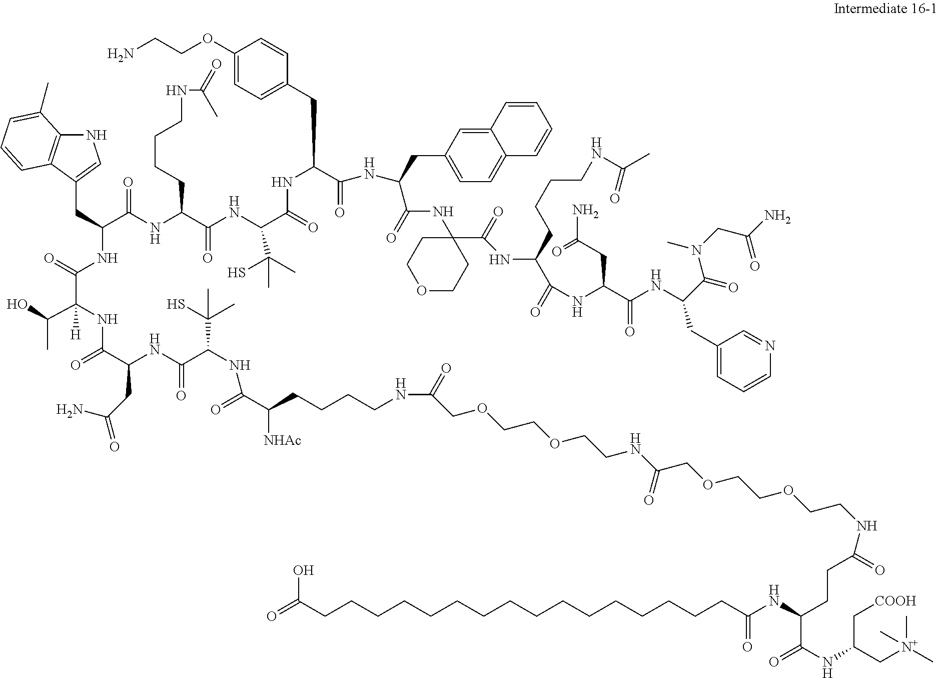 [SMILES: CC(=O)NCCCC[C@H](NC(=O)[C@H](Cc1c[nH]c2c(C)cccc12)NC(=O)[C@@H](NC(=O)[C@H](CC(N)=O)NC(=O)[C@@H](NC(=O)[C@@H](CCCCNC(=O)COCCOCCNC(=O)COCCOCCNC(=O)CC[C@H](NC(=O)CCCCCCCCCCCCCCCCC(=O)O)C(=O)N[C@H](CC(=O)O)C[N+](C)(C)C)NC(C)=O)C(C)(C)S)[C@@H](C)O)C(=O)N[C@H](C(=O)N[C@@H](Cc1ccc(OCCN)cc1)C(=O)N[C@@H](Cc1ccc2ccccc2c1)C(=O)NC1(C(=O)N[C@@H](CCCCNC(C)=O)C(=O)N[C@@H](CC(N)=O)C(=O)N[C@@H](Cc2cccnc2)C(=O)N(C)CC(N)=O)CCOCC1)C(C)(C)S]

### Synthesis of Intermediate 16-1

The peptide was synthesized by standard Solid-phase Peptide Synthesis (SPPS) using Fmoc/t-Bu chemistry. The assembly was performed on a Rink-amide AM resin (110 mol, 100-200Mesh; loading 0.33 mmol/g) on the Cem Liberty Blue microwave peptide synthesizer (CEM Inc.). During peptide assembly on solid phase, the side chain protecting groups were: tert-butyl for Thr and Glu; trityl for Pen and Asn; tert-butoxy-carbonyl for AEF. The D-Lys was protected by the orthogonal DDe protecting group.

All the amino acids were dissolved at a 0.4 M concentration in DMF. The acylation reactions were performed for 3 min at 90° C. under MW irradiation with 5 folds excess of activated amino acids over the resin free amino groups. The amino acids were activated with equimolar amounts of 0.5M solution of DIC in DMF and Oxyma solution 1M in DMF. Double acylation reactions were performed for 3Pya and 2Nal. Fmoc deprotections were performed using 20%(V/V) piperidine in DMF. Capping of the free amino group was performed manually using 10 eq of acetic anhydride in DMF. At the end of the peptide assembly on solid phase, the resin was treated with 100 ml of 3% hydrazine solution in DMF. The solution was drained, and the resin washed with DCM (5×5 mL) and DMF (5×5 mL). Further side chain derivatization was performed manually (PEG2, PEG2 and the gE ((S,E)-4-((Fmoc)amino)-5-oxo-5-(prop-1-en-1-yloxy)pentanoic acid) residues) using DIC-HOAT (3 Eq, 1:1:1) at room temperature. C18OH (18-(tert-butoxy)-18-oxooctadecanoic acid) was coupled using DIC-HOAT (6 Eq, 1:1:1) at room temperature and complete acylation was monitored by ninhydrin test. The resin was then treated with 0.25 Eq of Pd Tetrakis, 24 Eq of Phenylsilane in 5 ml of DCM Dry under N2 atmosphere for 30 min (process repeated 2 times); washed with DCM, DMF and a solution of 0.5% sodium dimethyldithiocarbamate (0.5%) and DIPEA (0.5%) in DMF. The resin was then manually preactivated with HATU (1.2 Eq) and dipea (2 Eq) and was left under stirring for 10 minutes. Amino-carnitine (2 Eq; (R)-2-amino-4-(tert-butoxy)-N,N,N-trimethyl-4-oxobutan-1-aminium) was added. Reaction was completed after 2 hr (monitored by test cleavage).

At the end of the assembly the resin was washed with DMF, MeOH, DCM, Et₂O. The peptide was cleaved from solid support using 15 ml of TFA solution (v/v) (87.5% TFA, 5% H₂O, 2.5% TIPS, 5% Phenol) for approximately 1.5 hours, at room temperature. The resin was then filtered and precipitated in cold MTBE (135 mL). After centrifugation, the peptide pellets were washed with fresh cold diethyl-ether to remove the organic scavengers. The process was repeated twice. Final pellets were dried, re-suspended in H₂O and acetonitrile 1:1+0.1% TFA and stirred overnight. The mixture was then lyophilized to afford the Intermediate 142-1 (73.6% yield). LCMS anal. calc. For C₁₄₁H₂₁₇N₂₈O₃₅S₂⁺: 2928.55; found: 1464.74 (M+2)²⁺.

### Synthesis of MeCO-k(PEG2PEG2gE(c)C18OH)-Pen*—N-T-7MeW—K(Ac)-Pen* AEF-2Nal-THP-K(Ac)—N-3Pya-Sar-CONH₂

Intermediate 16-1 was dissolved in ACN/H₂O (1 mg/ml). Saturated Iodine in acetic acid was then added dropwise under stirring until yellow color persisted. Reaction was completed in 30 min (monitored by UPLC-MS). Solid ascorbic acid was added until the solution became clear. After lyophilization the cyclized peptide was purified by reverse-phase HPLC using preparative Waters DeltaPak C4 (200×40 mm, 300 Å, 15 μm). Mobile phase A: +0.1% TFA, mobile phase B: Acetonitrile (ACN)+0.1% TFA. The following gradient of eluent B was used: 20% B to 20% B over 5 min, to 35% B over 25 min, flow rate 80 mL/min, wavelength 214 nm. Collected fractions were lyophilized to afford the desired compound (20% yield). LCMS anal. calc. For C₁₄₁H₂₁₅N₂₈O₃₅S₂⁺: 2926.55; found 1463.9 (M+2)²⁺.

### Examples 17-142. Synthesis

Additional compounds have been prepared according to the methods described above, with illustrative data as shown in Table 3 below. In all the Examples, * indicate that Pen-Pen form a disulfide bond.

TABLE 3
Compound Synthesis

| | | | Synthetic |
|---|---|---|---|
| | | | Procedure |
| Example | Name | MS Data | (Example) |
| 17 | MeCO-r-Pen*-N-T-7MeW-K(Ac)-Pen*-AEF-2Nal- | 1436.6 | 2 |
| | THP-E-N-3Pya-Sar-K(PEG2PEG2gEC16OH)-CONH₂ | | |
| 18 | MeCO-r-Pen*-N-T-7MeW-K(Ac)-Pen*-AEF-2Nal- | 1414.9 | 2 |
| | THP-E-N-3Pya-K(PEG2PEG2gEC18OH)-CONH₂ | | |
| 19 | MeCO-Pen*-N-T-7MeW-K(Ac)-Pen*-AEF-2Nal- | 1293.4 | 24 |
| | THP-K(PEG2PEG2gEC16OH)-N-3Pya-Sar-CONH₂ | | |
| 20 | MeCO-r-Pen*-K(PEG2PEG2gEC18OH)-T-7MeW-K(Ac)-Pen*- | 1393.3 | 2 |
| | AEF-2Nal-THP-E-N-3Pya-Sar-CONH₂ | | |
| 21 | MeCO-r-Pen*-K(PEG2PEG2gEC16OH)-T-7MeW-K(Ac)-Pen*- | 1379.1 | 2 |
| | AEF-2Nal-THP-E-N-3Pya-Sar-CONH₂ | | |
| 22 | MeCO-r-Pen*-N-T-7MeW-K(Ac)-Pen*-AEF-2Nal- | 1407.9 | 23 |
| | THP-E-N-3Pya-NMeK(PEG2PEG2gEC16OH)-CONH₂ | | |
| 23 | MeCO-r-Pen*-N-T-7MeW-K(Ac)-Pen*-AEF-2Nal- | 1400.8 | 2 |
| | THP-E-N-3Pya-K(PEG2PEG2gEC16OH)-CONH₂ | | |
| 24 | HOC16gEPEG2PEG2-r-Pen*-N-T-7MeW-K(Ac)-Pen*- | 1371.5 | 11 |
| | AEF-2Nal-THP-K(Ac)-N-3Pya-Sar-CONH₂ | | |
| 25 | MeCO-K(PEG2PEG2gEC16OH)-Pen*-N-T-7MeW-K(Ac)-Pen*- | 1378.5 | 25 |
| | AEF-2Nal-THP-K(Ac)-N-3Pya-Sar- | | |
| | CONH₂ | | |
| 26 | MeCO-r-Pen*-N-T-7MeW-K(Ac)-Pen*- | 1392.2 | 26 |
| | AEF(PEG2PEG2gEC16OH)-2Nal-THP-K(Ac)-N- | | |
| | 3Pya-Sar-CONH₂ | | |
| 27 | MeCO-r-Pen*-N-T-7MeW-K(Ac)-Pen*-AEF-2Nal- | 1385.3 | 4 |
| | THP-K(PEG2PEG2gEC18OH)-N-3Pya-Sar-CONH₂ | | |
| 28 | MeCO-r-Pen*-N-T-7MeW-K(Ac)-Pen*-AEF-2Nal- | 1371.2 | 4 |
| | THP-K(PEG2PEG2gEC16OH)-N-3Pya-Sar-CONH₂ | | |
| 29 | MeCO-Pen*-N-T-7MeW-K(Ac)-Pen*-AEF-2Nal- | 1148.1 | 24 |
| | THP-K(gEC16OH)-N-3Pya-Sar-CONH₂ | | |
| 30 | MeCO-Pen*-N-T-7MeW-K(Ac)-Pen*-AEF-2Nal- | 1162.1 | 24 |
| | THP-K(gEC18OH)-N-3Pya-Sar-CONH₂ | | |
| 31 | MeCO-Pen*-N-T-7MeW-K(Ac)-Pen*-AEF-2Nal- | 1147.3 | 24 |
| | THP-K(gEC18)-N-3Pya-Sar-CONH₂ | | |
| 32 | MeCO-Pen*-N-T-7MeW-K(Ac)-Pen*-AEF-2Nal- | 1315.6 | 24 |
| | THP-K(PEG6gEC16OH)-N-3Pya-Sar-CONH₂ | | |
| 33 | MeCO-Pen*-N-T-7MeW-K(Ac)-Pen*-AEF-2Nal- | 1329.7 | 24 |
| | THP-K(PEG6gEC18OH)-N-3Pya-Sar-CONH₂ | | |
| 34 | MeCO-Pen*-N-T-7MeW-K(Ac)-Pen*-AEF-2Nal- | 1220.6 | 24 |
| | THP-K(PEG2gEC16OH)-N-3Pya-Sar-CONH₂ | | |
| 35 | MeCO-Pen*-N-T-7MeW-K(Ac)-Pen*-AEF-2Nal- | 1234.6 | 24 |
| | THP-K(PEG2gEC18OH)-N-3Pya-Sar-CONH₂ | | |
| 36 | MeCO-Pen*-N-T-7MeW-K(Ac)-Pen*-AEF-2Nal- | 1321.6 | 24 |
| | THP-K(PEG2PEG2gEC20OH)-N-3Pya-Sar-CONH₂ | | |
| 37 | HOC16gEPEG2PEG2-r-Pen*-N-T-7MeW-K(Ac)-Pen*- | 1129.9 | 112 |
| | AEF-2Nal-THP-K(PEG2PEG2gEC16OH)-N- | | |
| | 3Pya-Sar-CONH₂ | | |
| 38 | MeCO-Pen*-N-T-7MeW-K(Ac)-Pen*-AEF-2Nal- | 1388.4 | 24 |
| | THP-K(PEG2PEG6gEC16OH)-N-3Pya-Sar-CONH₂ | | |
| 39 | MeCO-Pen*-N-T-7MeW-K(Ac)-Pen*-AEF-2Nal- | 935 | 24 |
| | THP-K(PEG2PEG6gEC18OH)-N-3Pya-Sar-CONH₂ | | |
| 40 | MeCO-Pen*-N-T-7MeW-K(Ac)-Pen*-AEF-2Nal- | 1229.2 | 24 |
| | THP-K(PEG2PEG2C16OH)-N-3Pya-Sar-CONH₂ | | |
| 41 | MeCO-Pen*-N-T-7MeW-K(Ac)-Pen*-AEF-2Nal- | 1242.8 | 24 |
| | THP-K(PEG2PEG2C18OH)-N-3Pya-Sar-CONH₂ | | |
| 42 | MeCO-Pen*-N-T-7MeW-K(Ac)-Pen*-AEF-2Nal- | 989.1 | 24 |
| | THP-K(PEG6PEG6gEC16OH)-N-3Pya-Sar-CONH₂ | | |
| 43 | MeCO-Pen*-N-T-7MeW-K(Ac)-Pen*-AEF-2Nal- | 998.7 | 24 |
| | THP-K(PEG6PEG6gEC18OH)-N-3Pya-Sar-CONH₂ | | |
| 44 | MeCO-Pen*-N-T-7MeW-K(Ac)-Pen*-AEF-2Nal- | 1141.6 | 24 |
| | THP-K(PEG24gEC16OH)-N-3Pya-Sar-CONH₂ | | |
| 45 | MeCO-Pen*-N-T-7MeW-K(Ac)-Pen*-AEF-2Nal- | 1151.2 | 24 |
| | THP-K(PEG24gEC18OH)-N-3Pya-Sar-CONH₂ | | |
| 46 | MeCO-Pen*-N-T-7MeW-K(Ac)-Pen*-AEF-2Nal- | 1328.2 | 8 |
| | THP-K(Ac)-N-3Pya-Sar-PEG2PEG2eKC18OH- | | |
| | COOH | | |
| 47 | MeCO-Pen*-N-T-7MeW-K(Ac)-Pen*-AEF-2Nal- | 1108.3 | 24 |
| | THP-K(PEG24C18OH)-N-3Pya-Sar-CONH₂ | | |
| 48 | MeCO-K(gEC18OH)-Pen*-N-T-7MeW-K(Ac)- | 1247.2 | 25 |
| | Pen*-AEF-2Nal-THP-K(Ac)-N-3Pya-Sar-CONH₂ | | |
| 49 | MeCO-K(PEG2gEC18OH)-Pen*-N-T-7MeW- | 1319.5 | 25 |
| | K(Ac)-Pen*-AEF-2Nal-THP-K(Ac)-N-3Pya-Sar- | | |
| | CONH₂ | | |
| 50 | MeCO-r-Pen*-K(PEG2PEG2gEC18OH)-T-7MeW- | 1413.4 | 2 |
| | K(Ac)-Pen*-AEF-2Nal-THP-K(Ac)-N-3Pya-Sar- | | |
| | CONH₂ | | |
| 51 | MeCO-r-Pen*-K(gEC18OH)-T-7MeW-K(Ac)-Pen*- | 1268.1 | 2 |
| | AEF-2Nal-THP-K(Ac)-N-3Pya-Sar-CONH₂ | | |
| 52 | MeCO-r-Pen*-K(PEG2gEC18OH)-T-7MeW- | 1340.4 | 2 |
| | K(Ac)-Pen*-AEF-2Nal-THP-K(Ac)-N-3Pya-Sar- | | |
| | CONH₂ | | |
| 53 | MeCO-r-Pen*-K(PEG2PEG2C18OH)-T-7MeW- | 1349.1 | 2 |
| | K(Ac)-Pen*-AEF-2Nal-THP-K(Ac)-N-3Pya-Sar- | | |
| | CONH₂ | | |
| 54 | MeCO-r-Pen*-N-T-7MeW-K(Ac)-Pen*-AEF-2Nal- | 1442 | 23 |
| | THP-K(Ac)-N-3Pya-NMeK(PEG2PEG2gEC18OH)- | | |
| | CONH₂ | | |
| 55 | MeCO-r-Pen*-N-T-7MeW-K(Ac)-Pen*-AEF-2Nal- | 1385.3 | 24 |
| | THP-K(PEG2PEG2DgEC18OH)-N-3Pya-Sar- | | |
| | CONH₂ | | |
| 56 | MeCO-r-Pen*-N-T-7MeW-K(Ac)-Pen*-AEF-2Nal- | 1369.1 | 24 |
| | THP-K(PEG2PEG2PC18OH)-N-3Pya-Sar-CONH₂ | | |
| 57 | MeCO-r-Pen*-N-T-7MeW-K(Ac)-Pen*-AEF-2Nal- | 1369.1 | 24 |
| | THP-K(PEG2PEG2pC18OH)-N-3Pya-Sar-CONH₂ | | |
| 58 | MeCO-r-Pen*-N-T-7MeW-K(Ac)-Pen*-AEF-2Nal- | 1454.9 | 24 |
| | THP-K(PEG2PEG2gETrxC18OH)-N-3Pya-Sar- | | |
| | CONH₂ | | |
| 59 | MeCO-r-Pen*-N-T-7MeW-K(Ac)-Pen*-AEF-2Nal- | 1468.7 | 24 |
| | THP-K(PEG2PEG2gETrxC20OH)-N-3Pya-Sar- | | |
| | CONH₂ | | |
| 60 | MeCO-k(PEG6gEC18OH)-Pen*-N-T-7MeW- | 1414.7 | 25 |
| | K(Ac)-Pen*-AEF-2Nal-THP-K(Ac)-N-3Pya-Sar- | | |
| | CONH₂ | | |
| 61 | MeCO-k(PEG2PEG6gEC18OH)-Pen*-N-T- | 991.9 | 25 |
| | 7MeW-K(Ac)-Pen*-AEF-2Nal-THP-K(Ac)-N-3Pya- | | |
| | Sar-CONH₂ | | |
| 62 | MeCO-K(PEG2PEG2C18OH)-Pen*-N-T-7MeW- | 1327.8 | 25 |
| | K(Ac)-Pen*-AEF-2Nal-THP-K(Ac)-N-3Pya-Sar- | | |
| | CONH₂ | | |
| 63 | MeCO-r-Pen*-K(PEG6gEC18OH)-T-7MeW-K(Ac)- | 1435.7 | 2 |
| | Pen*-AEF-2Nal-THP-K(Ac)-N-3Pya-Sar-CONH₂ | | |
| 64 | MeCO-r-Pen*-K(PEG2PEG6gEC18OH)-T-7MeW- | 1508.4 | 2 |
| | K(Ac)-Pen*-AEF-2Nal-THP-K(Ac)-N-3Pya-Sar- | | |
| | CONH₂ | | |
| 65 | MeCO-r-Pen*-N-T-7MeW-K(Ac)-Pen*-AEF-2Nal- | 978.2 | 24 |
| | THP-K(PEG2PEG2PPPC18OH)-N-3Pya-Sar- | | |
| | CONH₂ | | |
| 66 | MeCO-r-Pen*-N-T-7MeW-K(Ac)-Pen*-AEF-2Nal- | 978.2 | 24 |
| | THP-K(PEG2PEG2pppC18OH)-N-3Pya-Sar- | | |
| | CONH₂ | | |
| 67 | MeCO-k(gEC16)-Pen*-N-T-7MeW-K(Ac)-Pen*- | 1218.5 | 25 |
| | AEF-2Nal-THP-K(Ac)-N-3Pya-Sar-CONH₂ | | |
| 68 | MeCO-k(gEC18)-Pen*-N-T-7MeW-K(Ac)-Pen*- | 1232.2 | 25 |
| | AEF-2Nal-THP-K(Ac)-N-3Pya-Sar-CONH₂ | | |
| 69 | MeCO-r-Pen*-K(gEC16)-T-7MeW-K(Ac)-Pen*- | 1239.4 | 2 |
| | AEF-2Nal-THP-K(Ac)-N-3Pya-Sar-CONH₂ | | |
| 70 | MeCO-r-Pen*-K(gEC18)-T-7MeW-K(Ac)-Pen*- | 1253.3 | 2 |
| | AEF-2Nal-THP-K(Ac)-N-3Pya-Sar-CONH₂ | | |
| 71 | MeCO-K(PEG2PEG2gETrxC18OH)-Pen*-N-T- | 1461.8 | 25 |
| | 7MeW-K(Ac)-Pen*-AEF-2Nal-THP-K(Ac)-N-3Pya- | | |
| | Sar-CONH₂ | | |
| 72 | MeCO-K(PEG2PEG2gETrxC20OH)-Pen*-N-T- | 1476.3 | 25 |
| | 7MeW-K(Ac)-Pen*-AEF-2Nal-THP-K(Ac)-N-3Pya- | | |
| | Sar-CONH₂ | | |
| 73 | MeCO-Pen*-K(PEG2PEG2gETrxC18OH)-T- | 1405 | 2 |
| | 7MeW-K(Ac)-Pen*-AEF-2Nal-THP-K(Ac)-N-3Pya- | | |
| | Sar-CONH₂ | | |
| 74 | MeCO-Pen*-K(PEG2PEG2gETrxC20OH)-T- | 1419.2 | 2 |
| | 7MeW-K(Ac)-Pen*-AEF-2Nal-THP-K(Ac)-N-3Pya- | | |
| | Sar-CONH₂ | | |
| 75 | MeCO-Pen*-N-T-7MeW-K(Ac)-Pen*-AEF-2Nal- | 1133.2 | 24 |
| | THP-K(gEC16)-N-3Pya-Sar-CONH₂ | | |
| 76 | MeCO-K(PEG2PEG2gEC16OH)-Pen*- | 1729.2 | 9 |
| | K(PEG2PEG2gEC16OH)-T-7MeW-K(Ac)-Pen*- | | |
| | AEF-2Nal-THP-K(Ac)-N-3Pya-Sar-CONH₂ | | |
| 77 | MeCO-K(PEG2PEG2gEC18OH)-Pen*- | 1758 | 9 |
| | K(PEG2PEG2gEC18OH)-T-7MeW-K(Ac)-Pen*- | | |
| | AEF-2Nal-THP-K(Ac)-N-3Pya-Sar-CONH₂ | | |
| 78 | MeCO-K(PEG2PEG2gEC20OH)-Pen*- | 1785.9 | 9 |
| | K(PEG2PEG2gEC20OH)-T-7MeW-K(Ac)-Pen*- | | |
| | AEF-2Nal-THP-K(Ac)-N-3Pya-Sar-CONH₂ | | |
| 79 | MeCO-K(gEC16)-Pen*-K(gEC16)-T-7MeW-K(Ac)- | 1409 | 9 |
| | Pen*-AEF-2Nal-THP-K(Ac)-N-3Pya-Sar-CONH₂ | | |
| 80 | MeCO-K(gEC18)-Pen*-K(gEC18)-T-7MeW-K(Ac)- | 1437.4 | 9 |
| | Pen*-AEF-2Nal-THP-K(Ac)-N-3Pya-Sar-CONH₂ | | |
| 81 | MeCO-K(PEG2PEG2gEC16)-Pen*-N-T-7MeW- | 1363.4 | 25 |
| | K(Ac)-Pen*-AEF-2Nal-THP-K(Ac)-N-3Pya-Sar- | | |
| | CONH₂ | | |
| 82 | MeCO-K(PEG2PEG2gEC18)-Pen*-N-T-7MeW- | 1377.5 | 25 |
| | K(Ac)-Pen*-AEF-2Nal-THP-K(Ac)-N-3Pya-Sar- | | |
| | CONH₂ | | |
| 83 | MeCO-Pen*-K(PEG2PEG2gEC16)-T-7MeW- | 1306.5 | 2 |
| | K(Ac)-Pen*-AEF-2Nal-THP-K(Ac)-N-3Pya-Sar- | | |
| | CONH₂ | | |
| 84 | MeCO-Pen*-K(PEG2PEG2gEC18)-T-7MeW- | 1320.4 | 2 |
| | K(Ac)-Pen*-AEF-2Nal-THP-K(Ac)-N-3Pya-Sar- | | |
| | CONH₂ | | |
| 85 | MeCO-Pen*-K(PEG2PEG2gEC16OH)-T-7MeW- | 881.3 | 2 |
| | K(Ac)-Pen*-AEF-2Nal-THP-K(Ac)-N-3Pya-Sar- | | |
| | CONH₂ | | |
| 86 | MeCO-K(PEG2gEC18OH)-Pen*-N-T-7MeW- | 1379.4 | 13 |
| | K(Ac)-Pen*-AEF-2Nal-THP-K(Ac)-N-3Pya- | | |
| | N(4AmBenzyl)Gly-CONH₂ | | |
| 87 | MeCO-K(gEC18OH)-Pen*-N-T-7MeW-K(Ac)- | 1306.9 | 13 |
| | Pen*-AEF-2Nal-THP-K(Ac)-N-3Pya- | | |
| | N(4AmBenzyl)Gly-CONH₂ | | |
| 88 | MeCO-r-Pen*-N-T-7MeW- | 1385.8 | 2 |
| | K(PEG2PEG2gEC18OH)-Pen*-AEF-2Nal-THP- | | |
| | K(Ac)-N-3Pya-Sar-CONH₂ | | |
| 89 | MeCO-Pen*-N-T-7MeW-K(Ac)-Pen*-AEF-2Nal- | 1176.3 | 24 |
| | THP-K(gEC20OH)-N-3Pya-Sar-CONH₂ | | |
| 90 | MeCO-Pen*-N-T-7MeW-K(Ac)-Pen*-AEF-2Nal- | 1376.8 | 24 |
| | THP-K(PEG2PEG2TrxgEC18OH)-N-3Pya-Sar- | | |
| | CONH₂ | | |
| 91 | MeCO-Pen*-N-T-7MeW-K(Ac)-Pen*-AEF-2Nal- | 1460.3 | 24 |
| | THP-K(PEG2PEG2TrxgETrxC20OH)-N-3Pya-Sar- | | |
| | CONH₂ | | |
| 92 | MeCO-Pen*-N-T-7MeW-K(Ac)-Pen*-AEF-2Nal- | 927.6 | 24 |
| | THP-K(PEG2PEG2TrxgEC20OH)-N-3Pya-Sar- | | |
| | CONH₂ | | |
| 93 | MeCO-K(PEG2PEG2gEC10OH)-Pen*-N-T-7MeW- | 1499.1 | 10 |
| | K(Ac)-Pen*-AEF-2Nal-THP-K(gEC16)-N-3Pya- | | |
| | Sar-CONH₂ | | |
| 94 | MeCO-K(PEG2PEG2gEC18OH)-Pen*-K(gEc16)-T- | 1583.1 | 10 |
| | 7MeW-K(Ac)-Pen*-AEF-2Nal-THP-K(Ac)-N-3Pya- | | |
| | Sar-CONH₂ | | |
| 95 | MeCO-r-Pen*-K(PEG2PEG2PgEC18OH)-T-7MeW- | 975.1 | 2 |
| | K(Ac)-Pen*-AEF-2Nal-THP-K(Ac)-N-3Pya-Sar- | | |
| | CONH₂ | | |
| 96 | MeCO-r-Pen*-K(PEG2PEG2pgEC18OH)-T-7MeW- | 975.2 | 2 |
| | K(Ac)-Pen*-AEF-2Nal-THP-K(Ac)-N-3Pya-Sar- | | |
| | CONH₂ | | |
| 97 | MeCO-Pen*-N-T-7MeW-K(Ac)-Pen*-AEF-2Nal- | 1304.4 | 24 |
| | THP-K(PEG2PEG2gEmXOH)-N-3Pya-Sar-CONH₂ | | |
| 98 | MeCO-K(PEG2PEG2gEC18OH)-Pen*-N-T-7MeW- | 1555.1 | 10 |
| | K(Ac)-Pen*-AEF-2Nal-THP-K(gEc16)-N-3Pya-Sar- | | |
| | CONH₂ | | |
| 99 | MeCO-r-Pen*-K(PEG2PEG2PPPgEC18OH)-T- | 1039.7 | 2 |
| | 7MeW-K(Ac)-Pen*-AEF-2Nal-THP-K(Ac)-N-3Pya- | | |
| | Sar-CONH₂ | | |
| 100 | MeCO-r-Pen*-K(PEG2PEG2pppgEC18OH)-T- | 1040 | 2 |
| | 7MeW-K(Ac)-Pen*-AEF-2Nal-THP-K(Ac)-N-3Pya- | | |
| | Sar-CONH2 | | |
| 101 | MeCO-K(PEG2PEG2PgEC18OH)-Pen*-N-T- | 961.1 | 25 |
| | 7MeW-K(Ac)-Pen*-AEF-2Nal-THP-K(Ac)-N-3Pya- | | |
| | Sar-CONH2 | | |
| 102 | MeCO-K(PEG2PEG2pgEC18OH)-Pen*-N-T- | 961.1 | 25 |
| | 7MeW-K(Ac)-Pen*-AEF-2Nal-THP-K(Ac)-N-3Pya- | | |
| | Sar-CONH₂ | | |
| 103 | MeCO-K(PEG2PEG2PPPgEC18OH)-Pen*-N-T- | 1025.8 | 25 |
| | 7MeW-K(Ac)-Pen*-AEF-2Nal-THP-K(Ac)-N-3Pya- | | |
| | Sar-CONH₂ | | |
| 104 | MeCO-K(PEG2PEG2pppgEC18OH)-Pen*-N-T- | 1025.9 | 25 |
| | 7MeW-K(Ac)-Pen*-AEF-2Nal-THP-K(Ac)-N-3Pya- | | |
| | Sar-CONH₂ | | |
| 105 | MeCO-Pen*-N-T-7MeW-K(Ac)-Pen*-AEF-2Nal- | 1356.1 | 24 |
| | THP-K(PEG2PEG2PgEC18OH)-N-3Pya-Sar- | | |
| | CONH₂ | | |
| 106 | MeCO-Pen*-N-T-7MeW-K(Ac)-Pen*-AEF-2Nal- | 1356 | 24 |
| | THP-K(PEG2PEG2pgEC18OH)-N-3Pya-Sar- | | |
| | CONH2 | | |
| 107 | MeCO-Pen*-N-T-7MeW-K(Ac)-Pen*-AEF-2Nal- | 1453.1 | 24 |
| | THP-K(PEG2PEG2PPPgEC18OH)-N-3Pya-Sar- | | |
| | CONH₂ | | |
| 108 | MeCO-Pen*-N-T-7MeW-K(Ac)-Pen*-AEF-2Nal- | 1453 | 24 |
| | THP-K(PEG2PEG2pppgEC18OH)-N-3Pya-Sar- | | |
| | CONH₂ | | |
| 109 | MeCO-K(PEG2PEG2TrxgEC18OH)-Pen*-N-T- | 1461.7 | 25 |
| | 7MeW-K(Ac)-Pen*-AEF-2Nal-THP-K(Ac)-N-3Pya- | | |
| | Sar-CONH₂ | | |
| 110 | MeCO-K(PEG2PEG2gEmXOH)-Pen*-N-T-7MeW- | 1389.5 | 25 |
| | K(Ac)-Pen*-AEF-2Nal-THP-K(Ac)-N-3Pya-Sar- | | |
| | CONH₂ | | |
| 111 | MeCO-Pen*-N-T-7MeW-K(Ac)-Pen*-AEF-2Nal- | 1304.5 | 24 |
| | THP-K(PEG2PEG2gEpXOH)-N-3Pya-Sar-CONH₂ | | |
| 112 | MeCO-K(PEG2PEG2gEpXOH)-Pen*-N-T-K(Ac)- | 1149.2 | 9 |
| | Pen*-AEF-2Nal-THP-K(PEG2PEG2gEpXOH)-N- | | |
| | 3Pya-Sar-CONH₂ | | |
| 113 | MeCO-K(PEG2PEG2gEmXOH)-Pen*-N-T-7MeW- | 1552.1 | 10 |
| | K(Ac)-Pen*-AEF-2Nal-THP-K(gEC16)-N-3Pya- | | |
| | Sar-CONH₂ | | |
| 114 | MeCO-Pen*-N-T-7MeW-K(Ac)-Pen*-AEF-2Nal- | 1260.7 | 15 |
| | THP-K(DAP-(C16OH)2)-N-3Pya-Sar-CONH₂ | | |
| 115 | MeCO-K(DAP(C16OH)2)-Pen*-N-T-7MeW-K(Ac)- | 1346 | 15 |
| | Pen*-AEF-2Nal-THP-K(Ac)-N-3Pya-Sar-CONH₂ | | |
| 116 | MeCO-Pen*-N-T-7MeW-K(Ac)-Pen*-AEF-2Nal- | 1470.3 | 15 |
| | THP-K(PEG2PEG2gE-DAP(C16OH)2)-N-3Pya- | | |
| | Sar-CONH₂ | | |
| 117 | MeCO-K(PEG2PEG2gEDAP(C16OH)2)-Pen*-N-T- | 1037.3 | 15 |
| | 7MeW-K(Ac)-Pen*-AEF-2Nal-THP-K(Ac)-N-3Pya- | | |
| | Sar-CONH₂ | | |
| 118 | MeCO-Pen*-N-T-7MeW-K(Ac)-Pen*-AEF-2Nal- | 1371.2 | 7 |
| | THP-K(PEG2PEG2SP6gEC18OH)-N-3Pya-Sar- | | |
| | CONH₂ | | |
| 119 | MeCO-Pen*-N-T-7MeW-K(Ac)-Pen*-AEF-2Nal- | 1371 | 7 |
| | THP-K(PEG2SP6PEG2gEC18OH)-N-3Pya-Sar- | | |
| | CONH₂ | | |
| 120 | MeCO-K(PEG2PEG2gEC16OH)-Pen*-N-T-7MeW- | 1555.4 | 10 |
| | K(Ac)-Pen*-AEF-2Nal-THP-K(gEC18)-N-3Pya- | | |
| | Sar-CONH₂ | | |
| 121 | MeCO-K(PEG2PEG2gEC18OH)-Pen*-N-T-7MeW- | 1568.8 | 10 |
| | K(Ac)-Pen*-AEF-2Nal-THP-K(gEC18)-N-3Pya- | | |
| | Sar-CONH₂ | | |
| 122 | MeCO-Pen*-K(PEG2PEG2gEC18OH)-T-7MeW- | 1335.2 | 2 |
| | K(Ac)-Pen*-AEF-2Nal-THP-K(Ac)-N-3Pya-Sar- | | |
| | CONH₂ | | |
| 123 | MeCO-Pen*-N-T-7MeW-K(Ac)-Pen*-AEF-2Nal- | 1371.5 | 7 |
| | THP-K(PEG2PEG2gESP6C18OH)-N-3Pya-Sar- | | |
| | CONH₂ | | |
| 124 | MeCO-Pen*-N-T-7MeW-K(Ac)-Pen*-AEF-2Nal- | 1371 | 7 |
| | THP-K(SP6PEG2PEG2gEC18OH)-N-3Pya-Sar- | | |
| | CONH₂ | | |
| 125 | MeCO-K(gEC16)-Pen*-N-T-7MeW-K(Ac)-Pen*- | 1394.6 | 10 |
| | AEF-2Nal-THP-K(gEC18)-N-3Pya-Sar-CONH₂ | | |
| 126 | MeCO-K(gEC18)-Pen*-N-T-7MeW-K(Ac)-Pen*- | 1408.9 | 19 |
| | AEF-2Nal-THP-K(gEC18)-N-3Pya-Sar-CONH₂ | | |
| 127 | MeCO-K(PEG2PEG2gEC10OH)-Pen*-N-T-7MeW- | 1513.4 | 10 |
| | K(Ac)-Pen*-AEF-2Nal-THP-K(gEC18)-N-3Pya- | | |
| | Sar-CONH₂ | | |
| 128 | MeCO-Pen*-N-T-7MeW-K(Ac)-Pen*-AEF-2Nal- | 1555.4 | 24 |
| | THP-K(PEG2PEG2gETrxC20OH)-N-3Pya-Sar- | | |
| | CONH₂ | | |
| 129 | MeCO-r-Pen*-N-T-7MeW-K(Ac)-Pen*-AEF-2Nal- | 1392.4 | 8 |
| | THP-K(Ac)-N-3Pya-Sar-PEG2PEG2gDabC18OH- | | |
| | COOH | | |
| 130 | MeCO-K(PEG2PEG2gESP6C18OH)-Pen*-N-T- | 1456.9 | 7 |
| | 7MeW-K(Ac)-Pen*-AEF-2Nal-THP-K(Ac)-N-3Pya- | | |
| | Sar-CONH₂ | | |
| 131 | MeCO-K(PEG2SP6PEG2gEC18OH)-Pen*-N-T- | 1456.55 | 7 |
| | 7MeW-K(Ac)-Pen*-AEF-2Nal-THP-K(Ac)-N-3Pya- | | |
| | Sar-CONH₂ | | |
| 132 | MeCO-K(SP6PEG2PEG2gEC18OH)-Pen*-N-T- | 1456.69 | 7 |
| | 7MeW-K(Ac)-Pen*-AEF-2Nal-THP-K(Ac)-N-3Pya- | | |
| | Sar-CONH₂ | | |
| 133 | MeCO-K[PEG2PEG2gEDAP(pXOH)2]-Pen*-N-T- | 1578.4 | 15 |
| | 7MeW-K(Ac)-Pen*-AEF-2Nal-THP-K(Ac)-N-3Pya- | | |
| | Sar-CONH₂ | | |
| 134 | MeCO-Pen*-N-T-7MeW-K(Ac)-Pen*-AEF-2Nal- | 1492.9 | 15 |
| | THP-K(PEG2PEG2gEDAP(mXOH)2)-N-3Pya-Sar- | | |
| | CONH₂ | | |
| 135 | MeCO-K(GolAC16)-Pen*-N-T-7MeW-K(Ac)-Pen*- | 1212.8 | 25 |
| | AEF-2Nal-THP-K(Ac)-N-3Pya-Sar-CONH₂ | | |
| 136 | MeCO-K(GolAC16OH)-Pen*-N-T-7MeW-K(Ac)- | 1228.3 | 25 |
| | Pen*-AEF-2Nal-THP-K(Ac)-N-3Pya-Sar-CONH₂ | | |
| 137 | MeCO-K(GolAC18OH)-Pen*-N-T-7MeW-K(Ac)- | 1241.8 | 25 |
| | Pen*-AEF-2Nal-THP-K(Ac)-N-3Pya-Sar-CONH₂ | | |
| 138 | MeCO-K(PEG2PEG2GolAC18OH)-Pen*-N-T- | 1386.55 | 25 |
| | 7MeW-K(Ac)-Pen*-AEF-2Nal-THP-K(Ac)-N-3Pya- | | |
| | Sar-CONH₂ | | |
| 139 | MeCO-K(PEG2PEG2gEC18OH(c)-Pen*-N-T- | 1463.76 | 25 |
| | 7MeW-K(Ac)-Pen*-AEF-2Nal-THP-K(Ac)-N-3Pya- | | |
| | Sar-CONH₂ | | |
| 140 | MeCO-K(PEG2PEG2C18GolB)-Pen*-N-T-7MeW- | 1365.2 | 25 |
| | K(Ac)-Pen*-AEF-2Nal-THP-K(Ac)-N-3Pya-Sar- | | |
| | CONH₂ | | |
| 141 | MeCO-K(PEG2PEG2gE(C)C18OH-Pen*-N-T- | 1463.9 | 16 |
| | 7MeW-K(Ac)-Pen*-AEF-2Nal-THP-K(Ac)-N-3Pya- | | |
| | Sar-CONH₂ | | |
| 142 | MeCO-K(PEG2PEG2gEC18OH(C)-Pen*-N-T- | 1463.69 | 25 |
| | 7MeW-K(Ac)-Pen*-AEF-2Nal-THP-K(Ac)-N-3Pya- | | |
| | Sar-CONH₂ | | |

### Examples 201-492. Compounds

Additional compounds of the invention as shown in Table 4 below were prepared.

TABLE 4
Compounds

| Example | Name |
|---|---|
| 201 | Ac-[Pen]-L-T-[Trp(7-Me)]-[Lys(PEG12_OMe)]-[Pen]-[Phe(4-OMe)]-[2Nal]- |
| | [aMeLeu]-L-N-[3Pal]-[Sarc]-NH2 |
| 202 | Ac-[Pen]-N-T-[Trp(7-Me)]-[Lys(PEG12_OMe)]-[Pen]-[Phe(4-OMe)]-[2Nal]- |
| | [aMeLeu]-L-N-[3Pal]-[Sarc]-NH2 |
| 203 | Ac-[Lys(PEG12_OMe)]-[Pen]-L-T-[Trp(7-Me)]-[Lys(Ac)]-[Pen]-[Phe(4- |
| | OMe)]-[2Nal]-[aMeLeu]-L-N-[3Pal]-[Sarc]-NH2 |
| 204 | Ac-[Lys(PEG12_OMe)]-[Pen]-N-T-[Trp(7-Me)]-[Lys(Ac)]-[Pen]-[Phe(4- |
| | OMe)]-[2Nal]-[aMeLeu]-L-N-[3Pal]-[Sarc]-NH2 |
| 205 | Ac-[Lys(PEG12_OMe)]-[Pen]-N-T-[Trp(7-Me)]-[Lys(Ac)]-[Pen]-F-[2Nal]- |
| | [aMeLeu]-L-N-[3Pal]-[Sarc]-NH2 |
| 206 | Ac-[Lys(PEG12_OMe)]-[Pen]-L-T-[Trp(7-Me)]-[Lys(Ac)]-[Pen]-F-[2Nal]- |
| | [aMeLeu]-L-N-[3Pal]-[Sarc]-NH2 |
| 207 | [PEG12_OMe]-[Pen]-N-T-[Trp(7-Me)]-[Lys(Ac)]-[Pen]-F-[2Nal]-[aMeLeu]- |
| | L-N-[3Pal]-[Sarc]-NH2 |
| 208 | Ac-[Pen]-N-T-[Trp(7-Me)]-[Lys(PEG12_OMe)]-[Pen]-F-[2Nal]-[aMeLeu]-L- |
| | N-[3Pal]-[Sarc]-NH2 |
| 209 | [PEG12_OMe]-[Pen]-L-T-[Trp(7-Me)]-[Lys(Ac)]-[Pen]-F-[2Nal]-[aMeLeu]- |
| | L-N-[3Pal]-[Sarc]-NH2 |
| 210 | Ac-[Pen]-L-T-[Trp(7-Me)]-[Lys(PEG12_OMe)]-[Pen]-F-[2Nal]-[aMeLeu]-L- |
| | N-[3Pal]-[Sarc]-NH2 |
| 211 | [PEG4_OMe]-[Pen]-N-T-[Trp(7-Me)]-[Lys(Ac)]-[Pen]-F-[2Nal]-[aMeLeu]-L- |
| | N-[3Pal]-[Sarc]-NH2 |
| 212 | Ac-[Pen]-N-T-[Trp(7-Me)]-[Lys(PEG4)]-[Pen]-F-[2Nal]-[aMeLeu]-L-N- |
| | [3Pal]-[Sarc]-NH2 |
| 213 | [PEG4_OMe]-[Pen]-L-T-[Trp(7-Me)]-[Lys(Ac)]-[Pen]-F-[2Nal]-[aMeLeu]-L- |
| | N-[3Pal]-[Sarc]-NH2 |
| 214 | Ac-[Pen]-L-T-[Trp(7-Me)]-[Lys(PEG4)]-[Pen]-F-[2Nal]-[aMeLeu]-L-N- |
| | [3Pal]-[Sarc]-NH2 |
| 215 | Ac-[Lys(PEG4)]-[Pen]-N-T-[Trp(7-Me)]-[Lys(Ac)]-[Pen]-F-[2Nal]-[aMeLeu]- |
| | L-N-[3Pal]-[Sarc]-NH2 |
| 216 | Ac-[Lys(PEG4)]-[Pen]-L-T-[Trp(7-Me)]-[Lys(Ac)]-[Pen]-F-[2Nal]-[aMeLeu]- |
| | L-N-[3Pal]-[Sarc]-NH2 |
| 217 | Ac-[Pen]-N-T-[Trp(7-Me)]-[Lys(Ac)]-[Pen]-[Phe(4-OMe)]-[2Nal]- |
| | [aMeLys(PEG12_IsoGlu_Palm)]-E-L-[3Pal]-[Sarc]-NH2 |
| 218 | Ac-[Pen]-N-T-[Trp(7-Me)]-[Lys(Ac)]-[Pen]-[Phe(4-OMe)]-[2Nal]- |
| | [aMeLys(PEG12_IsoGlu_Palm)]-[Lys(Ac)]-L-[3Pal]-[Sarc]-NH2 |
| 219 | Ac-[Pen]-N-T-[Trp(7-Me)]-[Lys(Ac)]-[Pen]-[2Nal]-[2Nal]- |
| | [aMeLys(PEG12_IsoGlu_Palm)]-E-L-[3Pal]-[Sarc]-NH2 |
| 220 | Ac-[Pen]-N-T-[Trp(7-Me)]-[Lys(Ac)]-[Pen]-[2Nal]-[2Nal]- |
| | [aMeLys(PEG12_IsoGlu_Palm)]-[Lys(Ac)]-L-[3Pal]-[Sarc]-NH2 |
| 221 | Ac-[Pen]-N-T-[Trp(7-Me)]-[Lys(Ac)]-[Pen]-F-[2Nal]- |
| | [aMeLys(PEG12_IsoGlu_Palm)]-L-L-[3Pal]-[Sarc]-NH2 |
| 222 | Ac-[Pen]-N-T-[Trp(7-Me)]-[Lys(Ac)]-[Pen]-F-[2Nal]- |
| | [aMeLys(PEG12_IsoGlu_Palm)]-E-L-[3Pal]-[Sarc]-NH2 |
| 223 | Ac-[Pen]-A-T-[Trp(7-Me)]-[Lys(Ac)]-[Pen]-[Phe(4-OMe)]-[2Nal]- |
| | [aMeLys(PEG12_IsoGlu_Palm)]-A-A-[3Pal]-[Sarc]-NH2 |
| 224 | Ac-[Pen]-A-T-[Trp(7-Me)]-[Lys(Ac)]-[Pen]-[Phe(4-OMe)]-[2Nal]- |
| | [aMeLys(PEG12_IsoGlu_C18_Diacid)]-A-A-[3Pal]-[Sarc]-NH2 |
| 225 | Ac-[Pen]-L-T-[Trp(7-Me)]-[Lys(PEG12_OMe)]-[Pen]-[Phe(4-OMe)]-[2Nal]- |
| | [aMeLeu]-L-N-[3Pal]-[Sarc]-NH2 |
| 226 | Ac-[Pen]-N-T-[Trp(7-Me)]-[Lys(Ac)]-[Pen]-[Phe(4-OMe)]-[2Nal]- |
| | [aMeLys(PEG12_IsoGlu_Palm)]-A-A-[3Pal]-[Sarc]-NH2 |
| 227 | Ac-[Pen]-A-T-[Trp(7-Me)]-[Lys(Ac)]-[Pen]-[Phe(4-OMe)]-[2Nal]- |
| | [aMeLys(PEG12_IsoGlu_Palm)]-[Lys(Ac)]-N-[3Pal]-[Sarc]-NH2 |
| 228 | Ac-[Pen]-A-T-[Trp(7-Me)]-[Lys(Ac)]-[Pen]-[Phe(4-OMe)]-[2Nal]- |
| | [aMeLys(PEG12_IsoGlu_C18_Diacid)]-[Lys(Ac)]-N-[3Pal]-[Sarc]-NH2 |
| 229 | Ac-[Pen]-N-T-[Trp(7-Me)]-[Lys(Ac)]-[Pen]-[Phe(4-(2- |
| | (1PEG2 1PEG2_IsoGlu_Palm)aminoethoxy))]-[2Nal]-[THP]-[Lys(Ac)]-L- |
| | [3Pal]-[Sarc]-NH2 |
| 230 | Ac-[Pen]-N-T-[Trp(7-Me)]-[Lys(Ac)]-[Pen]-[Phe(4-(2- |
| | (1PEG2_1PEG2_IsoGlu_C18_Diacid)aminoethoxy))]-[2Nal]-[THP]- |
| | [Lys(Ac)]-L-[3Pal]-[Sarc]-NH2 |
| 231 | Ac-[Pen]-N-T-[Trp(7-Me)]-[Lys(Ac)]-[Pen]-[Phe(4-(2- |
| | (PEG4_PEG4_IsoGlu_Palm)aminoethoxy))]-[2Nal]-[THP]-[Lys(Ac)]-L- |
| | [3Pal]-[Sarc]-NH2 |
| 232 | Ac-[Pen]-N-T-[Trp(7-Me)]-[Lys(Ac)]-[Pen]-[Phe(4-(2- |
| | (PEG12_IsoGlu_Palm)aminoethoxy))]-[2Nal]-[THP]-[Lys(Ac)]-L-[3Pal]- |
| | [Sarc]-NH2 |
| 233 | Ac-[Pen]-N-T-[Trp(7-Me)]-[Lys(Ac)]-[Pen]-[Phe(4-OMe)]-[2Nal]- |
| | [Lys(PEG12_IsoGlu_Palm)]-[Lys(Ac)]-N-[3Pal]-[Sarc]-NH2 |
| 234 | Ac-[Pen]-N-T-[Trp(7-Me)]-[Lys(Ac)]-[Pen]-F-[2Nal]- |
| | [Spiral_Pip_PEG12_IsoGlu_Palm]-[Lys(Ac)]-N-[3Pal]-[Sarc]-NH2 |
| 235 | Ac-[Pen]-N-T-[Trp(7-Me)]-[Lys(Ac)]-[Pen]-F-[2Nal]- |
| | [aMeLys(PEG12_IsoGlu_Palm)]-[Lys(Ac)]-L-[3Pal]-[Sarc]-NH2 |
| 236 | Ac-[Pen]-N-T-[Trp(7-Me)]-[Lys(Ac)]-[Pen]-[Phe(4-OMe)]-[2Nal]- |
| | [aMeLys(PEG12_IsoGlu_C18_Diacid)]-A-A-[3Pal]-[Sarc]-NH2 |
| 237 | Ac-[Pen]-L-T-[Trp(7-Me)]-[Lys(Ac)]-[Pen]-[Phe(4-OMe)]-[2Nal]-[THP]- |
| | [Lys(Ac)]-L-[3Pal]-[(D)Lys(PEG12_C18_Diacid)]-NH2 |
| 238 | Ac-[Pen]-L-T-[Trp(7-Me)]-[Lys(Ac)]-[Pen]-[Phe(4-OMe)]-[2Nal]-[THP]- |
| | [Lys(Ac)]-L-[3Pal]-[(D)Lys(PEG12_IsoGlu_Palm)]-NH2 |
| 239 | Ac-[Pen]-L-T-[Trp(7-Me)]-[Lys(Ac)]-[Pen]-[Phe(4-OMe)]-[2Nal]-[THP]- |
| | [Lys(Ac)]-L-[3Pal]-[(D)Lys(PEG12_IsoGlu_C18_Diacid)]-NH2 |
| 240 | Ac-[Pen]-L-T-[Trp(7-Me)]-[Lys(Ac)]-[Pen]-[Phe(4-OMe)]-[2Nal]-[THP]- |
| | [Lys(Ac)]-[Lys(PEG12_C18_Diacid)]-[3Pal]-[Sarc]-NH2 |
| 241 | Ac-[Pen]-A-T-[Trp(7-Me)]-[Lys(Ac)]-[Pen]-[Phe(4-OMe)]-[2Nal]- |
| | [aMeLys(PEG12_IsoGlu_Palm)]-[Lys(Ac)]-A-[3Pal]-[Sarc]-NH2 |
| 242 | Ac-[Pen]-A-T-[Trp(7-Me)]-[Lys(Ac)]-[Pen]-[Phe(4-OMe)]-[2Nal]- |
| | [aMeLys(PEG12_IsoGlu_C18_Diacid)]-[Lys(Ac)]-A-[3Pal]-[Sarc]-NH2 |
| 243 | Ac-[Pen]-L-T-[Trp(7-Me)]-[Lys(Ac)]-[Pen]-[Phe(4-OMe)]-[2Nal]-[THP]- |
| | [Lys(PEG12_C18_Diacid)]-L-[3Pal]-[Sarc]-NH2 |
| 244 | Ac-[Pen]-L-T-[Trp(7-Me)]-[Lys(Ac)]-[Pen]-[Phe(4-OMe)]-[2Nal]-[THP]- |
| | [Lys(PEG12_IsoGlu_Palm)]-L-[3Pal]-[Sarc]-NH2 |
| 245 | Ac-[Pen]-L-T-[Trp(7-Me)]-[Lys(Ac)]-[Pen]-[Phe(4-OMe)]-[2Nal]-[THP]- |
| | [Lys(PEG12_IsoGlu_C18_Diacid)]-L-[3Pal]-[Sarc]-NH2 |
| 246 | Ac-[Pen]-L-[Lys(PEG12_C18_Diacid)]-[Trp(7-Me)]-[Lys(Ac)]-[Pen]-[Phe(4- |
| | OMe)]-[2Nal]-[THP]-[Lys(Ac)]-L-[3Pal]-[Sarc]-NH2 |
| 247 | Ac-[Pen]-L-[Lys(PEG12_IsoGlu_Palm)]-[Trp(7-Me)]-[Lys(Ac)]-[Pen]-[Phe(4- |
| | OMe)]-[2Nal]-[THP]-[Lys(Ac)]-L-[3Pal]-[Sarc]-NH2 |
| 248 | Ac-[Pen]-L-[Lys(PEG12_IsoGlu_C18_Diacid)]-[Trp(7-Me)]-[Lys(Ac)]-[Pen]- |
| | [Phe(4-OMe)]-[2Nal]-[THP]-[Lys(Ac)]-L-[3Pal]-[Sarc]-NH2 |
| 249 | Ac-[Pen]-N-T-[Trp(7-Me)]-[Lys(Ac)]-[Pen]-[Phe(4-OMe)]-[2Nal]- |
| | [aMeLys(PEG12_IsoGlu_Palm)]-[Lys(Ac)]-A-[3Pal]-[Sarc]-NH2 |
| 250 | Ac-[Pen]-[Lys(PEG12_IsoGlu_Palm)]-T-[Trp(7-Me)]-[Lys(Ac)]-[Pen]-[Phe(4- |
| | OMe)]-[2Nal]-[THP]-[Lys(Ac)]-L-[3Pal]-[Sarc]-NH2 |
| 251 | Ac-[Pen]-[Lys(PEG12_IsoGlu_C18_Diacid)]-T-[Trp(7-Me)]-[Lys(Ac)]-[Pen]- |
| | [Phe(4-OMe)]-[2Nal]-[THP]-[Lys(Ac)]-L-[3Pal]-[Sarc]-NH2 |
| 252 | [Pen(PEG4_Ahx_C18_Diacid)]-N-T-[Trp(7-Me)]-[Lys(Ac)]-[Pen]-[Phe(4- |
| | OMe)]-[2Nal]-[THP]-E-L-[3Pal]-[Sarc]-NH2 |
| 253 | [Pen(PEG4_IsoGlu_C18_Diacid)]-N-T-[Trp(7-Me)]-[Lys(Ac)]-[Pen]-[Phe(4- |
| | OMe) ]-[2Nal]-[THP]-E-L-[3Pal]-[Sarc]-NH2 |
| 254 | Ac-[(D)Lys(PEG12_IsoGlu_Palm)]-[Pen]-L-T-[Trp(7-Me)]-[Lys(Ac)]-[Pen]- |
| | [Phe(4-OMe)]-[2Nal]-[THP]-L-L-[3Pal]-[Sarc]-NH2 |
| 255 | Ac-[(D)Lys(PEG12_IsoGlu_C18_Diacid)]-[Pen]-L-T-[Trp(7-Me)]-[Lys(Ac)]- |
| | [Pen]-[Phe(4-OMe)]-[2Nal]-[THP]-L-L-[3Pal]-[Sarc]-NH2 |
| 256 | Ac-[(D)Lys(PEG12_C18_Diacid)]-[Pen]-L-T-[Trp(7-Me)]-[Lys(Ac)]-[Pen]- |
| | [Phe(4-OMe)]-[2Nal]-[THP]-[Lys(Ac)]-L-[3Pal]-[Sarc]-NH2 |
| 257 | Ac-[(D)Lys(Peg4_C18_Diacid)]-[Pen]-L-T-[Trp(7-Me)]-[Lys(Ac)]-[Pen]- |
| | [Phe(4-OMe)]-[2Nal]-[THP]-[Lys(Ac)]-L-[3Pal]-[Sarc]-NH2 |
| 258 | Ac-[(D)Lys(IsoGlu_C18_Diacid)]-[Pen]-L-T-[Trp(7-Me)]-[Lys(Ac)]-[Pen]- |
| | [Phe(4-OMe)]-[2Nal]-[THP]-[Lys(Ac)]-L-[3Pal]-[Sarc]-NH2 |
| 259 | Ac-[(D)Lys(Peg4_IsoGlu_C18_Diacid)]-[Pen]-L-T-[Trp(7-Me)]-[Lys(Ac)]- |
| | [Pen]-[Phe(4-OMe)]-[2Nal]-[THP]-[Lys(Ac)]-L-[3Pal]-[Sarc]-NH2 |
| 260 | Ac-[(D)Lys(PEG12_IsoGlu_Palm)]-[Pen]-L-T-[Trp(7-Me)]-[Lys(Ac)]-[Pen]- |
| | [Phe(4-OMe)]-[2Nal]-[THP]-[Lys(Ac)]-L-[3Pal]-[Sarc]-NH2 |
| 261 | Ac-[(D)Lys(PEG12_IsoGlu_C18_Diacid)]-[Pen]-L-T-[Trp(7-Me)]-[Lys(Ac)]- |
| | [Pen]-[Phe(4-OMe)]-[2Nal]-[THP]-[Lys(Ac)]-L-[3Pal]-[Sarc]-NH2 |
| 262 | Ac-[Pen]-L-T-[Trp(7-Me)]-[Lys(PEG12_C18_Diacid)]-[Pen]-[Phe(4-OMe)]- |
| | [2Nal]-[THP]-L-L-[3Pal]-[Sarc]-NH2 |
| 263 | Ac-[Pen]-L-T-[Trp(7-Me)]-[Lys(PEG4_C18_Diacid)]-[Pen]-[Phe(4-OMe)]- |
| | [2Nal]-[THP]-L-L-[3Pal]-[Sarc]-NH2 |
| 264 | Ac-[Pen]-L-T-[Trp(7-Me)]-[Lys(IsoGlu_C18_Diacid)]-[Pen]-[Phe(4-OMe)]- |
| | [2Nal]-[THP]-L-L-[3Pal]-[Sarc]-NH2 |
| 265 | Ac-[Pen]-L-T-[Trp(7-Me)]-[Lys(IsoGlu_Palm)]-[Pen]-[Phe(4-OMe)]-[2Nal]- |
| | [THP]-L-L-[3Pal]-[Sarc]-NH2 |
| 266 | Ac-[Pen]-L-T-[Trp(7-Me)]-[Lys(PEG4_IsoGlu_Palm)]-[Pen]-[Phe(4-OMe)]- |
| | [2Nal]-[THP]-L-L-[3Pal]-[Sarc]-NH2 |
| 267 | Ac-[Pen]-L-T-[Trp(7-Me)]-[Lys(PEG4_IsoGlu_C18_Diacid)]-[Pen]-[Phe(4- |
| | OMe)]-[2Nal]-[THP]-L-L-[3Pal]-[Sarc]-NH2 |
| 268 | Ac-[Pen]-L-T-[Trp(7-Me)]-[Lys(PEG12_IsoGlu_Palm)]-[Pen]-[Phe(4-OMe)]- |
| | [2Nal]-[THP]-L-L-[3Pal]-[Sarc]-NH2 |
| 269 | Ac-[Pen]-L-T-[Trp(7-Me)]-[Lys(PEG12_IsoGlu_C18_Diacid)]-[Pen]-[Phe(4- |
| | OMe)]-[2Nal]-[THP]-L-L-[3Pal]-[Sarc]-NH2 |
| 270 | Ac-[Pen]-L-T-[Trp(7-Me)]-[Lys(PEG12_C18_Diacid)]-[Pen]-[Phe(4-OMe)]- |
| | [2Nal]-[THP]-[Lys(Ac)]-L-[3Pal]-[Sarc]-NH2 |
| 271 | Ac-[Pen]-L-T-[Trp(7-Me)]-[Lys(PEG4_C18_Diacid)]-[Pen]-[Phe(4-OMe)]- |
| | [2Nal]-[THP]-[Lys(Ac)]-L-[3Pal]-[Sarc]-NH2 |
| 272 | Ac-[Pen]-L-T-[Trp(7-Me)]-[Lys(IsoGlu_C18_Diacid)]-[Pen]-[Phe(4-OMe)]- |
| | [2Nal]-[THP]-[Lys(Ac)]-L-[3Pal]-[Sarc]-NH2 |
| 273 | Ac-[Pen]-L-T-[Trp(7-Me)]-[Lys(IsoGlu_Palm)]-[Pen]-[Phe(4-OMe)]-[2Nal]- |
| | [THP]-[Lys(Ac)]-L-[3Pal]-[Sarc]-NH2 |
| 274 | Ac-[Pen]-L-T-[Trp(7-Me)]-[Lys(PEG4_IsoGlu_Palm)]-[Pen]-[Phe(4-OMe)]- |
| | [2Nal]-[THP]-[Lys(Ac)]-L-[3Pal]-[Sarc]-NH2 |
| 275 | Ac-[Pen]-L-T-[Trp(7-Me)]-[Lys(PEG4_IsoGlu_C18_Diacid)]-[Pen]-[Phe(4- |
| | OMe)]-[2Nal]-[THP]-[Lys(Ac)]-L-[3Pal]-[Sarc]-NH2 |
| 276 | Ac-[Pen]-L-T-[Trp(7-Me)]-[Lys(PEG12_IsoGlu_Palm)]-[Pen]-[Phe(4-OMe)]- |
| | [2Nal]-[THP]-[Lys(Ac)]-L-[3Pal]-[Sarc]-NH2 |
| 277 | Ac-[Pen]-L-T-[Trp(7-Me)]-[Lys(PEG12_IsoGlu_C18_Diacid)]-[Pen]-[Phe(4- |
| | OMe)]-[2Nal]-[THP]-[Lys(Ac)]-L-[3Pal]-[Sarc]-NH2 |
| 278 | Ac-[Pen]-N-T-[Trp(7-Me)]-[Lys(Ac)]-[Pen]-[Phe(4-OMe)]-[2Nal]- |
| | [aMeLys(PEG12_IsoGlu_C18_Diacid)]-[Lys(Ac)]-A-[3Pal]-[Sarc]-NH2 |
| 279 | Ac-[Pen]-N-T-[Trp(7-Me)]-[Lys(Ac)]-[Pen]-[Phe(4-(2- |
| | (PEG4_PEG4_IsoGlu_C18_Diacid)aminoethoxy))]-[2Nal]-[THP]-[Lys(Ac)]- |
| | L-[3Pal]-[Sarc]-NH2 |
| 280 | Ac-[Pen]-N-T-[Trp(7-Me)]-[Lys(Ac)]-[Pen]-[Phe(4-(2- |
| | (PEG12_IsoGlu_C18_Diacid)aminoethoxy))]-[2Nal]-[THP]-[Lys(Ac)]-L- |
| | [3Pal]-[Sarc]-NH2 |
| 281 | Ac-[Pen]-N-T-[Trp(7-Me)]-[Lys(Ac)]-[Pen]-[Phe(4-OMe)]-[2Nal]- |
| | [Lys(PEG12_IsoGlu_C18_Diacid)]-[Lys(Ac)]-N-[3Pal]-[Sarc]-NH2 |
| 282 | Ac-[Pen]-L-T-[Trp(7-Me)]-[Lys(Ac)]-[Pen]-[Phe(4-OMe)]-[2Nal]-[THP]- |
| | [Lys(Ac)]-[Lys(PEG12_IsoGlu_C18_Diacid)]-[3Pal]-[Sarc]-NH2 |
| 283 | Ac-[Pen]-[Lys(PEG12_C18_Diacid)]-T-[Trp(7-Me)]-[Lys(Ac)]-[Pen]-[Phe(4- |
| | OMe)]-[2Nal]-[THP]-[Lys(Ac)]-L-[3Pal]-[Sarc]-NH2 |
| 284 | [PEG4_Decyl]-[Pen]-N-T-[Trp(7-Me)]-[Lys(Ac)]-[Pen]-[Phe(4-OMe)]-[2Nal]- |
| | [THP]-L-N-[3Pal]-[Sarc]-NH2 |
| 285 | [PEG4_Lauryl]-[Pen]-N-T-[Trp(7-Me)]-[Lys(Ac)]-[Pen]-[Phe(4-OMe)]- |
| | [2Nal]-[THP]-L-N-[3Pal]-[Sarc]-NH2 |
| 286 | Ac-[Pen]-N-T-[Trp(7-Me)]-[Lys(Ac)]-[Pen]-[Phe(4-(2-aminoethoxy))]-[2Nal]- |
| | [THP]-E-N-[3Pal]-[Sarc]-[(D)Lys(PEG12_IsoGlu_C18_Diacid)]-NH2 |
| 287 | [PEG4_Capryl]-[Pen]-N-T-[Trp(7-Me)]-[Lys(Ac)]-[Pen]-[Phe(4-OMe)]- |
| | [2Nal]-[THP]-L-N-[3Pal]-[Sarc]-NH2 |
| 288 | [PEG4_Hexyl]-[Pen]-N-T-[Trp(7-Me)]-[Lys(Ac)]-[Pen]-[Phe(4-OMe)]- |
| | [2Nal]-[THP]-L-N-[3Pal]-[Sarc]-NH2 |
| 289 | [PEG2_Palm]-[Pen]-N-T-[Trp(7-Me)]-[Lys(Ac)]-[Pen]-[Phe(4-OMe)]-[2Nal]- |
| | [THP]-L-N-[3Pal]-[Sarc]-NH2 |
| 290 | [PEG2_Myristyl]-[Pen]-N-T-[Trp(7-Me)]-[Lys(Ac)]-[Pen]-[Phe(4-OMe)]- |
| | [2Nal]-[THP]-L-N-[3Pal]-[Sarc]-NH2 |
| 291 | [PEG2_Lauryl]-[Pen]-N-T-[Trp(7-Me)]-[Lys(Ac)]-[Pen]-[Phe(4-OMe)]- |
| | [2Nal]-[THP]-L-N-[3Pal]-[Sarc]-NH2 |
| 292 | [Hexyl]-[Pen]-N-T-[Trp(7-Me)]-[Lys(Ac)]-[Pen]-[Phe(4-OMe)]-[2Nal]- |
| | [THP]-L-N-[3Pal]-[Sarc]-NH2 |
| 293 | [Decyl]-[Pen]-N-T-[Trp(7-Me)]-[Lys(Ac)]-[Pen]-[Phe(4-OMe)]-[2Nal]-[THP]- |
| | L-N-[3Pal]-[Sarc]-NH2 |
| 294 | [PEG2_Decyl]-[Pen]-N-T-[Trp(7-Me)]-[Lys(Ac)]-[Pen]-[Phe(4-OMe)]-[2Nal]- |
| | [THP]-L-N-[3Pal]-[Sarc]-NH2 |
| 295 | [PEG2_Capryl]-[Pen]-N-T-[Trp(7-Me)]-[Lys(Ac)]-[Pen]-[Phe(4-OMe)]- |
| | [2Nal]-[THP]-L-N-[3Pal]-[Sarc]-NH2 |
| 296 | [Oct]-[Pen]-N-T-[Trp(7-Me)]-[Lys(Ac)]-[Pen]-[Phe(4-OMe)]-[2Nal]-[THP]-L- |
| | N-[3Pal]-[Sarc]-NH2 |
| 297 | Ac-[Pen]-N-T-[Trp(7-Me)]-[Lys(Ac)]-[Pen]-[Phe(4-(2-aminoethoxy))]-[2Nal]- |
| | [THP]-E-N-[3Pal]-[Sarc]-[(D)Lys(Peg4_IsoGlu_Palm)]-NH2 |
| 298 | Ac-[Pen]-N-T-[Trp(7-Me)]-[Lys(Ac)]-[Pen]-[Phe(4-(2-aminoethoxy))]-[2Nal]- |
| | [THP]-E-N-[3Pal]-[Sarc]-[(D)Lys(IsoGlu_Palm)]-NH2 |
| 299 | Ac-[Pen]-N-T-[Trp(7-Me)]-[Lys(Ac)]-[Pen]-[Phe(4-(2-aminoethoxy))]-[2Nal]- |
| | [THP]-E-N-[3Pal]-[Sarc]-[(D)Lys(PEG12_C18_Diacid)]-NH2 |
| 300 | Ac-[Pen]-N-T-[Trp(7-Me)]-[Lys(Ac)]-[Pen]-[Phe(4-CONH2)]-[2Nal]- |
| | [aMeLys(Peg4_IsoGlu_C18_Diacid)]-[Lys(Ac)]-N-[3Pal]-[Sarc]-NH2 |
| 301 | Ac-[Pen]-N-T-[Trp(7-Me)]-[Lys(Ac)]-[Pen]-[Phe(4-CONH2)]-[2Nal]- |
| | [aMeLys(PEG12_C18_Diacid)]-[Lys(Ac)]-N-[3Pal]-[Sarc]-NH2 |
| 302 | Ac-[Pen]-L-T-[Trp(7-Me)]-[Lys(Ac)]-[Pen]-[Phe(4-OMe)]-[2Nal]-[THP]- |
| | [Lys(Ac)]-[Lys(PEG12_IsoGlu_Palm)]-[3Pal]-[Sarc]-NH2 |
| 303 | [PEG2_Palm]-[Pen]-N-T-[Trp(7-Me)]-[Lys(Ac)]-[Pen]-[Phe(4-(2- |
| | aminoethoxy))]-[2Nal]-[THP]-E-N-[3Pal]-[Sarc]-NH2 |
| 304 | [PEG2_Lauryl]-[Pen]-N-T-[Trp(7-Me)]-[Lys(Ac)]-[Pen]-[Phe(4-(2- |
| | aminoethoxy))]-[2Nal]-[THP]-E-N-[3Pal]-[Sarc]-NH2 |
| 305 | Ac-[Pen]-N-T-[Trp(7-Me)]-[Lys(Ac)]-[Pen]-[Phe(4-(2-aminoethoxy))]-[2Nal]- |
| | [THP]-E-N-[3Pal]-[Sarc]-[(D)Lys(Peg4_IsoGlu_C18_Diacid)]-NH2 |
| 306 | Ac-[Pen]-N-T-[Trp(7-Me)]-[Lys(Ac)]-[Pen]-[Phe(4-(2-aminoethoxy))]-[2Nal]- |
| | [THP]-E-N-[3Pal]-[Sarc]-[(D)Lys(PEG12_IsoGlu_Palm)]-NH2 |
| 307 | Ac-[Pen]-N-T-[Trp(7-Me)]-[Lys(Ac)]-[Pen]-[Phe(4-(2-aminoethoxy))]-[2Nal]- |
| | [THP]-E-N-[3Pal]-[Sarc]-[(D)Lys(Peg4_C18_Diacid)]-NH2 |
| 308 | Ac-[Pen]-N-T-[Trp(7-Me)]-[Lys(Ac)]-[Pen]-[Phe(4-(2-aminoethoxy))]-[2Nal]- |
| | [THP]-E-N-[3Pal]-[Sarc]-[(D)Lys(IsoGlu_C18_Diacid)]-NH2 |
| 309 | [PEG4_Palm]-[Pen]-N-T-[Trp(7-Me)]-[Lys(Ac)]-[Pen]-[Phe(4-(2- |
| | aminoethoxy))]-[2Nal]-[THP]-E-N-[3Pal]-[Sarc]-NH2 |
| 310 | [Palm]-[Pen]-N-T-[Trp(7-Me)]-[Lys(Ac)]-[Pen]-[Phe(4-(2-aminoethoxy))]- |
| | [2Nal]-[THP]-E-N-[3Pal]-[Sarc]-NH2 |
| 311 | [Lauryl]-[Pen]-N-T-[Trp(7-Me)]-[Lys(Ac)]-[Pen]-[Phe(4-(2-aminoethoxy))]- |
| | [2Nal]-[THP]-E-N-[3Pal]-[Sarc]-NH2 |
| 312 | [Oct]-[Pen]-N-T-[Trp(7-Me)]-[Lys(Ac)]-[Pen]-[Phe(4-(2-aminoethoxy))]- |
| | [2Nal]-[THP]-E-N-[3Pal]-[Sarc]-NH2 |
| 313 | [PEG4_Lauryl]-[Pen]-N-T-[Trp(7-Me)]-[Lys(Ac)]-[Pen]-[Phe(4-(2- |
| | aminoethoxy))]-[2Nal]-[THP]-E-N-[3Pal]-[Sarc]-NH2 |
| 314 | [PEG4_Capryl]-[Pen]-N-T-[Trp(7-Me)]-[Lys(Ac)]-[Pen]-[Phe(4-(2- |
| | aminoethoxy))]-[2Nal]-[THP]-E-N-[3Pal]-[Sarc]-NH2 |
| 315 | [PEG4_Hexyl]-[Pen]-N-T-[Trp(7-Me)]-[Lys(Ac)]-[Pen]-[Phe(4-(2- |
| | aminoethoxy))]-[2Nal]-[THP]-E-N-[3Pal]-[Sarc]-NH2 |
| 316 | Ac-[Pen]-N-T-[Trp(7-Me)]-[Lys(Ac)]-[Pen]-[Phe(4-CONH2)]-[2Nal]- |
| | [aMeLys(PEG12_IsoGlu_C18_Diacid)]-[Lys(Ac)]-N-[3Pal]-[Sarc]-NH2 |
| 317 | Ac-[Pen]-N-T-[Trp(7-Me)]-[Lys(Ac)]-[Pen]-[Phe(4-CONH2)]-[2Nal]- |
| | [aMeLys(PEG12_IsoGlu_Palm)]-[Lys(Ac)]-N-[3Pal]-[Sarc]-NH2 |
| 318 | Ac-[Pen]-N-T-[Trp(7-Me)]-[Lys(Ac)]-[Pen]-[Phe(4-CONH2)]-[2Nal]- |
| | [aMeLys(Peg4_IsoGlu_Palm)]-[Lys(Ac)]-N-[3Pal]-[Sarc]-NH2 |
| 319 | Ac-[Pen]-N-T-[Trp(7-Me)]-[Lys(Ac)]-[Pen]-[Phe(4-CONH2)]-[2Nal]- |
| | [aMeLys(IsoGlu_Palm)]-[Lys(Ac)]-N-[3Pal]-[Sarc]-NH2 |
| 320 | Ac-[Pen]-N-T-[Trp(7-Me)]-[Lys(Ac)]-[Pen]-[Phe(4-CONH2)]-[2Nal]- |
| | [aMeLys(IsoGlu_C18_Diacid)]-[Lys(Ac)]-N-[3Pal]-[Sarc]-NH2 |
| 321 | Ac-[Pen]-N-T-[Trp(7-Me)]-[Lys(Ac)]-[Pen]-[Phe(4-CONH2)]-[2Nal]- |
| | [aMeLys(Peg4_C18_Diacid)]-[Lys(Ac)]-N-[3Pal]-[Sarc]-NH2 |
| 322 | [1PEG2_1PEG2_IsoGlu_C16_Diacid)]-[(D)Arg]-[Pen]-N-T-[Trp(7-Me)]- |
| | [Lys(Ac)]-[Pen]-[Phe(4-(2-aminoethoxy))]-[2Nal]-[THP]-[Lys(Ac)]-N-[3Pal]- |
| | [Sarc]-NH2 |
| 323 | [1PEG2_1PEG2_IsoGlu_C18_Diacid)]-[(D)Arg]-[Pen]-N-T-[Trp(7-Me)]- |
| | [Lys(Ac)]-[Pen]-[Phe(4-(2-aminoethoxy))]-[2Nal]-[THP]-[Lys(Ac)]-N-[3Pal]- |
| | [Sarc]-NH2 |
| 324 | Ac-[(D)Arg]-[Pen]-N-T-[Trp(7-Me)]-[Lys(Ac)]-[Pen]-[Phe(4-(2- |
| | aminoethoxy))]-[2Nal]-[THP]-E-N-[3Pal]- |
| | [Lys(1PEG2_1PEG2_IsoGlu_C16_Diacid)]-NH2 |
| 325 | Ac-[(D)Arg]-[Pen]-[Lys(1PEG2_1PEG2_IsoGlu_C16_Diacid)]-T-[Trp(7- |
| | Me)]-[Lys(Ac)]-[Pen]-[Phe(4-(2-aminoethoxy))]-[2Nal]-[THP]-E-N-[3Pal]- |
| | [Sarc]-NH2 |
| 326 | Ac-[(D)Arg]-[Pen]-[Lys(1PEG2_1PEG2_IsoGlu_C18_Diacid)]-T-[Trp(7- |
| | Me)]-[Lys(Ac)]-[Pen]-[Phe(4-(2-aminoethoxy))]-[2Nal]-[THP]-E-N-[3Pal]- |
| | [Sarc]-NH2 |
| 327 | Ac-[Pen]-N-T-[Trp(7-Me)]-[Lys(Ac)]-[Pen]-[Phe(4-(2-aminoethoxy))]-[2Nal]- |
| | [THP]-[Lys(1PEG2_1PEG2_IsoGlu_C16_Diacid)]-N-[3Pal]-[Sarc]-NH2 |
| 328 | Ac-[Pen]-N-T-[Trp(7-Me)]-[Lys(Ac)]-[Pen]-[Phe(4-(2-aminoethoxy))]-[2Nal]- |
| | [THP]-[Lys(1PEG2_1PEG2_IsoGlu_C18_Diacid)]-N-[3Pal]-[Sarc]-NH2 |
| 329 | Ac-[(D)Arg]-[Pen]-N-T-[Trp(7-Me)]-[Lys(Ac)]-[Pen]-[Phe(4-(2- |
| | aminoethoxy))]-[2Nal]-[THP]-E-N-[3Pal]- |
| | [Lys(1PEG2_1PEG2_IsoGlu_C18_Diacid)]-NH2 |
| 330 | Ac-[(D)Arg]-[Pen]-N-T-[Trp(7-Me)]-[Lys(Ac)]-[Pen]-[Phe(4-(2- |
| | aminoethoxy))]-[2Nal]-[THP]-E-N-[3Pal]-[Sarc]- |
| | [Lys(1PEG2_1PEG2_IsoGlu_C16_Diacid)]-NH2 |
| 331 | Ac-[(D)Arg]-[Pen]-N-T-[Trp(7-Me)]-[Lys(Ac)]-[Pen]-[Phe(4-(2- |
| | aminoethoxy))]-[2Nal]-[THP]-E-N-[3Pal]-[Sarc]- |
| | [Lys(1PEG2_1PEG2_IsoGlu_C18_Diacid)]-NH2 |
| 332 | [1PEG2_1PEG2_IsoGlu_C18]-[(D)Arg]-[Pen]-N-T-[Trp(7-Me)]-[Lys(Ac)]- |
| | [Pen]-[Phe(4-(2-aminoethoxy))]-[2Nal]-[Acvc]-E-N-[THP]-NH2 |
| 333 | [1PEG2_1PEG2_IsoGlu_C18_Diacid]-[(D)Arg]-[Pen]-N-T-[Trp(7-Me)]- |
| | [Lys(Ac)]-[Pen]-[Phe(4-(2-aminoethoxy))]-[2Nal]-[Acvc]-E-N-[THP]-NH2 |
| 334 | Ac-[(D)Arg]-[Pen]-N-T-[Trp(7-Me)]-[Lys(Ac)]-[Pen]-[Phe(4-(2- |
| | aminoethoxy))]-[2Nal]-[Acvc]-E-N-[THP]- |
| | [Lys(1PEG2_1PEG2_IsoGlu_C18_Diacid)]-NH2 |
| 335 | Ac-[(D)Lys(1PEG2_1PEG2_IsoGlu_C18_Diacid)]-[Pen]-N-T-[Trp(7-Me)]- |
| | [Lys(Ac)]-[Pen]-[Phe(4-(2-aminoethoxy))]-[2Nal]-[Acvc]-E-N-[THP]-NH2 |
| 336 | Ac-[(D)Lys(1PEG2_1PEG2_IsoGlu_C16_Diacid)]-[Pen]-N-T-[Trp(7-Me)]- |
| | [Lys(Ac)]-[Pen]-[Phe(4-(2-aminoethoxy))]-[2Nal]-[Acvc]-E-N-[THP]-NH2 |
| 337 | Ac-[(D)Arg]-[Pen]-N-T-[Trp(7-Me)]-[Lys(Ac)]-[Pen]-[Phe(4-(2- |
| | aminoethoxy))]-[2Nal]-[Acvc]-E-N-[THP]- |
| | [Lys(1PEG2_1PEG2_IsoGlu_C18)]-NH2 |
| 338 | Ac-[Pen]-N-T-[Trp(7-Me)]-[Lys(Ac)]-[Pen]-[Phe(4-(2-aminoethoxy))]- |
| | [3Quin]-[THP]-E-N-H-[Sarc]-NH2-[PEG4] |
| 339 | Ac-[Pen]-N-T-[Trp(7-Me)]-[Lys(Ac)]-[Pen]-[Phe(4-OMe)]-[2Nal]- |
| | [aMeLys(PEG12_IsoGlu_C18_Diacid)]-[Lys(Ac)]-N-[3Pal]-[Sarc]-NH2 |
| 340 | Ac-[Pen]-N-T-[Trp(7-Me)]-[Lys(Ac)]-[Pen]-[Phe(4-OMe)]-[2Nal]- |
| | [aMeLys(PEG12_IsoGlu_Palm)]-[Lys(Ac)]-N-[3Pal]-[Sarc]-NH2 |
| 341 | [PEG12_OMe]-[Pen]-L-T-[Trp(7-Me)]-[Lys(Ac)]-[Pen]-F-[Nal]-[aMeLeu]-L- |
| | N-[NH(2-(pyridin-3-y1)ethyl)] |
| 342 | Ac-[Pen]-L-T-[Trp(7-Me)]-[Lys(PEG12_OMe)]-[Pen]-F-[Nal]-[aMeLeu]-L- |
| | N-[NH(2-(pyridin-3-yl)ethyl)] |
| 343 | [PEG12_OMe]-[Pen]-L-T-[Trp(7-Me)]-[Lys(Ac)]-[Pen]-[Phe(4-OMe)]-[Nal]- |
| | [aMeLeu]-L-N-[NH(2-(pyridin-3-yl)ethyl)] |
| 344 | Ac-[Pen]-L-T-[Trp(7-Me)]-[Lys(PEG12_OMe)]-[Pen]-[Phe(4-OMe)]-[Nal]- |
| | [aMeLeu]-L-N-[NH(2-(pyridin-3-yl)ethyl)] |
| 345 | [PEG12_OMe]-[Pen]-[aMeAsn]-T-[Trp(7-Me)]-[Lys(Ac)]-[Pen]-F-[Nal]- |
| | [aMeLeu]-L-N-[NH(2-(pyridin-3-yl)ethyl)] |
| 346 | Ac-[Pen]-[aMeAsn]-T-[Trp(7-Me)]-[Lys(PEG12_OMe)]-[Pen]-F-[Nal]- |
| | [aMeLeu]-L-N-[NH(2-(pyridin-3-y1)ethyl)] |
| 347 | [PEG12_OMe]-[Pen]-[aMeAsn]-T-[Trp(7-Me)]-[Lys(Ac)]-[Pen]-[Phe(4- |
| | OMe)]-[Nal]-[aMeLeu]-L-N-[NH(2-(pyridin-3-yl)ethyl)] |

### Example 348. Biological Assays

IL-23 binding to IL-23 receptors results in the activation of the Signal Transducer and Activator of Transcription 3 (STAT3) by phosphorylation and downstream signaling events. Accordingly, the ability of the inhibitors described herein to block IL-23 action can be assessed by monitoring the status of STAT3 activation in response to IL-23. This may be accomplished in reporter cell assays or in intact cells such as peripheral blood mononuclear cells (PBMCs).

### IL23R Reporter Assay

Compounds were serially diluted in 100% (v/v) DMSO) and plated using an Echo acoustic dispenser (Labcyte) into 1536-well non-treated black assay plates (Corning #9146). 3 μL of HEK293 cells containing IL-23R, IL-12R(31 and a firefly luciferase reporter gene driven by a STAT-inducible promoter (Promega) were added to the plates (4000 cells/well), followed by 3 μL of 10 ng/mL IL-23 (equivalent to EC₉₀ concentration). After 5 h at 37° C., 5% CO₂, 95% relative humidity, cells were placed at 20° C. and treated with BioGlo reagent (Promega) according to the Manufacturer's instructions. Luminescence was measured on a Pherastar FSX (BMG LabTech). The data, provided in Tables 5a and 5b, were normalized to IL-23 treatment (0% inhibition) and 30 μM of control inhibitor (100% inhibition), and IC₅₀ values were determined using a 4-parameter Hill equation.

TABLE 5a
IL-23 Binding Data for the Compounds of Examples 2 to 347.

| Example | IC50 (nM) |
|---|---|
| 2 | 0.086 |
| 3 | 0.023 |
| 4 | 0.066 |
| 5 | 0.021 |
| 6 | 0.055 |
| 7 | 0.024 |
| 8 | 0.089 |
| 9 | 0.035 |
| 10 | 1.88 |
| 11 | 0.044 |
| 12 | 0.29 |
| 13 | 0.064 |
| 14 | 0.2 |
| 15 | 0.054 |
| 16 | 0.014 |
| 17 | 0.036 |
| 18 | 0.12 |
| 19 | 0.037 |
| 20 | 0.067 |
| 21 | 0.033 |
| 22 | 0.026 |
| 23 | 0.12 |
| 24 | 0.017 |
| 25 | 0.0078 |
| 26 | 0.025 |
| 27 | 0.059 |
| 28 | 0.021 |
| 29 | 0.06 |
| 30 | 0.17 |
| 31 | 0.052 |
| 32 | 0.021 |
| 33 | 0.046 |
| 34 | 0.065 |
| 35 | 0.19 |
| 36 | 0.14 |
| 37 | 0.19 |
| 38 | 0.021 |
| 39 | 0.048 |
| 40 | 0.023 |
| 41 | 0.078 |
| 42 | 0.029 |
| 43 | 0.049 |
| 44 | 0.02 |
| 45 | 0.039 |
| 46 | 0.26 |
| 47 | 0.041 |
| 48 | 0.045 |
| 49 | 0.046 |
| 50 | 0.1 |
| 51 | 0.18 |
| 52 | 0.15 |
| 53 | |
| 54 | 0.13 |
| 55 | 0.13 |
| 56 | 0.17 |
| 57 | 0.3 |
| 58 | 0.094 |
| 59 | 0.18 |
| 60 | 0.042 |
| 61 | 0.044 |
| 62 | 0.054 |
| 63 | 0.13 |
| 64 | 0.055 |
| 65 | 0.19 |
| 66 | 0.18 |
| 67 | 0.035 |
| 68 | 0.037 |
| 69 | 0.13 |
| 70 | 0.23 |
| 71 | 0.081 |
| 72 | 0.11 |
| 73 | 0.094 |
| 74 | 0.17 |
| 75 | 0.096 |
| 76 | 0.18 |
| 77 | 0.3 |
| 78 | 0.23 |
| 79 | 0.38 |
| 80 | >16.61 |
| 81 | 0.012 |
| 82 | 0.013 |
| 83 | 0.052 |
| 84 | 0.046 |
| 85 | 0.031 |
| 86 | 0.061 |
| 87 | 0.079 |
| 88 | 0.2 |
| 89 | 0.34 |
| 90 | 0.12 |
| 91 | |
| 92 | 0.18 |
| 93 | 0.12 |
| 94 | 1.44 |
| 95 | 0.14 |
| 96 | 0.13 |
| 97 | 0.0082 |
| 98 | 1.24 |
| 99 | 0.096 |
| 100 | 0.11 |
| 101 | 0.066 |
| 102 | 0.053 |
| 103 | 0.066 |
| 104 | 0.066 |
| 105 | 0.089 |
| 106 | 0.11 |
| 107 | 0.09 |
| 108 | 0.15 |
| 109 | 0.056 |
| 110 | 0.0096 |
| 111 | 0.048 |
| 112 | 0.018 |
| 113 | 1.11 |
| 114 | 0.15 |
| 115 | 0.082 |
| 116 | 0.15 |
| 117 | 0.1 |
| 118 | 0.077 |
| 119 | 0.058 |
| 120 | 0.38 |
| 121 | 0.9 |
| 122 | 0.083 |
| 123 | 0.073 |
| 124 | 0.083 |
| 125 | 0.075 |
| 126 | 0.16 |
| 127 | 0.17 |
| 128 | 0.1 |
| 129 | 0.24 |
| 130 | 0.02 |
| 131 | 0.021 |
| 132 | 0.031 |
| 133 | 0.031 |
| 134 | 0.037 |
| 135 | 0.016 |
| 136 | 0.016 |
| 137 | 0.024 |
| 138 | 0.039 |
| 139 | 0.0079 |
| 140 | 0.012 |
| 141 | 0.011 |
| 142 | 0.0092 |

TABLE 5b
IL-23 Binding Data for the Compounds of Examples 348 to 492.

| Compound/ | |
|---|---|
| Example No. | IC₅₀ (μM) |
| 348 | 0.94 |
| 349 | 0.98 |
| 350 | 0.7 |
| 351 | |
| 352 | |
| 353 | 0.37 |
| 354 | 1.28 |
| 355 | 8.38 |
| 356 | 3.26 |
| 357 | 8.51 |
| 358 | 2.74 |
| 359 | 0.12 |
| 360 | 0.0075 |
| 361 | 0.0051 |
| 362 | 0.056 |
| 363 | 0.25 |
| 364 | 0.1 |
| 365 | 0.0052 |
| 366 | 0.0092 |
| 367 | 0.006 |
| 368 | 0.056 |
| 371 | 0.014 |
| 372 | 0.039 |
| 373 | 0.041 |
| 374 | 0.041 |
| 378 | 0.016 |
| 380 | 0.039 |
| 381 | 0.0076 |
| 382 | 0.0035 |
| 383 | 0.0045 |
| 384 | 0.0086 |
| 385 | 0.016 |
| 386 | 0.12 |
| 392 | 0.037 |
| 396 | 0.1 |
| 397 | 0.071 |
| 398 | 0.067 |
| 399 | 0.06 |
| 400 | 0.048 |
| 401 | 0.044 |
| 402 | 0.017 |
| 403 | 0.076 |
| 404 | |
| 405 | |
| 406 | |
| 407 | |
| 408 | 0.052 |
| 409 | 0.046 |
| 410 | 0.064 |
| 411 | 0.08 |
| 412 | 0.089 |
| 413 | 0.072 |
| 414 | 0.062 |
| 415 | 0.084 |
| 416 | 0.059 |
| 417 | 0.074 |
| 418 | 0.063 |
| 419 | 0.47 |
| 420 | 0.52 |
| 421 | 0.19 |
| 422 | 0.1 |
| 423 | 0.24 |
| 424 | 0.05 |
| 425 | 0.086 |
| 426 | 0.21 |
| 427 | 0.0066 |
| 428 | 0.016 |
| 429 | 0.15 |
| 430 | 0.22 |
| 431 | 0.18 |
| 432 | 0.12 |
| 433 | 0.0051 |
| 434 | 0.0067 |
| 435 | 0.12 |
| 436 | 0.26 |
| 437 | 0.015 |
| 438 | 0.22 |
| 439 | 0.1 |
| 440 | 0.0094 |
| 441 | 0.075 |
| 442 | 0.0068 |
| 443 | 0.0044 |
| 444 | 0.0086 |
| 445 | |
| 446 | |
| 447 | 0.023 |
| 448 | 0.048 |
| 449 | 0.012 |
| 450 | 8.02 |
| 451 | 0.011 |
| 452 | 0.0057 |
| 453 | 0.018 |
| 454 | 0.0062 |
| 455 | 0.012 |
| 456 | 0.046 |
| 457 | 0.024 |
| 458 | 0.0061 |
| 459 | 0.016 |
| 460 | 0.021 |
| 461 | 0.014 |
| 462 | 0.011 |
| 463 | |
| 464 | 0.019 |
| 465 | 0.0068 |
| 466 | 0.016 |
| 467 | 0.0055 |
| 468 | 0.016 |
| 469 | 0.0055 |
| 470 | 0.036 |
| 471 | 0.074 |
| 472 | 0.37 |
| 473 | 0.11 |
| 474 | 0.037 |
| 475 | 0.2 |
| 476 | 0.045 |
| 477 | 0.0062 |
| 478 | 0.021 |
| 479 | 0.022 |
| 480 | 0.011 |
| 481 | 0.0066 |
| 482 | 0.009 |
| 483 | 0.021 |
| 484 | 0.0075 |
| 485 | 0.023 |
| 486 | 0.009 |
| 487 | 0.0025 |
| 488 | 0.0022 |
| 489 | 0.0084 |
| 490 | 0.013 |
| 491 | 0.0079 |
| 492 | 0.04 |

DB Cells IL23R pSTAT3 Cell Assay

IL-23 is believed to play a central role in supporting and maintaining Th17 differentiation in vivo. This process is thought to be mediated primarily through the Signal Transducer and Activator of Transcription 3 (STAT3), with phosphorylation of STAT3 (to yield pSTAT3) leading to upregulation of RORC and pro-inflammatory IL-17. This cell assay examines the levels of pSTAT3 in IL-23R-expressing DB cells when stimulated with IL-23 in the presence of test compounds. Serial dilutions of test peptides and IL-23 (Humanzyme #HZ-1261) at a final concentration of 0.5 nM, were added to each well in a 96 well tissue culture plate (Corning #CLS3894). DB cells (ATCC #CRL-2289), cultured in RPMI-1640 medium (Thermo Scientific #11875093) supplemented with 10% FBS, were added at 5×10E5 cells/well and incubated for 30 minutes at 37° C. in a 5% CO₂ humidified incubator. Changes in phospho-STAT3 levels in the cell lysates were detected using the Cisbio HTRF pSTAT3 (Tyr705) Cellular Assay Kit (Cisbio #62AT3PEH), according to manufacturer's Two Plate Assay protocol. IC₅₀ values determined from these data are shown in Table 6. Where not shown or it is marked as “0”, data was not yet determined.

TABLE 6
IL-23 Cell Data

| Example | IC50 (nM) |
|---|---|
| 201 | |
| 202 | |
| 203 | |
| 204 | |
| 205 | 0.038 |
| 206 | 0.129 |
| 207 | 0.631 |
| 208 | 0.056 |
| 209 | 0.07 |
| 210 | 0.0798 |
| 211 | 0.062 |
| 212 | |
| 213 | 0.13 |
| 214 | 0.433 |
| 215 | 0.0393 |
| 216 | 0.215 |
| 217 | 1.37 |
| 218 | 1.01 |
| 219 | 2.87 |
| 220 | 2.68 |
| 221 | 5.22 |
| 222 | 2.62 |
| 223 | 0.801 |
| 224 | 0.807 |
| 225 | 0.811 |
| 226 | 0.633 |
| 227 | 0.784 |
| 228 | 5.11 |
| 229 | 3.51 |
| 230 | 5.35 |
| 231 | 2.83 |
| 232 | 0.176 |
| 233 | 0.188 |
| 234 | 0.512 |
| 235 | 0.585 |
| 236 | >10 |
| 237 | 4.74 |
| 238 | 4.24 |
| 239 | 5.16 |
| 240 | 0.424 |
| 241 | 0.35 |
| 242 | 4.15 |
| 243 | 4.3 |
| 244 | 8.98 |
| 245 | >10 |
| 246 | >10 |
| 247 | >10 |
| 248 | 0.257 |
| 249 | |
| 250 | 1.22 |
| 251 | >10 |
| 252 | >10 |
| 253 | 4.58 |
| 254 | 4.38 |
| 255 | 2.01 |
| 256 | 3.37 |
| 257 | 3.37 |
| 258 | 3.97 |
| 259 | 2.07 |
| 260 | 2.62 |
| 261 | >10 |
| 262 | >10 |
| 263 | >10 |
| 264 | >10 |
| 265 | >10 |
| 266 | >10 |
| 267 | >10 |
| 268 | >10 |
| 269 | 2.63 |
| 270 | >10 |
| 271 | >10 |
| 272 | >10 |
| 273 | >10 |
| 274 | >10 |
| 275 | 2.37 |
| 276 | 3.42 |
| 277 | 0.526 |
| 278 | 0.607 |
| 279 | 0.277 |
| 280 | 0.498 |
| 281 | 0.316 |
| 282 | 0.481 |
| 283 | 0.417 |
| 284 | 0.626 |
| 285 | 0.283 |
| 286 | 0.0719 |
| 287 | 0.0806 |
| 288 | 5.38 |
| 289 | 1.99 |
| 290 | 0.625 |
| 291 | 0.106 |
| 292 | 1.07 |
| 293 | 0.121 |
| 294 | 0.0746 |
| 295 | 0.278 |
| 296 | 0.224 |
| 297 | 0.42 |
| 298 | 0.298 |
| 299 | 0.473 |
| 300 | 0.293 |
| 301 | |
| 302 | 0.692 |
| 303 | 0.12 |
| 304 | 0.879 |
| 305 | 0.126 |
| 306 | 1 |
| 307 | 7.36 |
| 308 | 0.158 |
| 309 | 5.09 |
| 310 | 0.615 |
| 311 | 0.058 |
| 312 | 0.154 |
| 313 | 0.076 |
| 314 | 0.0266 |
| 315 | 0.129 |
| 316 | 0.0567 |
| 317 | 0.165 |
| 318 | 1.34 |
| 319 | 1.03 |
| 320 | 0.634 |
| 321 | |
| 322 | |
| 323 | |
| 324 | |
| 325 | |
| 326 | |
| 327 | |
| 328 | |
| 329 | |
| 330 | |
| 331 | |
| 332 | |
| 333 | |
| 334 | |
| 335 | |
| 336 | |
| 337 | 2.63 |
| 338 | 0.137 |
| 339 | 0.0701 |
| 340 | |
| 341 | |
| 342 | |
| 343 | |
| 344 | |
| 345 | |
| 346 | |
| 347 | |

PBMC pSTAT3 assay

Cryopreserved peripheral blood mononuclear cells (PBMCs) from healthy donors were thawed and washed twice in ImmunoCult-XF T cell expansion medium (XF-TCEM) supplemented with CTL anti-aggregate wash. The cells were counted, resuspended at 2×10⁵ cells per mL XF-TCEM supplemented with penicillin/streptomycin and 100 ng/mL IL-1β (BioLegend, 579404), and cultured in tissue culture flasks coated with anti-CD3 (eBioscience, 16-0037-85 or BD Pharmingen, 555329) at 37° C. in 5% CO₂. On day 4 of culture, PBMCs were collected, washed twice in RPMI-1640 supplemented with 0.1% BSA (RPMI-BSA), and incubated in RPMI-BSA in upright tissue culture flasks for 4 hours at 37° C. in 5% C02. Following this ‘starvation,’ a total of 6×10⁴ cells in 30 μL RPMI-BSA was transferred into each well of a 384-well plate pre-spotted with peptide in DMSO. The cells were incubated for 30 minutes prior to the addition of IL-23 at a final concentration of 5 ng/mL. The cells were stimulated with cytokine for 30 minutes at 37° C. in 5% CO₂, transferred onto ice for 10 minutes, and lysed. Cell lysates were stored at −80° C. until phosphorylated STAT3 was measured using the phospho-STAT panel kit (Meso Scale Discovery, K15202D). The results produced for several compounds with PBMCs are provide in Table 7 below.

TABLE 7

| Example in | | |
|---|---|---|
| Discloure/ | | |
| Compound | PBMC pSTAT3 | |
| No. | IC50 (nM) | SEQ ID NO: |
| 2 | 0.50 | 2 |
| 4 | 1.2 | 4 |
| 3 | 5.7 | 3 |
| 11 | 1.3 | 11 |
| 5 | 0.16 | 5 |
| 6 | 0.7 | 6 |
| 12 | 5.0 | 12 |
| 8 | 0.21 | 8 |
| 14 | 0.78 | 14 |
| 13 | 0.27 | 13 |
| 9 | 0.23 | 9 |
| 10 | 4.8 | 10 |
| 7 | 0.42 | 7 |
| 15 | 0.42 | 15 |
| 16 | 0.18 | 16 |

Although the foregoing invention has been described in some detail by way of illustration and Example for purposes of clarity of understanding, one of skill in the art will appreciate that certain changes and modifications may be practiced within the scope of the appended aspects.

LENGTHY TABLES
The patent application contains a lengthy table section. A copy of the table is available in electronic form from the USPTO web site (https://seqdata.uspto.gov/docdetail?docId=US20260000659A1). An electronic copy of the table will also be available from the USPTO upon request and payment of the fee set forth in 37 CFR 1.19(b)(3).

1-9. (canceled)

10. An interleukin-23 receptor (IL-23R) inhibitor comprising an amino acid sequence of Formula X 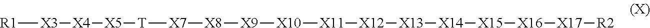
or a Pharmaceutically acceptable salt thereof, wherein:
R1 is hydrogen, C1 to C4 alkyl C(O)—, or C1 to C4 alkyl C(O)— substituted with Cl or F or cyano, 7Ahp, 6Ahx, 5Ava, 6Ava, AEEP, GABA, succinyl carnitine, cPEG3aCO, ClAcPEG4CO, 1PEG2_1PEG2_IsoGlu_C18, 1PEG2_1PEG2_IsoGlu_C18_Diacid, PentCO, PEG12_OMe, HOC18gEPEG2PEG2, PEG2PEG2gEC16OH, PEG4_Decyl, PEG4_Lauryl, PEG4_Capryl, PEG4_Hexyl, PEG2_Palm, PEG2_Myristyl, PEG2_Lauryl, Hexyl, Decyl, PEG2_Decyl, PEG2_Capryl, Oct, PEG4_Palm, Palm, Lauryl, 1PEG2_1PEG2_IsoGlu_C16_Diacid, HOC16gEPEG2PEG2orn, or Z;
X3 is dR, dK, dK-Z, or absent;
X4 is Pen, aMeC, Abu, or C;
X5 is N, L, Q, K, E, aMeN, dN, dL, dQ, dK, dE, K-Z, or dK-Z;
X7 is 7MeW, W, 3Pya, 7(2ClPh)W, 7(3(1NMepip)pyraz)W, 7(3(6AzaInd1Me))W, 7(3CF3TAZP)W, 7(3NAcPh)W, 7(3NPyrazPh)W, 7(3NpyrlonePh)W, 7(3UrPh)W, 7(4(CpCNPh))W, 7(4CF3Ph)W, 7(4NAcPh)W, 7(40CF3Ph)W, 7(4OMePh)W, 7(4Paz)W, 7(5(2(4OMePh)Pyr))W, 7(5(Ina7Pyr))W, 7(6(1)7dMeNDAZ))W, 7(6(2MeNDAZ))W, 7(7(124TAZP))W, 7(7Imzpy)W, 7BrW, 7EtW, 7PhW, 7PyrW, A, DT, or D7MeW;
X8 is KAc, dK(Ac), dQ, or Q;
X9 is Pen, C, aMeC, or Abu;
X10 is AEF, F4OMe, F(4-CONH2), TMAPF, AEF(G), or F;
X11 is 2-Nal, Phe(2-Me), Phe(3-Me), Phe(4-Me), Phe(3,4-dimethoxy), 2Quin, 3Quin, 1-Nal, unsubstituted Trp, or Trp substituted with cyano, halo, alkyl, haloalkyl, hydroxy, or alkoxy;
X12 is THP, aMeL, Acvc, Acpx, aMeK, or aMeK-Z;
X13 is K(Ac), dK(Ac), E, dE, L, dL, dK-Z, or K-Z;
X14 is N, K, or K-Z;
X15 is 3Pya;
X16 is Sarc, K-Z, NMeK-Z, or absent;
X17 is K-Z, dK-Z, or absent;
R2 is —OH, —NH2, —NH(C1 to C4 alkyl), —NH(C1-C4 alkyl), or —N(C1 to C4 alkyl)₂, wherein each alkyl is optionally substituted with Cl, F, cyano or Z;
Z is group comprising a lipid moiety; and
wherein the IL-23R inhibitor is cyclized by a disulfide or thioether first bond between X4 and X9, and an amide second bond (i) between X5 and X10 when X5 is E and X10 is AEF, or (ii) between X13 and R1 when X13 is E and R1 is 7Ahp, 6Ahx, 5Ava, 6Ava, AEEP, or GABA.

11. (canceled)

12. (canceled)

13. A pharmaceutical composition comprising:
(i) an interleukin-23 receptor inhibitor or pharmaceutically acceptable salt, solvate, or form thereof, according to claim 10, and
(ii) a pharmaceutically acceptable carrier, excipient, or diluent.

14-18. (canceled)

19. A method for treating a disease or disorder associated with Interleukin 23 (IL-23)/Interleukin 23 Receptor (IL-23R), which comprises administering an effective amount of the IL-23, or a pharmaceutically acceptable salt thereof, according to claim 10.

20. The method of claim 19, wherein the disease or disorder is associated with autoimmune inflammation.

21. The method of claim 20, wherein the disease or disorder is multiple sclerosis, asthma, rheumatoid arthritis, inflammation of the gut, inflammatory bowel diseases (IBDs), juvenile IBD, adolescent IBD, Crohn's disease, ulcerative colitis, Celiac disease (nontropical Sprue), microscopic colitis, collagenous colitis, eosinophilic gastroenteritis/esophagitis, colitis associated with radio- or chemo-therapy, colitis associated with disorders of innate immunity as in leukocyte adhesion deficiency-1, sarcoidosis, Systemic Lupus Erythematosus, ankylosing spondylitis (axial spondyloarthritis), psoriatic arthritis, psoriasis, atopic dermatitis, acne ectopica, enteropathy associated with seronegative arthropathies, chronic granulomatous disease, glycogen storage disease type 1b, Hermansky-Pudlak syndrome, Chediak-Higashi syndrome, Wiskott-Aldrich Syndrome, pouchitis, pouchitis resulting after proctocolectomy and ileoanal anastomosis, gastrointestinal cancer, pancreatitis, insulin-dependent diabetes mellitus, mastitis, cholecystitis, cholangitis, primary biliary cirrhosis, viral-associated enteropathy, pericholangitis, chronic bronchitis, chronic sinusitis, asthma, uveitis, or graft versus host disease.

22. The method of claim 20, wherein the disease or disorder is Ulcerative colitis (UC), Crohn's Disease (CD), psoriasis (PsO), or psoriatic arthritis (PsA).

23. The IL-23R inhibitor of claim 10, wherein:
R1 is hydrogen, C1 to C4 alkyl C(O)—, or C1 to C4 alkyl C(O)— that is substituted with Cl, F, or cyano;
X3 is dR or dK-Z;
X4 is Pen, aMeC, or C;
X5 is N, L, Q, or K;
X7 is 7MeW, W, 3Pya, 7(2ClPh)W, 7(3(1NMepip)pyraz)W, 7(3(6AzaInd1Me))W, 7(3CF3TAZP)W, 7(3NAcPh)W, 7(3NPyrazPh)W, 7(3NpyrlonePh)W, 7(3UrPh)W, 7(4(CpCNPh))W, 7(4CF3Ph)W, 7(4NAcPh)W, 7(40CF3Ph)W, 7(4OMePh)W, 7(4Paz)W, 7(5(2(4OMePh)Pyr))W, 7(5(Ina7Pyr))W, 7(6(1)7dMeNDAZ))W, 7(6(2MeNDAZ))W, 7(7(124TAZP))W, 7(7Imzpy)W, 7BrW, 7EtW, 7PhW, 7PyrW, A, DT, or D7MeW; X8 is KAc, or Q;
X9 is Pen, C, or aMeC;
X10 is AEF, F4OMe, F(4-CONH2), or F;
X11 is 2-Nal, Phe(2-Me), Phe(3-Me), Phe(4-Me), Phe(3,4-dimethoxy), 2Quin, 3Quin, or 1-Nal;
X12 is THP;
X13 is KAc, E, or L;
X14 is N or K;
X15 is 3Pya;
X16 is Sarc or absent;
X17 is K-Z or dK-Z;
R2 is —OH, —NH2, —NH(C1 to C4 alkyl), —NH(C1-C4 alkyl), or —N(C1 to C4 alkyl)₂, wherein each alkyl is optionally substituted with Cl, F, or cyano
Z is group comprising a lipid moiety; and
wherein the IL-23R inhibitor is cyclized by a disulfide or thioether first bond between X4 and X9.

24. The IL-23R inhibitor of claim 10, wherein:
X7 is 7MeW or W;
X11 is 2Nal or 3Quin;
X16 is Sarc; and
R2 is —OH —NH2, or —N(H)C1-C4 alkyl.

25. The IL23R inhibitor of claim 10, wherein X7 is 7MeW or W.

26. The IL23R inhibitor of claim 10, wherein X11 is 2Nal or 3Quin.

27. The IL23R inhibitor of claim 10, wherein the Z group of X17 is selected from the group consisting of PEG2PEG2gEC18OH, PEG2PEG2eKC18OH, PEG2PEG2gDabC18OH, dK(PEG12IsoGluC18Diacid), and dK(Peg4IsoGluPalm).
